SEQUENCE LISTING (1 sequence)

SEQ ID NO: 1 - DNA, 359853 bp, unidentified
  misc_feature 1..359853: note = Myoviridae bacteriophage
  source 1..359853: mol_type = other DNA
aattatggtcaaatctttactaccaccaccaaagcgtcacaaaatccaagaaatgaaagatcaacgtacattaaagatgtacaacaatttagttaagttactgtgtaaaagtccatcgctcaaagcagagtttaaaactgatgatgaccgcaaagcactaattgagatgaagtatatgctcagtaaaatgtccttttacaaatttgtaaaagaatacgcatctgacccatttagtcctcgttggagaaccaaaccaagagagttttttttcggtggttcatatactgatatcattcaagatactgtgataggcgtagagttcgagattgattatgatccgaatggtgtggaaaacttgctgattgagaaacgtttaccaagtgaagaagaaatttatgtttatgcaagaaaactttcaactttcagttcaagaattactctaggcaagttagattcatttattattttctatatacgtgttggcgtagataaaatggtaactaaactcaatgaactttcattacaacaaaaacagattaatgcatacaaagacattactgatttatactaatgaggaatgaatatgttttttggcccagttggaaaacaaaacagtactttaatttggcatgaaattatctttaaaattatatcaaaaagtttcacacttaaagatatcatcaatacagatcaagatcgtcgtgttctgttagaagcaaaatataaattgagcaaacaccctttgctaccattaatcattgattacgttaatgattatgaatccagtcgttggaagtttgaagaaggcggtttatttcgtggaaaaacattctacggtgcggaacgatatgtattcagtgatgcaacaaatggcgttaagtttgctattagaaactttaactccaaagaaaatgattatcttgaaagtggagtgaaagtatatatcgataacaatagcaatgttctaacatatgacgaatcactaattatgttacaaataatgcgtggcgttgaaggtgtaagacacaatcttactaatttgaaaattcagcaaaatgatatacataatagacagaaaattaagggtttatataaatgatttcatttttcaaaaagaaagaaaaagttgatgaaactggattagaatcgtttctatggactcttatcatcaaaaaaagtccattgataaaagaatatgccatcaatgatactaatagaaaaactatcattgaatacaaatacgaattatcaaaatgtgatccgattgtttaccactttgtaaagtactgttctaaaaatttatcggatatcaccattgattatgagtcagtaccaatgagttttccttatggagattgggtaaatacttatgatatttttgatgggtctaacggattcaatttttatttcacatcaaataaagcatatgttcgaatcaaaacagatataggttgggcattacatagctcaaataaagaagattataatttattagctgatgtatacagaagtgttaagaattcaaaggatattgatgaaagaattaaaaagcaaaatcgcatttctttatctttagaaacatataagtcaaagtacggtggttaattatgactacaatcgatgatatctacagtactatacgcaatatgagtacagatagaaaagtctttggtgacaaagaaaagtccaaatgttatggcatcgattcttttgatattatttcatacgaatataataacaacgggacaagttcactaaacattgtgtgcccacgagatccagaaaataatttcatgtacgttgaaattaacaatgatggtatgttctttactaaagtatataaaggtgggaaagaatatgaaccaatcgaagtaatgaatgaagagaaattcttccaagaagatactatcataaatcttggatttagttacaatgactatcaagtactggatcatgattttaaacgatactataatggttttctaaaggagttaacgatttacttatgaaaaacaattttctagaacaaactattgtgaaacacttagcaggttcacaggcgtatggcacaagcacaccagaaagtgatacagattatcgtggtatcttccttgcaaacaaagaatatatcctaactcctttttttaatgttaaggaagtttcagatacttctgaagaagatactaaattttatgaaatcaatcagtacttaagtctgtatactgaagctaacccaaacattcttgaatcactatgggttaaaccggaacacatcgtagaaagttctgaaatctatgactatctacgctcattcaatcagcaattattgagttcaaagattgcacactcatacagtggatatgcatacaatcaggttaagcgtatgtctaatcaccacggctggctcgataaggaacgaattgctgaacgtaaactaaatgaaattttagaagaacatccagttgacgaagtactttcatggatgtatgatacattcccgcagtatttggtagaccgtttgaataaatccaatcttcacccattcaatgctacaaaaattgattttgaaaaatacatgcgtgatacttcattacaattagtttcaacacaagcattgaaacaatatcattttgtacgtttagtacataattatagtactgataaagtattagatcgtgatttcaatttattgaattacaattctggatatgaactaatccattacggtgaaaacatctttggggtaattatcaacccgaatgaaagtacagtgaatgctgatggttcactacgttacttcaacaaagaacgtactacagaagaattgaaagttcgtcctgaactcattgtaaaattcaatgaaaaggaatacaaagaaaatagtgagaaccgtaagagttatcacaaatggaagcagaatcgtaatgccaaacgtgctattcttgaatcacaccacggttatgactgcaaacacgcaatgcatacagtaaggctgctgaatacagcacaggaagcacttgaaactggtatcatccaagttcatcgccctgatgctgctatgttgatggacattcgtaacgggaagtggttgtatcaagaagttatggattatttcaacgaaaaggttgactacatccgaaatgtggcgtatcataagacccatctaccgaagcgaccaaacataaagttggctgcgaaggtactattagatatccgtgaaatgcaatggtacagtaaaaaataagaggattgttatgctaaacgaacaaatcaaagacgcactacgtgtactgaatcgcgaagttaaccgcatccataaaagtactgatcgttataaactaatgcatgaacttagactttcgttggaagctatcaataatggcgaatggtatcttgaaccagaatatcgcacacgtgaactaactgacgttgaaaaagcaattgtcaaattcattacaagtcgcgacaaaaatgcatccgaaaaagatgtatcaatgaacttccatcttactaaatgtgaagtcgtggaaaaatttaaattcgattggcttgactggattgaaatgatcatggaacttgaagaagaattcaaagttgaagttgatttggaatacgagaatgaaaacactaaactgtataaactcgtagatcatattcaaaaaatgttgtaaacatataatatagtacactctaaataatattaccaactataggagatgttattatgagtgtactagaaattattgtttcaaaagttttaataccatattggaaaccaattttggtagtaggagtaatggcaggttgtgtttacggtggatggtcagcaaatacttattatactggttataaagaaagtattgaccaaaaaatagaaaagaaagttgattctgcactcgctaagatgcagcaagaaaatgcaaaaaactatatagaaaccaaaaagataatgcaagataatgctaaagttatcgagacacaagtgcctattatagtagaaagagaagtttacaaaaacgtatgtctagatgaagatggcgttaaacttctgaatgaacttaaacagaattcccttgcagcgaggaataaattcaaatgaaatacgctttagtatttgcaatttcttttctattaattagtggttgtgctgaagaaattaagtatcaacctaaaccaattgtagaacaagaaatcttacttgaacgttgtgacgaaaatacaccactaccaagtaaaatcatggtcgataaaaacgggttcagaggatatgatggtgctgaaattcttgcagtactcgtaaaatgggatgcaatgtacaatgactgtgcactaaaacatgatgcattaattaaaactattcgtgatttacaaaatcaagatgttaaggttaaaattaaatccgaggattaaaaatgtcaaaatatcaggtctactatgttgccaaactatcacttcttgatgagttaaatcaaagaagtacgcagatgaaacgctatacatataacggtgttactttccgatacttcccacaaaaagtacttacattcgttaagaatcgtaaatgcgttgcgtgtggtattgaagccactgaagttcgcattgagcgttcacatagcactgatccggtatatggtaaacctcacttaaatgtctatgcagtttgtggtctatatgaagtactgatgacagtagatcataaccaactagcatctaagggtggacctgatttagagtcaaattacaacaccatgtgttcacgttgtaacagtcttcgttctaataaaactgaatcagtagatgaattcctggaaaaagttaagggtagaaatctactggaagaacatatgaatgattttttcaataaagataagaagaaatctaaaattcctacgccagaagaattggaagaacgccgtttacgtacagaactatgggaaaccgaccatatatggcatgttcgtgattttggacgacatatgaaagctctaaaaaaagaacaaaagaaattgaataatcacaattaatatgttatgatagggacttgagaaatcgagtccctatttttttggagaaaataaatgaaatctaaaatatttttaccgcagttaatgtggcatctggcagagttagatgaagatgatgaatatactgactatggtacatgggatgacagtactgaatcattcaatccagatgttaagtacaaaatcattagtaatgaaagtaaagggcatagacgatggacagaagaatttgatctagtttttcttgacatgaacacgaacaaatattatcattgtacgtatgaacgaggtttaacagaagatagaccagcagaaccattcagaaatgaaactgatggtgttaagtgctatgaagtttctgttaccagagaagaaattatcacttatcgaactttgtggaaatacttgtaatgctaaaagaattagatttttcaaacttagaaatcatcagtgctgttaaaaatatctctgactttggagatgagaagtcagtactggtaactgttacattcgtcaaagaacaggatagctatagccatttagatggtattgaagaactaattcacaagactaatctacaaaacgatggtaaactcattaagcttcaggcttcctatacgcaaaaatcgtcattttttgtagacctaaataatgtagtcaaggtggtatacaatcaccctgaacacaaatacaggaacacgacaagaaacgagaatgatgctatagactccaatatcaaagaggatgtcgaattcatcaacacaatttttgatcgttgttccaaggatttgaaaaaggtatacatgtaacgggacataacacatcctagtgatataatgtatattgtaaaatgacaacggacattgaaaaatggataatgagaacctacttctcttgaaaatgtaaaggatagatgagctttaccgttgtagcgaacagcaaaagaaacctgttagagtactggcaatcaaacaaacacgagataagaccacgaatcacggcgacacggctgtataaagaactccaataagcacacagagagttcctagatagatgtataaggttcgcaccttacgaatttgctaagggagttcaaggattgtggacgcgattttacaagaaagttgcgtccaaaatttgagaacaggtactactttgatcgaatagagtttagtactgagtgctgttgatgaaaagaccagcgaccgttgcatatgactgcgatagagagaaaccgagcaacccgctttcgctctctaaattttggtacaggaaagatccgtccaaaatttgagaaattcgcaattcgataatatccgatgccatcatcgtcaagagagacttaattgtctcttttttattaccttcattcccctgcggggggatgaaatagtcctcaacaaatcccgtcaagcttttaaagtacattatcattttcagaaggaactagtttacgaagtaaacgtagtgacttcttgagttacgagagaacgaagtgaatgagtaacacaacgaaaatatccaatacatctgaattaacacaaatgttattcaaagaatattttgagtatcaaatcaaaagcgatcataatttggttcagacctatttgaagagaatcaaagatccattaagtttaaacaaaggaactgaattttataacagtattttagactttattaatttagttcatacaaacagacctattgtagttttcttctttgataaatcaattcctgtagaagttcctgataatgtattttttgttactgaaactgatttaagtaatcaaagtactgtaagtagattaaggaattgcttacttataatttgtgattcttcattatataacaatagaatactatgggatagaagtaagagaaatgcggtttcagaaatgaatcctgaagttaccttcattattaactaaataccatagaataactctaaggagagatttatggaaaatattaacgacctacgacatgtaatggactttatcggagatactgaagaagatagttcttatgatgaagtactacttacatttattgagaatagtttatttttaagtgcggctgctcacaactggcatttacagagccatttctacactaagcacctacaattggatgaactatacaaagagttacctgagtacgttgatgcatttgtagagggtattatgaacactcgcggggcattgcaggtaggtactggtaacagttatcgttttacccctatcgaggattgtgtaccaattcttgaagaatatgttagtcaatgtaaagccattcatgctatgcttgaagaggctgaagactatggtagtactaacacgctagaagacattatgagtttttgtgaaggaatgctttacaaacttaaagttttacaataaaatatatggagagtcattgtgactctccttttcttttggagaatatatgagtgattaccgcgtaacatctggactaatggcccgtcttattgaagcagttcctgaagatgattatgaagctatttcagatcgtctttttgacgcagattctaaactccatattacatatgatggtgcattgatttacagtactgatattgatatgactagctatgaatttgagatttcttttggtaacaaactaactactgaagaattcgctcaaaaatgtgagcgtgaagggttgtgtattgatattagttcagttcagccatatacatcactgtggtatgatggtgctgatagtccaatgtctactacatctttacaggattttctaaatgcaaatcaaaaagattaaatttcaccagcattgttctatacatgaatgtgatgattgtggtatgaacatgagtgacatttatacagtttcctgtaaaggattcaaattcactcacggtgaacctgcatattgttatggcacagaagaaggtgatatcgaagtagtactgaaagaattgttccagtacttgcaaagtgcaggtcatgatgttacaatgcccacatattacgaagatgattatgactcacatgtagaagaaaatacatattataatcaattcgttcatggtaatggctttatcacataccttcatgagaatggtattgaggtaaaaactaagtacactagtgatgattacgaaagtgatgactctatggagattgaagattgaaagcaaaagacctagcagcactgttgatgcagaaccctgaaatggaagtgtatcatgagcacatgcatattgatgatatgtgtgaatcgtactattcaccagaaaatgtgtctgggattgaaatcacaccaagtggtgtattcatcagtactgaacgtatggattacaattcccagcaagaaatcaaaactccacataataaacctcgcattggtgctattgaacttgtatttcaacaggatcgtctggttgagtacaacgaagacgatggtactacccgaactgatgttgagaatgttgtttttcctttttataacgcctatgatgacccaacaacaggtgaagacttcttccgtgagttatcagaggatggttatgtttatggtgaaaatgggactatttttgtactggaccctaattatgagccagatcacaacacttggcaattctatacacctcaagatgtaatggaagttaaagtacttacagatattgacgataattggaacccagtatatacttcaatgacatttgaagaagcaaaaaaccatttcgtttctaaactaaacattacaaaagaataaggatttgaaatttatgaaaattgtcgcaagtgcatggatgaaactaccagatggtactactgcttttgaagagtatggttttgttgaaagtgtagaagaattaaagcgtttcaaaagtgaaaatgatgatgatgtatttgacattgtattcacctatcaccatattccaaatgatacagattatgaacattattattacaatccagatctgttggaaaatctagtgtgcgacaatcaacatgaaattgatgaaaacactgctctactaatgctgagtaccgtatctccggtataaggagttatatgtataaccttacaattatccgcgaaccaaatgattatgaaggtaatacaatctatggtgaagttttggtatttcaaagtactgaacaaagtggtattgaacataaattaaaagattatttacgtgatcatttatatctgtaccgtactgatgcaaattgtaattatggtgatataatgtttattgtaacgcacaatggtgcggtaattgaacatgtgaacatgacaaatgtaatggatttttataattacactgatacttcttttccattggataactatttcagtactgtttttgatgcgttacgtaagaacgttgctaaaaatgatagattctgtactaattttagattattagaaacaaatacaagaaagaatctttctaagaagtatgatgaagaacataagaaaaatcaggaaattcaacgtttacatgagttaatgaagaaatatcccaacgaggtcaaacatggctcttagaggacaaactcaatataagtgtattgaagaaatcagaaagaaagatggaacactttttgacaggaaattaaaacaagaatataaagacctaagagatgttatccgaaatgaaagtttcggtgggaagcgtagacagaagatttatgaatttctgaagaatggtttagaaattcaagtcaatggtcttcattgtgtacaaaccaaaaacgatccagatattttacgtcttttaaaagagggtaaaatagaaatgttcaatatcccaatttcatcctatggtaatagattaaaatgtaagcacacgtacttaaggattaaaaatgggtaaattagtatattactatggtagtatgtctagtggtaaaagtactcatcttttacaaaaagcatataaccataagaaaaatggaatgaaggtaatttacttaaccagtgcacttgatgatcgttatggaacaggaaagattacttcaagaattggcatcgaagctgtggctgaagtaattccaaaaaatgacattagtgttcttcttaaagttaaagaaacactcaaaaatgacgttgacattaacagtatctatgttgatgaatgtcaatttttaaacagagaacagattgaccatttagctgatatagcagataattatggtgttgatgtttcctgttatggaatccgaacagattttgaatccaatctatttgaaggttcacaaagactgttcgaaatagctgatgacatacatgaacttaagaacgtgtgtgagaatcagaagtgtggcaaagaagccactatgaatgcccgtatagttgaaagtactgataaagtctttattggtggtgatgaatcatacaagagtctatgcagaacttgctatatggattatatgaggaataaaaatggttgattcatttatgaaacgcgttttgaatgatgtactatctggatttaaaaaagctactagtcttatttttagttcagtagtgatggttattaccgtaatggcatttgcacttgtactgcattttgtaccattctggttagtattcattgtactcattgttcttctaggtacgcatgtttattatgtttactgtgaagctttggatgatgataacgtttaaaagtgtttgacctcactatgtggggtctttttaaggtatctatatgcataacgaaactgaagtcttcaaacgtttcattgatatttgtttacacacgaatttggtatatttgggtccaggagtaaacttcagaactttatcttcaattttaggatacactgaaccatcatgcttatattgcgatgggaacatgatccatctcactgacggtgattttaatgttcagtacatttcacaatgtgatatgtatgtgtttaatacaccagaatataatattattattgaagaacgtactaaagataattacccaacccaatatgtaagttgggatagaacagattgcgtatatgccactggtccagtacagaaacctatgaactatcctactattcatttgaatgttatccatgatgaattgaaagattactgtaacagtatttcagattcggaaacaaaatactttcctattatattttttaaggtttgattatgagatggcgtaaacgacatgtaacaagatttaataaaataatcaaaaagtatttctcgcataagttagatccaatcacacttcaactaagaagtagtatgtttgatagacgtttcccacagtgtttagaatcgtacagtcgtaaacgttacaagaatgaatacctggtatatcgttatgatttatcagaattctttttgaatactcgtgtatctgtacccatcagactgtactcaagggtaaaacgaagtagggtccaagatatcacttatggttatcttatgcttgagattaatgaactcaatgaaaaaacaggtagaaatttcagtactttatgtctggaaaacaatgatatcataaaaagaattatagaagatttgggcaagtaggattttttatgctataattggagatgagtatgaaatacatcgacaaaggtacagttgaaacacagtactggaatattcatattgctggtgatatccaaacagcaagagagtacacaagaaagtttgcattcaaacttggattttgtgtacaattgattccgtgtgaatatatctacacgggtggtcttgaaagtggaatgtgtgctcgtattattcagtacccacgcttcccaaaagaagaaatatatctcaaccgagatgtattaaactatgctaagggtttagctgaagagttgtgtcaaaagagttttacaattgaacgaaataatgatacaatctattatgaaagtaccgaccctttacacaagaaatgaaaggaaaatatatgaatggactaaaaaagtttggtaaagattttctaaagtgcatccgatacacatatgatgcaaatccagaaatgactgccatcgtgggtgtgtttattacactcgttgtattaagctttattgtggtgtaaaatatgattgaaattaatgctgatgtatattgtagtgcatcaccaaactacttcatttatgcactaagtacatttggtgaaactgaaggtgaaacttttgttttagtagataatactacatggtacaaaacaagtaataaaattccgaaagatgttttggaaacttatgttcgtattgaaaataaaaacatatatgtaaatttataggtaaaatatgattgaaatggaaggaactctggtgtatgatccagatcgtggcactatgaaaaagaagaaggacttctggttagtactgcaacttccattacagttggttcgatactaccagtacttcatcagaactgacttacatattgatattaaagacccacaatggggagcacatgtcagtatcgttcgtggtgaagtgccaccaaatccagagtactggaaaaagtacgatggtatgaaagttaagattaagttcgaacctttcatcgaagaagtaaaagacaataagaaacctgggtcattctttatcatagagtttgaagatgatttcattgcacaaattcgtgatgaacttggtttagcatctaaatatttcaaatatcacatcaccattggtagaacatactacgattaatgtggttatttggtaaataggtcttgatataactaaaaagtatgctatactacttaaatgttccaaagaacaactgagtactgtagaaagtccaatgaggcttttgcaaatggcctctttgccctctttgtgattgctcattgttaacaaccaacaaaggaggcgttttgaaaaaagcgaaaaaagcactgaaaactaaaattgccgtaggcgtattagtttcatcagccctattcgctgctattggattttatggtttttctcaaagtgatcatgcaccatcattagacgaaagtcaggtgcataatttaaaaccacagtttactcaaattcaaaagcaggaactaccaaaggtatcagtaccaaaagcagttccaagcagcgagaaacattatacggttcaacctggtgatagttattattctattgcacagaaatttaaacctaaaaatgtacagattgaagattatgtcattttgttgaagaacatcaacttaaatgcaactcttcatacttctcagggtatctttatcccaacaaaagaggatttgaagcacgtattgttaccagatgtgactgtactgtttgattataaagatccagaaattgtcaatcacatcaaagaagctgaaggttctattgaatttcagtccaagaacaaacgacgtttacttggtgggaaagttggagttccatatagaaattctaaattttatccgtatcgtgatataaaaggtaacttcactattgggtacggacattatcttggaaagaaagaatcagatgcaagtaagtatcgtaatggtattacaaagtcacaagcacataaacttttattgtctgacatggaacgaactaataatgatttagttttattattgaagaaaagaaatgcaacaaaccttacggctgaacaacaacgaattttatttgagatggcatttacattaggagttgataagttaaatactttcaataaattgtggaaaagcgttgataatcacaatgataacaagttcaaaaaagaaattaagaattctctatggtatcgtcaagtaggaaaccgtgcagaattgttagttagtaatctttaaaactaaaaaaaaaggtaggtaaattatgctagatttagttatccaaggtaatgttgatattactccagcaatcaatgacgcgattcatgcagaagcaaatgtactaacgaaatttagtgattcttcagtactttatatcggtgttcatgtgaagaaagagcataagaatcaatattccgtaactttaacatcaggtggtaagattgtcaccgccgatgatgcagatttatacaaagcaattggtgattcatttgaaaaaatggaaactgtcttaggtaaagacaaaagcattaaattgcatcgccgtaaccaccccgaacatccagttcaagaagacttgactgaagacgaagagtaagatataataaagggacagagattattctgtcccttttcttatggagttttaattatgaattattcctggatgggaattgtaaaatctttaatatcaacacatgatatggacttacctgaacacctagttattaatagatggtactatccatatccagataaccgttgtgttgggattggattttttattaatcaaaaatctcaatttgtcggattaattgaagatgatacaaaatacaaccttgtgttgaatgctgatcacataaaatactacacccataaaattgacgaagactatgatgttataatcccagtagagatagacgaagcttcattttttcagttatcattaattcatgatatatctgatattacttatgataatgtgaaagacctttttatattgtacaataaaatgtataattactttatatgattgaggtgtaagttgagaaatacattaattaaagttaatttatcacatttcgacccatatcagaaatatgaagaaagtcgaagatataatattaatgaagatttcacaatatatttcaatagaacatatagcataatcatcattactatttgtaatcatataaaaatttgtataacaaaagacacaataactttacacattcaagacaaagaaactgatattccaacagatttggatgaagatggatatgctcaagacgaattcttgagagatcacttgttgattgagtacagcacttatgcattgattaatttctacaaagtaaattacattaagtggttgtctggtgaaagtgatgacggtataatagagtataatatataatgaatacaattatacttgaagaagatttgggtgtttatgaacaagaaaccgacgtgaacaaagaaccagaatacaagcctaatggcttattggaacgtctctttgattggctgatgggggagtgatggaatatacaatatgtgctattatttgtatcttagctttaatctgtggcgttggtgtgatgatttattgtgatgcagaacccagtactgaagtaggaacaacaataacacttaaacctggtgttaaaattaatgatacctattgctacaatccaaacacaaaccgctatgagatttgtttatgagaacattcaacattgattatataattgattcgattgcagaacatatgaaaaacataagtaaaaacgagcctggtttttacttcaacttagataaactccatcgcaataccaatgtatctagttttaatcgtgctaagaaactttacatccgtatacataacctttggtacagtgacgatacgttgatgatagatattactgttgatgaagacataacattaagatacattgacgattttaaatcagatgatattcgtgctggatcattaccaattgatgcttcatatgaagaatatctgcaataccagttcattgatgacacatttggatttgactttgaactatataatagtataatgaacgtatatcatgtatatgaggattacattggtggacgcaagtaaaattccaaaatttaatgaaacgttaaagtactttgctgaacatgaaatcagtactgaaagacgtggtttaatttcatacaatcatcatcgtgttatgtacactaataatttccagggtacagttcgtattaaatttcgtaatgcactcgatgacaatgatgaattgctaattttactacgaaaaagtaagattgaattgacatacgcaactcaccatattgaaagtgagttgatgtggcacaccctatcaaaaaaagaagggacattaccagttgttgataatggaccagaaacacttcttcagtacatgttcattgatgatacatttagtattgactatccatcatttcagtgtatactagatatacatgcaaaatattacgaccgtctacgtgatagattaatttaattggaggcattgtgattacagtattagaagaagattggggtgtttccgagcaaggaacagatgaatatgaatatccagaatatcagccaaaaggcttctgggaacgtctacgtgattggttgatgggggaatgatatgaaaaagtatttggttggaggttatacgagagatcgtttaatgggcatccgtgcaaaagacaaagattatgtcattgtcggtgcatccgaatctgatattaagtacttgcaaagtattggttataagcaagtaggtaaggattttcctgtgtatctcagtccagagggtaatgagcacgcattggcacgtactgaacgtaaaactggtactggttacggtggcttcgaagtgaatactaaaggcgtaacactggaacaagatttgtatcgccgtgaccttacaattaatgcaattgcgtatgatccagtactccagattcacatcgacccttatggcgggaaagctgatatcaagaacaaagtacttcgtcatgtatcagaacactttaaagaagatccattgcgtgttctacgtttggcccgtttcgctacgcgttactccgactttacaatccatgatgatactatgacaatgtgtaaagatatggttagtactggtgaactagaccatctagttccagatcgtgtttttgttgaattcgaaaaagcactttctgaagaaaacccttccaagtttatcaaaactctggtacaatttggttattacgaaaaggcatttactacagttccagttaccaagaaacgtctggaattaattgattctatcgctgcaaattgtacagaagaatataaaccatatttcttatggacagtacttcttgaaaatgaagaagtaaataatgattcagtacttaacggttccattaaggttccagttaagtacgtgaagtttagtaattacatcaagcgtttccgtgaagatattaaaaccttccgtaagaagaccccagttgaaatgggacaaatcttactggatatgaatatcaaaaacaatggtggtgaagatttcctttacaaagtactggaaaacttcattttgaacaaagaaattgattctgaacttgaagatttgattattaaagtttatgatcgttatactgatgttgaattacctgatattcagcagttagtgaaagatggtgaactccaaacatcagagattcgtgattttgttgctgaattgcgtattgaagcaatcaagcaaatgttctaaaagctgggacaagttaacatcttctgatataattactagtatctgaacatcacttgatgataaagagataagattatgcgttatagctgtggtataatattttatcgtgatggtcagatacttatcgggcatacaacacggacagaatcactgggacttacctaaaggtaaaactgaaggtacagaaacatttaaagaggctgctgtacgtgaatgctacgaagagacaggatatgaaatcgatgaagatgatcttctgtttcttggtgatgtatcgtatagaaaggggaaacgtttagttttatttttctatacagccaaagataagcctataccagaagagttggagtgtaaaagtacttacacaaacagatatggtagaagtcacccagaattggataagttcatgtacattgatgtactagattgtcaaaagtatttgactgaacgaatgtgtaacagtataataaaggctgttacgatgtttcaacgaaataaagggaaaagaaatgaaagttaaaatttatggtcgcactactggttgtaaattctgcgacactgctaaagcagtatgtgaaaataacaaatttgatatggagtttatcgatattgttaccgctggtattgatggtgctaaactatctgaactatgtggtgagccagtacgacaggttccacaaattttcgtaaacgatgtttacgttccaggtggctgtactggtttcgttgaaggtcttaaagaaggtcgctggaattaatgaatattgataaagtatcatctcgtgcaaaacatttagctgatacattgggtgttgaatttaaaatgcctgatatggaagctgcaaaagttaaagcacaacgtaaaattcacaaaatgaaagaatacactactaagaaagaacagtacgataaagcggttgcttatattattgaagctttcagtaaagatgatattttggcattcatcgcttcagtagaggtttcgagcaaccctaaataataatgaagaacacaagttcttcgtaacaatttgggagatactattatgctcagttttcgtgaacaattagcaaaaggccaattcgctgctaaagctcaaacatccattcctaaaaaatctgctttcttagggacgattacagaagatggttattgtgattatgatgcaccagaagattcatggtacattacgaaagacacaaaacgtggtaatagacttcacaaaagtaccattgctcgcaaacaacgaaaagaattgaaaaaaattcgattcggtgaagaagaataaaatataaattaaggactctgattacagagtcctttcttttcggagaatataatgatacatgaacacctaggtaatttgtttgaattattcaacactgttactgatgatcaaaccgcgttgcatataatcgctcatggttgtaatgcacagggtaagatgggttcgggttttgcaaaagaactaagaaatcgttatccgatggcatatgatgattataaagcatatgaaaaggaaatgggatttctaagaacaggacaaatttcatattcttctataagtgatgatttgcttatcgcaaactgcattacccaaaagtactatggtaatgacggttctaagtatgtaagttatgatgcaatcgatgatgttttcaaacaactcaattacttatgtaacgttgaacgcactgttcatgtacacttcccgaaaattggagccgatttaggcggtggtaactggaacgttatcaaagaaatcattgaccacagactggttaatgcagaaaaacacttatacattctaaaataaacaaaaggacttgacaaaaatgactatgaccgttaaactactaaacatcaacagcgttggcccagtactagcaattctacaaaatgatgcaccagaagcagtagtagttaactacccagcacagctaggtactgacgaacaaggtgaaatcattctacgtgattacctagaattcattagttctgaaaaaggcgttattttttataaatccaacatcattagcatcagcgatgcaaaccctgcactagttgaagcttatgttgaagcaatcaaggctatcgaagaaaaaccagcactgattgttcctgataacaaaatcatcctataatagaggtatctaatggctgctcaaaacgttaaagttaaaactatccaagctatcgtagtactgaaagattctactccatcaccaaaagaactggttatggctggtatgacaatgctacatcagtacaatccagaatcacctaactatgtactggaatacgaagcgaagcagtttaaagaacattggcagtcatggattaaagataatgaaatggaacaggttgtttatgctgctgatgacattttcgaattagaacaaatcgaatatctagcaggacaacaatctgtaccattgagtatcttcggtaatggagaagaagttttagtactgggaccatttaccgctgatgccatcatgcatttactaagtaattgtaaaaaagttggctaaaaaacaaatattatcaggttaccgtaataacacgttgcactacaatgacttaaagtactgggttgacccacacaacaataagtcattgttgctgtacagagataaatttcgagatttgtacggtatatatgacacacagttaatatacttagttgataccgaacattttgctcctatggtatacttgtattgcttgtataattccaaataactattaacacctctaaataatgtatataaacattaatgaggtgcttttttatgtcttacgattatatgggtggaaacccagatgctccaaactttgatcatcgtgaaaaactgaaagatgttatcactcaaatgttaggtgctggtgcagttgaagttgaaattactgacggtcaattggaaattgcaattgataatgcggtatcaaactaccgagcatggagttctgcttcaaaagaagaagcattcttgaacatgaaattctatgattcacaatctgtatacatgcttcctactgaagttgaaattgtcaaacagatttatcgttatggtaacggttatctaagttctggtagtggtaactcagtagaccctttttcactcgcatttaccaacacttaccttttaacgtctgcacgtaacgctggtggttatgacttaactacttatgattttagttttcagttccaagaattagttgggcgtatgtttggtagagaaatcatcttccagttcaacccaatgaccaaaaaactaataattgaccgtgatattcgtggtactgaagaagtgctattacatgtatatcataatatacctgaaccattgctattccaacgtcaacaaagctatccttggctacgtgactgggcattgtctgaagctatgattatgttgggtcgcaaccgtgctaaatttgctacattgcctggaccacagggtacattctctatggatggtgatacattacgtaatgaaggtgcacaacttcaagaacagcttaaagaacagttgaagagatacaaagatggtggtgatccacttggaatgattatcggataaaacaaaaaaaaagactcctatatggggtctttttttatatgttgtgatataatgataatattaatctaaaggaacctgacatgaataacaacatagatgcagtgtatgcaatgtataacaaatatacttgtggagcacctacattggattatgaatcaaccgaaaagaactggaaatttataaaagaatcaatcgtagatgttttcggtagagatcgaataaacgcgtgggcgagtatatgtcgtgatgataatggagaaacattctacatcacttatgttactatatattatgggagtgagtttatagttggattacttgaatgtttagtaggaaaggatttagtagtacttatataatatacatggagaagtattatggaaagacaaataattgcaattaacggtactatcggtagtggcaaagatacttttggtgaaacttttatcaataatggttacacaagaatgtcatttgcaactaatcttaaagattcagttgctgctatctttggatgggatcgtgaaatgcttgagggttcgactaaggaaagtcgtaaactccgtgaagagccagaccagtactggtgtgagaagttggggcgtgatgatgtttcccctcgttgggtattacagaattacggtactaatattttgcgtaagtactttcataatgatatttgggtccatagcttagagaagtcaatgcaatctgttgaagggaatattatcattactgactgtcgattcccaaatgaactaaaaatgattcgttcattaaatggtattgtgattgaagttcagcgtaccctcccacactggtatggtttagcagcatcttttaataaaggtgatattagcatgaaaccaattgagttagaatcagttcatgaatctgaatgggcgtggattggtatcaataatcctgatatcattgtacaaaataatagcacaattgaagcgttgcaccaacaagcattgagcgttatttatgccaataagtgaaattaaaacaattaggacttgacattcaaatccttcaataaataagactatacgaatataattcgaataaccaattatttttggaggatttaatatggcaggagatctactctcacctggcgtttccatttcagtaactgatgaaagtatgagtactgctgctgctcctggcacagtaccactatttgtaatcgctactgctgaagacaaacttgttcctggtagcagtgcaatcgctgcgggaaccactaaagcaaacgctggtaaactacagctactaacatcacagaaaagctcactagatacatttggctcaccaattttccaaatgaacaatggttcagttgttcaaggtgacgaactaaacgaatatggtctacatgcggtatattcttatctaggcatcaccaaccgtgcatatgttattcgtgctgatatcgatttagctcaactatcaccaagtgcaactgcaccaactggtccagtaccaaatggtactctatggtttgatgctgctcaatcacaaattgaagcttatgttgctaagaaagacagcccaagcagcttctatgactggcaattgaagcctgttaccatcgtaagttctgaagaagttgacgatgttagcctagttggtctaaacgttgatgatctagttctacgttataagccaagtaatggtcaaatgcttatgtacaaacgtgcagcatctggtctagtacaggttgaaacttatctatcaccaatcaacaaagttccagttggttcaactgttgaacaaggtaacgtatggattcgtgatggttatactcaaaatggttcaaactattttggtactcgttttgttctaaaacgttatgcatctctaactcaagtttggaatatcgttccagcatatactggtaattctttccaagaagttgaagctaagagtggtgtactaaacaacagttatattgcttgccgttttgatgacgatagtaagagtttctctttctatattcaaagtgcaaaactatcagcaattagtgctccagcagtaccactaggtgaaactgttattgagcaagctggtgaagatagttatttgactttcgcttacctaaacaaatctgttaaagtacttgcagtaccacaaggtggcgtaattaatactcaaactatcatcgataatctaaatgttagtgctgatcttatcaatgatggattcaaattcaaacgtgaatctaatggcggtttggtagttacaaacaccaaaggttatagctttaaagttacccaaagcggtgatgatatttttgcagcgaatgaactagctaaactaactgtagatagtaacaactattctctagttaacgttaataacttacgtgttagctcttctgttccacgtagtattgctaaagacggtactatttggtatgatttcggtaattccgataacctaactgttaccatgtacattgcagatgttgcaaatgacgaatgggatctagtacaagaagataacaaatatgttcaagttgatgaacctgcggcagaccgtctaatttgggtacgccctctatctcaaggaattgatggttatgatttctatcgtaacgttgatggtgagtgggttaaactagacaatactgaccaaagtactctaaacggtatgattttcgctgactttaacttccctgaacaagttgcttctgctgctctttatcagaatggtatgcttgcaattgacctcggtaataccgaaggtgttgttaagaaaatggttaatggtcaatggcaagttgttagtggtgttagtcttgatggtgcaggtctattcggtcgtgcagcacaacgcaaactaatcgttgaagcactagctgctgtaatcgtcggtaatgatgatatccgttctgaattcatcgacttcaacctaatctgtgttcctggttatgtagaaactctagatgaatgtattaccctaaacactgatcgtaaagaaacagcgttcattgtaactgatgtacctgctcgtttgaacccaaccggaaacggtagtgcagttcaagaatgggctactaacgcgaataacgcaccaagtaatggttcagtaggccgtacttctgcatatgattatgctgctcagtacatgggctggtgtctaagtactaacgttgatggtgaagaagtagcggttccaggcagtactgttgcaatgcgtacctatgcgtacagtgatagtgttagttttgtatggtatccacctgctggtactactcgtggtgttgtaaccaacgctgcaagtactggtttcattaatagtgaaggtgaatatcaaccagtagtatacaaccaaggtcaacgtgacgtgatgtataccaacaacattaacccaattgcaatgcgtcctaaccgtggcttgctagtatatggtgataaaacactatctgctaaccaaacctctgcactaagccgtgttaacgttgctcgtttggttgtttacattcgtcgtcaactagaaatcatctctgaaccattcctattccgtttgaatactccaagtactcgtcaggaattcagtggtgtagtgaatagcttcctagctgaaatcgttcaacaacaaggtctatataacttcgcagtagtttgtgacgaaagtaacaacacttctgaacgtattgaccgtaacgaactttggatggatattgcaatccaacctaccaagtctatcaacttcatttacgtacctattcgtattgaaaagactcaataaaacactaagggaaccttttggttcccttttttattgtttaaaattaaatacgttatgttattaattcaacgatcaggaataatatggaagaaatacttaaaaaaacattacaagatgtaagtaaaactgtagcaacacaggacgatgccgtaaaaatccttaaagcttttatacaaaatttggaacattgtcggtataattcgagaatacaatgtttacacaaatggtaaatggtcggttggtgtaaactatacagatcataaacaagaaaaaaagtacttgtattgtgtgaatatagattggtttcctgttgatgagatacttcaataaaagtgtaagtaatagataaataatataaagatgtcattccataacaggagaatataaatggctacaatgcttgatcgatatggcgttccgctcgctggcggtggtcgtatctctatgaaacaacctaaagcaaaatataaattccgtgttgttttctttggcttcggtgctaacgacgatggcaatatgattaccctagataccaacaccgttggtgctgtatctgtatcacatgaaaacgtcactgtgcatagttataacagttctgctcactataaaggtaagtatacttgggatactattgatgtggctttccgtgattcagttggtaatgcttctgctaaagctctttataaccaaatgcgtaaagaatttaactattacagtcaagaatctcgtatgactgattcccagttcaaatttgaaatgtggattcaagaactagatggctccaatagtgaaaacacttctaacctttttgaaggtactttgagtacttgggtatgtcaaggctgctttatcgaatctagtaactttggtgactgggattattcctcaagtgaagcacaagttatttctatgacaattcaaccagacggctgtgcaaaactcggtcctaatggtgaagcgttcggtgatgaacttactggtttcggtaacccatccactgctacctctattagtggtgcacaaaccaacattagtagtactactctaccagatagtaacttctacgaaaattaatttttcttttaaacgtaagcctcgaactagttcggggctttttttatgctaaataaagtataataccttataaaataaaagaggttgctatggcaaataacatgattaataaatatggcgttcctctaccatcaggacgtaaccgttcaatgttacaaccgaaagcaaaatacaaatttcgtgttattgtatataatttcggtactgatatagatgagcgtgatcacattgcattagatgttgataaagttgatagacctactgttactttcagtactcataaactatatcaatttacaacaaatacatcttacattggtagtcacgattgggacccaattactttaactatccgtgattcagttggtaatgcatcagcaaaagccttgtcacgacaattacagaaacaattagattttcaacgaagaattacttccagaactgaccatgattatggttcatataagttcggaattgtcattcagatgttgaatggtcgcaactctgatgatacactagaagacttgggaagaaacacattagttaatgcaacaactgccgctaccaacaatactggattagtcaacgctgttgatcagttcattggtggttcatcatacaatagtgctggcgttatggagtactttgtactaacaggatgtgttatacaaaatatatcatatgattcacttgactatagttcaagttctcctgttgaaatggttattacaataaaacctgataattgtatccaattcgataatatcgaagaaatgtacagtactagaataaattcacttttacctgaaaacgtaaaccaaggattagatatcattgaccgtgttttcggtagcattggattataaggagataacccatgagtgatttttttgattcatttgtcagtactggaactagtatagttaatggtgttattgacaatatcaacatcattagagaactagcttttgagaattataacgctgcacaacgtgtgtttggtacagcaggtgaaatgggtttttcattctataatttaccaaaatttaagaacagttttattatcgaatttgtactaaccccgttcgcaaagaactttattaaaacccaactcccagatacacatactgattttgatgttaataatgtatcgtgctttgtaaaagatgtaacacttcctagtttttcttttagtgttgaaaccgtaaaccaatacaacaaaaaacgtttagttactggtgatattgtttataaacccagtaatgttgttttctatgatactgttgatggtgctggatatctcctaatggacgcatatcgtaagttctactatggtgacttctttgacaagaacccacaatcttaccgaaatgacgtactcagtacaccagaaaatttcaatggtttaggtggaaactggggtcgttctgtaatgaacaatggtaactacgacaaccagtactttttcaaatcaattaatatctatgaaattgataatgaaacctatacagttcataacatgataaatgtattcatcgaagatatacaattagaaactaaatctatggaaagtgaaggtgacccagcactattatctttgactatgcgttatgaagcgtgtaacaactttggtcctgaagggtacttgagtattggtgtccctacaatggaaattgcacaactcatcaccgatactgaaggtcttggtgtgagtggattctttaagtactttggtgaacttgatgacaaaactaaaggaatttcgacagttggtaaaattatccgagcaggaacatcagttagtgatataatcagtagtgctggtgatatccttcgtggtgacattactcctgataccatccgaaacataggttccgcagtgacaagaggtgcaaatgctattggtttaggaagcatcgtttcttctgcaagctcaaaattcggattaggtaacatattaggagatttttaaatggcatttaaccagcagtttaacccagcgaagattaccaattccaaacaggctcaaggtatcttccctataaaaaatatacaaaagtataaaagtaataaccaacctatataccgtagttcttgggaaaaggatatcatgatatctttagacaacaaccctgcggttatagagtggagtgtagagccatttagtattccttacgtgaatccagttgataatgcagttcatcagtactggcctgatttttatgtactttatattggtgcagatggtaagcaacatgctcaagtactggaagttaaaccgtacaaacaatgtcacatggaaatggctaaatccaagaaagacaaaatcaccgttgcgattaaccaatcaaaatgggcatatgcaatggaattttgcaaaaataaccggattggaatttaaaatcattacagaaaggaatctatataaaaaatgaaacctaaacaacaagtagatgaagcactgggattacctagtgctaaacaatggatggaaatgacaggccaggagcctgaagaagatacagagatggatttgtatcaggatgacgaagatgaacagagtgaaactgaaatttcactagaagaggcaaaagctgcaatggaaaaattgaaagctatgcgtgttcagttgcgtgaacttccagatattacagcaagaaaaactcaattagatcgtttagcacagatggcagaacgtaagtttgaagatattttcgaccgtgctttcaactgtgaagaccgttttgcagcagaaatgattaacgctgcaaactctatgttaaaaattgctctcgatgctcatgcaaaaatcattgattctgacgttaaattagtagatcttcaactcaagaaagataagatggaaatcgatctaaaccttaaaactaagaaacctgaatcagttggtgacccaacagccatcgaaggacaggcggtagcacataaaacccgcaatgaaatgcttgcagcaatgaacagaaataaataacttaaacgaccttatggagttattaaaatgaatagattacaacaaatcctaacggataaaaaaccagtttttgaataccgagtcaagtttgcagttgaacctaccaatgagcaactaagtaaagtatcacaacgcctaatggatcgttatgatgcatttgatattggtgcactaaagaaaactattttcatggaaagaccactagatttttatgaattagattgtggtgaaatttggatgtttgatttcaaatgccaccgtggtgttcaatcaaacgtactaatgtatgaaattggcaacctactaagtatgactgaagctctaatccgtgtacgtagtaaagatgaaccacgtcaacaagaaatgaccgaactcgaagatgatattgactttgacgattacgaaccagtaattggtgttgattttggcaaaccagaagacagccatgatttagctggtgaagaacgtgctgtaaccgcagttaaagatgcgattgaagcagttaaaagtgacaataaatactctcaatatatgagtgctggttttggtaaaaaggattgagacatgagtatacttaatgaaatgttagactataatgatgaagagctaaatgaagctcgtccatatagtcgttcacaaagtgcagtagatagtgcaaaaggaaaagtaaaaggctttttcggtagtggtcaagtagaacaaggtgcacaagaagttggtgcagaagcaaaccgtctttgggctgaatttaaacgctatgttggtcgcaaatacggtaaagcacagccatcagtaccatatgctgatgttgcacaattcttcaaaggaaataaccttgatataaaacacctgggaaataacagtcgtcgtacttttactcctaaagatgttggttctgctctattggcagcagcacgtgacttgatgaatgattatcgtgatgatgataaaggtgaagaacaacaacgtcctgcaccacaagctcaacctgacaatgctccacaaccagcaccacaggaacagccaaaagcacaaggttctagtatccctgatgctctacaaaaattaagtccagagcagcgtgaacaacttttgcgtatgctttcttaaggagaatcaacacatggataattttttcagtactgaaactaccacagtacctaatggatatgtatatacttctcacactggtggcaaatatgtatttctagaaggtttctggtttaacgacaataatatgttaatggtagaccctaaaaaatacccatccatgtacgcaagtgctcgacaacagattgttgagcacaatctatcttctagtgaatataaaattggtaacaaatatattcttgaaggtaaagagtgtacatttcttggtgaaaatcgtttcaattctaaagatggaattctactaactgaaagtgaaattatcactgaactaatggcagatgaccaccgtgtatataatgtttcaaaagaaactctacaaaaagaatggaactccctaaaattctatatccttccaccatatgctgatgatatttctattccagcaggtatggaagttagtggatttaaattcatccctaaaaatcaccgttttgttgatgcaaaatctggacgtccagttgacccagcaatgacccgtaaactttccgatgaaggtatgcgtattgctcgccaattagcaccaaaaaacaaactaattccaattaaatctactttagtacaaggtaatactcgttccgaatggaatggtaaagaattttgtgataatgatggtaatgttgtaattccagaacaaaatgcacatgtaatctttaatgcatatcagcaatttgttaaggctaaccctggtgattttccttcactaactggtgaacatgaatcaggtgaacaacgttttggatcaggtgaaaagccagaacaacctaaacaaccagtaaaacagccagtattcgcaacaaaagaatccattaatgaagcagatgaagagattgaagaaactgaatctgttgtggttccaaatggttaccgcattacttcaaataaaggtaaaacatacattaaacaaaaagatgcatggtttgatgtagaaactaaaaaacaactaaatgttagtgctgcaaaaagtgttgaacgtgctgctcaacgtgatataattcgccataacagttctggtgcaatgaaaatcggaactaaagtaaaatctaagaaaggcacagagtattcttatatcggtggtaaccgctttattagtgctgatggtaaattgattccaaaatcaactgctgataacattcttgctcgttatgatgcagaacgtgctgataagaaagaagaagaacctgaagataaagtagatgctgaagtatcaagtactgatgtagttgagccaaaagctgaacctactaaagaaccagaacctgttgcagagccagaacctgaagttcctgctgctggggatgaagttcaatcattagcttcacgaattaaagaatcaccttacgctaagaaaattacagtactcttaacgcgtggtgatgaggtttctctaattgctgctgacattttactatctggtaaaaaggatgaaacagtgcaaattttaaaatcattaaataagaatgattgataactaaaaggatttatttaaaatggctaaaaaaattaatgaagcacaagttaaaatcgatatcgcaggtctagaatctgaagatctagaaacactatctcgtatgcttgcattagctggtcaagcagagcgtcaagcttctcctgttgctgctccagttccttctatggggtctatgaccccttctctaccaccaatcgctccactcgacatggattccattggtgcgggtgctgaagaagaagttggtggtacaatggatggtgcgattgatgcaccagttgaagacccattggaagtagctgctgactcactatctgctgaagctggtgattttggtggtgattctgaagaagtaagtatggatgcagagttcgaagagcctggtgttgcagatgaagaagagttttctttagatctttccggtcaaccgcaactagaatcagttgaagaagaggaagaagaagaagaaatcatcgaagaagaattcgatttcagtcgtatgctatctctagctggtcttcaagacgaaccagaagatgaatattcagtagaaggtaacccagatactgaagttaaagttgaagaaaacactttcctaccagacctaaccatctctgaagatgaagaacaaggtgacagtactgattttggcccttacaacactgaagctgatgctgttcgtgacggtcaagctaaaactaatggtgtagaaggtgaccactttgttgttattaccaagggtaacaagttctactggagacgtgtcgttgatgaagatgttgacaatcgtcctgaaccagagtactatgacaatgacggtaacgagaaccgtgttcacaaatttgaacctaagtttccaggtactgctctaggtgacaacccacttaatgttaatgagagtgatgagtcagtggaaggtaacgaagaactagaagatgaaagcgatgaagaagtcgaagacattttcgaaagtctccagaagaaatatcaacaatttctaggggacaaataagatgggatataattatcgtgaattggttgaagcattagatgaaatcacttctagtgaagaatctactgaagaagaaagttttgcagttgagactccttctgatgctgttgccaccgatttacttaaagatatcaccgaatgccttatccacattgtggataaggttaaatcaggtgaagatcaaaaagaaatcgtagaaaagcttcaaaaagcgtgtgacattttatcggatgttcgatataatgatgaaagtactgaagaagttgatgaatccactgattgctgcttcggaaccaaagatatgggtccgaaaattggtaaagatggtgtccaagactataaagttaaagatgctccttcatatgcagcaagacagggtttcgtggggatgtaatttatgatccctgaaccacacaacagtactgaatttcaaaacgatgtacttttaaaagttagagaagtacatggatcagacgctgaattaacagaccgtatgcttctgatgtcaatttttgaaaatttcagaactggcggtggtttaagacttagcaagtttggatatacaatttgtaaagaaaacattctttacgaatttacaaaaatccccttgcaaagagagttcaaaagttctataatctttacatcgcttgataggatatgtacttcaccctactatgttgaagggcagtacatatacctctctgacgcaatgatagttacagagttaacattgtgtggtgatgattttagaatgttattttcgatgcacatgtagcttcgaaaataaagaaaaaaagatgttgacaacgccgaatgatgtgttataatgaccaccacgaagcaagttttcaaaatcacaagatgtgagattgaaaaagaaataaaaaagttgttgacaagcctgaacgatgtgatataatgaccacatcaagacggcaaagagaaatgcctttttaaatacacggatgattcatgaagtaacaaaggaactgataagttaaaaattcttttaacacagttcctttacttttgcccacggattggctataatagaaacctaaacgattttatccaacaaaaggaaaatatacatgtctactcaaaacaacgatatcctgaacgtaaaactgactcctaagaaagctaaagaagcaatggcactggctgacatgattcaagatagcctgatgatttggggtagccctggtatcggtaaatctgctatcgcacgtcaatacgctgatgagaactacccactgcgtaaagataacgtggcaaaaattgagttcatgaaacaacaaatcgaagctgatactgaaaatgctgatcaacttcgccgtgaactggcagcgtttgaagccaaactaatcgaccaagacactaactttatcgatttccgtttgtctcagattgaaccaagtgaccttcgcggtatcccagttccagagaaagtgttccttgatatggatggtaagcaactgattgaatctgaactggctagtgcacaagattacatcgctgaaacagcagttgtgtgggcagcacctaaagtactgaaactgtctccagattggaaaggtgttattctgtttgacgaaatcaactctgcaatgccgattgtacaggcagcgtcttatcagctaatcctcgaccgttgtgttggtgagatgaagctacctaagactgcactgattctcgcagcaggtaaccgtgaaactgatggcggtgtaacgttctctctagctacaccactgcgtgaccgtatgacccacatcgaaatgattccttatcatcaagattggattaacgattatgcgattggtaagcgtttgaacccagcaactgtagcgttcatcaacaacactggtgctaaacacttcaacacccttagcccacaagatccttctcactgtggtggtacttctccacgttcatgggaagcagtagcacgctacgaagatctgaagaaagttaagaacattgataaagaagtgttccgtgcgatgattgcaggtcgtgttggtgaaggcgtagcaactgaatacatcgagtatgttgataacgtagctaaactaccagataccatgtccatcctacagggtgagattaaagagattgatgcttcgcttgatatttccgcgaactacttcatcactctgaatctggtttacaaaattatcgacctctacaatgagaagcaagaacaacgtctaccagtggaagattggggtaagctgtgcaacaacttcatcaccttcctagaccataacttctctgcaactcaagcagaactggcagtactggcgattcgtaccatcacgcttcataaagtagcgattacgattaaagatgtaccagcgtttaaagacttcgttatgaagtactctgacctagtacgtaaagcacgctctcttggttaataattaaaaagagtgttgacaaggaggataccacatggtatccttcttttatcttaactaaacgaggaaatacaaatgtcagataaacgtttcagtgatgttactccggctctccagaaagaatgtcaagatcgtctgatcttagctcgcacatctttaatgctaacattcccattctttggtgtactggcttctcgtctgcgtccagttgcaaacaacacatggtgtcgtactttggctgtagatggtaagcatttattctacaacgtagaattcatcatgggtattcaggaccaatccaaaaagtcagcatacgaagaagcaattcgtgagaaacatcctgaagtaactgaagaacaacttaaagaaatgacagaaggtttgaccgatgaaaacctgattgcagttatctgtcatgagattcttcactgtgcatatgaccacttcttgcgtcgtggtatgcgtgacccgcagcgttataaccgtgctgctgactatgcaattaaccagattcttgttcgtgataaaattggcaaaatccaaagccaatggctgtacgataaaaagtacgatggtatgactgctgaagagatttataacattctcgaagaagaagataagcaggatcaagacggagaaggtgaaggaaatccacaggctggtagtggtgagggtgaaggtaaatcacgcggtacttttggtgatatcaaagatggtaaaggcaaaggtacaacaatcgaccaacatgacgttccacaagaaggtcaagaaggtggttcaagtgataaacaaaaacgtgattatatggatgatttcaaaacagcaatgatgaatgctgctcgtgcaggtggtactcctgctgaaatcgcacgtatgattaacaacttcaaagaacctaagattgactggcgttctaaacttcaccgcacactacgtagctggttgaagtctgatgcaacttatcaaaatcctaatcgtcgttcatggggtgctggttttggtagttctggttcattctacgggaatccaatcttccctggtatgaaaccagatgatgatattgatattgcaattgcactagatgcatccggttctatctctgaagatatgctccgtgatttcatcagtgaagtactgggtatcactaagcagtttaaacaatttaaaatccgtatcatgacgttcgatactgaagtttatactgttaaagattacaaaactggtgaagagagtaagattctcgaatatccagtgaaaggtggtggtggtactgactttgaagcagtatgggttcatatgaagcatgacaactataagcctaaacagcttattatgtttaccgatggtgagccgtggcagtcatggggtgatcctgattactgtaagacactgtttgttatccacagtaatcctaacaaacaagcaccatttggcgaaactgtacactacgaatatgaagctgacaaaaagcgtcaacgtagctaagtactaaataatgagaacggaaaattccgttctctacttttttcagcgggacagcttggaaaaaggtgatataattatatttgtaacaacatacatggagagtattaaaatggaacaagaaccagaaaaaattcaactaacactaatcgacctaatcaacgctgctaaaatcatggaagctgctgtggctcgtaattcatttaaagtcgaagaacttgttgatgttgctccagtcgtatctaagtttgttgagtttgctaactctgcactcgaagatatcaaagcacaagaagaagctgatgctgcacaagctgctgctgaacaaaataaaggagaataataatggctattcgtcacgttggtgtagtacgtaatacgggttcacgagtatttgtggtatggcgttcactccctgatgatccagaacattgtctcattgtttatcgcgactcacttcctgaagcatatgctcaatctgttgcagatttagtaatgaatcaaggtcaatcgaatgtagatttgtgggaagttatgcataagattggtcatctagacggtcgcaaaatgctagatgttcttcataatctacaacatttgcgtcgtgcacgtacttgtgatattgatatgcatgttggtggaaacagtaagatttctcttgatgttctaaacaacgcaatcaatgataatgatgctaaaaaacatgaagatggtaaagttaaagaatacaacccattcaagccacaagaagttgatacttctgttgaagaagctaccattgttgagcaactacttaaagatgcacaacgttatgagcagttggctaaagagcactttgaacgtgcttacaacttagaaccatctctacgtccacaagttcaagcaacacctgtagcacctgatagcaaaactttctatattgaaatcccagaaggaatttcacaaaccaaagcaattgagaaagttaaacaggcatttgcagaacgtaaagcaaaataatataaatgaataaaatagaattagatgaagcaatcaatggtttatctaatgtatgtttcagtggcggggcagcaggtgctgaccgccttttttctatctatgcaaaagacaatggatttgaagtaataaatttttcattcaagggtcaccaaagcagcgtagatgaaaatactatattagaaattccatccaatatcctttgtgataattcaatattaaaacaattaaacattgcatgttactctttacgccgtaaagttcctaagcctggttcgtatgtatataacttacttgcaaggaatagttaccagatacttaatactaaaagagtatattgtattgcaaatattacatcacctacgcaagtttcaggtggaactgcatgggccgtacagatgtatatggattcagttgacaatccagaaatatattgttatgatatgaacactcgctctgtattttattatgatacagtactgaaagagtttactgaaacattctcaataccagcaccacacggtaactggactgggattggttcacgtaatgctacacaaaaacacatggaacactttattacatttttcaaggacgtaaaatgaaacaactacaagaactatatcaaagaattctggatcaaggtgtcgaaagcgttgatcgtactggtgttggcactatcaagctaatcggacaacaaatgcgttttgacctaaaagaaggtttcccagcagtaactactaaacgtttggcttggaaagcaatgacttcagaattgttatggtttattgaaggttctcacgatgagcgtcgtctgtgtgaaattttacacgggacacgtgatgtagaaaagaaaacaatttggacagacaactaccttaatcaaggtgtaagccttgggtattctgatggcgagcttggtccagtttatggtaaacaatggcgtgaatgggaagattataaattcagttccagtactgaagaaattcaagaactaaaagataaagggtataaagttattgattatactggttcacatggtgttattatgcgtcgtgtaactgaccagctaaaacaagttattgaaggtatcaatactgatccatacggtcgtcgtcacattctaagtgcatggaacgttggtgagcttgatgctatgactttgccaccctgtcatagctttgtgcagttttgtgttgtacagggtaaactaagttgtattcttcatcaacgtagtgctgatctattccttggtgtgccatttaatattagtagttactcactactaactcatatgattgcacaaattacagggttagatgttggtgaatttatttggaatggtggcgactgccatatctaccaaaaccatattgaacaagtaaaagagcaactccaacgtgaaccacgtacattaccagaattatggctagatcctactattactgatattgaagatttcactatggattctttcaaattagttaattacgatccaatgccaacaatcaaagcaccaatggctgtataaaagaaaagggagacttatgtctcccttttttattatcttaatgtaaccagtgtacgtccggcaaatctcacatcaggagttgcaggaatactgaacactacgcttgttccattgaatgtgacactatcaacttcaacacgagtgtaaacaccagtacttacgagttcttgaatttcagcagttactatgttactagcagttactgttaatgtgtaatatgagccgtttaaaacccacgctgacgcagtaaacgggtcagtacgtacagaaccagaaccaattggaacccattgtccttggaccttctgcatcgatgttcgttcggtatttgagtatgcaatcataccatcaacaccaactgagttatcaattgtagccagtactggtaattgaataccatatcggttgaaacgagtacgaataacaccgttatcatatatacttacatcagcataacttcccatagtgaaacgtaagtctgcaccaagtagttttatatcactttggtttgtagtactttctaatgtgaatacaccattctctagtaagtttatgtttttgaatttgttattagttccattgaaattagtatttgcattataagtactaacaccattaactgtaatagtgtcaccagtagtgtcgcctagtgttgtattacctttaacaactaaattcccgctggcagtgatgttacctgcgtttaaatcaccagttagtgatgttgatccatctacaataatatttccatgaaaatgtgattcaccatttttaaattcaacttgaactttattcattgaacttaaagaaataatattactacttgcacttatccctacgtttgtgtttgatgcattatcaaaacgtattactggcgtagctgctggtgtagcggtagtttggaataatgttaatgttccattattaattctaacacgctcagttccggctgttgtaaaaccaattgcatcatctgctggatggaatataccagtattagcatcggaccacttagatgggtcattacctgggttgttcgttgggtcaaacggattattattaaatgtcaatgctggtgctgctgcactgccatacaccaccccaaaacgcccagagccgtctgttccatcattatacatggaatcgccagttctacgtaaacactcaatcacaacgttatctatggatgtttgaacggtccctgcgtttgttctaaattggcgatgatttgcaatcattacagtattgtcatataatactgaagcagcggaaatgcttgttcttgactgacgaagacgtatattcttatcttcagttataataatccaatcgccatctgaaagtgcagtttggttcattggaacaccagatgcaaatggtttggtcaagtcagtatctgttgctaaattttcaaggccatcatgtccagtactgggaatagatgcagtaataattctagcaaaagcgtatgttactagtttagcaccagatatatttggattagagtcaacataatataaaatttcaatccaattaccttgtccagttggatttgtaggtgtaaatgtaacaggacttgtttgtggattatttgcttcataatgtgcttgtgcggcatccattataccacgaacgacataataatcaccattaagcgatggtgatggtgctggtagtggagtttgtgctaaaggtggatcgatatcatctgggatgggattatatgtaccaacaaattgtaaaccactagttactgaatctggtaattgggaagtaagaagtttaccgtttgcatctaggtttgcaaaacgtcgatatgccattgaaccatcttcaaagtctagtacaccttcaaatgctttccatcctttattgtatctaatcatccctgatctaaccacatcagcggtttctttagtcggtgctttaggtaattctagaaatgcctgactatctataattacaccaccatcacctctaattcgatttatactcatttaaatatttcctcagaatagattatatattaagtatttattttagataaaagaaaaggccaaccatagttgacctttaaaaaaaatataaattaacgaatacgctcaggaatatcaataccttcatatacccatacagccgcctgttgtgctttataaattgcatcttcatcagcatcaggaactgcatcaatgtcagcacattgcttattacactctgcaaatagggattcaaccatagcaaatgcttcagtgtatagttgagtcatttcagcagcagatagttttacaattgcgtcatcaaggaaaatcgaaacgctatcagaaccggaagttacttgtgtataataattaagaagcatttgacgaccagctagatcagtactataacgatgaccgttaaacatataaccagcattaatacgatcactacgagatgatactgtgcgtgctttcagatcagatttaactgctgcaatgtccatatcaacataattataagttttcagtactgttaactctttatattcaccttctgcttcttctaagaatgtgatgactggttgattactatcttgtagtttaatatattccaaattctcattaattgcgttcacattaccaacattcttatattccaaccaccagaaaccctcttctttacgaacagcgttattaggttcaatttcagaaccatctttacttgaaataccataaagtgcacgaatatcgctgtacaaataacgatatgtttgttcatcatctggagttgctcctgggcgttcttttaaacccttaacaccaactgggttggagatgatgttacccaattcataattttctttagaactaccttttacatatgtaaacatataactacctcacttgttaagatggtgtcaccataattgatgacaccgtttttttttattataattattaagcagtacgttgtgcaatgaacaaatcagtaactggacttacaccatcataaattacttggtaaatggtcacgctaccagcagttttgtttagttcaccatcaacaacagcataagtaccatcagttgtaaagtttctgttaccagttgtatcttgtttaacatagatgtatactgatgcagctttaccgccaccaattaggccagtaattggagccaacgtggtatcttggtctagagtgatgaagtacacgttacttgtaccatctggagtccatgttccggttggagcaattactgatgatgtaacttgagttgggattacatcatactttttggtagttactgttgtagtagttaagttagcattagtagtggttagttcaccagttactgttactgccttatcaaacacagcattttctttaaatgatgaagttgcattaacggttagaacgtcagccgctgcgtcacctaatgttacgttaccattcgcagttagtgctcctgttactgctagtgttccacctactgttgcgttaccagtaacggatgtacttgcaagtgtagccaaacctgatgttgtaattgtagaaacggttgttgcaccgaatccagcagtaccagtcgcggagatattaccacccactgttaggttaccagcagtaatacccactgtaccagtgatgtttgcaccacttgacatagttactgcaccaccaaatgaaccagtagtagtaacttctaaagtattacctttaagtaatccagttgcagtgatggaagtaccagcagtaattgcagtacctgcacttaatgtggttccagcagctacgttgttagttgcagtcaaagatttaacagctacatcggaatctgcttggttcaattgtgcatttagtacgccagtaacagtgaagttaccgttgtttacaacgttaccattaacgattaggttgttgttttgtgaaccgacttcaccaattgttgcagaacttgaaataattgctgcaccagcagtaatattaccagcagaagataaagaatcagatgctattactgcaaacggttctagtgtttgtccaccaacatcgatatctgcgttaataccaccctcaacacgcagtttaccagcaatataagtgtctggcataatctgtacagtttcaggctgctcacctaatggtgtaccaactgttagagttgcaccagttttaacagaacctagtgttgtttgaccaactacacctaatgtaccaccaacggttgcgttgttagttactgctagaccagttcctgaaccattcaaggttgcagatggagcagttactgcaccagacgttagtgttccaccaattgttgctaaaccagttacacctaatgttccaccaagggtagtattacctgtcgcatttaatgtttcagatagagttagtgttttagcagagatatctgcaccagttagatctaagctaccaggagttagtgttccaactacagttacatcgtttccaaatgtcgcagcaccagtaacgccaagagtagtactgaaagagccacttgttgcagatacaccagaagttactatagaaccaagtgatgcttgaccagttacacccaatgtaccaccaatagttgcattattagttactgctagtgcagtaccagtaccgcttagggttgccgatggagcagttactgcaccagttacacccaatgtaccacctactgttgtgttaccaccaaaggaagctgcaccatcagagtgaatagtttcagatactgacaaactattagctgtaatatcagatgttgttaggtctaaaccacctgctggggtgaatacaccttcaacagttacgtcattgtcaaatgtagctgcaccagttacacctaatgttgaactaaatgtaccagtactaccagatacagcaccggaaaccaatgaaccaacagttgttacaccagtaatattcgcaatacctgttacactcaatggtccagaagttaatgaaccagttacacccaaattaccaccaacggttgcgttgttagtaacatccaaactaccagcattcactgtaccagatgcagtaacattagtagtacttacaccaccacttagtgtgcttagtccagttgcagtgattgttgaagcagttgcaagactattagcagaaatatctgcaccagttaggtcgataccaccttgtggaaccaattcaccttcaactgttagtttttgaacagtactatcaccagatacattcaaagatgcagcacttagtgcagcaactgtactttgaccagttacatttaaggtagtgtttacagtagtgttgtttaaagttgtatcaccggatactaatatagcgtcggtattaataccacctgcaactacttgaataccaccagtgcttacagttactttcttaccgatatttacgatatcagttagggtagttgcaccagttacttccgcagtacctgcaactgaaaggttaccagatacagatgggctgccagttacaacaccagttgttagtactaggttaccaccaacggtagtattaccagaactagcaagggtaattgctgaaacattggaagaacttaaatcaattgaaccattaacaactaggtcaccaccaattgtagcaccttcagtagtacttagggatttagaactaatatcggtagtacttaggtcaattgcaccaggggtcaatgtcccagatacggtcagatcaccagtaatatctgcacttttcgcaattgcaatagtatcattggtagaagtaatattattaacacttaatataccattaatgtttgcatcattgtcaacacgtaaaccttcaccaactgaatttaatacaatacttgtagctgttacttcagtagcagatacttgttttagtgtacttgaaccagttgtttcaatagtagctgcatggacagcaccaatagttgcatcagtagtaattagttttggtagagtgctagaattcactgtatccgaaactgtaataacaccagcacttgtaattgtctctgcacttgcaagagatttcacagacacatcagcagcacttaggtcaacagtacctgctaccacaagatcgccaccaattgtagcaccttcagttgtggttagagacttagaactaatatcagtagtacttaggtcaattgcaccaggggtcaatgtcccagatacgtccaaattaccagtgattgtagtgttattgttaattgctacaccatcagcattgctgattgacgcagtttcaacagatgcaccttttaatacaccagtagcggtaatgttagttgcagaagtaatagcaccagaagcgttaacaccagctagagttgatgtaccagttacatttagtgtggttgcatttgcagtggttgcagtcactgtggtaatattagcttcagtactggaaagtttaggtagaacactacttactgtaccttcagaactaataactgcaccagtactggttagagatttaacagcaacgtcagcagcacttagatcaactgtacctgcaacaactaggtcaccaccaatgttagcaccttcagtagttgttagagacttagcatcaatatcagtagtacttaggtcaattgcaccaggggtaacagttccagttacagatagattacctgcaattgaagtgttgtttgttattgacacaccagcagtgttgctaatgattgcagtttcaacagaagatgcctttagagcaccagtttcaacgctaccagatttagaaagaccagttactacagatgatgcaccagaaatttcaccagttgctgtaatagtagagccagaaattgcagtacctgcaattgcaccagtactattgattgaagttgctgtaacaccagcaagagttgattcaccagcaacagatagagttgttgtaacatcaactttgtttggagcaatatcaagaccgtcaacgtcagcgattgcagttacaccagttgaagttccagttactaccaagtctttaacagtgattgtttgaccagttacggttacatcaccactgaaagtgccagctaccgcagataaatcgccactaaaggttgcgttatcaccgctaacatccccagtaaaggttgcacttgtaccggatacgctaccactgaagatacctttagaaccactaacatcaccactgaaagtaccagaggcagcagataatgcaccagtaacagttgcagtaccaccaacttgtagagatgaacctacagttgtttgaccaactgattcaagagtttgacctttgattgcttcagcagcaactactgatttaggagttagatcttgtccactcacatcaacagttaggccagttacagtacctgtaaattcaacatcattaagaattgctttaccaccaacttgaagttgtggagtagcagggttttgtgttgcgttaccagtaattgttagataatctaatttgtttagaccattaccagtaataccataggtagatagtgcagcagaaccgaaggttgcagtttgtagaacgttcaacttagcaggagacattagttcagattgaacttcgtttgctaacaagactgttggacgtgcactaggtgcagtatatgcaaaacctagcttaacgttagcatcagcggtattttgacccaaattgattgctttgaatgttgcagtacttgcaagaccagtgatgtttgcacccaatgtaactgcaccagttagtgctgaagcaccggatactgataatgttgttgcttggatatcatctaaatcaaaactaccaccaatagtaccagatacgttgatgttgttaaagtttgcagtaccagaactttcgatattaggaatagttgctacaccagttacttctaaatcaacaactttagttttacctttagttacttcaagtaaaggagtggttggtgattgaattgcgttaccagaaatagttacataatctaatttgtttaaaccattacctgtaataccgtaagttgaaagtgaagaatcaccaaatgttgcagtttgtagaacattcaacttagctggtgacattagttcggattgaacttcgttctgtagaagaacggttggtcgttgtcctggaacggtgtaaccaaagtttagtctatatgcttgacctgcttcattaggcccgatacgaatatcacggaagtttgcagtacttgcaagaccagtgatatttgcacccaatgtgactgaaccagttagtgctgaagcaccagacacattaagtgttgtagcattaatatcagaaagattaaaagtaccaccaagtgtaccagaaatgtttacatctttaaaagttgctgtgccagttgaagtaatatcagttgattcaacagaatcaaatgatcctgaattagcttctaagttatcaaattttgcagttgtgccgacagcagcagtaatgtttcctgcaaatgtcgcagcacctgaaacatcagcaccaccggataaggttgccttaccagcaacatccaatgcacctgtgacattcacagaaccagcggtaattgcagtgaatactgcgttagcaaaagacacttccccattaaccgttaggtcattactgaatactgcattacctttgaccaaaagatccttacctaccgtggaattaccagatacatcaagatccgttttaatcgagtacatagagtcccctcctataacgatgaactatatattcataacccatcttttgttaatatacttatatttagtttaattcacaaaaacactattatctaatacaaaagaaaaagccaactattttagttggctttaattattaatttaaaatgttatacacgttgtgcaacgaaggtatcaaacacagtactgaaaccatcatagattacttggataatacttgtttgaccaaccacagcacttactgtaccagaaactacagagtatgcaccaccgaaggtgaaagtagtatcagcaacagttggagttacatagatatacactgaacatgcttgaccactaccaattagacccgcgatggaatccattgtaacactttggtcaagagttaagcgatatacgttagtagtaccatctggtgtccaagtaccacctacagcagcagtagaagcagtaacactagcagggataacagaatatttcttagttaccatgttgtttgtagtgaagtttgcagtggtggtgttaatagaaccagtcactgttaggttcttatcaaatgttgcgttctctttaaatgtagatgttgcatttacagttaaagtatcagcagcagcatcaccaatcacagtgttaccatttgcagttaatgtaccacctacagttagagcaccagttacagatgcacttgcaagggtagctaaacctgatgttgtaatggtagaaacggttgttgcaccgaatccagcagtaccagttgcagagatattaccacctactgttaggtcaccagcagtgataccaacagtaccagtgatgtttgcaccacttgacatagttactgcaccaccaaatgaaccagtagtggtgacttctaacgaattaccttttagtaagcctgatgcagtgatggatgttccagcagtgattgaagtaccagccgcaacgttattagttgcagttaaagatttaacagctacatcggaatctgcttggttcaattgtgcatttagtacaccagtaacggtgaagtcaccattagttactacgttaccattaacagttaggttgttgttttgtgaaccaacatcaccaattgttgcagctttagaaacgatggttgcaccagaagtaatgttaccaacagcagatagtgttgcagcagcttcaattgcatatggttctagtgtttgaccacttacgtcgatttcagcgttaataccaccgattacacgcaatttaccagcaatataagtatctggaatgatctgtacagtttcaggttgttcaccaactggagtaccaacagttagggttgtaccagttttcactgcaccagtagctgttagtgaagatacagtagttgcaccagtaacacctagagttccagttacacccaaattaccaccaactgatgcgtttgcagtagaagttaaacttccaacagttgctagaccagatgcagtcacagtagaagtactaacaccaccacttagagtacttaggccagttgcattgatagttgtcgcagtagttagactattagcagtaatgtctgcaccgcttaggtcaagaccaccttgtgggatcaattcaccttggacagttagtttctgaacagtactgtcaccagaaactgttagtgatgtcgcagatacacctgctagagtagaagcaccagttacacctaaagttccagtaatggaagttcctgctagagtagaagcaccagaaacgttaattgtgttagtattgataccaccatcaactacctgaataccaccagtactcactgttactttcttaccgatattaacaatatcagttaggttaacagcaccagttacaccaagagttccaccgattgttgtattaccagagatagctgggctaccagtcacaacaccagttgttagtactagatcagttccaacagtagtagaaccagtacttgctagacttgcagctacaaggttagttgcacttaagtcgaatgcaccatctacagttaggttaccagcaatgtgtgcgttacctagtgaatcaagagatttaacggttacatcagtattgcttaggtcgattgaaccagcagttagtgtgccagatacagctaaatcaccagtgattgcagtattttttgcaatattaagagaagcagcagttgttgttaaagaaccaacagttgcaatgccaccaacagataatgcaccaccaacgtttgcatcattatcaacttgaagaccaaggccaccaccagaaaattcagcactagtagcagttacttgagtagctgatacactttgtagtacactgttgccagttgcattaatagtacctgcatttactgtaaagagtgttgcatcagttgaagtaattttaggtaacgaactagtattggttgaatcggtaacggtaataacaccagcacttgtaattgtctctgcacttgcaagagatttaactgatacatcagcagcacttaggtcaaaagtaccatcaactactaagtttccaccaacattaacatcagcagtagtagttagtgagtttgcagtaatatcaacagttgttagatcaagaccaccttcaggtttgaatacaccagtgacagttagatcttttgccgcgacaacattacttgctagatttagtgtaccagcaccagcagcttcagcttgtaaagttggaactgttacagaagttgtgattgttgcagtaccagaagacaaacgcgaaccagatactacaccagtactagttagtgttcctactttcgctgcaccagtagtagttagtgtaccacctgaaacgtcaccagttgcggcaacagaagatgctttaattacaccaaatgtacttgggttagttgcactaactgattttgttactacatcagcagcagacaaatcaatagcaccagttactgcaagagaaccaccaacagttacgttaccagtagttgttaaagacgcagcacttacatcagtagtgcttaaatctatactactcggagttagtgttccagtaatagttacgttattagcaaaagtagtgtcaccagtaatacttggaacagttagtgaagccatagaaccagcaccagttactgcaagagaaccaccagatagtgcacccgccgcagttactgtattaccagatacagacgcaccagtaacatcaccagtactggttactgaactagcagttaggccagctaaagtagtgtccccagcaacagatagtgttgttgtaacatcaactttatttggggcgatatcaagaccatcaacatcagcttgggcagttacaccagtagttgtaccagttactactaaatcacgtacagttacagtttgaccagtaacgctaacatcaccactgaaagtacctttagcagcacttacatcaccagtgaaggtagcagcagaaccagttagtgcaccaacaacattagcagttccaccaacttgtaggttagttcctacggtggtttgaccaactgattcaagggtttgacctttgattgcttcagcagcaactactgatttaggagttaggtcttgtccacttacatcaacagttagaccagttacagtaccagtgaattcaacatcatttagtactgatttaccagcaacttgtagttgtggggtagctggactttgtgtaccattaccagtaatagatagataatctagtttgtttagaccgttacctgtgataccataggttgcaagagatgcatcaccaaaggttgcagttttttgtacattaagtactgctggagatactagctcagactgaatttcattttgtagaattacagttgggcgagcaccaggggcagaataagcaaaaccatacttaacgttagcatcggctgcgttttgtcctaaacttactgctttaaaattagcagtactattagcaccggaaatatcagcacctaaagttacaccaccaacaaggctagttaatccagaaacagttagagtgtttgcttcaatatcatcaagactgaaagtaccaccgagtgtaccagatacgttgatgttattaaaattagcagtaccagtactttcaatgttaggaatagttgcagttcctgttacttcgaaatcatgaactttagttttacctgaagttacttgtaatagtggagttgttggatcttgaacagagttaccagatactgttaggtaatctagtttgtttagaccattaccagtaataccaaaagtggatagtgacgaatcaccaaaagatgcagttttcaatacacttagggtagctggagacatcaattctgattggatttcgttctgtagaattacagttggacgaagacttggagcagaatagttaaagctcaaacggtagttagtgtttgcagcattggaaccaattttaatatcacggaagtttgcactactattcacaccagtaatgttggaacctaatgtaacaccaccttgtagactggttaaaccagaaacagttagagttgttgcattaacatcagatagactaaatgttccacctagtgtaccggaaatattcacatctttaaagtttgcagttccagttgatgtgatatcagtagttgcagtgtttgtgaatgttgcatcagttgcagttattttgtcaaatgctgcggtagtgccagtttcagcagtaatattacctttaaatgaagaagttccaagaacatccattgatgccacttgggtatcttttagtacagcagtgccagcggtttgtgttaggttacctgccattgatactgggctttggaattgtgcattggaattaatcgttgcagtatcagaaccaacaggcaaattaccaattacggaattaccatgaacttcaattccaccattcaatgatgctttaccagcaacatctagtgtagatgaaatgtctacagtttctgcttcaattgcagtgaatacagcgtcagcaaaagttacagttccattaactgttagatttttattgaatactgcatcgcctttcacaagaagatctttagcaaccgttgcaccaccagttatatcgagatctgttttaatcgagtacatttagttccctccaaataggcaaaaagcataaaatacatttgaccttgttaatatacatatatttagttattgatataaaaaatccagactagatgaatcatagtctggactattttttttatttaccggaaattactattttaccactaaacgcttcgtcagaagtaaatgtaacattaccacttgcatcaatttgagtttcggttattaccagttcaaatggtgatgctgctgtactgcctggtgttttatttgaatatatagaaatatacaaattgtcaggtgtttggttatgttctgacgctggaacagtaatggtgtaatcatcaccagatgctacccaatctgctatattaattaatttactataaggtactgtcattgttgttgctcctatgatgttgactttgacacttgtatattcagtgatttgaccaataacatcaatcgtaatgtcaccaataccactcacagtgacgttagtttggaatacagtgtcaagtgggatgttaccaggatcaccctgtagctgaaccataatatctggtccacgaccatgtactgattcaggtaaagaataagtatattgatcagttgttccaggaactaatgtaaatgccgatacaggaacgatttcagtagtaacgtaagaactatcagtacctggagttgtgctttggagaggcttccaaacaccaaggtctttaatatagatctgaataatattagtactgtcaactaagaagtaagcgtcaccatctacaccgattgattcagatggtgcaatagttccacgtaaaataccggaaccaccaccaccaccgctatgtgtattcaaataacccaatgtaaccaagtgtgtatttgcggttgggtcacctgctgaaacaggaagaagttgtgtacccgtgtagttctggatttgaacaacattaccattagtggcaatttgtgcaccatttttaccaagtatgaaggtcttcttcatggttccttctaaattagaaaaaactgccatttttataatctcctttaagatagggtgatatatgttattggttgagcagctttcaataatgcccctgctgcggtagcagtaacaatcggatcggcaatatcgatacgatacgcattattaacaacataaactgctctagtgatatatgtttttaaatcttcgccattttcccctgctcctgtgattaggatttcggttccagcagctttagcatattgattatcaccccatgatacacccaatgcttcgatataagtttgtcctgcaactgtttgtctaattgtgtcatatcgaccaaagaaagaaccacctactgtaacaataaattttccacctgattgtttatggataattgttcctgctgggaaatatctcccaccaggaaaaacatcataaggcacgtaatactgttcaactacaggtattactacaggtccacgatgacccgttccatcaattgcagtggttactttaaagttattagatgattggtcaatacggtcaacatacattcttgagaaactatattttgatatgttacaatcatcttgaacaacacaattagtccagttggtatatgtatatttagagcgagtatataagccccctccttcaccttcagttgcaaggtgttgtccaataacatcaagttctttagtactgatattattaaacttggaagatgacggcaatccacgccattgaattctactacatgtaatattagaaagttcagtagtatttagccaattatcagagcctgaagtatacacaataatagacgactcaggaatgtggagattacataatttaaagtttcgtggaggaccagcaaatttaccagatgttacgccccagttaatagaaaccatacggttagttactgatggatcatttaatagatgcccagataggccatcaataataaaatcaacagaaccataagtaattacaccagcggttgtcaaaccagtaccaattgataccaacgttgatggataaacttcattattgagaattttaaagtctttacccaactcaacgtgaatacattgacggacattgtacaagcggttattttttataacgaatttttttacatattgtgaatcatcagcattcacatcataaggaccagaacctgcaacaccgataccaataccccagtttgaaagaggaattggtgcatttgtaccatctacgttttcaatcaagttaccttctattacaaatccgtctgggtaatgtgagttgactacgttcaattcaattgcatcaccatatatatcatggatgtggttgtatgaataacgcccaacggtcattttctcaccagtgccttgctgaagaacaccgtacttacattgtgtcatttcattaaagcagaagtcgatagtacctgattcagtcataccttgcattagaaatgcagcagtacttgtaatattaataaatttattaaaataaaatttaccattaccaccaaagaattttaaataacatcctgttaatgttgcttgttggattgtactattcagtaccgataccattgtgccaccgttaccagtgattgtaccaccaaaccattgaatatcattcaaataaacagcagttctgatacggtaattaccaccagcttgaatttggattttgtgctcttttgcataaactgcacatgcgttcaccgctgctgtgtcatcagtaatgccatcaccttttgcaccaaactcttctggagttatgtaaaactgtgcctgttgaagtgttcctccagtaataagtccgatataatcagcaccgttaacactggaaaggacatttttattaatgtataatgattttacccaaacatcatttgtttttgcaaagatatcaacaataccctcagaatcaacttgaaaataagtgttgttatttttaccaatgtcatttgttggtggaagtacgccaaataaagcatttgggttatcttcaagatacctaacgtttactgcggaatcatcgtcattaggagttcccaccttaacaggtaggaacgtttcacctaaaaagtctttgattgcgacagaatcgtttacaaaattaatagaagcaccacgtttacccaacttgaatgttggcatcatggtcccttctaaattggaaaaaactgccatttatcaaccctcttttaaatgttatcttgaataggctgaataggtgcttgccatgataaagtacaaacgatcttcatttcaccaaatgaactgttgtttacggaatcattgaatacacaagaaatgatttcatctttacttgatacttcgtggttggtttcgttaacaatgtaacgcattttgcggttgatatcgaaataatcaacaggtgcaattacagtaccaccaatatcttcttgtgcttcaccaccgaaacgtacaaacatttcatttactggttcatcatatgcttggttaatatcaacttcgatattagttactaggtatccagcagggcatgtaccaataactaacaatgcgttagtccatgaatgatcaatagtgttgatgataacaactgtatctttacctggttcaccctgaataccttgttcaccttgcttaccttcttcaccgtcttttccacggaataagataccagcactccactctttagttggggttgatgatgtagttacgatatcgacataatacaaattaccatctgtcgtgttatagaagcaataatcttgtggtagattatcaatgcttgggaattgactaccagtactatcaatatggaaacgaccacgtagaccttgttcaccacggaaaggaataccatcactccatgttgcagttacgctttgatagaaatataatttaccatcttcagtattatagaatgtgtaattattgaaaagaccagttgttggaaacaccgtaccagaactgtcaatgtggaatggtttaccagtttcaccagtatcgcctttaccaaactctacacggttccaagtactaattgcttcttgtgcatcgtttgatttaaaatataaagtactagtgccatttgcaagactaagataacaccaccctagttcttcaccaacacggaagagttcattaggaacttcaaaacctatctcatttggataaaagtttctaccgttagcaccattttgtccaaccactagacctaggtcttggattgtattgtcggaataggttacaattaaatgtccagcagcgttaataactaaacttttgatgccagaattattacatgaaccacctgacccattgttgaatgaaattgtcataatatttcccttgtatgagttaatgtcatataccaatatttatcagtacaaaatacatttcgttgccattaataaataaatgaaagttcactactaaaagggcttaacaatggcattaagatcaaaagacttcctcttcaaaagaaaaactaaagatggttcatggcaagaatttcctgaatcagatagctggtatcctgctaacttagattatcattggaacgaacaaacattaccattagctagtggcggctattatggtagtactacaggattacatacaatatcctttaccctacgtaacttcattggtagaatttacgtactcgctacactggcaagtaatccatctgacgatgattggttcccaattaaatttaccgaaagctgcaacttctatcttgaatttactgataatgtcattcttaacccaaatggcggcgaccttattagagaacaaggtacaactggtacattcgctgaaaccttaaccggaaactttacctatctgaaagttggtattgaccgtgattacattagcaagtatccatctgatgtacaaaaaagtttagctggtaagttagaacaaattttaattaactactaaggattagaaatgaaattagaaacagaacaaccaaaactatttgttaagggtcatgtactaattactgacgttactgatccatctaaccaaaaaatcgttttggataaatcaaacgcaattcaccctgaaaatatgagtactgctattgctaatgcgttggcttataatcaagatagtactggtattcctattggttctattagtgagatgcgttttggtaatggtggaacagtagttttgagtactggtcgtatcacatataagaaaccacgcgtgacttctttcggtgggctatactctcagacttatctgaaaaaaatcaacccacgtatctctgctggtattgattcacaatataacaacattacaacggttcacattccaggccaagcttacactgatatcgtctgtatttgtacattaggtttaggtgagccatctgaccaaggtgttagctctggtactagcatggatgggacatatgtatttgacgaattgggtttatatacaaatggtgatagcggtattccacttacccatattatttttcacccagttgagaaaagtgctaaccgtgttttgcaaatcaaatacacagttcgtgtccaactacaataataaggtgatgatatgagtgtaagttatacaatttatagaagtaatggtagcgtttatgctaccattccaaataacgtaatattaggaccaaaccaacctggtagtaacccagtaccgttaaactttgcaggtcgaaataaagttggttacggtcaagcctggaacgaaaacttcctccacttggttgagaacttcagtggagctactgcacctcgtggtaacgttaatggtcagatttggtacagaaatactggcaacactggtgaactacttcttgcattagttgataatgctcgtcaaccagtagatactgcatctgaagcagactgggcatctatcccaatgatttcaattttcaacacagtaccagatggtgaaaatagtattatgggtcgtatggttcttaccagcaatggtgattctcttcgtgtcctaatgaaaaataaagaatggcgtgaaattcaaacatctcgtcctgaaaataaagaatactccacactacttgatatcaactatgatacaggtaagaaatacattgaatttacacaaagtaacagtactaagccattagcttacttcaatatgggtggcacttcacaggttgattcaaataactggacaacattcacaaatggtactggggtgtttgaatttggtagtaactatttttatgaattgaaaattatgattcgtgaagttagtgattcaacaggtgaaattgtttctcttccgtataattcaaaaacatttttcattaaaggtcagttctatgttgataaccaaggtacatttgttcccggttcaattggtgcaagtcagattccagatccacgtcgcattacacaactagaatcaatcaaggatgtaattacaagtagtcgcccgaattgggatgtttctatggtaattaatgatgttgacccaactctcccaaatccgaatggtatcacaaaagcggattacgaaaactgggtgattaactctctcaactctaataaaaacttagggttccgcgtcgatggtatagtaaacaacctttcggcaggtgagtcagttaagcttcaagcatctgttctacttaatataacaggtatcccactgattggggtataaaaagaaaaaaggagccaatggctcctttttttattaatatcgatatggtaagctgttgacaaactgtgtaccttgtcgtatactttcccacaatgaataaaaatccttcattaaatttgcgttgagtaatgtgttttttgcaccaggatgtaatggtttgggccatgagccaatcttaacccatgcataacccgaactctcatgatttaaagtaggtacgaattcattttcaactacaatgacaaatgaatagtatttaaatttaccattcttagtgcagaacacatcaaatggtataatatctattatcgttggaatagaatcacctagttcttctttaatctcacgcataagaccctgtaaaggagtttcattatactcaagcttcccaccaacaaatgcccagttgttagaatacgtacttctttctgatctcaagtttaacatcatgtgtccagtacttttagataaaaatatagcacctactgcttcattcataacaacacctccttatttgtcagtgaaagttattttccaatacccctgacgatatactgacataatactcttatgccatttgaatttgtcattatatctataatacccactatcttccatattataaacatagttgtgtactagtgatggctcaataagttctgctccatcattggtaattttaaacaaatagttcgctgggatttcagtctctttgtaaatgatgttatgtatgctattatttacataatattgttcaggcagtgcgttatataacaaagagcttttactaatgaatccatgtattgtataatcaacagtataattactattatcaagctgaactacaatcttgttagattcagcaccttcaccaaatgaagagatgtgtccttttagtgggttgtcctcttctatagcttgttggaatttgataccaaccgtcttgtcttcatattctgggtccaagtacttaaggtatttttcccatgaataaattccaccatttggtgatgtttctgatagtgattgactaacaagagtgagttggtaagtatctggaatattaagttcagagtttgttatttcatcaactttaatacataagttctttggtgtgaatacatcagtaatgatatccgagtaattaccaatttcaaaatcaagttcatcacgagcacctttaatattggtggtaatttgttgaataagttttggtttctcaacaatagctggcaaagagaaccaaattgtggtttcaaatgtcaaaaccattatatccaaatcactatctgttccttgtgggaaaccacgagtagtaaattgacagtcaattaactccacatcactcaatgatgtccaatccaatgggttctcggagagttgtaattgaatactaggagcaaacaatgttgaaatttgctcaaatagttccattttggtttgtgtttgtgtaacccaaatattaacatcaaaagtaaatgtccaaggtactgggttatgacgagttactttatactgtttatctaagtctttagtatattcgttaattccaggggacttgttagtccccataactaatgtttcatttgcgttcgaacgaactgacttgcgatctagtttaagttggtttacactaatggtcatgattggtgctgttggaagcacgttctcactgttaccattaataattgctgctgccatacgtgaaatatctgcatagcggcaaggaactttcttatattttggttttccgtcagcatcaacaccattttgtacttgaaattcaccaaacactctgataagatgtttcagtactcgacgaaattgaccatcataataataatctattttcattctgaatccttatgtatacgatacactgtattaggtttaactgcatcatgtaatggtacacggttcgggctattaacaaattctgttagagcaagatctgcaccttcccacggttttctgccgccgtaatcgaaaattacccaagaattcttggacgcactgtattcccatagggttactggtgtataatcggtacgatagtagtaatcaccatcaactgcatcacttgggaacatattaccttcaggaactgtacctcgtgctggaggaatcttaggacgataatcttgttgtgaatcgatatttccataaacactattagaagtgaatgggtcattaacgtaagttacttctggttctccccatttaccatttaagtactgataaatgtgtggttctttgtattcaatataaaaatcatcaaccccttgtggtggtgtgctttctgtagtatactcaacatcacaatttagccactttttggcagatttatcgtattgatacaatgagaagccagtaacatcagcctcgttttctttaaagaagaagtcaccatccatcggtttgcgaggaaagattgttccacgcttgacttctacagaaatccatgtatcattagatagttctttcagtactgggtaagttttcttgaggaaatacattttatttgctggttcctctggtaattcgtatccatttagcatatacttagacaaatcaacagtatctggtctgtcatcgaacaagtgttcgttagtaaaatgaatgtatggtacttcatcgtcagcttgcataatgatacggttcataatatccatttcattaccatagctgctgttctctgaagagccatcatcagtattaccattgccgttgtttggatcatctgggtcatcagggtagttattgttacctagaaggtcagagtactctggtgaatcagtaagaggtttaacacggagtttgtaaatgtgatgttgccatgtaactgagtaaccttctgacgttctaaacccatcttgtactacatagaagcggtttaaaccagaatctttaccaagtacatcaaaatcacgtaagttaggaagttcgattacgtcacctggcataatttttcttcccaaacgctgaatcattgtgttataatgaattgtgatgtccattgtgttaaaattgaagaacaatcctgggattttcaagtcaggagttgcttcttggacttggtagacgcaacttaaggtgatagcttcgagattatagttacgacttggagtgtccccaaaaattaaatcaccaatgcttgacacaacggtactatctgatccataataatcaccgccgtatgataaatcagtgttatcagcgacttgttttggtccgacataggcataaagccacatgtcaacaccaccgatgttgtattgttctgctatggcagagtcaatgaacttgtaatcattggttctaccttttttccacattctatatgccatgtttgtctccaaaataaatcgggtatatctttatttaccttgttgacaaacgcaaagaatgtgatataatgtttattaattagagtgactactcatttaggagaatacatgaatcttacccaattattcaaaaaaagcgacgaactggcaacttctctgggtcatacattcttgactattgaccatctaagtgctacagtactgacaatcgatagcatcaaagcagtatttgaagatatgaacatcgatgcagacaacctacacggtcgtttgattgcatatctgaagaatacaacattcccacatgcacctaaaagtgctgatggtgatgaagcgaaccgcacatcacttctcgtgaaccgtgtagttcaggaactttctaagaaatcagtaattgagcaacttaaacgtgatgatttctcaattgatgcttattttgtactgtttgagtgtttgtccttcccaaatactgcactagaagcagcattgcgtgaagagggtatcacttgtactgaagcagcacgaactctacagaagcacgtagaagcaatcgacaacggaatcaaccttcaaagcattagtgatgagccaactggagagaaaaaagtggcacaacagcaaccatcgtccaaacaaaaaggtttagaagattacaccactaacctaaccgcacttgcaaaagaaggtaaacttgacccaatgattggtcgtcagaaagagctttctgcactgattgaaactctggcccgtaagactaagaagaatgccatcctgactggtgaagctggtgttggtaaaactcaaatcattgacggtctagcacaggcaatcgctgatggtgaagtaccagagcatcttaaagatgttgaaattctgtctctgaacgtaggtgcactgactgctggtactaagtaccgtggtgagttcgaagaacgtatcgataacctactaaaagaactgaagaaacgtgaaaacactattgtgttcatcgatgaaatccatattatggttggtgctggttcagcaagcaacggttctatggatatgagtaacatgctcaaacctgcactatctcgtggtgagattcgtgtaatcggtgcaacaacttacgatgaatatcgtaaaaacatcgaaaaagatagtgcattaactcgccgtttcatgaaaattgatgtagttgagccaacaattgatgaaacccgtgcgattgtaaatggtttgaaatctacttatgaaactttccataacgtagagttcagtactgaagctatggatgcagttattacactaagtgataagttcctgaagaacaaacacttccctgacaaagcaatcgacctaatggatgcggcagcagcgaaagtacgttctcgtgaaactgatgataacatcatcactgttgcactgattcaagaagttgttgcaggtatcgcaaacctagatattaacgtggttgcttgcactgaaagtgaacgtatgcgtaatctaccagatgcactccgttctcgtgtatacggtcaagaagaagcagtacagaaactggttgataacgtgatggtagctcgtgctggtctgcgtgataaatcaagtgttcaaggtgcattcatgtttgttggtccatctggtactggtaaaactgaaattaccaaagcactggctgatgcaactggttctgaactgattcgtttcgatatgtccgagttttcacaggaacatactgttgctaaactgattggttcacctccaggctatgttggtcacgattccggtaacggtctgctactcgacaagattgagcagttcccgaatgcagtactgttgcttgatgaaatcgaaaaagctgatcgtaaagtactactaaccttcctacaggttctggacgaaggacgtttgactggttcacaaggtaaaactgtttacttcaataacgtaactgttattatgacaactaaccttggtgcagcagattcaaccaagcgtagtattgggatgagcggtagtgatacagcacaagataaagcgattaaagcattcttgccacctgaattcatcaaccgtatcgatagtattgtgacattcaaagacctaagcagcgaagcgattgacagcatcattgataagttcatgggtagtctgaatgaacaaatcaaagaccgcaaagttaaagtttccctgactaaagcggcacgtgagtggttggcaacaaatggtgttaaagttgggatgggtgcacgtccaatgaaacgcactattgagcaaaacatccgtgttccactagctaaagagatgttgtgtggttctctagttaatggtggtaacgtgacgtttgatgtgattgatggtaaattggctatcaaacctaaacgcactaaagcagtagaaaaagttattgcgtaaaaaagaaaaggagagcatttgctctcctttttgttatttaacttctaatagatttttaatgtcagtaccacttatttgtaaactatcggagtaaacagatagttcgattggataactgttaacactataacgttttagtaaattaactttacctaagttcaagccattaaatgaatatgtcacattctctttaaagtcgtgttctgcactatacattaccccacggagtaagttatttgcaaaacgtacttcaactttttcaaaaccaagagaaataatatcattcatacttgcaccagcaccagatagaacattattagtaacactatatggactaactgaatctggttgtgaaacataagtagcattttcttcactgatagcagtaatgttagttactgttccaccaactacaccaatatatgtatcactaactagtggatatgcatattcccataccccttcagtattcagtactggttgaataataccaacatcatactcgaacacgttaatttccacactatcagtcattttggttctcactaactcactaagaacacctgcaccagtaattggtaagttatatccgttatcaatggtaacaccatcaacttcaaatatctcgttagacactacccaaacatcaacgtcatctggtgtaattccatcattataagaaccaccatatagtgttgtgtcgtagaatacttcatagttcattgcgtcgatagcatagtttctatttgtttcatacaacttaacaccatcaaccaagaattctggatcagcaaaggttctaatacgaacgatgttcaatgctagaggtattgctggttcattctgttgttgtttagtgatgttatcataacgaagtacaactccagtactatctagtagttctacagttccgttcgcagtgtttcttactgtgtacaaggttggtgtggtgtcagttttgatttttgatacactcaagatggtttcccatgcattgttttggtatctcacgtaatcttcaccatcagttgtgaatatcataaccttatcaccatcagatagggtattgtcaattacttcagcttctgtatcactaacaattgatttaattttatagaagtatttttcatcatacttactgtatgatttactaatatcattttcaatcttagcgtaagaaggaatactaacaattgcttctgttgttttataagggttgataataaccataccttcagttttatatctactattgtctctcaattgaactagtgaaacactattgaatccataaccatcaactgtgaagcgaatctgaccaatctttcttcctgatgggttaatatcagaaatgtcgattatgtacggatctacttcgtcaccttcaacttcccattttaactcggaagtttgtgattcaagtgctaggttatcaaatgcattcatatcatcgtagtacttagcatcggtgatgctatctaggatgtgctgataatcactgtacattgggttatttctatcaagaggtaagtcatcaagatttatttctgccaattgaactagttgaccgtcagttatgaatttgtcttccatgaaatcatcaatgctttggtcatctgggatactctcgtatacaccattaacattaacataaacatattttcgtggcatcatagtaaatgcatgaatatcaataggcattctacttagtatttcttcagccttttcataatggtcggtagcatctttgtcatacaaaccacgcataccagacaatacatgaattgcgtcattgtagaatcgttgatttccatcttcatctacatggcgatatgaataaacaccatattcaacttcaagtataattctaacttcatcatcagttgcagtagtattgttttctttaattaaagaaacatactttttatacaactcactactaacttcaaagtaaccaaagttataatcagtactggatgcataaccaaacggtagtctggtacttggattatcatcataggttaacccactaaatccacatccaacttcatcatgaattacttcatcatctgcacctgtgtacaatttcaaacggttagagagtgtgttatactgaacataatgatttacgtcattaaacatttcattttgtgttgattctttatatacacgattcaacgtttctgtgaattctgcggttgctaccaaatcatctgcatggtctgcataccattggttaacaccgtaagcaatccctagagttacgttcaattctgggtaatacaattgcatctgataagcaaccaataggtcgaaatcttcatcactgtattctgggaatgtatatttgtaacgttgtaattcaaaattactaacaacatcttgtgcattagattgatatttcttattcacttcttccaatacatcaacacttgccaaattatcatttggtttacattgagtgttgtcgtagtgcatgataacatcaatttcttggaaacggcgattaggtgttacgaaatcacttggcaattcaccaacttgctcccaacgttgagttacctcattgaattgagcatattcataatcccaaatgtctttttccgttttaacccaagtcccatctgcaaatatatagccattaggatctaaatcattacacaaacctttaattaaatcatcatctgaagttttctgttcaatctgggaacgaatcttagttctatatggtttaacttcttcaaagtactcaacgatttgattaatcaagttgtcgttggaatagttctgtactaatgattgttctaaacctacaatataaatgtaagatgttttgaatacccaatcaacaacgctttgttctacgtgaacatacttaaccatatcaaacacaatttcactctgatgtaagtcgaatgagttcttcaataaagctagtgctttacgaacgttaatctcacgattactcaacgaaccattagcatcacgaactgtatagtcagcgtataacatatcattcaattcaactgtactattttcacgaccaattgctttccagtaaactgaatcacgtgattgtgctttaatgtacaatgtccagatgccatcaacatctgtaattctgataatgtcaccaacttgatagttcttagtgtttgcatcttctaggctggattcttgtgtatatgcagtatttttatcgtagccaatatcataccaatctgctagtgaccagtactgggtcatgtccaatgaagttcctagtgcttcaacatagtctgattgtttaatcattttctgttgaccttcaccacggttttccaatgtaatggtaaccacactaccagcacttgatggactattcattagatgaatataaccattagcatcaatatgcacaaaatatacagagctagttgatagtgggttaggtaatacaccactagtacttactagtactgcgtcgttttctttgaacttgtcactttcaggtgcaatgactaattcactattgtaagttagtacacgatatgatacttcattcgggttaacttcagtagttttaacattaaacacttcgtccattagaacagtgttagttgttatattctgtgagcgataatagttatttgcactttctacaaagtttctacgagcttcaattacatcttggaaccaagtcttattagaacctaaaccgtttggtactaaaggatatagtaaatcaataacagttcctgattcagtagtttcttgaccaatcatagaattccacatagaagtccagattgatggattgaagttatagctatcaccttctttacataattgatactggtcatgtttaataacttcagtattatccatgtaataagaaacagttaaaatacttgtgtcatcgttgacttcaaattgcatattacttagaatgaacgagctttcattaattggtgcaaaccaaggaatgttaagctgtgctggttctttaataatacgagcaatatcagtacaagatttagttctggaatgcaatggtggtacatagattgtatcacgaacccagaaatagtattttgtaacataagtatcagttgtgctatcatattctaagaacgtactataattatcttccaatgcggttccgcttggtaggtacatagtgttgtttttatttttgttagcaacgtctttgcagtatttgtcccagttatatggttcaactggtgaacatacccattcaagaacatcaacagtactgtgcggtagcaatctgccccagttataacgtttatattcatttgttgcatcaacatccacattaccatcagcatcatagattgcacgagtgtaatctaagtagaaagcagtgctggtgttccaccaaacctgaccaactttatcagcaccccatgcttgaatcaattcaccattgctgttgtattgtgctgggtcaactggagatgtgtaggtaacgttctgcatcgctgcaccaggaataacaccgttgattgggtcaaacacgtttaggtacaacgcagtaatgttagtacgctcatcatatgtggttgggcggtcaaacaaagtactttgtggagctttaaaactatattttctgtcaatgtattcctgacgcatctcttcagtactgaatcgtgaaacataacgatacaagcgtgggcgagtatcaggatcagtgaagtcaatagtgattgaagtgtcaaacggaactaccataactctagggtcattacccgcagtataatattgactttctatagtcaatgtatctggcataaagttagtttcatcaacaaagtaataataatcaccatcatttagtgggatatcatcacttagagttactttacagaattgttctgcatatacatcggtattttcaattggaacgatagagagcatctcaacttcagtgtctactagttcgaaaatattccacaaatcattaccatctttaaacatccatacagaatctgggatttcaccactgttattcatataattgatacgatcattatatgcagtactgaaatctttagtaacgtatgaaatttcatctagtgttacaggaccaatgtctggaagttggatattctcacttcttccactacgcattgggaatgaaaacttaccatttggacgagtaatccagcgtgagtcaaccccttgttcaccgtaataaacaataccattaccagtaccatttgatgtagcattaacatcaaacgaaaccatctgtggttgttgtttaaattggtcattgattagtttaaattgtagatttttcttaccattgatattaccataagtaccaatcttaaatgcccattcctgaagtgctttatattcatcagtgtcgtaagatttagaaacgcgtagtactttttccattacttggttagtacctttgtcatgaatgaatcctttatagaaatcaacacgactaacatcactactaatcatacgttgtagataatcacgagtttgatagccaatcaagtgatttgataactcttgaagctgaacatcactcacaggagtttcactattataatataattcaaaatcagttactaatttttcaaatgatggtaaagttcctgcttctaggataatgaaaccaggtgcttgtaggcgaccagtccatgacccagtaatgtaaccatacattttaatcaattcttgaatagcaccataagttggttggtaaatgtaatcaccaaagatagttttatcatcaaaaacaactgcgtgttcatatgaacttaggctcaaacgaatcaacgccatacgtttttgtgattcggatgtagttctcactgtgaatacgttaccgatacgcgaagtttcgatttcatattgacggataccttggttttcatcatcaagtaatgaccaaacaccaccactgttctgtgtaacagattgaactgaaccaaaatctgcaccaaatttagcattagctgcacttggagtacaggagatgaactcaccagtacttaaattattctgactccaaataacaaagtctttagcaatagttgtccagtcttgttgagtaccactatcatttacatcttcaaaaatccagccatgttcttttagatattcaccatatccacaaatgaaagtataaacttcttggtatgtattgaatattttaccatattctaatgtaccttcagatttccattcattgtaatgaacaacagttctgtcttccaatttaagagttacttttcttccatttttaactggaattttatatttaaatttagcaccaactaaatcataaccatcaatttgatatgcactaccattccaagtaatttttactgcactcatttgctcatcgcgaatgttattggaacgaactagaccgatagtttggttttcttgtgaaatcaaaccgaaagatttagcagtgaatgtcaattggtctgctttagtaaagccaccaattttgtgagcgacattgacataagaagaacgaacaacgtcaccgtatttggtggtaatgttcttattctgatacatcaaatagtcactaacaaacatttgataaccgatagtacgggtatttttctcaccatgtactaatgtagtactgtcaaagtttagacgcttacgagtttcaccataaaggaactgtgaaccatcagtataaactggtttgaggttatcattacgggtatcccagtaatagtttacaaactgtgctggttttgctttgtatagaagaagcatttggtcataagcaaacatcgcagtgttcatgtaagtgaattccatgtcacccatgtcaccataagaccacggttctgcacgatgatctaaattaccactaacaagacctaaaacttcaggtgcaatatattcaacttcacaaatatcaataccagtactggttttctcgccagtcttagtgattttaaatggctgtacaccgaaacgcttgtatttgtcatattgaccacggcgttcacctgacaaaatagtaccagtactgctatcaccgatagtaccatcaatatcccacaatccacttgggaccaaatcaccattttcattcattactagtgcttcatacacataaacaacatcaccctcacgttcaccaacttgaactgcggttggttcgtattcttggttccaccaagtaggacgttgagagaagcccatcatttcccacggatgggttgctggacgatatgtaccatattggtcagtatagattgcacgccatgaaccacgagcaggaatttcagtacgttgaccatcaacaacataataactaccagtatagttccaagttctccagttatttgcatcataatcatgagtactgttactaataccattggttgctgtgaagttaatgtaaccacgtagtaatagagttttcttttcttctgcactataatcagtaccagtgaaatatggagttgggaataggtcttcactgtttagtactggtttataatcaatatccttgaaagtaagatcaatactattaaagatacgattttccaattcaaaaactacgtcatccattttatcaacaccattgataacacggtatgcttttgaaataacaccagtatgactgatattataacagctagatttactaccagcagttaagaatgttgctttttgtggtgtataagcttgcaaaatacccaagaactgtggagttggtgggacaaatgcacgaataaacccagcagtgcttgcaacgttgtcatagtagaatgggaatgaactatcacgtcccatattaatagctgttagaatcgcatctacgatgtttgatgcactattaccaagaaaagcacttgggtcttcttcatacaaacgaagcatttgagtgttgaacttgttaatgaagcggaagtattctgcttgcataaacatgattgattcaaacaaatcaatatcttcattagcggtatgtgccattagtggtaacatggacgcttcgttttgaataattttagtacctacaccattattaaccaaaccagcagccaaacgttcttcataattgttaaagtcatcagcagaaccagatgtaatattcttttgaataatatcttggaagtgcagggtgtaatcaccttggttgatgtattcaatgtttgaattatatgggttattttgtaggttaattggaacttcatatgcacccaactctttattaggagtgatagtgctactataagttctgatttgtacaacagaatcttcatccaaatcaacagagctatcaattgtaatactttgtacttgattattaactacagttttggtgtattcttttatttccacaccatcaacaactagttcgataatatcatcagaattatctagtactttttgtgccaattcaaatgtatgattgtacttaacgctttgtaccccatcaccaacgataacatgccaatcaagatatgtttcacgttctgctttgtaatattgataagttctgtcaacttcaattacataagcatacattccttcaactgggtctttgaaactttccatgtcataaacagtactgaacacgcggaacattactgtacgttcaacctgtggagtttgtttaacatattgtttggagtattcagtactacgaacccaatcagtttctaggttcattgtattaccttcaccaattacgttatagtaatatagtccatcaatttcaacttccacacccaaatgctcgtaagtataacgagttgattgtaggaaattctcatatgtgtaataattgtaatcacctagttcaatgttataaccataaacgctatcagttgcataattatagttaattttaaaaccaaatagtgaagaaccagtaaaagtacttgatggataggtagtagtgttgtttagtttaacaccattacggtcatacaattggaattgtggaaatacgttcacagaagttttagactgtgcttgaatccaatgagaaccgtcccaatacatggaactgttaccaaattcattacctttgataataagaactacgtcaccatcttgtggttcaccactcggacggctttcagattcttcgtctgggtctaatgctagaccatacacacgtgttccgtcttgcatacgtttaacatatactttataaattcggttgttttcaccattgttaccaatactggtgaaaagaatacgcataccatcttgtggaataataccatcaatacgtataccgctgttaatatctttttgtacgatatcttcaggtttaataatatcagagaacaaatcaactgtcccacggaatttagtaccgtagttcattagttcgatatttttattgtattcgataattggagcacgagcacgaacaaaacgggaatcttgcattagttcttcagttaggattgtaccatcttctaaagtttcacctaatgtgtaccagaagttttgtagtgaccaagtgttaccattttctgcaccacgttccattacaatataatctggagttgtgatattatagctattcttggaatcccataggtatgaactgtagttgttcattttatttgggttaattggtggggtccatgaatagaacttaccattaaacaaacggctttcattttcggttagtgcaccgtaatgattcatataaccaactagatcatcataaaattgaattgaacgaatgctggagtcatcatttcttgagacatacgcagaggacaactgatatagttgacgtgcgactgttgactcgtgtaagaagatgttccctgctgctgccggagaacccatgtcaccaatgtagccgtcaactaattcgttttcagccggattaaaccattgttggataacatcatcactgactttctgaatatcttctaagccttgcatccactcaggcaatctttttatgaaaagcttcttctgtactgcttcagccatctgtgtaggttgaaacttaaaattattagtcatttttggactccatataattcgttcattcatttgatttatttatgatataaaaaagacgttccgaagaacgccttaatgtgttatgaaccaatgttaatgttactcttagccaaggaattgactatctgtacgttatcaactgatgcagtacttaagaaaatctggtcagaacgtgaagggatttcaaatagtgagccaaatttagagttagggttttgagctacgataacaatagtactaatatgtcgtcctaattgggagtgaatgtatgtacacaagtctgtgtaatagaaactttcaccaaaatcccagttgtcaacattaaaatactcgttagttagtgctacgacattttgacgaatttcatcatcactcattgtactgttaggcaatttgacgactttgaaatctgctttataatcttcagcagcactcgcaccaaataatggtagatattctgcactatgccaaataatactgtcactgatgactattttttgttcaacttctttaaaaatgtctttcaattcgacagatgttggtggtttaggcatattaccttgtgttccattcttcaaccatacatcaacttcagttttgtatgaattggttaatacatacatgtcaatcaagtttgttctacttgggtcaatgcgattatcatctggtgcataatgtttccagtggaagtcaatacaagttcttgcattcgtttttacggtatattgtgaagttacatctttataaccataataatctaacttttggttagcatcagcattagtaccgatattaacaatagcacgataaaacgcaagttcaggagttgcagcagttgtatcatcagaataggtcaagttctcaattaattggtttaaacggccattattattaccaacagtttgttctatgaagtaatatttaccatcgtatttaaagccaaccataaagcctgaacctttactatagataccactcaatgttgttgagcgaatagcattgttgcgaacaaattcacttctcttatacgcattagtttctgcaagtgcctgatccattgtataagcccatgaagaatcaagaatagtgaaagtatcgttcggtaatgacagtactgatatagtgaaatcatcgttttcttggaaaaccaatataccaccatatttaactgaatcttcaccagtccaatttggtacaatttctctgaattgattaggttctagtgggattccattactatcaacgttttcagaggtgataaccacacgagaaccatcaagatacccgttttcttgttgaattgtgtcataaatttggaaatctatgttacttgtcaacccagcattactattgcttgtggtatttgatgataaaatttcaattttgtcactttgaacactaccagtactaatatcagttatctttttagaagaaacgaagtaaaaacgaactttatcaccaccaacgaacacatattcaacgccattcgcatcaaattcccatgtttcagcagtatatttgatgttaatcataacaagaacttcatttccattataagaaatagtatcttcatcagtactgatttctaatggaatccaagaagttgtttctttatcatgaataataacaaaatctgtctttttatcaagcattgcagcgattgcttgcacttcgacactattaaaacgtgttctgtattgtaaaatgtactcttgtgcttgcatatttgatgaaactgaagtacttagaacgtaatatccattatcaagaacactaacaatactagcccacacagtagttccggcatttgaaaacttaatcaacgtaccaacttgataatttccgaatacgttacctgttgcccccataatacgaccatacgtattagttgcgtaattagaattgtattcaggtgtccatttttgttgaatttcaataccaattgtattcggtggatatgcttgataacgttggattacagcattctcaccaatcaatggctgaatattatcaattacaatactagcatttgttttggtagttggtaattgtaaagatttattaaattgtgtgttttcttgatacattgaaccatcatcactaaacacatcagtgttttgatacttaccagttggatcattaatatcaatgaatcgtgactgtccactatataaacggttaattgctttagatttaagaactgtgttacccaaactcaacggagcaatgttatagtcttcgccgttaacaaaacggttttgagtgtaatacatttgtggagcacgagccttaatttgttcaattgtttctgcactttgtgcacgatctgattcatattgcaatgtgaaacgaatatccaaagtactggattcatagcttgattgtgttttggttctataggtgtattttacacttttgttctggatttcagacgttttaatgttgtatgtttcaccattactaacacgataccatacacgatacgtcccacgaggaacaacaccactacgagaatcaggatacttaatagtaatctgatctttatcacgagttgtgacagcaaagattgtttttagttgacggttaacagtattgtatgcgatactttccatagatggaactttggtccatttagtacgtacaataccatcttcgttgatttcctgaacccaaacatcagtttcgttgatattatcaacattgatatcttgaattcggttttcttctcgaatattatattggtaatcttgatatgtcaatgtcccttgaacaaaataaacaaagaaaccagtatttggtgaactaaatccgttaccatcattctgataaacgatatatttttgctcctgtggttcagggtgacgctcttcaacaccaactgctgtaatatcagggttgacaacttcaaatgtcatcgattcaccattaactgtagctgtgaatggaagacttggtgctgacattggaattgtattcatacgatatagctgacgagaaacaccattgatatcgatacgtttaaccggattaccaaactggttagtactgacaaatgaactgttcataactagaatgaagttctcataccagtttgtgtccattgggtcattccaacgaatgattttatccttaagtgattcgttaaaactattacgaacgtcttggttggtttttatttctgttaatttagcaactgcacgacctggatatgaacggcgtggagagtaagaaatcatttttgcgagtttcagtactgatgcacgacgagtagcagtatctaggaagttctcacgagtgtttaaatccatacggaatgctaagttctgtcctagcatagaaatggtatctagaataaagatgaactggtcgttttcgatccagtcgttgaaagaatctgggtacgtagcacgtagatagtcaatcatactttgacggattgtatcaaagtcataagctgttagattgattttataaaaactttcatatacttgagtccaatcctcaccactatataaattagactgtcttttttgttgtgacattattttaatcctttaataatatgttaaaattatcataaaagtacttttttagtactgaaatatagtcttcattatacttcaatcgaagcaaatttattccattctccgtacagtacgtgtttttaattttatcattgtgttgagtttgttttagtgattttgcaccaccccaatgttctgaacattgaaaatgttgttcaccatcatattcgatacacaagtttaaatcttcaatataaaaatcaaatggcaatgttaaaatattcttacaattatcaaatctcttttctctaacatatttaaatgaagataatatagtttctatatctgatactgccttagattgagtactatctctcatacatttttcacacccgtatctactattcaagtgattgtcaggtcgctggtagaatataccatgtatgggacaacttattgatatatgtgttttattattaacataattagccatactataatcaaatttatttttaaatttaaaattggcttttttcttgaaatttaaaaaattctgtggacttttttgtctatttgtattatattcaatagaacatttcatgcatcctttaccacatgtatggtcatttggtgtttgtagaaaaaatccatgtttttcacagccaatccatacttttgttttagcattatgatatttaaccatactatagtcatatcgtataccatgaacttttattgaattggttacaaatttatattttgtcgatctagtggttgcatatttcatacatcactccatgatttatatagaaatatttattcaaaaagaaaagcactccgaagagtgcttttgtaattataaagacgatactgaatatgttgccattctgttattacttcttgcatcaaaaactatagttaattcttcaagagtattgagaacaacatattcaagtactatatccatctgataaccgtactcaattcttgatatttgaagtttctttaagcttaaacgtgggtccaagctaataattctacgggcatctgcttccaaaaggttgattgtattataaccatccaattcaaatagcaaatcccacgcaatgaacccataatcagcatcaaatgcacgttcacctttttttgtattgaaatgattcattaaatcctgacgaaccaattcactgtcatacaatgtttcagttcttccaactactggagttgaaaaacctttaaaaacaattgccattatgtgtttcctttattatacggattcgacgatgcgagccatcttctccgctctggatgtggtttgtttgtaccacgaactatcacgcatttctttaccagcagttttataatcaccacgaccgagagcagccatcgcttttttgaaatttaaaacttttgatttacccatttggaacaccataaaccacaatacacgctgaacctgtactggagcagtcattccaaattgttcatagattgattttgcgtcattgtaagcggtctgagcatcttttataaggagagcatcagcttctgcttcagtgagtccatttttgaaactttcacccttaagtacaaggtgaccgtaaccaatggtatcaagacctagactgtctttatatacccaaaacttatcatttttaaaataaccaagttttttctgatacgcaatagtaccttcaaattctttaatttctttaaaaaattgttcgtcggtatttttgatttcccatttactcataaagatttcctcattattatttatagtactgaaatatttattgacatgcctctaatctacgtgccattaattcagcacggcgtgtagtttgacgataccaagctgaatttcgaatttgagcagcagcttgcttgtagttaccttttgccatttcagatagcattgacttaaatttcattacaccacctttacccatctggaataccatttgacatagaacttgttgtgccatgtaaggtgttttcatcttataagaagcgtaaatggattttgcgtcatatatagccttactactatcacgcaccaacatatcattggcttcctgttcggtaatcccaccagagaagttttcaccactaacaatcaagtgaccgtaaccaatagttgggaacccacgactgtccaaatatctccagaatttaccattgcggtaatatcctttagcttgttgatacgccattgttccttcatattctttcaattcagaaataaattgtgaatcacctggaacaactttccaatcaattgcagtcttatcaacattacccacattgtatgattcttgtgcagtaggtgctggagtaccacctgaacgttgtgttcttctacgacgaggaacatgacgagaaccacgttgagaatacccagtaccgatagttttaattttagttgttacaactaattcatcactatcaacagaaggttgaacaacagctttcggtgctgcttggttcgttgcttgtcctgcaattaatgttttatttcccgcttcagaaccagtaccagtctgggatgcatagaataatccagggtcaactgaaccactaaatcctagtacgttggcagaggtaactcttatttcaaattgaataacaccattagctttaccgataacttgacctttttgaacaacgtctttattgagcaaatctgattgaatattataaaatacagatttatatccagacttatgttgaaccaataaagcaccattagtgataacaactacaccgccatctggagctacaacatcacccttgcactgtatttcccagccacttctatcagctttaccaccaccactagtcgggacaccttttccccaataaccgtattggcgaatggttccactacacggcatgaacatatgaagaccttcatacgtaatgctataaccagtcataccaggatcagaagtcattaaatcttgtgtttgaacttgttctgtttctgttgtataatttacatattgaattgcttcagtgtcctgtactggagctaaagttggtgctacatcattagcaggttcaggttgaactggggattcactattaccactaccaccaccttgacgtggaggttcaacagtactgccgtgattctcagcatcaacccaagccgatatatcatgtatatgatcattcaagccagcagatggggtgcggaagtttggtgcattaaattcagcagtaccactattaccaccactgttaacagtaagagtttgtccaccaccactaataatagctgatgaagaaccaataagattagcactggtagcagcagagattgacgcattagaaccagcagttatatcagtactgctaccactttgcatacgtaatgaagctgctgttactaaacgtaaatctgctccagcgttaataacaacctgaccaccaactttagcatccaatgcaccaaccaaatctagtttagtgtttgcaccaacacgagcattgaagtctgctccactttcaatattatatgagcgtccagcaaccatgttaatatcttgacctgcacgaatgttaatattcttatcacaatgcatttcaatatcaccttcaccagtgaatttaatacgtgcagtaccatcacggttaattaaaagaacatcaccagaggcttcagataacaacaactgtgcaccactacgtgttttaattacaacgccttcatttttacgagaaccaacacgagtatcgttcactgggttattgaattgggtatcctgataaccatctggatcttgatcccatgtaggtgctgttaattcttcttctgtgtaaccatcatcaataataaaactgttaccacgaggtgaggaaagaccataaacagtactggtggtctggcgagtactgctacggttagcaacacccgcttcctcatcataaagtaagttttgttttttcagacctgcaataattggaacgtttgcatatttgtcttgagtactggtagaaattgtattacggtcataatcagttacgggtaatggcatatcagtacctgcataattgcttatttcagtagccatcccaggaaccatcgtattcatacgttggtcataaacacaaccaatccaatacgcttggaatgttcttgcttgtgcaaatataacaacaacggtacacccaacatcaggtggaaccgcccaaaaaccatatccctttggatattgaccataagcatcagcacttggtatccctggagttttaccagcgaatggagacatgtatgatgcttcaatccatgactcttttgaatttttatcactttgtgaaccttgaatccacacttttagcttaccggatttagttgggtcacggttatccatcacggttgctagttgtggatacgaagggtcaaaacgacccaactgttgttgggcatatttcttacttgtatctgccatcttaaattattccttattcaattatactatcgtgacgtttgagtgcacgaattacaattgcagcctgaatttgttgtgtaaatgatccacctgcaccaaaatcatgaacaattttgtttacacgataaacagtttggttaaatgtagttggggtattaatatcaagaacaccagtgttatcatttatagaaactacaggttgtaatttcaataaaagatatgcttcagatgtaatgtagtttgctacatggtaatcttctggattaatttccatatcttctccattaattacctgttcaagatattggatatatgaataatcactccaacctaaccaatatgggtcaccaactacacgcatatccaatttacacaagaatggacgattgtaatagttgtcacgaattttacggcggtccatttcatatgtactactgttatcgttcaatgatgcagataatgttgtttcattattggttgcttcagttggcattgtgaatacttcaccatatcgatgaccaattccatcggttccaccgtcattttcttcatatgtggttacataatcttcaatatactgtggtagaacgtttacttgatagttattataacgatcctgttcttcagcattaccaacctgcatttgttctcttgctaaagtttctactcgatttaaaagttgctcttgtgcctgtgttaatataccaccaagatcacgtgtgacgcgtaaatcacttgtttttgcccacgcatctggtatactgttgtactgaacaccttggatattcaattcttgtgatgtgtgtgtaccagcatggttcgcaacgtcctgtattggaccagggttacgagttgtagctacaaagaattgctgatcaaacttcaaatcaaggtctagaatctcactattgagtccactaaactgataattgtaagttttcatgttaatctttggtttttggtcatttgggtctacatatttctcggcattttcaaagaaatactcaaatgtaaagttttctggctgtaaatcggattcatgaacaatatcaccaattttaaaacgaactatatagaaaacaactttataaactgacgtgtttcggtaattatcaaatccaacatactgtttaagtgggattatattaacaaattgacgaacactagtactatcatcagcctgtacagtacaggtatctattacgtcatcgataattcgagaaattggagtacttggattccatgaaaagtttttagaaccctgtgcaccactttttgtagctccagcttctgccatctgtttagaatcatagtttataaacaaatcctgtaaatctggtgcaactttgaattcaaagaacttcccttctgctaactcaccaaatttataacgcaaataaccatattgctggtctgccatttgattcaatttatcttccaaatcaaaacaaaaatcaccaaagttttctgctgtgcaggtaaatggttctttaagagtccattccacattatccactagagccgcacggttatgaacaagactcatagaatacagcatagtactaccactttgtgatgcactagcggtaacactgttaatcttcacggaccatctacgagttgtttctggtatagtttcaggtacaccattagtattgaatccaataaatttcaattctaatatcaaaggtaagtccatgaacttatgataccttagtttattacttaaagaaattagttcatcatataaactcatactattgttttctgaaaggttcagtactgctgtgttgaagcaatagttggttgtcatccctggagttgaaggaccagcttgtgtcatctgaacacttgtgatattatactttccagttacaccactttcagagatgacatgacgttgaacttcagcatctgggtaaagccagaagtttttgaaatcatccttgtctaatgcgaaaagggaaaagtgccaagttggattgtcatactttaacaatttattgtcataaggaataacaaactcacttagatccaagtcttcatacacttggtcaacgcttgcactttgctttggtaaagactgtacgttactaaccttagctttaaaattgttttttactttttcaagatcattttgcaaagtattagcagtggcttttaagctttcaatagtacttgttgcagtttcttttgctgcctttgcagaacttgtgatgtttgaaatatcatttttaaaattatcaacagaactttgtaaatctgccatactttactccattaacctatgttacgagtaccgttcaaatagttttgcactcgttgatttgagggaacacgaattgtgataccactattaaaattgtatattgggtcttctcctaatatatcaggattcaatagggcaaatacataatacagacgttcattaccatacatttcgtatgctaaacgccaaggttgatgctcatacttcaatggaattacatagaagtcgtcagtatcagaaggaaccatatcacgtggtaaattgtaaccaagataccacgtttgtttaacctttgcataaggagagtattgactataattggacattagataaatcctccattacgtaatagttcaccacgcttaaatgcatttaggttgaactttttagtcatgtaatcagatgttggttgttcttcaagttgaagcattacagatattttctgtggaacccatacctcatcttgtggattcatatatccagtacttctaagctgttcgaagaattgacgactatccatatcagataaattaaaagtactatcactactattcaatggaacctgaatgtaagaaacatcatttggatatgtaaaattatatgctttcaaaataacaggaatatcattatatgtataacgaccatacccacttaatagtaaaattggcggtggtgcacctgcaattgctagttcgggattattagcagatgctgcttgacgaccaaagtccattaaggacacggaacgaataaaatgaacacaagccaatgcatacattgcttcagcagcagtgtttgcataaaactcaccttggatagtgaaaattgtgtttgttgtattcttgtagttgttgtagttactgttagtatgtataggggactgtgtttcatattgaaccgacctttgttcagttacagttggggttacagtccaaaccaaaccatttgttggacgcagaaacgacagcaatgttggctctgaactgttgccaaaatcaccatacacatagttagcacctgcggttttaggtcttaatcggattctacgatcaatcatctttttgattctccataagaagggaacatttgatgtatttatacgtgggacaaagacgtttttggtgctataataacattgatggtacaaggaatgtacctattggatttaaaacatggagagttgaaataatgacaaataggccgaagcgtaaaaagtatcttaacaatgctgatttacttcgtgagatacaaaaaagtaaaatctcatattgctggttacttgataagtgctacagtatgtacgattatatcgtttttgatgaatccgaaatcacagcagaacttttagataaagctaaagcagcacgtattaaacgtctcaacaatgaaatgcttgatgcaattcgtgaaaaagaaggtatgagtgcgaagaaagcgaaggaatacgcagatgagcacaatttagtactgactgatgtacctatcgatgaggtggttattcgtgtaattaccgatgaacatatcccagcagatacaagagttaattttgaaccattcaaacattacattgtagatgaagaaggtattctgtacgaagtcgttcgttctcactggaaaggtgatcttgaagacggagaattcagtactgttcatggtaaaatcactaatgaactaggtaaaatgttcattaaactggcagaggaaatctcacacaaatccaactatagaaactatacatatttggatgaaatgatgggtgatgccagaatacaattagttaaaaacgcacttctctttaaagagagtattctttacaaaaaagataaaccagcagtacagttgaatcctttctcttactatacttcgttcgttaacaatagtttccgttcgattcttaatagtgagaaaaatgttcgtaacatccgagacgatttacttgagatgcatggttttacaccatcacacactcgtcaaatcgaaattgaaatgaacatgattgaaaggaaaaacgaagatggctctatttgataaagcaattacatttactgatattcatttaggattaaagaataacagcaagcagcacaacgaagattgtttgaacttcatcaagtttatgattgaacttgctaagaaaaatggtattactgtatgcgtgtttatgggagattacttccataaccgcagtaatattaacattcaaacattaaactatggtttacaaattctacaattactaaatgacaactttgataaggtttacttcttagttgggaaccatgacatgtatcataaaaataaacgtgacattactagtattaatatggcacaagtttatgagaacatcgagttcattaatgaaatcacaacatttgatgattgtactttcattccatttatgattgaagatgaatacaaagttctacctcaacttaaaagtaagtatgtttttggtcacttagagttacctggctacttgctgaataaaatggttgagatgccagatcatggtaaagagactgaaggttcttttggtggttgtgagtatgtttttagtgggcatttccacaagagacaagcaaaaacaacatccaaaggaactcgtgtaatttacacaggtaactgtttcccacataacttttctgatacatgggatgatgcaagaggtgtgatgattttagaacatggtaaagaaccaactttccattcttggtcagaagcaccaaaatatagaacatttacactttcagaactattaacagatccaagttattatcttcaagacaatactatttcaaaggttcaagttgatattgctatcactactgatgagattagttttattcgtgatacttttactaaactttataacattcgtgagtttaacgttgttactagcaagcaaagtatcaaagaacttattgaagatggtgaaagctttgaaagtactgagtcagttgatgagattgtgttacaacaaatcatggaaattgattcacctatgttcgataaaaatcttctaatccaaatttacagatcactataagggaaatttattgtgctaaacattacacgtatatcaatgaggaacttcttttcatttggcaatgcaccacaagtaattgatttgtctggtgctgatatatcgttagttcttggtcaaaacaatgatgcagttattgaaggtggtgatagttccggtcgccgtaatggtgttggtaaaagtgccatcattcaaggtttagtttttggtttgtttggaaaaagtattggtaacgatatcaaaattccaaacttagttaataaaactaatgctaaaaactgtgaagtatctgttgagttcgagaaagatggtgtacaatatctcgtcgagcgtgggagaagtcccacgtatttcaactttattacattaggtaaagataataaggtcgaagatgagtcaagaggggagaagaaagatacacaagaagatctaaacgagattcttgggatttctcaactactatttgaacatattgtagtacttaatgctaacgtagagccgtttcttgcattatcatcacagaaacaacgtgatatgattgaggaacttctgggcatcacacaattaacagagaaagctgaactattaaaggatatgtataaagattctaaacgtttagctgaacaagaaaagttcaaaattgaaactgtggctgcaagtaatgaacgtattcaaaaaagtattgaatcattacaagaacaagctaaccaatttgagattgataaaaataatagagtatctaagttgactgaagaattgaatatctacaaaactttagacttcaacgatctctatgatttggcagaagtgaaagagaaagctcttattcataatgctgctcgtgatcaaatagaaagacgtgtgcaagagttagctttgaagtatgatgcatatgaccgtgaactagcaacagcaaaattagcattaaatgtaagtattgataagctaaaaactattgatatcagtactgaattagaaaatcacaatgagttggagatatggaaacagttggagggtattctaaaagagaataataaccaaaaacgtttcaaagaacaaaaacttaacgcattaaaggtggctttaaatggtcatgagagttcgttacgtgctgaggtggacaaattagcaacagtggagagtagtaaatgtccactttgtatgtccgaattagaacataatgaacatacttcgtctattaaagataaaattcaaaatactattaatgaacttcaatcgaaaattgatagttccacttctgaaattgaaacaatacttcaggaaattgatagtattgagatttttgatatgccacctaagcctgtgacattctattctacactaactgaagcacaaatgcatgagcataagcttcaagaacttattaaaaagagtgaagaagagcaaattaacatctatgctaatgaacttatgtcatcatatgaagaactgaacgcacatgttcttcaagaagtaccagatacattgaataaatctgaagtaaaagagttagaaatgacagtaacttcattatcacagcaattagatcgtgaattgaaaactactaacacatttattggacaaattaatacacttacgaccgattcattagtaccaatcgactatagtgactataataaccttgataagatggcaaatcaccaagatttcttggttaaattgttgttaaacaaggattcttatgtccgtaagcgtattattgaacaaaacatttcgtatctgaatacacgtcttcaatattacattgatatgtgtggttctgaacataatgttgtgttcttgaatgatttgagtgtagaaattgagaaaagtggtcagtactttgactataaacagcttagtcgtggtgagcgtacaagggtaaacattgctttatcttgtgctttccgtgatacatatgagtcattgtatcaaagtatcaatattcttatcattgatgagatgattgaccaaggtcttgattcaagtggtgttcttcgcgtgtggtctatcttccaagatatggctgctgtgcgtggtaagaatgtatatgccatttctcacagagaagaattgctttctaagactgagaaaatcctaaaagtagttaaagaggatggtttcagtactgttgagtatacaactcttgatgatgtattctaattaaaaggaaagggagccttagctccctttttttattatattaatcaaatcttcatataaagacggttctatcgtttcggtatacccaacttctaaaatagtttttgataaaaaaacacttttcatgccagcttttattgctttttctatattatatggaacattaccatcttcatcatatagttcataaacacaattaaattttaagttagagtgatactcatggtaatattttctattattaatagtaccattagttattccatatttgtatgctatagttttgtcgtcttgtaaaacttcatatacatataaacgacctggtttacttctcttgtaaccaccaactccacattctgaacaacctttacctgatatatgttcatgtggcctctgataaaaagaaccatgtttattgcatataatttcaacttttttctttgaagatttatatatcacatttgagtaatcatataattcaccatgaactttcatggatttttttatgaagtcatctttagtatcgtgcttgctttctaagtaacattcattacatccacttccttttaaatggtgatttggagtttgtgaaaacataccatgtttcttacatataattttaactttagtgtaattatttacatactcaactaatgtatattcatatttgttgttatgttttatatttgctttatctataaaaatatcattttttgtattcatttatattgagaattcctttcttaataagctctctagctctctttgagcgttcttttgtattaggtaatggctctataccgtataaccccaatatctcgttctgacgcgttgcagggagccttttaaacttgtttacatccacagttagtgccgtgtgatgaatcacctgttcttccagtgggatattattgttcttttcaagataatctgcaaggtttcttagtacttcatgtaatggcatatcagttaaatgctttccatatcggttccatgcgttggaaaccttaccctcaaaggcgtttatctcattctggagtactccacgacagtgttgattctcgaagtgatagtgatcaagtactggcttagttattggttccttaattataggatcaattcctttttgcttctttagtaattcatttcgatagtttgcagcttctttactatcttttaatcttttcttcataaatccccactaaactaattcttatcaaaatatttatgcgttacttcaatgttcaagaattatgtactttttataaattttttatcataatagtgagacacttttgaatttggtgctataatgttaattgtagtgatgacaatcctgttgaatgcagaaatgcatataaattagaacctgatcctatcgaaaatgtagatggcagatgagcagttcaacagtgcgatgagcagaagaaacccgttagatgtgggcatagtttgaacccctcacggcgacacggctcaataaagaactccgataagcatacagagagttcctcgatagatgtataaggatcgcaccttaccagatttgctaagggagtttattaagtgatttaataaacagcctgaagtttgattgtatagactttcaggtgagtgtcaggatgattaagactgacgaccgttgcacatgactgcgatagagagaaacagagcaacccgctttcgttcttaatgaattttaagaaattcgcaatacaataaatgctgaaaccatcagcgtcaagaattaagagacttaattgtctcttttttattgtcttcattcccctgcggggggatgaaaatgtcttcaacaaatcccgtcaagtattaaatcttttaaagttctttataattaattataagaaatgaatatgagttacgtagtaacgaatattcttcagctaacgaatgaaatgagttagatgagttcactataaaataattaattcctttcataattcaattcaatcaattctgtagtttaaaaccaaaaaaaaccagcaacaatgtgctggctccttgtgagttagtgcttctcgttttgttttttaattttcttattaaaagacttaactaataagtctctatcaattagtggcatattaagtagtgttccgtaatcaacactttccatataaacagaacactccattaatgcagttttaatatctgacttattcaactcaaattccttaagtacttcttgtatttcttcaggagatgagtggagaagcattatgcgaaaaaatgacttgggtcatatttcagtccattaatttcccattgatgattacagtcactgcattcaacttccatagtatgggaaacaccaatttcaagtacttcatccattttttcacgaattattttaagtgttcctttggactgctgacctagccattcagaaatgaattcgttttcagttacttcatcaccttcaggagtaattactttagcaataactcctgcgatcatatcaaagttgagttgtgccatactttcaaatacttcaccaaacactttaactttttcttcctgctccatttcggcagtttcaaggaatttagttttttgttgttctgtaaacattctttctgcgaagttagaatattccttccaagtaagtggtctaaactctaaacttagtccattatccaattgtacagtaggaacttcactgaaagtttgtgctgtttctagtagatatgataagctacgctcaaaacggccttctttgttacattcaggacatgtcacattaatatcatatgattcttctttagaagctactttaatagcgataagtagttgctctacgtcatttacataaagttcatctgcatcaagtacgttaggtacgcagttttcaataacacgagcaattgcatcaccatttagaagcatatctggtgtattgagtgtcatttcatcgcgtgcactacgagcacaaatgccacatttgatacctttagagccaagaactccttctttataaggaatcacgccatttgtgaccaattttgtaccaagttcttcaattttagtatacttagctagtgggttcattgccattttattaaatctccatgtaataaatgtctctgtagtatttattaccaaaaaatgggacaaattggacaaatgttatagatgggtcttttttattcgttatagattcataaatatttctataatacatctattaaaagggaatgttatggctgtcaatacaagtgattacctactacagcagttggttaatcaaactaaagataacaatgatagtttgactgctatccgtgagttattagctgaaaatataaagaatgataagaaaaaatcaggttcaaatagtaaaaagaatggttccagttccagttcttccagtaatcgtggaaatcgtggaagtgcacaagccgatgatgggaaagcatttggtaaattgttcaaatcactatcttctgaagtttctaatgtaagcaaaacaatgatgaactccagtaccaacgtaagtactgtagtaaactcattaggcgttagtgcatcatcatttgctggttcattagcaatgatgccaggtccaattggtgcagcggcagcagcgtttaaactcgttactggtgctggtcttgcggtatacaatgcccttaatgcacaattggaagtttataccagaattaacagtgctggtgttgcattggctgaaggttatggctcagtactgaaaggtgcaggtgcatctatgttgagtatggatgcattcagtgaagctctacaaacacatagttctgtagtagctcaattggatggggcatatggtgatggtgttgctacctttggtaaattattaggaactgtccaacaagctcaaaatgaacttggcctttatggtatttctcaaaaaacattaactgatttaactgcaagaaacattaagttccaaaaacaatatggtggtcgtgaagctcttcgtagtatggaccaagctaaaagtactgaacaatttatcatgagcatgactaaattcagtaaaagcatgggtgaatccgttgatgcattactaaacaaaacacaaaacctagataaatcactcgatactcgttctatgcaaattgcattgcagaactactctggactatccagtaaagctgctgtagcaacatctaaggcgttcaacgaagctgcgggtgcaatgggtgatgcgggtgcagatttcctccgtttgatatcacacagattgtctattggtggtgtaccggatgacctactcactccagtactgaaccagttcgctgatttaggtgaaaatcttgtgcgtagtggtgtaactgattcacaagaaattcgtcgccaacaattagaattcgttaaaaagaataaagaattaattaagcaagatttgcttaaccagattcaattaggaaacacaactgctgcaaccttctaccaacaactattggatatggaagcaaccgtaaatgaacctaaaaataatcccgctgataaattaactcaatttactaaccgcttcaatatgtggattagtaaagaattcactgaaccatttaatacaatgtatgctaatacacaaaattcaattgtggattatctttctgacattgcagacagaactgatggtgcatgggagtttattggggaattggctcttgatggatttgggttagtacctattgctaaagatgcaattgcttaccttgatcgtattggtgctgaatttacagcttggttggatggattgggtgcagatatattcggttcaggttactctaaagtgtcagaagcatttgatacctttatgaataacttaatggatttacctggtaacctatggaattctgttaaaagttggttcggtggtggttctgataatgatataaaactcaataacagtactgaaaccccagcagaaagcgtaaacaacatcattgacaacggtgtaaatagtgttagtgattattttgcagggatgtgggataagttcaacaaatggaacacctcacgtaactacaaagaaccaaaaccactaccatctgattatgctgacagtttattcaaccgtccaggagtagaacgtattcgtcctgttattgaaaaaaccaataacgttgctccagaaataactgtatctcgtaaagattttgataaaactgcatgggcaccaggtgaaccaccagttccaccaaaacctgacgatacagaggcaattaagaagatgataaataatggtagtgcagtgacatcgagaactggaccaccagcaccgatttctgatgaaatgaaagaaatactcaaaaagcttgccgatgttaatgaacaatcactatctgctgtacaacagacaaataattatctaagaactatctctgaaaacacccaaggtgagagaaacagttaaggacgaacaataatgagttggaaacgacatatgacaatggggagatcgaagttatcggtcgtccctaatcagtccagtaatagcaacaaatcaacaggaacgaaaaccaatttcgcttcctatcttccagtagtatacaatggtttagctaaccgtactgaacgttatagtcagtatgaacagatggaccaagatcctgaaattagaacagcacttaatatcattgctgacttctgtacacaaagtactgaagattttggtcttccatataaaatcaaatataaagatgatattggtgatacagaagtaactactattgaagaccgtttggatgcatggactgaacaaaatgattttaaaaccagaatgtatgaaattgtacgtggcctattaaaatacggtgaccaattcttcattcgtgacccagaaacatttgactggtactgggtaagcccaaaaaacgttgaacaagtattcgtcaacgaagcaaccggaaaaacccctgtaatttattatatccgtgatgtatctttgaatcttaaagataaagtgtttagtaacttgcaagttggttttgacaaaactgcatatcctggtgcaatccctaccacattaggtggcggtggttcagtagcagcaggtggtgcatctggctttactaaccaaggaaactctgctgacccattcagtactaacggtcgtcttgatgttattccagttggtgcagaacacgtaattcatctaaccctaaatacaggacaagatggagtttggccttttggtactagtatacttgaagatgttttcaaagtctataaacaaaaagaattacttgaagatagtattattatctatcgtgttcaacgtgctccagagcgtcgtgtatttaaaatcgacgttggtgatatgcaaactcaccaagcaatggcatacgtagaacgattcaaaaatgaaattcaccaaagacgtattccttcaaataaaggtggcagtacttctctaatggatgcagcatataatccattaagtattctagaagattacttcttcccacaaacttctgaaggtcgtggttcagatgtacaaacacttccaggtggtgataacttaggtcagattgatgacttgcgttacttcaacaataaattgattcgtggattgcaaattccagcttcatatctaccgcaaggtccagatgatggtggtgtatcactattcggtgatggtgcaacacaagcaatggcaagtgaattacgttttaataaccaatgtatgcgttatcaacgtttagttgcaaaactatttgatgcagaatttaaacgttttatggttaaaaatggatataacattagtacttcatcttttgaagtaattatgaatccacctattaacttggcttcttatcgtaaagcggaactagatgctaaactaattaacacctatctcccacttaacgatctgccatatatggcaaaacaaactattcttgaaaaaatgggcttctctaaagctgatattgttaagaatgagcaaatgtggctccaagaaaatccacaagatggttccgctccgtcaagtggtgaagaagatgctgctgggttgcaatctgttggtatagatgctcctaacgatcctggtatggatttgggtattgatggtgacggtaacttagaaagtgccgatgctgaataccaagaacaacaaggaagtgcaggttttaacccgaataatttaggcgattctttttaatcataaataacaatgtataagaatgagggtacattagttgccctcattataatgcatattcaaaggagatgaaatgttaaaggacatctttgaatctagcagtgattctttatacgaccctgaagatgatcagaacgtatataaaattactgattccagaaaaccaaaactaacattacaagtacttaataatttaagaaagtaccgtgaatttaaagctaatgaagaacaacgacgcaaaaatgttgtcgctgttgtttatgcagcagcaccatccggtgatggttcaggattaatgtaaacaaaaatcgcactttttaaagcttttaaaaatggcgaaatttttcagtactaaataatatcatactttatatgaatgtaatatatctatggaggatataatggcgaatttaaaactactacaagaagcaattgaagcgtttgcaaacggcgaacaagatgttgctgataaaaaaatgcgtaagtactttgttgagcaagctcaagaaatcaataaagaactagaagaaagcatggacgaagaagaatgtgaagttgaagtcaaagacgaagaaaaacttgacgaagactttgatgttcaaccagacgctggtctaaatgctgaaatcgaatacaccctaggtgaagaagatgaaagtgcagaagaagaagttgaattctctgctgatgctgatggcgaagaagttgacgctgaagcagaagttccttccgaagaccaatgggaatctattcaagatgctttccgtggcctacaacaaatgtttgacgaaatcggtggtgatgaagttgacctaggttctgacgacgaagaagttgaagttggtgcggaagaagaattcggtgctgatggtgaagaatctgaagacgaatttgctgatgtacaattcggtgatgagaaagttggtgagtctttcaaacttaagcaagttgctgcggttgataagaaagaaaaagctggcgttaataccaagtccccagttgcaccagaagctaaatctccagttcaaggtgttgctccagtgaaattcgctaaagcttccgaaaaacaagtagattatgattttgaaaacgaagatgctatgtcccacactgatgaagacctaggtaaaaatgctcgtggcgaaatgaaagatgttgcgaagccaaaaaataccgcttctaaatctcgttctccactaccaaaatcacaaccaaaaatctaacactaaataaagaaaaggaaaggagatcattatgagtatgattcgtgaatggtcttcattcaatgattctaaagttgttttggaacatcgtgataatggacttggtgggcgtgattgctatcttcgtggtattgctattcaagccgatcaaagaaatttaaatgagagaatttaccctttagccgaaattactaaagctgttaataatatggctcaacgtttggctaaaggtgaaagtattctagttgaatgtgaccaccccgaaacactaacaattaaccttgaccgtgtagcagcaatgattactaaggtctggatggaaggtgctaacggcatggcagaaatcaaactacttgatactgctcatggtactaatattcgtaaaatgctagaagctggtgtgaaacttggtgtaagttctcgtggttccggtaacgtggattacaatggaatagtttctgattttgaaattatcacaattgatattgttgctcaacctagtgctccagaggcataccctactgcggtttttgaatctctgatgagtaagtacggatcaccttgtaatagtcctaaaaaaatcacagaatctaaaacagtaaataatgtagattctgaaatttacgattttttcaagaaattaaaaggttaacattttaaaaatataaatatttgtatgttgaagtacagatatttcagtacttaaaggagagaacgaatgtctaaattagaaaagcttctaaaagaatcatctctttctgatgaggcaaaacagctaattcaggaagcatggaatgaagaaaaaaacacacttgccgcagaaatgcgtgaagaaatgaaaggccgctacaaacaagatttgtccgagctaaccgaaggtctaaatactatggttactcaagttatcgctgaagaaatgggtgcagtatatgaagagaaaaaacgtcttgtagaagatcgtgtaactctccgtaaaaccctaagtaatttctctgcgttttctaactctgttttggctgaagaagttaaacaaatgcgtaaagaacgtaagcatcttcacgaatgcatgggtaaattcgttaatttctctaacggtatcctagcagaagagctaaaagaattccacatcgagaaacgtcaactagttgaaactcgcgttaaactactagctgaaggctcccgtcaaattgctgaagcacgtaacaacttcatcaaacgtacctctgaaagtgctgctaaatacattgcagaaactactcagaaaaacctatacgctttgaaagaagatcttgttgaagcaaaacaaaatatgtttggtcgtaaaatctttgaagcgtttgctcaagagtttaatgtcaaacaatttaatgagaattcactactacgttccctaactgaatcaattaagtctaaagaagacgaaattgcacaagttaaacaagaactaaatgaaactgtaagtgaagttaaagctgctaaaaccaaactacgtattatggaagattcacaagctcgtaatgcgattatgtctgaactagttaagccactaactacacaacagaaacaaattatggaatcccttctagctacttctcctacagaaaagcttaaagacgatttcagcaagtaccataagtcagtacttaacggtactaaagtaaatgaatctgctaccactcgtcaagcagttcagtcttccgctaagagaactcttgcagaaggtaaagtaatcactggcaaccgtcaggctattgttgaaagcgaagaaatgaccgctgacgatatggcattcctaaaagaactcgatgttctttctggtatcaataaaaaataaacattttaaagcattttaataatcaaatttttcagttctataaataatatcatactgaaaaacgaatataacatctttaaggagataataatgtcacaactactaaatgaatccaaatgggtagcagttaaagagaaactagttgaaggtctacaaggtactcgtaagtccgtaatggaaaccgtacttgataaccatcgtaaactaatgctaaacgaatctgctacttctggtgcaaccaccgctggtaacatcgctactctaaacaaagtgattctaccaattatgcgtcgtgttatgccaaccgtaatcgctaacgaaatcatcggtgttcagccaatgactggcccagttggtcagatccacaccctacgtgttcaatatgctgacaacgctcctggtgttatcgctggtgaagaaatgctttctccatacaaaatcgctaagtcttatactggtgacctaaacgcagacaacagcaacccacaagctgctcctactagccaactagaaggtactatgggtcgtcgcgttaacatccgtattctacgtgaaactgttgaagcgaaatctcgtcgtctatctgcacgttggactgttgaatctgcacaagatgcacaatcccaacatggtatcgacgttgaagctgaactaatggctgcaatcgctcaagaagttaccgttgaaatcgaccaagagctactaagccgtctacgtgcactacctggtgctgctgctgtaacttatgaccaatctaaagttactggcgttgctactttcgtaggtgatgagcacgctgctctagctaccctaatcaaccgtcaagcgaacgaagttgctcgtcgtaccaaacgtggtgctgcgaactgggctgttgtttctccaaccgctctaaccattctacagtctgcaactacttctagttttgcacgtactactgaaggtactttcgaagctccaactaacgttaaatttgttggtactctaaacagcaccatgcgtatttttgtagacacgtatgcctcagatgtcgaggacattttgattggttataaaggtaatgctgaaactgatgctggtgtgttctattgcccatacatcccactaatggcgtccggtactgtaatggaccctaacactggtgaattggtcacaaccttcctcactcgttatggttacgtacaactcactgactcaaccagtagcttgggtaatgctgctgattactacagcaaaatcgctatcaaaaacgttactttcatgtaataacaaagaatttaaatatactagggactcttcggagtcccttttctttttttttatttttgtggtataatattatctacaatgaaaattaacccacaaggtaggattatgaacaagcatgaacaaaaatttataaacgatgttaacgaaaaatatggaatggggaaatatacatttgaaaagatggtatatgttaacgcccataagcaagtcgaactttattgtactgattgtaatcaatattttttaattacccctatgcaaattgttagaaaaactgctaaaggttgtaatatatgtcataaaaacgtcccacatcaaactgacgaatttattaaaaaatgtcaagaaaaatatggtaacaaatattcttatgaaaaaaccgtttatgtaaatcaaaaaactaaagtgattcttttctgtaatgaacatcaagattatatcgaagttaacccagcaacattgttgaataaaaattgtgattgtggtgtatgtgctaatcgttttggtataaccactgaactgtttattaaaaaagctatagcagtacatggtgacaaatattgctatgacttagttgaatataaaaatagtgatacaaagattattatcgtttgtccagaacatggtaaatttactcaaattccaaaggcacatgtcaacggtcgtgggtgtccatcttgtggagaatatggataccagccatcaaaacctggatatttttacattcaaaaattgacaaataaaaagaaaacagtttacaagtacggaattactggtgatatgaagaggcgaattcatgaacaaagtcgtgattctgattttgaacacactgtactagttcagcgtttctttgaagatgggcataaaccattagtactggaaaagtttttgaaacaatacattgatagtggtgtagtaagtactgaagagttaccatcaggatttactgaaacttttgatgctaagtatcttgaaagtgttttggaaatcgtaaatacttttaagtaaacaaccataaggagttgacacggatgttaacagtatacatttttaagaaagaagagtacttaaagagtcagtactcaaaaattgagattaacgttccatacgggactttagttggttatgatgaagctagtgaaactttgtattataatgatgatagactggactatagtattgattcatttattgaagacatacttgatagtaacactgaagatatgttatatgaagaatacttagataagtacaaagaacgtatgtcaaagttacatgctgatgctccagtactgaatatttcaaaaactccttctgaattcgagccacaaaacccattccaacgcctaattgatgctggggtgaatttccgaattacttgattaataaatatcttcatattactgatggagatattttctatgtcacagatatgttatgttatagtaaatgatgcatggcctgattatgttaagataggatttaccagtaaatctgaaatgacagaacgtttaaggacatatcaaactgctacaccttttagagattttgtggtataccatgaagtacactttgaagatgcacgtttagcagagaaagaaattcacaaaagactaaaacaaatgaacgcagtaaggcaacctaacaccgagtggttcaagatgcctaagaagatagcagcgaacatcgtcgattctgttcttgacgatatggaaaacgacttgctatagtaataaatatagtatatattaatcattaaagggatttatacatgagcataagttttaataacccactggatactatgactagtactggtgctaatggatctttagaactaatagtgcttggtggtacaccaactaatccaagacctattcgttttaattcatcttctgtcattttaccagtacaggcattgccagcaggtgagcaaggtgctatggtatttgatagaaatgagttggtgttaaaataccacaacgggacaagttggattgcagtatacgatactgatacaattcttgaaccaatccgtatttcactcgatgaagtgaaccgtaaactgaatttaaaagttgaaacagtaacatatagtagtagtgctgttccaagtgcgagcattagtggtacgaatttaaatatcgtattccctattccatcaagtagcggtactggcccaacaggtctttatacttcaatgaagcctggtgcaattacccagtactcattgacttctggacaaactgttgctagtattcgtgaacagatgtctggtgtgaccaatggtcaaagtggtcgtaatggttcacaaggttctccgtggaaaacttctgatggttggtgttttgctgatggaatgtggtggcaatgggttggtgaaaatggtacaatcacaaaacaagttccaaatcttaaccaagaagcatacttgaaaggcattaccacatcaggcgtaactaaaactgatagtgtgattgcaagtagtgcatcaattagtgggactgcgatatcaattgctcaacttccaccgcactcatttagtttcagtggaactactggtgctgctggtcgtcatggtcatagtcaagcactaaaaggtatggacagaagtggtcttaatgcgttaacatccagtaacgacaacggaatcccaactgtaattaataacattaacgaagcacctgatcacgtgcatagtttcagtggttcaaccaataccgttggtagtggacaagcacatacccacggaattagcaatattgacgtaccacactttaatgtggcatacttatataacattgctgagtctaacgttgcattgagtgaaaaagttgcaaacacccgttatgtacttaaaactggtgatacaatgtctggttcattgagtatcgcaaacagtgcaacaattcgttcgaatgatacttcattagtactttacttccgtaacgcggcaggaactactgaacgtgcagcaatttatcacagttcatcaacaaatacattacgtttgagaagtaatggtggttctgaagttacaattaactcttctggtgtactttatgctccatcagtaactggtgcatcattgtctatttctggaaacacagcaactgttgctagtaaaaacatcgtcagatctattaacggtaacaacgctgatgcaagcggtaacgtaactttaacactgggtgctgtacaagatgttcgtttaagtaaaagaacatctacgcgacgtgttcctgattcttcttggatctatgcaccagaaggtgctacgcttgatgggttgtttatttccgtaaatgggagttctggtacacacgttgaaggttatagttggagatatacacaaaaattagtcaatggtgtatggactaacgtagtagtggagtcttaatatgaaaacatatacacatttcaaaaaatatatcccagtagaaaagaaacatggagataatgttctttatctattgtcagatgagggtgttgactggtatgattgtcaaaacgaattcagacaagatactttaaaagttcagtactctgacgatggatatattgcacaagcatcatatgatgtaacattgttagtaccgtatggatacagtgtttctgagttggagcatgatggagatgaagatgaagtagttggtaaaatctatgatggaagtaaaatttctgattatgttgaatctactgatgaaaagatcaaacgtctatcatctgagcaccaagaattaagaaccgtagcaattaatgagttgaataacattaagatggcgatagatcttgaggtagcaactgattatgaaatagaactgtttcctaagttgaaagcatatgttcttgagctatcatctctaacaacatctgaatttagtaagaagaagttttcgttccctgttaaacctgtttaaaagtttttttggaacaaaatcgtaagtagtgatacaatgtagtcatcttacagaggttgtgagatgacatttttttcaaaacacatggagaagcaaacaaatggcaaaagctaaaaaagaagttaaagttgaaattcgtgacctaaatgatgaacaacgtaaacgtctaggtgatgcactaaacaacgcagtagtacaacttacttttattgacgacaccaaagcatcctatgcagatttcattagttccatctctgatgaactaggtattgatgcatccgatatcaaacaagccgctcaacgtatctacaaaggcgattactttgataaacaaaacaaactcgataatatcgaaactatcctaagtgttactaacaatttgccacaagcagatgacgattactaataagtactaaaaactaaactttatagcctccaatgaatgggggctttatttttaacttaataaacgtaaaggaatacaaacgatgtatatcgatgcttatatggaacgtaaacgtactggtgatattttgcacgtagcagaacgtaaaaacggtaaacgtattctacgtcagttggacccagtatatgaatattatgttgaatctcctactggtgaacacaaaacaatttatggaaccaacgcaaagcatttggagtttgaaactcaatcacagatgaataaaagcgttgagcaacataaaaatgaaggtctacgtatttttgaaagtgattgcaacattaccttcaaaacgttggctagagaatacatgggtaaaggttcgccggatttgaacgtagcatttttcgatatcgaaaccgacttccataagaagtttggttatgctccaccatcagatccattcaaccgtgtaactgcaatttctctatatcaatcatggactggtaaggattatgtactgactttgccaccaactgatatggaaatgtacgaagcacaagatattgttcgtcagttaaacgaagaaactaacgaccctaacagtactgtcatcctttatacggatgagaaagaaatgtttatggagttctttagtctaatcgatgactctgatgtaatttcaggttggaacagtgaattctttgatattccatatatggtcaaccgtaccaagaaaatcttcaacgaagcaactactgctcgttggtgcttgtggaataaaaaaccaaatgctcgtgaagctgataactttggtaaaactgtagtaacgtatgatcttattggtcgcgtacacttagactatcttgctctttataagaaacatgctggtcaagttgaacagtcgtacaaactggattatattgctgaaaaagtaactaaagaaaacaaagtatcctatgaaggttcattggataaactataccgtgaagattacattacattcttgcgttattctaagcaggatagtattcttctaaagaaaattgatgctaaatgtgactttatcaacctgcacaaccgtcttgctcataaagaatgtgtactaattagtactacaatgggttcggttgcactaatcgacactgctatcatcaaccttgctcacagtcgcggtgaagttgtttttaacaaagcaccgaaagatgatattgagttagatccagagatggagtactatggtgaagaaggtgatgaagaagatgagaaaatcagtgcaaaagctgctggtgcatgggttcaagatccagttcttggccttgttagcgatttgggatgtacagactttaactcattgtatccaactgtactacgtacacttggcatgagtactgaatgtattcttggacaacttcgtcaagactacactgatgcatatcttgctgctaaagtacaagagcaaaaagctaaatccaaatctaaagcgtttgaacctgattggaccgctgcatggcatggtttatttgctgctgtagaatttacaatggttcatgaaaaatctgacaaaactttgattgttgatttagaagatggtagttcgttcaatgcaactggtaaagaattgtatgatattatctttgccgaagacagtactattgtactaagtgctaacggaacactttttgataagtctaagtatggtgtaattccagatattcttactcaatggtattctgagcgtaaagcacaacaaaaaatggtaatcgactacaaacacatgtacagtgatgggtgggaattcaatggttctgagaaggttgaacctttgctatctaagtacacaagtggtacaatgttccgtacaaacttacatgacaatgatcctgtttatctattacgtattgctattggtaaaggtcaatgggaagaagttgcaaaactcattgtggaaaatggtttagtactgaaagatggtcgtattttctgtgcaacggaagataagaattacttcaaacagcaaagtgcattctggaaacagaaccagcaaattcgtaagattcttctaaacagtctatatggtgcactattgaacaaaggttcacgtttctttgataagcgtttaggtcagtccgttaccttaactggtcgttctatgactaaacatatggcaagccgtatcaatgaaatcactactggcgtttataatcaccaaggtggctctgtgatttacggtgatactgactccgtttacttcagtgtaactcattacctgaaagaacaaggtattccatataatctttcgaaagaagaaatcgttgacatgtatatcaaaatcggtgatgaagttggtgaatcattcccttcattcatgaacgaaatgttcaatactggtattgagaaaggtaagattgttggtgcagacctagagatggtcggttcccgtggtctattcctgaagaaaaagcgttatgcaattctcaagtactgggaagatggtttccgtaaagatgttgatggaaagcctggtgaaattaaagcgatgggtcttgaaattaaacgttcagacacgccaaaatacatccagaacttcctagaagagactctaattgcattgctggtgggtgagtctgaagctcaacttaaacagcgtgtacgtgactttaaagaggtatttaaagacatgcctgtacacgaaaagggttcaccgaaaactgttaagaactataccaacttagaagcagaatatgaagtttctggtaaatgtaaagttggtcacgtattagcaggtattcaatggaaccgtctacgtgaagtatatgatgacaaaactgtacctgaagtaactgacggtacaaaagtaattgtatgtaacttaaaagataacccgatgggtatcaaagcagtaggttacccaattgactgtgtggaatatctacctgaatggttcttagaactaccatttgatacagagaaaatggaagaaatagttctaactaaaaaattagaaaacatttttggtattctggatatggatattggtattcatgagaaaagcacagcagccgttaataacggattcttcacctggtaaggaaaatattatgattattgcagacgaaactaaactaaaatatgacgaagtaaatgacgtattctctgatgaatcactagcactgtcagaggacagtgttaacgaggcgtgggaacagtacgttttatggagtgcatcccaaggagagtaatatggaaaatatctataagcgtgttggtgcagtactggaagatagtactatcgtaaattcagtacttgcatacaataatgatgatgaagttgttctaatgtctgctggtttcaccagaactggttctgttgaaattgacggtgagattgttgacgtctattctattgaaaagaaacgcaaagttgtcattgttgaagatgttaacacgttaaaagacttagaacatctacgttacaaattcaacattggtgcaggtgcaaccgctgataagccgtaccttaaacgttggaacacaaaatactgataaaaaattgagacaagcatcattcacgagatataatggtgcttgtagtaatctgaagcatacaaaaacatttttgattaacaaataaaacggagaattatataatggatctacgcaacgtactacgtgacctagttaacactaccaacggcatcgacttttcttgtatcgcagttagcagtgaagaccgtggtgatggacagcgtgtttacatggaagcatacaccgatgaccgcaccctagttatgcgtgcatatactaaagaagacgtaccagaagttcaaggtcgttttggtcttggtaacctaggtcttctacagggtctaatgtctctaaaaacttatagtactgaaggaactactatcacttctaatttgaaagatggtgcagttaagagcctagcattcaaagcagaagatgcaagcactactttcatggttcaaccagaaaaactaattccagcccagcctcgtttccacgaacaaccatatgacgtacaggttacacctagtgcagctaaagttcaggaactaaaaagcttctctggtgtgtttaaatctttcagttctctagtaactccatacactgaaggtgatactctttacttccacgttggtgagaagaacaaaagtaaccacgttggtgcactagcattctctaaaactgaaggtgaactaggtcttggttttggttataacattgaccgtgttctacaagctctatcccgcgtttctaacgcagaatctgttacccttggtattaccaaaactggtatgctaaacgtaactctagacactggtattgcggtatacaagatttacgtaacaggttgctaatataaataatggggaacatggagttccccaaatttttgatttaaaaataagtaaaggaagacaaaaaatgccattatatacatatcgttgtgaaaaagaagaatgtaaacacagtttagaaaagattgtaaaaattgccaatcgtgaagaacctcatgattgtcctcaatgtaatggtgaaaagactatgaagttggaagtttttacccctggtgataaaggtgctaatctttatttccgtggtaaatggtttgcaaacactggaggttactgatatgaaaattaaaatgattaacgattatgtactggttcgtcaaattgaagcacagaataaaagtactggtggtattatcctaggaaacgtggaaccaccatgtgttggtatggtgatttcagttggtgaaggtaaagtactacctaatgggcaaattgcagaacataatatttgtgttggtgatatggttgtattcggtcgttcatcactaaacactcctatcactgaagatcttggtggtggtgaagacacatattatgtgatgaaaattgaagaaatttttggtaagaaatcaaatggctaaaaaaaggtttattcctttctggatggtccccgctcattggggactttctggtaaagcgaaagagattgcgaaagcatactactataacgatgattatgaagctgatatgttagttgcagaacttacatatctaacaccatatgaagttgatgtagcacagttggatatcaaaaagaaatacaatgtattagaaactcttgattatctaatagcacaggcagatgtacaattgaagtattccaagattacccgtgatgagtataatgtaaaattacttgatgctaaattaagtactggtgcaattactcaaaaagagtatgatatggaagtaatcgaactaatgcctgatggtgatgacaaacgtcttaaagctatcgagtttgcatatcgttatgaagagattacacaacaagagtattcaaaggaaatctttactttacgtaaagaaccttggattgatttggatgtagtacttaatccagaaacaaatgaagttgaatttgtttttgattataatgaatacttttggaaaaagttaaaagcggaaggtcatccaggcaatgacgaagaagaaattattgataacttcgttagagacatggggcgtaaactagctacggatgatttcagtgatgattatgacgtaaagctagtgaatgcgaacgaagggttagagaatctacctggtttacctgaagggttccgttcatacaaataaaaaaaggagagcaaaagctctccttttcttttatctttaatttattaaaccatcgctgggatgaatttgaatagtttgataccgaagtcaccagcagcatcagtagctttggaacgtagaacaacttcagtaacaccatctttagcgagtagtggaactgcggttaggtcatcaattagtagacggccttcttcgatctcagtacttggaacgccttggaaagattgaatatctttatcaaatacgttagcacgttcaacgaggaaagtaactactgaagcactagcgatatcaacagacccaacacttaggataccgtcagcagcgttggtaaaaccagtaactgcaaatggctcaacgtcgttgccagcgatttcggaagtagcgatgataactgcacgttgttgaacaacatcaacgatacgtttaacgttagtttgtgcagcgtaagcagcgttataatcagcttctagagttgctgcttcaaatggagtaccatcgatacggatgatacctttttcgtttagatatgcaacgatttcatcagctacatcagtgtttaggaaacgttgtagaccagcgaaagaaccaactgcaccagtaacacgcacgttaacattaccaattttactaattgcgatagatgctagggcgtttttgccacagaagtaacctagaattttatcaaaggaataaccattgatacgttcaaaagaagtattagtattagacatattgtagtccccctattatagttgtactggtagagcagcacgaactttaattgcaacagcgtcagcggcagcagcatcaccaacaccatcagtacctagagcaacataagatttggtacggaagaatttagcttgtgcaatagctttagctagttcaacagttggatcaactggaacagagtaagcagcacctgggcgaacttcttgtttgtcaaatacgtcttgacgttcacagatgaaggttagagcaacagaaccagcaccacctgtaacatcagcaccactagtaccaaacgcgattacgttaccaccaacggttttaaagtcagcagggataacaacagcggtgtctagagctagtacggaagtagtaactagtactgcacgttgttgtagtagttgggtaatcatgaattggttgtattgagcattgctgattgcagtttgtagtgcttcatcttgaatttcggaaccgtcagaagtaacagcggtagtaccaacttcaacatctgctggtagatcaacaacgatggttacgaaaccattgttaccaccccagtattcacgagcagtagcagacataccatagttacggttaaaagaattaagactcataaaaatctccttgtcaaattttgtgcaggaacaccctgcttaatattatttatcaaaaaatataaaccacatttaaatttaaggcaatattccctgatctctatgatattatttatcaaatctgggaaagtgattaaagatgtgctataattatcaacgaaaatggagttttttacatgcaacacacgttcaccgagaaaaggattttacgatgaaaaaattatatctacttgttgatactcaaaactgtttccaccgtagtattaacgtttcttctcaaagtattgatatctggaccaaggtaggtcttgcacttcacatcactttgagtggattaaagaaactacaagaaatgttcgatccacaccatgtagttttttgtgcagaaggtaagtcctggcgtaaaaaattcgacgaagattacaaacgcaatcgtgcagtgaaagcggcaggtcgttctaaagaagagaaagaagaaatggaagtaatgtttgaaatgattaacgatttcattgacttcgttgataatcaaactaacagtactttactacgtgctccatcagcagaggccgatgattttatcgctcgttggattcaaactcacccagaagatgatcatatcatcgtcagtactgacaccgacttccgtcaactactagctttcaatgttcgtcagtacaacccagtacaagaaatgatgtatactactactggtgtatttgataagaacaataacccagcagaagataagaaaggtaatctgattgaaacacctaaccctgagttcttgctattccagaagtgcatcaaaggtgatacatcagataacgtattcagtgcttatcctggtgctcgtatgaaaagtacaaagaaagctgttggtattgaagaagcattcaatgatcgccatactcgtggttatgcatggaactcattcatgaatcacaagtggacacgtcatgatggtgttgaagtttctgttaaagatgcatatgctcataatcagatcttagttgacttaactaagcaaccacaggaaatcattgaaatgatggatgatgttattttcaaccgtgaaccaaaaagtactcctatgattggtgtgcattttttacgtttcgcagcgaaatatgatttagttcacgttcaacgttcacctgaagcatatgtgaaactattcaccaaaaaggatatttgatatggcagtactaaaaaaactaactgagtattcttatatagcagaatctgatggttctgaaaagattggaatacttattgaccacagcaaaagccctactgagtatagtggggtggaattcttcactagtgatggtgtactgaaatttgattctataactgaattggaagagttacttggaacaccattcaagtatgaggaagtacaagtcaaagacaccaatactaaatttattggtgactatccagttaacgatactgatagtgtttatgatgtacaagaaggcgagaacggcattagtactttccgtaagagtgaaaaatctaagaaacgcttcttccctggctggtggcttgttaaatcagaaagtggggtatacaatcctcgttgtactatcagtactgatacgtatgacaatcataaagatgatatctatggtccatacaaaacatttatggaactaacgtatcaacagaaaaacttgtaaggagaatggggagtactttaacgagtacttccctttatttgttatgggcaatttaataactttcaataaattaactaattatgatagttcaactatctttgaaagaatgttagaaaattctgataattcgtatgtggcagtagttccaccaagagttggctcttctgaatcaatttgtgcctatgtactgtacaagattttaaattcaagtgaacgtccaagtattgttttatcgacaccagtttcgtttaaaaccaataggcaatcaatgaattattattttgaaagtatgatttcaaatattaataaccatttagattcaaatttgtcaatcatgcatggtacatccagtagtactattaaatgtaaaggtacttttattggtgaaatattatatagagcgttgtcaggtgataacttgcgaggttttggtagaagtgaaattgtaattgatatgtcaacatatatgagggatgagcatttcatatctatgactgatataatgagatatactcataatatgaaagtaataataaattctcgttcattgtcgttctttacattgttggatagattgccacaagtagagtatatcccgtttttcttgcattctagtattgatatttttgaatatcaaacagcaatgctaaatacattaggtatgaataaatttgaacaatactgcctaatgaaagggaaatatgatgatagacaatttagacaactatgaagacgaacaggagtatagtcgaattaagaagcctggtttaaaaatagagtactctgtttttgaacaggatgaacttgcaaaatgtgcggctgacccaatttattttatggagaattacttttacatagtaagtaaaggtggcgagatactttttaaaccttatgattaccaaaaagaaatggtaagaaacttccaaggttacgaaaacaacataatgctcactgcccgtcagatgcggaaaaacaaccgttgcagcggcattcatcttatggtatgctatgttcaattcaacaaagacaattctattgttgggtaacattttagcaacagctaaagaaatcatggaacgtattcagttcgcatatgaaatgtgtcctgattttattcgtgatggtgttgagaagtacaacgtaacagaagttcgttttgaaaacaaatcaagaatcattgcacgtgcaactacaccatctgctgctcgtggtctttctgtaaacttactttaccttgacgaatttgcattcgtaccagaaggtttccaagcagaattttggtcagcagtatcacctacactcgtatcatctaagggtagatgtataataaccagtactccaaacaccgaatatgaccagtttgctagtatttggaatgagtcacaacgcttcgttgatgaaggtggtaatgagtacgatgaagatggtcctggtataaacgactttaaaggtattatggtacgctggtcttctcaccctgaccgtacagcagagtgggcagcaaaagaagagtacaagattggtacatctcgatttaagcgtgaatataactgtgaattcgttacatatcaagaaactctaattgatggtattaagttaagtgaaattaagaaacgtagtgttcgtgatcctattaaagaaactgatgatgttaaatggtacaaaccaattgagtacggaatgacttatgtagtaggtcttgacccaagtggtggtactggtggtgatgatgctgctattcaggtatatgaattaccaacattacgtcaggttgctgaatggaaaaataacaagtacttgattacagatcaggtaaaacttttaagacgcatactccttcagattgcatatgaaatgcaggagatgggagctaggaatatcgagaacagtttgttctggacattagaaaataacagtataggtgaggcagctattcttgcggtacaaacattgggtatcgaaaacttcccaggtacattgataaatgaaccaagaagaacacgaactggtcgtatccgtaaaggtatgacaacaactaagagtacaaaaaagactgcttgtatccacatgcaaaaactgatggaaacgttccgtatggatgttgccagtcagtatcttcatagacagcttaacgattttatacgtgctggtactgaagatggtgtgtttaaagcaaaaatgggttgcaaagatgacttggttagtgctacacttcttgtcgttcgtatgatagacattatatccaagtttgaagaaaacactgctgaagttattggtgaaactcttgatgaattagatgagtcatattttatgcctctcggatacatgatgagttataaccgataaataatattataaaaaatgaaggagacaccgctatgatgaaaggggttctttgttcagacatttataagattttggcagcaccacaatacgaatatagtattacaatgtttaatgatgatggtgaaggtacgattagtccacaagatgcaaagtggttttatattcgtccagtaaacttcatgattcaagcaccaagtggtgatgacacagtacgtccagaagtatatctatggaaaagtactgatataaaagatgaacaaacattacaagttttaaaacgacttaaaaacgtttcaaaccaatatggttatggcttcactgtatacgactttggatctgggaatttacctaaaaagttttcacatatcgcaatgcgtaatatggaagaaaccaaacaaggtgatttaaccgagagcctttcaggaagtgcaatgcgttcttattacaaactgccacgagctaaaatggttgttgttcataagagtcgtgttgaagaaggggtcaagggttcccgttcacgaaacgtaaaagaaattttcgtggagtccaacggtgaacgtagacgaatgagtactacaaatttacatgctgcaaaagcaatgacccatcatttaaatgagggtggcgagtatggtgataaatttgggtcttatatagaaggttcagcacgtgaccttgaaaatctaaaagcgttgctatcagaacttgaaattggtggtcgaagccattatgcttccaaaactatgcaatacattaatgacttaaaaaataacttaaaactcgctggaactccacgtggatatcacgcccaattagggagacacaaggaactaccgagaattggtaatacatatatagataattttgcgaataagttaggaacgattggctccgatcatgaccacaatagaagttttgctaagtattttctacttgatgagtgtaacaagttagctgattatttaaatactattagacaaaatatcagtactgatgttcctgatcctaaagaaataacacgaagtgcaaaacgtgtatgtttgggttgtgtgccagttgatggtgaattcgaatttggtgatacaccagatgatgaaaacaaagtccttatgtttggtaataaaatcgtggatttaatccaagatggtattattaaggacgcagtacaggggatatgtgagaaaccttacatggaacctgctgatgctaaatttattattgcacttgggaattcacttctaggtaataaacctaagaagcaaattcttcttgaacctgaacttgaatcactaaaggaatgggcaactggtaaaaaaagcgagacagacaataagtaggtgatataatgatgtttgtcagggcaatgagccttgacaaacagagtaaaaaaaccttgggacggttaaaaatcccatgatataatgtaaattgttgatgagtcaatgaagacaaatcaacgttaatagcccgttaaaacttaacaaacaaactaatgacaaggaagcccaatatgcaaactctagctgaaattcgtgcaaaactaaaagctgaacaagaaaaagcagaagcacagaagaatggtacatttacaggtgaacgtaaagccgattctttcctagctttctggaatgtcccttacaatgaaccactaaacctacgttttctaccagatgctgatgagtctaacccattcttctggcgtgaacgtgatatgatcaacctaacgttccaaggtattgttggtatgcacaccgataaagttaccgttcaagtaccatgtaacgaaatgtgggttcctaaatcatgtccaattcttaaagaagtacgtcaatggttccaaagtgcgaatgaaactggaaacgaagaactgaaagcaatggctggtcgttactggaaaaagaaaacctatctaatgcaatgtttcgttgctcctgatagcgtagcagtaaaagacgatattgctccagaaaacccaatccgtcgtatcctagctaacaaagatatctttgataaagttaaatctatcctgctaaacccaggcatcaaagaactaccaactcactacattcacggtcgtgactttggtattgttaaaactaagaatggtggtggttacaatagctatgacatgtctcaattctccatgtccgagcgtccactcaatgaagaagaactggcagcgattgaaaagtttggtctatataatctaaatgattttatgccaaaacaaccaactccagaagaactaaaagctatcgaagaaatgttcgaagcatctgttgatggtaaaccttatgaccctgcacgttgggctttcgcttaccgtccagcaggtgtacaaaaaccaacaactggtactattagtgaaaacaaacctgctccacaagctccagtacaacaacctgctcctgcacaaactcctgctccgcaagcaccagtagctactcctactcctgctgctgaaccagtagctgctgcaccagcaccagtagctgaagctccaaaagaacaactttctgcatctgcactagttgctcgtttgaaacaacaaggtaagtaatagtatttgaaaagttaaggggcaacggagtttgccccgttttatttcataaaattcaaggagcgaattatgaaaatgacaagtggttttgcaaacattctaaaaaatagtactggtagcgttaaaaagaatactggtgttagcgtaggctttcatgatcctgatacatggattagtactggtaactttgctttgaactatcgtattagtggttattttgaccgtggtattccactaggtaaagtaacaatgtttgctggtgaatctggttctggtaaatcatttatcgtttctggaaacattgcccgtgaagcacaacaacaaggtatctttgttgttatgattgattccgagaacgcattggattcagattggctacaagcactaggtgtagacacttcagaagaaaaactactaaaagtaaacatgggtatgattggtgatgttgctaaatttacctatgagttcatcgaagggtatcgtaaagaatacgaaaatactcctaaagaagaacgtcaaaaagtattattcattatcgactcaattggtatgttgaatactgaagttgctaatgaccagatgaaagctggtaacttgaaaggtgatatgggtcacaaacctaaacagcttaaagcttacatcaccaactgcgtaaactcgattggttcacttaacattggtatggtttgtgtaaaccatagttatgaatcccaagatatgttcaaccctgatccaaaagtatctggtggtagtggtttgatttatgcatcttctatggttatcgcaatgggtaaactaaaacttaaagaagatgaagaaggcaacaagacttcagatgtaagtggtattcgttctaaatgtaaagtcatgaagactcgttacaacaaaccatttgaagatgttgagattaaaattccttgggataaaggtatggacccacgcagtggtctactagatatgttcgagaaatatgaacttatcgttaaatctggtaaccgtctaatgtacgttgatattgacactggtgaacagattctaaaatatcgtaaagaatggatgcgtaacgaagattcttgccttgatttagttatgaatcaattcaaacgtcaccctttgattgaagaagtcaaagaagaagaaaaaggtgatgaatcatttggtactgatattggaagtagtgaggaataatgaaaccttggcattggatcatcaaagatgataaatctaaaataatcgacatgattgaacacttcgaaaaagaaactgaagaagctcgtcgtgaagttaaaaagattggcatcattgaaaagattgctcaggagattccaggctggcatgagactcgtctcagtcagcttcaagaactagaagcggtacttgaaatgctagaaattgaaatgcgtcaactacaagctgaaaagtttaaatatttccttgaagcatatcgtcgagcactatcatcttctgactgtaaaaagtatgttgaaggtgataaagaagtttgtgaattatccgaactaataaatgaagtatcatatatccgtaatacgttttccagtattgttaaatccttagagatgaaagcttttcagttaaacaatattgtgagactacgtgctgctggtattgaagatagccgacttgactaatataaattaaataaaagacagttttatttaaactgtcttttttgttaggtaatacggttatgggttcaaataataaaaagcaaacaacagaacaatttatagaaaaatcaaaatcaatttatggtgatatattcttatatgataaaaccgtatatgtgaatgtaaattcaaaagttattgtaacttgtaaaatacatggtgattttttacaaagagcaaatgatcatttatcaggaaagggttgcgttaaatgtgcaatatctgataaaatgtccaaggcagttaaagatatacgtgacgttttggatatgaaaaatgtatcgtatgaaatggaaaaaacgtttgatgattgtaaaaatatacttcctttgagatttgatttttatcttccaacgtttaatatgtgtattgaatatgatggtcttcaacattttgaatcatgtggttggggcggtgaagaaaaactaaaactaactcagaaaaatgataaaataaagaatgattattgtatccaaaataatattaaattgttgagaataaaatacgatgaagatcatattaaaattgttctagaaaatatatcataaggacgcattctgcgtcctttttctatgcgagaataaaatgacaaaattagatgagtttctatcaaacgtatcagtactagacaccgaaacgactggtgtagaaagtgaagatgatattattgaatttagtatttcatatcctcacggtgcacatgagaatattgatactattgataactacactttgcgttataaaccactaaaagatataccaccagaagcaagtgctgtgcattttatcagtactgaagatgtagcaaactgcattggttataaagatgacttagaaaacatcgacgcactcatgggctgtcgtgattattttattggacacaacgttcaatttgaccgccgaatgatggtagataacgaatataaatatcgtaactcagtttcacagtacttgctcgatgaagataaatggatttgtactcttcgtttagctaagaagatgtttgcagaagacactgaattcaaaaacttaactctaagttatttgtggtataaatttggttgctatcgtgacgtacatcgtgcagtgaatgctcacgcagcaaaagatgacgtattcatgtgttaccaagttctaatcaaattgattgaagttgctattgaacgtggtcatattgaccctaatggtgacattggtgaacaaatagtaacattctgtaatacaccaatgcgttataaattcatgccatttggtaaacacaaaggcgaaccaatggaaactgttccattgaactatattgaatggatgattactaacagtgatatccttgatgcaagtaacccaaactgtgataaagatttagtatatacatttgagtacgaatataatactcgaactaattaataaaggtattcatggatgaaaacttgtaagctaaccttactggacgaggttaattgtaaatttgagggactagatccctcaattcgtcagaagatgattcgtgcaacagaatatgtacttccatacgctaagttcacccctgctggtcgtattggtcgttgggatggtaaggtacagtttatgaacttaggtggtagtacttactaccacatgttggataagttgttgccaatcttagatgaagcaaaaattgatattgaacttgaagatcatcgtgtcaattacggattcaattttgaaccaattgatgaagatatttttagtgatgttgtgtttggtgctggacaccacatggaaggtaagtcaatcaagctcagagagcatcaggtgaacgcagttaatgcttgccttgcaaacccacatggtttattattagcagctacaggggctggtaaaacgctcatcacagcggcattatccaagaaggtacaagagtatggacgttctgtgattatcgtacctagtactgatttggtgcagcaaacatatgatgactatgttatgtgtggtcttgacactggagtgttctatggtgcacagaaagactttggtcaccaacacacaatcactacctggcagtcattcaactctttgtggaagaaaacaaagaaaggtgaaattgaactgactgaacaagacgttcatgactttattgatggtgtggtttgtgtgattgtcgatgagtgtcaaggtgctggtggtgatgcattagttcaagtactctctaaagttatgaataacgtcccacttcgctggggattaactggtacaatccctaaagaccaagttgtcgctgcgaagattaaatgtaacgttggtgatgtgatatttaaaatcacagctaaagaattacaagataaaaaaatactaagcacatgtaatgtgaactgtattaaacttaaaagtaatttaaagttcgcgtcatatgcagaagaattgaagtacttagttactgaccgtgatagaatggcatatgtagcatcactgataggtgcgttagcagaatctggaaacactttagtactggttgatagacttgaagctggtgaattgctttgtgaatttttgggtattcctaagtcagaatttgttcgtggggaaaccaaaaagaaagaccgtgaatcttcatatggtgaaattcgctgggctgataacaaaatactcattgcaacctatggggtagcaagtactggtatcagtatttctcgtttgtataatgtcgttctaatcgaaccaggaaaaagcttcgttcgtacaatacagtccattggtcgtggattgcgtcgtgcagaagacaaagaccacgtggaaatctttgatatcagtggtacaaacaaatatagtgcaaaacacttacgtgaacgtttacaatactataaggaagcacaatatcctcatagtatcatagatgttaaggattgggaaaatacagcgggataatcccgctgtacatgatataatgtgtcgtatgacaacaacaaggaagtaaaataaaaatggaattaagatctagtattgcttttatctgtgaccaagaaacgttagatgtaattgatgtattgttgaatgatgatggtattatggatgacatggtatcttcacttgactatatacctgaactagaaactgacgatactaatgtttggagatgtaatattgacactaattcatggattaatatctatgaattgcaaaccagaattgactcgaaagttgaatcattaaaatatgatatcatctatgcaagtactgatactacaacgtttattgatgttgttagtcttagtggtaattttgattcttacttagataccgatgttgataagattcaagaaatgtgtgaaacgtatggtcttgaattctatggtacatccgatgaagatgaagaagattactacgatgaagatgaagattcccagtactaaggagatgagatgaacgtattaaccccagaaaataattcatttgatatggatttagtaactgaacacattccatcagaaatgtattgtgttttagatttgagtccaaacggtaactctgacttttattttcgccacattttgaacactgtatcatttaatagtatcagtgctgatttgcagattggtgaaaagattatccaagtaccattgggttggcaaatcatgttgggtgatgaagagacgggtatggttgagatgtgtaccattgaaaacattcttaatatgaaagaacctaaagcttttgtttacaatccaatccgttcgatgtatcctaagtttgaacctgtaaaagtactgcgtgtattcgcactcactactaagtggcagatccctatgctgcctaagaagaacttactatgtgttcctctgcacagtggtgataatccaccttgtgtttactttgcagatgagaacgagaaaattcaagatttgtatctaggagtgtaatagtgttagatttcagtaatattaaagctaagtcagtagacgaaaacgataaaccaacccagcatacattttctattgctgaatttctaaaacatattgaccgtgcagattacaaatactataatgacatgaccgaagatgagcgtaaggaatttacgccttatactgtacttcgttgggtaagttcacttgatgactcacttcaagtgactttccctagtagtaaagtggaaaacgtatttggtaagtggaaagctggtggtaaagaagcactaactgaacttgtcaaagaactcaatagcactggtgttaatgtaacatcagttactaagtactgtcacagtgactatgattggcgtatcaagtttgccgtacaaagcaatgatgatgctaacaagttgattgcagcaatgactgaatttagtattgctggtggacagatcatctcattggttgacagtgacgttgtacgttatcagttgattatgttgaatgatatggttaatgatgggttctgggatatgaaagaacaccctgaacttgtatatcaactactatgttctgttcatgatatgttgggtggtaaggaattgccacgaaactggttaaacttttccaagagttttaagcgtgctaataatgaattatttgaactcattaaaagtacacagccagattcaatcgcaactcaaatgaacgaagatgaatataaaattgttttgttgcaatataacaaaaaaacattcgagtctttccttgaagacataggtttacaagagaaagaaatgaaatcaatgatgaaattatttaaagaagaatgtaagaaatatggcaaagaaatcaagtagtcccgtatacaaatgtaagttctgtgataaagaatttaaacgcgaagacacagttaacacacataagtgtgttaaacgtgaccgctacgaagatcgtgatagccgccccatgagggaggcttttcgcatttatatgcaattcatggagttaaaccatttccctttcaagaagggagttgaaccattaatggcttttataaaatcaaagtactttagtgatttttatgattttgcaatttattatttgaacaaccgtgatattttagatattgatggttttgtaaaaagtctaattgtaagaaaccgtcctattatggaatggagtacttataaagcaaaggatgaatggatattaaattgtattcgttcagaatctcccaatgctgcggtttcccgtagtgttgttgcgatggtggaatggggtcagaatctgaacttggagtggaataagtttttttccaatgtgagttctgcccgtgcaattgcatggattgagagtggtaagatttcaccttggattatctggttagcaccaaaagaaagtactaataagtttttcaagaatctctctaactctgaaatcgagtatatacttaaatacattgatcctagttactttgaggttaaaactataaagtacaaaaaggaatgtgatgaaataaaaagtatgctacgtgaggctggattatgaatggaaaaagtatatataataaaataccagaagggtatgtacctgtgcctaaagcagataaatctaaatctttatcattgcttagacagggttctataactgaaattacacttgatggtaaaacctttcaggtgcacgatcctgttcgtgtagaaaaaattatcagtatggttgaaagacatgaggaaagattctttggtctaagtcaagagttgtcaaagaccaaacaaaagtgtacaatgttagaaaatcaggtaaccaaattatctaacactgtaaacaaattgcaggagcaaatcaaaaatgcaggattcgacaaattctaattcgtaccgtaaagcacgaacagatatcgacattgactttaaagatagtaagtcagtgattgcacagcttccttgtgtgcgaagtacagaaaaaattacggaggatggtttaatcccccataacagtggtgttcattttgataatataccaattgaccctatcactggtttagccagcatacaatataaagaagccgaacgcttgggatatcaaaaggtagatatcctgtcacaatcggcttaccagtatgttcgtgatagagcacatttaaaagaactaatgaatcaggaacctaattgggatcttctgttagaaccagaaatagttgaacatctttcacagattaaaaaacacgttacattattaaatgtgtggaaacctaaatccattgatgaacttgcaatgtttattgcaatgattcgtcctggaaaaaggcagtgccaaagtatgaatacatggagtgaagttgaatccactatatgggattacagtactattggcacagaccaagcaggaaataagttacgatatttcaaaaagccacatgcttatgcatacagcttaatgattgtagttcaacttaacgctttagttgaacatcttacttcttcttaagagccacattacgacgcttgatgcgtttttgtggcaaatcacttaaagaaactactggaccacaaacgaaattcaccgcaggggtatcgaatctctgtgtgtaaggttcaaatttctccatctcttcttggaagaatagacagactggaattgacctgttgctttgccaccaccaatcatgtgctatacccaacaattcatcttgcattggttcatccatatcatcaaagcagtaaagagtagtatactgtttactttttatcttgataataccaatgaaagttttaccatcacactgaactaccgtgataaatggtggaatagtattatattgattcatatgagttccataaatattagtataaatttatacctattatttatggaaggaaaaactatggctcatcacggcgtgtttatgtacccttataaccaagaagtatcattgggcaattttaatatgccatacagcaatgagaaacttacaatatacagaggtgcaaataacaatttatcattcacagtgcataacgctgatggaaaatatacattactaaaagataacgaatatctaacattcatgatttttgatgctagaaacaatactaaattattcgatgtagttttggataaacaaaaaccatcatggctatcagaaagtggacaagcaagacaaacattaggcaatagtaaaaaagtatattatggatgtattgttcctgctggtgtaattcaggatactagtcctggttcaaaatatagatggagtattactaaagtaacaatggatggttccttgattgaacctacacagtacttatacactggtcttgattatgaagcctctgctgaattgatcatttccgaccaagctgctcctgtgtttgtgccaagtattgaagtatctgttaataaaaacccagcatggactaccatcaaacgcttgtatcatgaaccaatcgctcgtggcgttattggtgaatttgatataatggcaacatctccccttcctgcacaaatgcagtacggattagttgatggtcttagtactattgcaatgtactttgataaattcattggtagaattcaattacaaggatgtttgtgtaataacgtaccaactgataacgaaaactataaatggtttattgttaaattggatggtaaagaatatatcgaaaatgatattgatgaaaatggattccctaaagcgttagatgggattcaagcatttaattttaaaggtaattacatgtggttgcgattggttgtttgtattcatcctaagatcgttccagttccttatgactatgcggcagtaagaaaagtctatgacccactactgtcagttcctaaaattttgattcgcaggtgagacaaaacaagtgtttgtgctataatcatttgtgagaaataacaaggattaaattatgaacgctgtacaaaacatcatggcacaaatcatgaatgacccagtaccaggaattggtatgtggacacatcacaattgtcctgcttgtgtgttccgtggtcattcacgccccgatactaaaaatcgcggcaatcatatgtttaacgcagatggttctgtttcttataactgttttaactgtagtctaaaaactgggtggcaacctggaaaatatatgagcaaagatatggagtctttgctcatttcttttggtgcacaggataaggaaatagaactagctaaattttacattaaagaaatggttgatagtggagactttgcagaacaagaaactactgcatctaaactatatcaacgtgtaactgttcgtgaattaccaccgaactcaaaaacattctgggaatgggcatcgttaccagaagagcaaataccacaagaatttattagtgttctgaatcatgtgaatgagcgaaatccatatctattagatttagaattgtattggagtccagctaaagaacataaaatgtatgaccgatttatcataccatacgttatgaacggtaaaattatcggatatactgctagacacaaacttaaagacccaggaagtacctatcgttttattaatcaggtcagtacaaatattctatataattatgatctgttacaagatgaacgaattaaaactatattagtttgtgaaggtccaatggatgcagcactaatgggtggtgtttctgcaaataactttaaattgtcagcagtacaaatagatgtacttaaagctgcacaagaaaaaggtaaaaagattgttatcgtaccagaccgtgataaggatggattacaaacaatcgaacaagcactagaatatggatttagtgtttctttcccagattttggggcagtacgtgttggaagtactatacgtcacattaaagattttgacgaagcatgtgctagatttggaagattattttgtttacaactgatacatgatagtatagttgacgagcctttctatattaaagcacaaacggataagtggatgtaacacaggatataaatgagggaacataaatgattcaaagcacagtggacgtcaaacagtatgggattaatgcacaacaatacttactatcccaagttttcagtaatccagatttatttgtacgttgtaaaaatattctaaagccagaatattttgataaaacatttcatgaaagtattcagtacgttcatgattatgcagcaaaatatagttcatcccctacactgaaagatatcgaagtgaaaggtggttatcagtatgatcattatgaagtacaagaacttcataaacaggcagtactggatacctgtagtgagttttgccgtcacaaagctctagctctagcagtagagaaaggtatggaattagtacatcagaaaaaatatggtggcatcgaagaactcattcgtgaagctatgttagtatctgtacagaatgagttgggtcttgatatctttgaaaatccagaagataccattcgttcattgagcactatgcaaggttcattcaagagtggatgggaaacccttgactataaactattcggtggttttggtcgccgtgaacttgaaatctttgcagccgcgtctggtggtggtaaatcagtagtactacagaacttagcagttaactttgctaaggctggtatgaatggtgcatacatttcactagaactagcagaagagttggtagccgttcgtatctatggtatgatgactgaatctaagctaagtaacgttaagaacgaagctgaaaagattggtctacgagttcaaatggaaggcaaggatagtggtatattgcgtattaaacgtttaccagaatccgtaacaaccactgatgatattgaaagttatcttcgtgaattgcaaattaaaatggaagtcaaattagattatgtttgtgtggactaccttgacctattaacatcaaatcgtcttggtcctaatgataaatctaatacatttacaaaagataaatttgtatctgaagaattgcgtgcactagcaaccaaaatggatttgaccgtcttcactgcgtcacagtttaaccgtagtggtatggatgaagataagaaaagtcaaagtcagattgctggtggtatttctaaaatctatactgcggataacgttatctacattgaagcacgtaaagaaactggcgaactttggtttgacttccagaaaacacgtaacagtagtgcagttggttcacgtttgcgtatgtcatatgatatcgattcacttcgtgtactagaccacgcaaccacaatccaacaattcgaaactgatgggtcaggattccgtcacagtggcatctctaaggtcggcggtggtaacatggcaatgccaccaagtacaatgcaaacactgcgtcatacgctttctggtgctcctaaaccaagcactacaccgagtgttgaaagcgtaacactaaatacagatagtgatccaatcacttctacacaaactgaatcccagcctcaacagcctcacattggtactgatcgtgtaaatgcaatgaagcaatggttaaatcaaaggaaataatgaatgtctagtaaatccaataaaaagaatctacttgatagtatagttgactatacaccagagaaagatacattacagagttttggtaatcgtgcagatcatgcaatcaactctgttattcatttattggagagtatcgaagatgaatttaaccaaactgatgctgaagatttgaagaaacgtttatttcttagtattaagaaccgagattataaaaagtttcagaagggcttggagaacatagcacttgatgaaaaacgtaataaagggaattgatatgaagaagaaaactaaactaaaagaaaatgcttctgctggtgcaacatcagcaggtgcagtagcatcagtcagtactggattaaattacccattacttcgaagaatgcctcctacaaatttctttggatataaagttgttgagactaaaaataaggataaggaatgaaaccatatactttctacacaattaatactgttgtagatataggtgatggtacaactccaacaatcatcgattctaagttcactggtgctatgaatttgtcgagattgttggaggctattatggcacatgattatcccatgatggtaagtgtagtaaaaactgaagtagatctatataaagataacaacgcatctttctataatcttccagtttcatggggtaaatgtactcttagtactttcaaattcgcaataccaatggatgttgacttgcgtttgaatggaataccattagttttacctacagaattaaatggtaaaagaattacaaaatttatcacaacaggtaatgatacaaatattagcatcagcaaacagaccttttcttaattgtgataaatatcataaagcaagacagaggtcttttttattttatatggagtaattatggatataaaccaaacagcagcggagctacagaagtttgctgacgcacataatttgaatgttgatgtttctgttgtactggataatatggatgcgggtgatttcatcacactaaatgccgctatggataattcagataaccgtgaaatttttcaaattctacaaaagtacaaagcacaaatgagtgaaagtttctcatacttcagtactgataatgtaactaaacatattgttgaaagttctgattggcataagacagttgattctatgacaatgaaagacttagttgaagcatacaaaagtaacttcacatactctgatgtttctaagttgagtattcaagaattaaaaactattgtgtttgaagatttatcctctaatttggaaaatccagaaaatcaattaaaatctacacagattgctaaaatgaatgtgtcacagcaacaacagcaagtgaacccacaaacccaagctaaacttaaacaagcagaactacaacgtaatgctaataacattaaagttagcgtccctgggaatcaaaacggtactgattcaattgaacctgttgttggtatcgatgctggacaaaacccacagcaatctttagtagtaactaaaagtggcactaagccaaacgaattgaatgttttccctttggatgatgtaactccagttgacaatcaaaatcaaagtactacaattcgtgaagagaaatcaccacaagaaattaatagtgaagtgatgggtgagcctggtatggaaccagacggtaatccaaattcaccatctccactaacacaacacgcaccaggtatgggtgaaatgattcatgcaatcgctgatatcgaaaaagaagatgatatctatggtgatgaaagtgtaatggcaaactctgcacaacaagagaatgatgagatggtagatcaaatcattgatttctgtcaccgtatgcgaggccgataatgagaactgtacctaatgttcatggtcacaacactatggtttccaacgaagaatacagactattacagaaaattaaggcacgaggttccgtgccagttgaaaaagtaaacgaatactatcaggaactagcggataaaatggtatctcgtggagtcctaaatattgttgagaaagatgacggtactgaagagtattcaactgttaggagtaataaatgaattacgagtacgatcaacaatcagttaatcagatggctgatttgttaagcggtttgaatgacattgaagctggtgcaacccataatgcgtctggtgttccttacaagtcaggtggtgatatcggtgcaatgaccgacattttgacatcattaaatgaagctggtatgaattatggtgttgatccagaacagacacatccacatgttgttccattccacgccccatacaatgaacacatgcttggtcaccaactagcacaacctgttgtacaacgtgcacctgctaaacctaaaactaactggagtttagtagaagaaaaaattcaaggtactaaatcaacaaaagtatacagtgttaaatgtaatcatagttctcaagttatcatgaataatattatgatgtatgaagctgcactaacactagtaaaccttctcaacgaaggaagaatgttgacagatgcaaaagttcttggtatcatcagttccggtttacaatacaccaatgtaattaccgaagcattgaaggctgcacagaaacgtcaacgtgttctgaatgagtcgaagtacgatgaagcaaaagatttagatatcatcatcgctgaaaagaaagctgaagcatacaaactcaaagagcgtgtactgaactttctaaagagtgaaggttatataaagtaaataaatttagtgatattttataaggaaattcaaatgtctaactacaaaaaactaatggaatctgcttcacgtgttgaagcaatgaagttcatcaaattgaacgaatctaatattgagaaagctgaaatcgttctctctgttaaaggtgaaatcgttgacaaactacaacgtcagtctgaagtaatcaataacatgggcgttgacgttctaggtccactacttgaccgtatcaaagcggaacaaggtcttgatgctgcacagaactttagtgatagcgttcgccacctacttcaacatgcagttactactctaatggatgttaaagacaaaatcagtactgaaactttaaaactaactggtgatatcactgatggtccagcagtttctgatattggtagtgaagatgttgatatcgacattgctgctgacaccccaacgttcgacatgggtgctgatgctgatttcgaagaagatattatttctgagccagtaccaactgagcgtgaaatgaaagaatctgtaaaaccaactcaaattggtattaagatggaaagtgttcgcggtactgtaggtactaaattcttcaaaaacaaagaagaaatgaaaacttggctatctgagaacgagaagaaagttaaacgtgtactaggatacgttaaaaaataattaaggatgaatctaaatgagatttcaagatcttacagaagaggctcctggtaaagacatacgcaatatgattactactattgcgttgtctttagtatcatcgggtcgcgaagaagtgaacctacaatctttaatcgctgaaatcaaaaagcgaaccaacattgatgttccttataatgttatgatggatattctaaactctttgccatttgtgcaagatgcgaattcggatgttgtaacatttactggtaatgattcagaaagtactggtgaagttgaacagaactctgaagaccaagttgaagattgggcaacaaaagctgctgttgcagcaaataagcaataaaaactgagacaaacaaaaatacgatgctataattaggagtacacggatgtacacaccaccaattgttagcagatatacatacgttgattacaaaagaattgaaggcggccccgaaggtcgcctttattcaactccaacagggaatctgccgtcagtaacaacaattcttagtgccacctctgacaatgaggggttgaaaaaatggagagaatttgttggtgaagagaaagctaatgagattacattagaagcatgtactgttggtacttttatgcacgagaacttagaatatcgtctggatggaaagccagaccaccaaggtggtatgccgatacgagttctggcccgtagaatggctgacaaaattcaagaagaagcatggcctagaatcaatgaagtgtggggtcaggaagttccattattctatgaaggattatgggctggaacaactgacttagttggagttcatgatggtattccttctatcatggactataagaattcccgtaaattaaagacatgggaacatatcgaaaattaccgtatgcaaatcgctgcatatgcactatcgcataatcaaatgtttggaacaaatattgagcgtggtgtaattttcatctgtgtaagaaaagatcctaacaatttacaatatcaagaattcgttattgaaggacaagacttcaaggatgcggttgatatgtggattggtagagttgaacaatactatattttacgtggagaaattgaatcaaatggaacagcataaacaaatgaaacaagacctatatacaattcgtgcactagaaagtaaaggctactccttagatgaagtaatcaaactaagtgatgaagaaattgacaaactacaactttcaaataaacttctagttaatattaaagattataaatctcgcggtgcaaaaccagtagaagtaatcaaagagaaaattgctgaagatattagtgttgatgaagtatctcgtaatgttgcaccagaagtaatcgaatcatatgtagtagatactgaagaacaaattaaagtatctgaagaagtacaagtaatcgctgaagaactagcacctgttgttgaagaagaagacgttgttgttattgaacgtgttgaagcttcaaaagaagatttggatattatcaaagaagcactaaaaaccaaagaactaaaaagtgttgcaaactatgtaaaacacttgaaatccctagttcctgaatcaattctttcttccgttgatagctctgttgtatctcaactaatcaatgagcgtattgctgaagttaaagaaaacgctaaataatcctataatactataggagatagtttttatggcacaacaacaaattgcgttacagcaagtaagaactggtaagcaacgtgacttaccagacgcgttggaaaaaggccagataggattcagtactgatgtgggtcgtgtattcatcggcctaccttcctcatcagacccagcaagcttagttgctgggcgtacatgggaaactgatccaaactctggtaaagagaacgtggaaataattaccgaattctcaccgtggagaacggtatccaaaatcatcaaccgtccatttgaagtagacattccacaaaaccaagagagtgttgaagttattattgaaagtgaaagtcgtgtattcatcgactacatcgcgtataatagcaatgagactattttagaatcaggcacagtgcaaattgttgctgctggttcttcaactatgattgcacagtcaaataacacaagccgtactgatgcaattttatctattctgtttagtaatccagtttatgattcaagtactggaaatatgcaatttgacatagttaatgcggattatttaaattcaggataccacatagaatttgtatatcgtgggtgggacaaaccttaaaaggcgtcccgcctttttcttccacaaaatttaagaactgtataaacaatataacaaggatgacacatgattaacgtaaccaagcgtaatggaaaaaaagaaccgttatctttggataagatccatcaaattttattttttgctgccgaagggctgagtggtgtatctgtttccgaaattgaaattcgtgcacaacttcaattctttgaaggtattgaaacaaatactattcatgatattctgattaaatcagcagcagaacttatttctgaagatacaccaaactaccaatacgttgctggtcgcttactaaattatagtatccgtaagcaagtgtatggtgactatgaccctatcaagtacactgcacatctacaagaaatggtagatacaaaaatctatgatccagaaattcttgagaaatacacacttcaagaaattgaagagcttggacgttacatcaaacataaacgtgatgataaattcacttatgttgctatgcgtcagtgggaaggaaagtatctatataagaatcgcgtaactaagaaaatctacgaaactccacaaatggcatatatgaccattgcagcaatcatctttatggagtatgatcgctctgtacgtatgtcttatgttaagaagttttatgatgctgttagtaactttgatatcagtctcccgactcctatcatggctggtctacgtaccaatacacgtcagtttagttcttgtgtagtactaaacgttggggatagtctagatagtattggttcttctaaccacgctatcctacgttatatttcccgtaaagctggacttggtatcaatatgggccgcattcgtgctctggactccgagattcgttcaggtgatgcagtacacactggtttgattcctttcattgcatcaactgctaagaccgttaaaagttgctctcaaggtggtgtgcgtggtggtgcagcaacagtccacttcccattgtggcatttggaatttgacaatcttgtagtactgaaaaataacaaaggtactgagttcaaccgtgttcgacaacttgactattgtttccaagtgaacaaaacactatatcaacgtttagtggatggcaagaatattacattctttagtcctaatgaagttcctgggctatatgaagcattctgttctaaccaaaaagaatttgaaaagctgtacaagaagtatgaagctgatccaagcattcgtaaaaaatctgtatctgctgttgaagtattcagtactctaatgactgagcgtgcagaaactggtcgtatctatattcaaaacattgaccatgcaaacacacactcaacctttattgaatcaattgcaccaattgaacaatccaacttgtgtcaagaaatcgcactaccatctgtgccatttgatgatattaatactggcgaaggtttgattagtctatgtactttgagtgcaatcaactggggtaactttgaccgcccagaagatatggaaactgcttgtgatctttctgtacgtgctcttgattctctattggattatcaagactacccagtagttcatgctgaccgtgcaactaaaatgtatcgcccattaggtgttggtataattaactttgcatacttccttgctaaacgtggtttgcgttacaatgaagctgcattggaaacagttaacgagtgggctgaagcatggtcttattatctaattaaatcttctgttcagcttgctaaagagaaaggtaagtgtgaaggttttgacacactaaaatatgctaaaggtattttaccaattgatacctataaagaagatattgatgcgttggtatctcctgaactaaaacaagattgggaaacattgcgtcaggatatcaaacaatatggtattcgtaacgcaacaactatggcactgatgccagcagaaactagtgcacaaatcagtaacagtactaatggtattgagaaacctcgttcacttgtaactaagaaactatctaaagatggtattctgaaacaagtagttcctgaaatcaataagttgaaaaacaaatatgatttattgtgggaccagaaagataacatcggttatctacaaatctgtggtgttcttcagaaatacattgaccaaagtatcagtgtgaataccagttacaatccagcacacttccctgatggtaagttgccaatgtcaacactactaactgaattaatgtatgcatacaaagttggtttgaaaaatctgtactatcataatactaatgatggttctgatgaagttgatgttgagaaagtactaaaagaagctgcaaaaccagaatcaggacagtttgtgttgcctacactacctgacttagatgaaggtgattgtgatagttgtaagatctaattaaagatcccgcttcggcgggatttatttcataggagtgatataatgaaagttgactatcgtagtccaatagatattggattcatagaagaattttgggatgatcttgatatggatatgattgttcgctatcaaaaactcccacaagactttataaaaaaacattttgataaaatgaaccctaatatgttagctcgatatcaaacaatgacaattgattttcttaaagaaaagtgggagtggtttgataaaaatgttattgcacaatatgtagtcatgccaatggaattgattatggaaaaatggaatgacttcaaagataactttctggatactgttgctaagtatcaaaaactaacacaagattttattcaagacaaatggaatgaccttgttgaatttagagatggtgtaatgtcggaaatttccagataccaaaaactaacacttgatttcattaaagataaatgggattatattgataagaactatatttcatattatcaaactttaacggttgattttatcaaagataatattactgcattaaatgttgcagcagtatccgcatttcaaatactatcggatgatatacgctcattgatggggttagaagcaccagcaaaaactattacgggtaatcaaatactcgcgttggacccatgtgccgatggtatggctcgatacaatgctcatacacctgacaataatacagttttgacatggaatgaactacttacactacacttgggttcacaagaaggaattagtgatatccattggctatcatataaattgggtcataaaattaatacataaaggaatatatatatgtcagttttcaaaattaatgagaagtctcacctagaaaagaatatgttttttgatgaaagtgttgatattcaacgatttgaaagtaacaaatactctacacttgaaaaactaactgaacaacagaaaagtttcttctggacaccaggtgaagttgacgtatctaaagataaaattgattttgctaacctcaatgattctgagaagcatatctttacatcaaacttaaaacgtcaaatccttcttgattcagttcaaggtcgtggtcctaatctaatgctactgccatatgcatctctacctgaacttgaaaactttattgaaacatgggcgttctttgaaggtgctattcatagtaaatcatatacacatattattcgtaacatttatcctaatccatctgaagtttttgatactatgttggacattccagaaatcactgattgtgctgctgatgtttcaaaatactatgatgaccttgatgaactaggaacattgtatcgtgcattaggtgaaggtactcatactgttaatggtaaaactattgaaattaatatgtatgatctaaagaaaaaactatggttatgtttgaatagtattaacattcttgaaggtgttcgtttctatgctagttttgcttgttcatgggcatttgcagagaacaacctcatggaaggtaatgcaaagattattcgtctaatttgtcgtgatgaaaaccttcatctcgcagcaacacaaatgattttacgtcttctaaagaaagaagatccagatatggtaaaaattgccgaagagtgctatgatgaagtactggaaatgtttatgtctgctattcgccaagaagaagcatgggcagatttcctctttaaagatggttctattattggattgaactctgaaattcttaaacagtatgttcgctggattggttctaagcgtatgactgcacttggtatcaaatgtccatatactgtatccaagagtaacccactaccgtggactgaacactgggtaagtggtagttctgtacaggtagcaccacaagaaactgaaatcagttcttatatcattggtggtattgtgaatgatatccaaggagatacattctcgccatttaaactataaaataaaactaacagggattctatgaatcccttttcttatggaatttatatgagtgatatttttacaattgagcgtaatgttatctatgacttctacctaactggtggatttaaggtaactggtgcagtatctgatgtttgtcccaatggtcttgtactggtagatggtactagagtcgttcaggcacacattgtcatgtttgtaccaaccgaataaaatttaggacaaacaccagaactgtgctataatgagttttgttaaacaaaaggagatataaaatgaacgttaacaatctaaatgaaatggtagaaaagaatatcgacgctgcggaagaagttactgggtatgaattaactcctgaagacctagagttatcgaagaaaatcattgaacaactttctgggtattatgtacgtaaagaccagaaaattaaagaacataaaaaagcaaaagctaaaaaccgtgctcgtagcaaaatggctaacaagtctaaaaaagcaaaccgcaaataaatgaggcattgatgatgcagtatatattgtctaaaattaatgagaattttcctaagtcgaacgtgaaaagaaccaaccgaacacttaaaaaaatgcatttggatgaatatgctgtgtcgttggcttcaatcactattccagtatatctatttggtctagaacaaactcaaactgaccgctgccttgatgtgatttatgaacacgatgttgggtgcttcgtgtgtggtagtgatgatggcttcactatactacatgaatttgaatcattggcagatgatgccagtactgaagaatatattaaaaacttcactgaaaaccttattcgcgaattatcaaatattgaaatggaattcgcaactacacaaacagtcactatccaatacggtgacgcatattatggagaatggtaatggcaactacactagaacagtataaacaagtaactgaatttatggaagtgtgtaatcaagaagtaaatactacccgtaaatttcaaacagcaaaagttgcaaaccttcgtgtgaatctaatccatgaagaaatctttggaactggtgaattagttgaatcagtaaattacgataataaaactggcattttagatgcattgtgtgatattttgtacgttacttatggtgcaatcgcaacgtatggttgtgtagatgcactaagtgattatgaagtatccatcggtctaccagaagaacgtgaaggaattttactattcaaacatgcttcctcatcttatattcgtgaaatctcaaactactttgagcagttcaaacgtggtgttgagagtggtgatagccgaactattagtgatggtctagttcgcattgtcgcttcatgtgttgaatttgcacgagttagtaatttcgatctatccggtgcatttaatgaagtacatgaaagtaacatgagtaagttctgtgcaacggtagaagaagctgaagaaagtattgctttccgtttagctgaagctgaagcacttgaaaataaagtgaagggtctagaacaaacgcgtaactacactgatgcttatgtggagaagagtggtagtgtttatgtaatcaaacgtaagattgatggtaagggtcttaaaggtaaagacttctttgagccagatttggctaagtatgcataattaaaaaaaagaaagggagcaacacaagtttgctcccttttattcatttaatgttttgtattgaatcatcaaagttcttcatatatggtcgaatattgttaattaagttctgaacttcatgctgattaaatgttctattggttgtaatcataacatacccagcaacttcaaactttttcttatctaggataggaatagttattattgtgttataagtataacgaatgtgtttaacatcatcgcatgaataatctttggtattatcatagaataaactacattctagttttgaaccaatgattgtttcataatgaactttattaagaaatatttcttgatatgctctatcgcttctgtctaaagtgtacatatcagtacgcattcttcgtacaccagagcgatctctaagtgctagtccgaacttggcctttatacattttagtgtcttcatcaggtacaaatttgtatagaaccaccgtactaatatctgaattctttgatagaatttctataaaagactcatttaccttatccagctcaaaagaacttttgaaaaaatatttttcaatactgggtattaccgttttggtaattctattttctatctgtgacctattaagaaagagaaaaatactcaaagtgatacaagaacatacaattaacactcttagaattgaactacaaattcttttatgtgttgacatgtgtttcctcttttatttaaaatgagatataatatcctacttcattaactactttcgttgccatttgttgaaacttttcagcttcttcgtcattttttggtacacgtttgaaataaacactaacatatccctcgattgaagtgtacttgattggtacagaaacaagataaacaccaccgtcttgaactattgctgggaagttaatcggtgatagatattcatttcgtgcttcattttcaggatttaaatattctgtgtaaattttagaaatctcaacacaaaatattttcccccttagtaatgtatttgtttcagcactaaaagcactaattggtagccaatctaaattgtatggtttaatattgatacccatagatgcattagtttcatctgtgacaagtactctaccttgatagaatgtgttgttatctggaacaaacttataaaccaaaaccattgcaatatcttcagagtttgcagaaacgtacttataaagacttttatcaatactttctaatttctctggagaaattgtgtatttgtgtggtttaaacgattcatattgttcttggttgtttagtttgttgttgatgtaagtttcccatgaactggaagttatatatactacaaaccccaagacaagacacagcgttactactattgttctaataacaggcattccgggagataacttcaaggattttgttaattatactactatctaagtcgatattcatatacccgttaatctccaatcttattgttatttttataaagtatttacccaaatttacgttttttgatagtttatatggagttaaagtaataaatgcttattttacaagttatgtggattgatattatattaatctttgtatgtgcagggtatagcactaccgctagtatattatactgcatatcgttgatttttcttgcttttatggtaatgttccataaggatataatttgtgtatacaaactaagagataaatcattgataaacaattttgaatcaattgctcatatctatccaattggtagacgtactgttataaacataattatgtcatctgttattaacggtttcatagcactagtggcattgaactcacttgcaatcaccattgggattgtggtattatgcgtatatcgcatttataatgaagaacaactggagaaattagtatgtcaaagatgattctatgcactgacgaaaactttggtattggatgtaacaactcgatcccgtggcacagttcagcagactttgcccatttcaaagaagaaacaaagggacgcattataatcatgggatataatacttggaagtctctaccgaaaaaaccacttccaggtcgcatgaatatcattctaatctcacgtgaatgttatgacattgaacagtatgcagatgatactagtgtgatgtttttacccgaacgttgtttgcctgaattgctaatgttcaaccctgaaagtatcgttatcggtggtgcgaagatttataaagctgcacttccttatgtacaagaggttattcacagtactgtaaaaggtacatacaaatgtgatacgtttgttcgattcaaagatgacaagcgtgtaaaactatctcgatatgaagccaaaacattgtcggatggtacgcttgtagattactatgaaatcgttcgcgtgtatgatgattgggtggatgcatatcaatgaaaatagacttatatactgatggctcatgtcttggaaaccctggaccaggtggtgtgggtattgttgttattgttgataacgaagtccaggacaaactatcatttggtgatataatgactacgaataacaagatggagctacaagctaccatcgctggtattcaatatgttaaagatgtgtacgataatccagatattactgttttcactgattccaactacgttgtccgtgggatgaacgaatggattgttggatggaaaaagaaaaagtggaataacgttaagaacgtagaattatggcaaattttggatagtatcagttcaggttgtaaatttgaatgggttaaggcccacgcagataatgtatataataaccttgctgatgaattggcacgttctgctgctgaggaaattaagaagcagtcaacttaaagtgtaataatcagtcgagggaatgatgagtaaaatcaagcttacggataagcaacaagaaatttttgataattttgtagatcgtgtttataattatgcaggtaaaactatatctgtactgtcagcaccagcgggtacaggtaaaactttcactactgctgagattgttaaagcactatgtaaaaattacaatattgcagtaacatcgccaacacacaaggcaaactctgttattcgcaatatgctaatcaacaatgctgggttaactaaagaagatgcacaggttagtactattcactcatttctaggtttaaaactagtatatgaaaagaatcgtcaagtactgaagcatgacccacaaagtaaagttagtaatactaatgttgatattctaattgtcgatgaatgtagtatgatttcagaagaactgtttcaacacattcaggatcaattgtggcgtgttcgccgagcggttttattcatcggtgatcgctgtcagttgcctccagttgaatcagaaggaacaataggagaaactaagctttcccctacttttgatgttccagtacaatatgaattaacagaagttttacgtcaagcgttggataaccctatacttgcaattgcaactgcaattcgtgagtgcataggtacatccaagaacccgtttgatattatgcaccagacacatggtttgagtactattacaccaattgaagatgaagatcttttcttggaagtttaccgtgaatttattatggagaacaaaggttcagctaagaagatatttgattttgttaatgaaaataaaattttggcatacactaactaccgtgtgaacttggctaacatctacattcgtagccaattgtttgaagtaccagaggaattcacaaaaggtgaaccgattgtatttgaaagtatcactgagaacagtccatatgtggtccagcaaatcatccaatgtcctgatgtgaaaaccgaaagtttcatgggaattgactgttggcaattccgtttaccaaatggtagctacatcttaggtgtagggccagaaacgagaattgcaatggaaactaaacttgatgaactagttaaaaacattgagaaaaaagaaatcaacccattgacaaagaaaccatatatgtggcaagattactatgtgattaaaaacaaattacatattatcaactatccttatgcaacaactgttcacaaatcacaaggaagtacattcgataacatctggtttgatactgattttattgagcgtattggcactaatgatacaaaatgtagaatcctatatactgcaatgacacgaccgcgttataatgttatgttgcgtcaaaatgcacgattctaatagactaaatgatatacacaaataattttgattcttaacaataaataatcaaaagtgttttgtattatggagaagaatatacaatgaacaatttactacaagtattcaaaattgaagttgcagggacaaatctagataatattccttattatatgtatgaagattatgattcatcttccccaacaactgcaccaaccgaccaacaaacaattgacaagtctgtagcactactacgttacctacaggttacacgtctactatcaacatattctgttccagtgtattgtactgttgaatttgctaccccaggcacagcagcaactattccaactggtgcaaccatcaatgttggttatatcagctacgagccattcctaagcactctaagtgttgaagaacaagctgcactaaccactgttgatcttaaaactgctaaagcagcagaagttattcaagagttgctaaacgaatccctaaaaggtgacggtactaacgcaattgagaatgaagaaatttctgttcagaaaacaatccaacgtgttaaagatgcactatctaccaccactgttgatttcattgagtacgagaatgtgtacctaaacgtaccttctattactgatgcttctgtagtttcttacgtaaaattataatttaaccacatggagttcttgacatgactgacaaaaccttttatgatcgtattattgaagacaagggtgttttcgataaagttgtttatgacttagaacttatgatcaaagacgcaccaaatccacgcgtgcgtactcaagcagagtttgctaaaacttatctagtgcaagttcaagcaatctgtgaagcaattaaaaaacataatgatagtgcttgacagaacattagttcttaactataattactatcgaattgaacattaaaaccatatcggagcttttattacatgaaatctattattgccaataacgaaatcaccgcaaaaactgttcgtctagttacggaaggtaaaagtgaagtcatggcaatctctaaagcacttgctttagcagaaaatgaaggtctggatttggtggttattggagcaggtgatgaacctgctgttaaaatcactaatctgaacaaattcaaatacgaattgaaacaggctgaaaaagcctctcttaagaaacagcgtcaaaacactgttagtactaaagagattcaattcaacttcgggactcaagagcatgatttgcttattaaagcgaaaagtgcaggtaagttcctagaagaaggtaagcaagtacacattgtaatgaaaaccacaggtcgcggaacaaacgcaactgttatccaaaacaacgttgatgcaatggagtcattcgttagtcgtttgggtaacgtaacgtatgtccagaaagttgaattccagggtaagaaggtaacctgtactgtcaaagtaaaataataaggacgattctatgaaacatactatcgaacaacaagtgttttcacttcaacagttcagcgttgtgaaagctacatataaagattcagtttactacttagcagggagttacaacatcactccttatgtaactaaactacaatcagaacatagtaaagttgacaataaagtacttaatgctggactggaactatttctaaaagaacatccagaaaaaaatcttttggctgttcctaagttttactttattttgttagatgacccaaccacctccgctgaatttgttgaatgtgtatgtagtgaagtttttcatcttagctacgatgaaacaatggatgttattgatgcattaaactcatcatcacattcgtacaatatgggtacattcacagatgaaatgtgtattacattttcagctatgattgacaatggcaatgaacaattgcgtcaaaatcttaaatatgacaaaatcccagtacagggtaatggaaaagattatgatgcagcactggaagcactagaaactctaattcgtggtgactatccagacgatatttaacagtatataacctaaaaaactccaagattagatatctgataaatatctaaaacaaatcttggagtttttttattatgaattatgaagaattatataaagcaataatggaagcaatcgctcctaccgatgtagagtcttctcacttaaaaactattgaacacaatggtaaagacttgtacattacttttaagaatggaagtacttatgaatatgatgatgtaccagaatcattagttcgtcatatgttaactgtagattcaaaaggtcgttttttgtggagatatatccgtgataaatttccgtaccgtaaaacagcatctatttctaaaaaaacatatgcttccaacccaaatgcaattaaacagcgtttgcgttatgatgttgatactggtgaatgggaagatgcgttaacccctgacgttattaaaacagttgatattccagttggttatgaattccgtgcacctaacaatcaagattatgtgtttcagggaaaacaatggagaagtaaacaaacaggtaagattgcacctaagcaaattggttctaaaataactgatattgctaaaagattgataaaattaaaagggaacgatcaggaagggacagatgaaatataatcaattacaggagtctatcattaaagttcccagtactactatgacacagattaacagttatgtggcaagtgttctgtactttaagataaaacaatttattcaacagtatgatattcatgtggatgctgaaacacgtcagcatcctgccgctgaaaaaattaaagctaatgttattgcagagtgtaaaaaaacactaaatgtattgagttcaaagtatggggctaaaaatattagttcacaatcattcaatactattgcaaaccaatcggttaaaattccgtttgatgcggaacaatatattaaagaactaaactttaaaaagttagctccagaaattgttgatgaaattaaaaagacaaaactatcattagttattgcgaatgattcttctgctaaagaacgtggctcaatgactgaaattgcagataatgcttatgttattagaataaacattagattatcaccgaaagaaatatattcatcttctatgcaaataatgagtactgcatatcacgaagcacaacactttgtgcaaagttcagtaatcaagaagatcaatagtaaatcaaaccaattacaggttaagaaaaactatggcaaagaccatgatgaatacttaagttctggtattgaatatactccacaacttggtaatgtgattgatgcaatgcaaagtgaagcacaactgctaattgatgataataatttaccacaagacccaaaagaagcatttaataaagtacttcaactcgtgctctcgcgtgatgataagtataatgctcgtggatttattattagtatctataaaaattcacaaaagaaataccgtactgttatgagtactttatataaaaaattcatggatgcatttgaaaatattcaagctaatagttctgaacatgaagaagcaccagaagaagaattgggtgtttcagttaatgaaatgaaaactctaagtgacgtagctgagaattctagttatcttgaaacccgcttgtatggtcattcaagagatgaaattactaaaattcactcacaaagtattgaacacggttgggagatgattgttactaaaccatcagcaaacggtgatgcaattattattcagattagaagcgattctgaaaatgaacgtaataagatgaattacaaacaggctcaaaactttgtaggttcagttgcgaacatggatttcactgttgatgaagttgtagaattccttgatgaaattttacatatggatgcagatgaagcgaaagctgttaatggtattgttagtaaggcagagcaatgggctggtatgattgggctaaacttcgagaaaacaaatgaaggatttaaattaaacgatactgaatttgtgattgagccttatagtggtcgtatggtaatatcgtcaccacaactacccaatatgtatctagttggttcagataaaattatcactgacattttgaaatacattttgcgtttcatgaaagagcagccagaaaaattactaaaaacattgaatcaagatatctcaattgttaagatgagaagagagttagggagtttataatgccaagtaaaagtaaaagccaagcgaaattcttcgctgctgctgcccataatcctgaattcgccaaaaaagcagggatttcacaaagtgcagcaaaagagtggaatgcagccgatgaaaaagagggaaatcttaagaaaggttcaaaactccctaataaagtaaaagaatctagtatgcgtggtttggttgatattgtacaagatgcagaattagctgatacagaagacagaactagtgatataatggattcatgtgaagaagaccataaagagaaattggaagaaatgccacaacgtttcgatagtttccaaggccaagaccgtgacgagttcgttgacaaaactgccgagatgacaggtaagcacaacatggttaagttcgcagaacacaaagattatgatgtgtttatgaataagaatggtactgggtttatttcctatgataaagcgggtaaacaacttgctatcgttagtggacatttgtcgaatgatgcagtggttggtgtaccacaagtgtttgagatttctgctacggcctctaagactggtactaaaggtatcatctataatatctttatggatatggtttctcatggtattaaagtactaagtgacagtcttcacacagatgatgcaattaaattctggacccgattgattggttcacaccaagtctacattgtgggtggtggtgaagtactggctaaggctagtccagagaaggttcacaagtactggagtgatgatgaatttagtcctagtgctgaattacaattacttctggtaaaataataattgtcgggacatttaatgtcccgatatttttttgagacaaacaaagcattgatgatacaatgttgcatgacaaagagacattaaaaaggaattcaaatggctaagaaagaaagtgctgttcttcaggaacatgatgtacaacgtctaaacgattatcaacatgctcgcttacgtactgagatgtatctgggttcacgttctgttcacgaaagtcaagtactgatctttggtgagtctggatatgaagttaaaaacgttgagtgggttcctgcaattctaactgctactcgtgaaattatcgataacagcttagatgaatttaaaaaagctggaacaaaagagccagtactcaaagttgatttcaatgaagaagagttatcctttgagatcagtgataatggtcgtggtattccaattgactatgttcctgagtatcagaaaaacctttgtacaatggtaatgactgaaactaaaactggtcgtaactttgatgattcgaagcgtgatgaaacaatcggtcagaatggtttgggtggttcagttgcaatgattgtaagttctaaagctgaacttgaaatccatcgccaaggtcgcccgtataaaacacaaagtagcaatgacggatatgacggtttttataagttcaatcagaagttcgaagaaggaactattatgtttcctgaacttcatgtacatgaaccaactattcgtaaggttcaagcagataagactggaacaacaattcgcttcactctgagttctgaggtatttcctaaccgtaaactgccaaccgaattgttatacagtattttgaaagaaatgtctgctgctaaccctacctataagattttccttaacggtaagcgtattgcaattaaaggaactactgaaaaagcactattcaacgcatctaaactgatgcaattgaacgttaaagatgaatcattcaactttaacagtactttctacattgttcctaatgcagtatcgggtatgaacactaacttccatatgcatggtttggttaacaatgccccagcatttgatggtggtacacatcttgatacattcaagaaattcttctcattaggagtaggaactgctctagagaaagaatcaaagaaacgcaagatgaaacctaaccgttctgatattgaagaaggtctgttgatttataacgtaacaacgatgaaagcaccatcattcaactcacagattaaatcaaaactaactaacgaaaacgttattcgtccaattcaacaagcgatgaattctgaatggttcgatgaattagtcaagaagaacaaagagtggattgatgaaatttttgaacgctgtgctgctcgtaccaacaagaaagatgcagaagaagatcgccgtctagctaaaaaactactgaaaggtaaagttgcaaaactactggacagtacaggtacggatcgttccaaatgcatcctactaattgctgagggtgattctgctatttccaacatggcgagtgctcgtgatcctaaagttcatggtattctaccattgcgtggtaaaattatgaacgtcagtggtaatgagaaaaccaaagaattaatggattcaatggcattgcatgatataatggcgagtatcaacctgattcctggtgtaaaagcggaaagagcagaccttaactatggtaagctttatatctgtgctgacgaagatgaggatggaaagaacattggtgctttagtatgtaacttcctatacaaattctggcctgaactattccaagacccagagaatccatttgtgtatatattcaaaacaccattcatcattctagagaaaggtaaggaatccaagtactactatggtcatgatgtacatgaatacaacccagatgattggaaaggttggaaagcaacccgtgcgaaaggattgggtacgttagaaaaagcaaacttccgtgatgcattcactaacagtgttgcagtagcgattacagatgatggtcgccttggtgaaactcttgatttgattttcaacaagaaacgtgctgatgatcgtaagaactggatggaataatatagattaatggggaacatggtgttccccttttacaaagagacaatggaatggctaaaagtaaaaaaaccgaaaatcaaagcatcgataaccaaacactaactctactagattataacagttctgattatgtgaataccacatcactagaatattcaatgtacacactggaccgtgcaatccctgggattgatggccttaagagttcacagcgtaaagcaatcttcaccctctcaaaagttagtggtgaaatcaaaactgtatcatgtgctggtaaaatgattagtgatggtatctatctacacggtgatgcatctgcatctggtactcttcagaacctagcatcaccagtcgtaaacaactacccactcattggtaaacgtggtggttttggtacacaagtaaacagtactccagcttctgctcgttatacttatgttaaaaagaataaaagtactgaaatgttagtactgcgtgactctgacattattccaatgaaagagaactatgatggtactacaatggaaccaagtttctttttacctattatcccactatctttgtttggtaacgatagtatgtcagtaggttataagagtacaattctaccacataagattgaagatgttattgataactgtattcgtgctattgataaaaaaccattaaaagaaatgactccatactatgtttcatgtggtgcaagtgatatcgtaactaagcttgatgaaaacaaatatgagttctatggtaaagcacaagtaattgatgccagtactgttaaagtgacaggtctaccaccaaactacagtattgagaaattcatcgagaaactcattaagatgagcgatgatggtgatattcgtgattatgataatgatagtactgatgttattgatatcacaatcaaactaccacgaggtacatctgccaaatgggtagaaaaagacgtaattgaatactttggcattcataagaagatgacacaaatcctaactgttctaggtgagaatggtaaggttaaagtctatgataacacaatggatttgattaaggactttgttgaattccgtttcaaatactatgtgaaacgctatgagaagaaacttgatgattgtgctgctgaagtacgttataagattctaatcaaagaatgttttgataatgatatgattgctaaggttaagggttttgctaaccgttcagaaatggttgagtttgttaaatcattaaacactgaaatcgaagcaaccgatgagaatattcaaagcatcgtaaacttcccatcatatcgttggacccaagacaattatcaatcagtactagctgatattcaacataacttggatttaatggatgaatataacaatctcctgtcgaatcatgatttgatttggaacatctataaagatgagttgaaagaactacgtaagattgacttcacgccaaaaactgaagattgattataaacagggaagacctttatgggtcttccttgtcctttatataaataatctatagataatggtagcttggctatggagaattataatgacaacttttacccctagatgggatataaaagccgatgctcgatatatacaagaaggacaagacattcttgtgcgtttgagtatcaacaaagaaaccacaacggatgatagttatatacttaatgaaaacgaatacttagagatgattctaactattcagggtttggacagttctgatgcagtatttcaaccagaagatcactacgcagtgtatgttggtaatgatgaatctaatctcacacctatatcatcaggtattactggtaacaagttgattaatggtaagcacaaatgtaaaattgataaaactcaatttgatctatcggcaagcactaataccgaagatgtttattttattaaattgcattgtaaagatgatggtacatttaacaactctgaacagattaacttctatcttcaagcagtaaatgttatgtactatggtaacatttcccctagtggtgaaattggttcaaatgaagtaacaattgaatatgatagtcaggaagtgtgggttactcctggaatcgaaatcggtgatgatgacaaattatattgtattggtgatattgaatacggttccaacagtactttatttgtacagggatttggtcgccgtcgcccatttattttaggaactgatacatcaactgacctatctcctagagtattttatccagcgtatcgtctactcgatatgaccccagcagaagttcctgctcgccacatcactgatgattatgcaatcctacgtcctattgataatatatggcctgtatttggtattaataaagaaattaaggttaatattcaaactagctctggagagttactagattcaggattggttttcaacccaaatggacaatttctgcaaacattcctatatgactctgatgcatgtgatgcatatttcaaagttgaaattgttgatattttgagtacatatagttatgacactgaccgcttcgctataaaccaacgtttcaaagcatatcaaatgtgggtatgtccagacaatgtagatggtatatacgctgatggtgataaacctttacctaatcatttgtatcagttgaaagataacgttgtatatccatatgtaatctctggtgaatatgatctaggtgaaacaaagccagtgtttaaaagttcagattggattgatttaggatattatgattcaagcttaggtgagaatattataggtgaagagaagatgttccgtgttctagtgaacgcagaagaaggaaactcaatagacttcacaactgatagtgatttaggtagtattcatgtgggtgaatatttcggtcacagtacttatccacaaatacaaactaccagtactaatcaggtatcttatgtattagatggtggacctgatgatattcgcaaatatggtttggacctcaccgcagatgggttcttggtcgggacggcatatgcaaaaagtttagatttcagtgcaaatgatgatataaaattagaattcaatatcaaagcatatgacactgaaggtggtgttgcgtccaagttatttaaactgaaaattgtacgtggttttggacaaaactacttaagtgcacatattgtaccttctgtttattttgaacgtgaatggtttaagtgtattagtacaccatcatttacaaaacaagctttccatcgtcaatcagatgatagatacgggttacaaaaagtaccacgtattctattaaaagaaaactttttgagtcagaactatacttacactacgttgagtgatattaaaaaagtacttagggaaaatatcataaaccaaactggtgcaactatacctgatggaaaatttagaatggtaatgggcaactataaagtaatgtcagcacttgacaaccaaggtaacttgcagtatgatatattataccgtgaagttttacccgcaggttcattggtgtcggtttctaagaatccaagaagttacacgcaaattgataacggaacactcggtgaaatatttggcttgcgtcaaaatatttttgcagcaattggtgaagacacaactaaccttgtgaaagacccaactgatttacaaaaccgtggtttgtatgtagatgctataccaggaatttctgatgagatgctagacactgttcctcgcttcatgaatcacccatacgaagaaaatggagttgaaaagaaatttatgccaatcattccagttgcatactgtgcaccaggaaagggtgaagcattctttaacactttacttcagaacaatgaacatggtaagatggttggtgttgagtttgaaatcacaggagttgagtttggattctttgcacatgaattcgaaaactacgttcatgatacatttttgatttcattacaaaacagcaatttaggaggatgatatgttcaatccattttttgatccatcaaccatgaccgagaaagagttacaattgaaaattaacgaacaatcgatgcgtattacgtctgctcgtgcagcaggtatgagtgatatgattgttggtaacatgcaaaccgttctgaatcagtgctatgctgaattagagattcgtcagggacaaaaagaaaaaacctcctatgatgaacaagacccatgtatatttgacatgaactcttacttaacggataacgaagaggatcagaagaagaaaaatgaaagtacaaggaaacagatttataagtcccaatggtgatgtaaaatgtaaccttaatgcagtcgtgtatgcaatcaactgtggtgcagatattcagtacttacagctacaggagattgatgaaccggaacgattgaaatttaacgataattgtaaaaaatttggtttagaacagcgtatgctggattatacaatagaacatgataaaaaccatgagctatggcgttatgatccagtgtacgataacatcaacttgcgtgagtactttttacagaaatgtacatcggatgaagcccgtgaacgtgtaatgtatgagattgatttgtttgaacattacaacatgactaagcttttaagatgtatgatctggttagttgattatatggaagagcacaatattttttggggtcttgggcgtggtagttcagtggcttcgtattgtttgttcctgattgatttgcatatggttgattctttgaagtataaactagatgtaaatgaatttttgaaatggtagaaaaacatgtgatataatgttgtttgaggcgttatatcactcacatagaaaaaggatttttatatgtcaaaacacacaacttatcgtggaaccacaattgatatggatatgctaaagtatcaaaaccagcaccaagtagcactaggtaatgcaaacctaaatgctcgtggtgataagattggttcaggtggtacggtcattaaaacccgtgaagaactaatccaagaaaaagaacgtgaaatgcaaattcctgatttcattcctgaacaccaacttcacagtgctgaatatagtactgaagaagtatgggatgatgaaagctttgaagttcaaccacaagaaccagaagttgaagtatctgaagagaagaaagctccacgccgagcaaaaactaaagatcaggagtaattcatgaccaataaagtaatgacagcttgtaaaggtaaagttattgtaactgaactagaacatggtgagcgtaaaaccgcaggtggtattgtactgacaagtgatgatggtaagatacgtggtattcgttgtcgttgggctaaagtatattctgttggtgaagacattaccgatatcaaagaaggacaatggattctaattgaccacggtcgttggtcgagaactattgagattgatggtattaaaatgaatctcgttgattatccagcagcagtaagtttggtatctgacactgatccacgcatctctgctcaaacatttggtcgttaaaacataaatctaaagctcggatatattatctgggctttttatttggatgaaaatacatgataactgatgatggaaaatatagtttttctgacgttacgttagtttctaaaaatggacgatttatgtttgaagatagaactaacacgaagtacgtatatgaagtttctcatgatatgtttaagtacttacttatcgtatcagcccaggccgaagcaatgggtgtgaagaacaaagcaaactcatttaattttttgtttaatcaacttgaaatgacagaagacttattcactgcattgttgtatgattatgcagattggatttcataaacacctaatatacaaggaaataaaacatgactacattatggactgaaaaataccgcccacaaactatggaagattatgtatctaagaacgaagccctacgtcagaagatggaagaatggattcgtactggtgatatcccacatattggattttttggaccagcagggacaggtaaaacttcagcaatcaaagtactactgaatggtctggaagctaacggacaccttgaccctagtgatgtaacaacactgaatatgtcagatgaaggtatcgatgctgttcgtgataaaattgacgcagcagcaaaactatcatgttttggtcgttatcgtgtatttgttctggaagaaatggaacaaatgaaccacaaagcacagggtagcttgaaacgtgtcatggaagattaccatgaaaatgctcgtttcattcttaccagtaatgaaccacataagattcagaaacctattatctcgcgtgtccaggtcatcaccattgagaagcatgatatggaacaatttatgtcacgcatctacaacgtactattagcagaaggcgtagatctaagtactgatgaaagtattgaacaagtacaaaaatacatcaatgcttcatatcctgatttccgcaaaacgctaaacgtattgcaaagttcagtagtcgatggtaaacttgtaaagcttgaagacagtattgagaactctgctgggtatcgtactgccattattgatgcactaaaagctggtaacattcgtcaaatgcgtgagcagatcgtgcaaaacattcctgatgatgagatggatgcattcttctcttatctgtatcgtaatgcagaaatgttcagtactgatgaaattactctaatgaaaattaaggttaagattcgtgacgctgttgttaaacaagctaccgtgtctgaccgtgaaatgaatctcagtgcattactgtgtgagattgacctaatctgttcggggaacatgtaatgaaagaccgttgtattgtattcgactgtgatgaagtactactagatcacattggttcattaggagaatacgcaaaactccaatatgatatcaacccaacaacaccatacccactcgcatacgatttatctgaatggtttggtgtttcacgagaaaaagttcaagagatcattactgaattcaatcaacgttcgtatatgtttggattactaaaacctgtagaaactaacactgtaagtattatgcatgaactgcgtgatcgtttttacaaggaaaaattcatagtactgactaagtgtggtaagaaaggttatagcgaggtacttcgcaatactaatcttatgcatgtatttggtgagacatgttttgatgaaatcatcattgttgaaatgaatgaatctaagcatgaagcactagtagaattaaaagaacgatatgacgtaaaactgatgattgatgattatatcaataatattgaaacatcaatggaattgggtatcccatcagttatgatgcgagcgtcccacaatcatatgtataaaaatggttatgggtatgactatgttaacaactgggatgaactacgtttaaaaatctattcagtactgtcgaaatattaccaataatatagtaaaaacaatggggattatttaatccccttttattgttttttaaaagtagtaaatagtattatatgtgctagttttttaaggataatataaaacatggatgatttagattatacagataatgcagtccagcagttgtatgataacagcggtatgctagatatacttctggctgttgaaaaatattttgatgataatgatttgtacgcatatcatggtatcatcgaaggtgaactcgttgaaggtccgattgttagtaagtactggtgtactgtgacattcaaatactatcgtgagaatttccctgatcctactgcattctcaattcttgagaagcacgggactaaggtatatgtgaaagcagattatgaaattacacctattcaagatcctaaccgtcaggctgatatgaagagtgtggcaacgcaatctggttctgttcgtattccaaaaaatgaacgaattccagttctattggttcgtgtacaaattcctagaaacctaatcaaccctgaaagttttgatgaatacaaactaatggcttccgagttcaatagtaatcctatgaatgcacaagaaccatcagatgagccaattactggtgaagatgttgatttagatgtagaagtatctgatgatgtacaacaaggtggtatgcaatgaaaaagaaactaaatgaacaaggtttacgtccacaagtactaaaggatttgattcatcctattattagtgtagatgaatatactcctaagattgaagatagtaatatcgttgttatgtttcaagtactaaacaactttgatgctgcatatgacctgtcatcttttattgagaagtctccaattgatgcagttgatactgaagctgctgaaacacctaacctggatggtcgttatcaggtattcgttgaaatggaacgtgatgcggaatttccagaaaacctacttcgtcttatcaaaactattgaaaacatttgcccaaatcctggctggaagattcagctttatggtgtgaatgatccgattgatgttgattttgatgaaattcaacaacgtatcgatttgggtattcctaaatccatcagtgaatttttagactttgctggtgtatctgttgttaaagaaggtcacatgcttgcactaaacactgcatatggtaaactacattacagtactggtagtggctttgtgtcagaagcatacgtaaaacagcttctatcgaacgatacgcaggtagatacatctcgcttaagttcggtactaggtgaaagttatagcgtcctacgtagcggtaatcagtatgtagtaggtaaagatgggaagtatttggtgttacgatgagaattttaaacatgcttaaatctctccatgagactaccaaacttcaccaagacgacaagtatcatcgtagtacaactaaagatttggcatgtagaatgtatatgagacgattcgaaatttctcgaatcggtaaaggatgggacgatatcaaacgtgaagatatcgaccctgatgcggtattcaaagcatacatggcacgaaaagtttttatcaatgagaaaactttttatattgacagcgataaagaacttgaagtaaaatttacgaaaggcttgtggaagatttggtgcttagagacgttcggtgaagtgttaccagagccaccagtaccagaaacttacaaagatctaaaagctatatttgacaatgaacatatacatacaaattactcactatatgaaactgtttccgaagcggtattttatggagagttgaacggtcataacaagtactttgatagtatggtgagttttgatttattctgtcatctccctggtatctctagttggacatgtgaccagcatagagctagtaaggtagacaagcgtaagttctctgcccaacgctttatgcaagctgctatgggtatgtacaaattggattatcttgggtgtccagaaccatataaacatttttggaaagaacgtgcacaaaagtgcatgaactattgtataattcgtttaatggattcatacattttaaaattcagaaatgcaactgtgtatgatatgccggaaccaccaaagaatgaaaagtacccaccagtaaaaacatttgtgtggtacaaggacaagagtaacaaaaggatgcactaaggtgcgtcctttttttctttaaaaaatggtttgacaaatacatcgtgtgtatgtagaatgtacgtatcgtatcaattttaattaattaagaggttagtaatgaaaggaaaatattttttcattgacaacggcaaagaagttgaaatcaaagaatttaacttcaactacgttcttaactcactgttaaaacagtatgatcgttcatcgaatcgaaaatatatgtataccattgttgtggacagaacatacgattcattgggaaatccacatttcgacacccgttcactttcactcgacgagatctattttgataatcgccgtttgaaggtaatggatttgatgaaagataaaattcatttctcaaatgtgagaatcgttccagatctcccaattcctccgttagagaaggagtaccgagactggaaggaaggcaaacttcgtccaaacatggcagcagtagctacttcaccagaagttccggctcctattgttcttaagaagaaacctatcgaccctgaatgtgagatggttgctgacattgaacgttggatggaattcccagatacaattaaaaaagattcggtaaaagtactggattggtatgttaacctcccagtatgtgcacaagaagttggtatgaagattcagaagatctctgagcgacaattggatttgaacgcagcactacaacaacacagtgattatatccgtttattaatgggtaaccttacaacagcactggaaaagttattcccgttgaagcctacctttgcacagaagttgtttgggaaagcagatatcaaagttcaaccatctgatttaccaaaagtaatgaaagcattgaatgaggccgtgaagtacgatactaacaagtttagtggtattacgtctttattcactgaaattcgtgatgaagttcgtgaaatcaaaaatgaacttgagtatggtaagattgggtgtgaatatgcaattcgtcgtttggaagatccatttgaatatgaactaacgcttgaacgtattatgaaaatgggtatttcaacagcaatgactgaaactagtattgttggtgttagtaagcagtatcagattgatatgagccgaatgattgatattcaaactatcactatcccattgattataggtcgtttacaagctcaaactggtaaagcagtcgatgaagatactgccaatatcattcgcaacctagcgtatggaaataaagagtaaaaaagtagttgacagtactgagtgttgcctgtagaatgtttatcactgagcaacactatcttaaattaactgaggaatacacaatgtccgagaaagaaatcaatccgaaaaaagcaacagttaaacaagccaaagcaaccaaaactgccaaacgtactaccaaaacagcagcacacacaattgacgcaactgatatcttcgaacaacagtccaagtcacttccagtactgaaagatactaaaggcaacagtatggctttaactgtgcagaatattaataccatcggaaccactgctggggcaggtgtaggacgtttggctgatgaaatcatgtcaaaagttaacgtcactgatgataataccgaattcggtaagtctattactactatcttgacattgactcgtgcagttgatattgatagtctacaggatgataaatcaatcgtaggctgggtcaaaaaactttttgttaatgctaaacagaaagttggcgatcagtacaaaaacagtaaagaacaaattgatgaaatcatgaacactcttcaggttggtattaaccgccagcatggggaatgtgcatggctagacactgcttacaacgaaaacaaaaactatctctttgaactgcgtgatacgttagttgagttggaagaagttaccaaagcacaagatgctgaattggtagcaatgcaagcgaaccctgacgttgattcttttgcaatccaggagcaaaagatgatttgtgaagctctttctaaacaggaagataagattcgtcgtcttatcatgatgtgtgaactgagcggtcctcgcctgatgagtatgaagaaagtaaacctcaacactgtttctaagtttgaggatttgaaatcagttgttattccagcatggaaatcacaattggctgaacagttgatttccgaacgtcaacgtaaagacaatgagttgggtaatctgattgacaatgaaactaaccgtttactcgaagtaaactctaaaatggtagcaactaacatgttggatgctgctaaagcaaataatcgtggtatcgtggatattaaaacattgcgtaatgtacacaaaacaatgatcgatagcatcgaagcagtactgattgaagagaagaaaggtaaagaagagcgtgagcgtgctaagattgaaatcgcaaaactctctcatgaacttgaagatactcttcgtgaaattgctgaaaaagctaacaagtaatagttgacacaagagtgcgttttcaatataatgttcttattgaaacgcactcttttttaatatactgataaggaacgaaatatgaaacgtcgtcatgttagtgttgatattgaagctctcggaactcctgaaaactctggttatgatattattattccaaattatgcagcagtagttgttccaagtaaacctgaacatgatatcagacaatttgtatatgttaagttaccataccaagcacaagtcaacgctgggctgaaatcagatgcaggtgcaatggatttctggatgaatacatgtcagaaggaatatcctaatgcatatgctgaagttgccagtactatggcacttgttgcaccagaattgtacggtcctaatggtaaatccaaacaaatctttacttctaatggtattgtcgatttctggaaagatatccgttttgattgttatggtaacgaggttgaacattcaatttggggaaatggctgcaacttcgactgtagtattttacaagctaatcacttaaaactattgaatgttggtgatttgtggaaatatgatgctcctgaaaacgcacgaaccctgaagcgtttattgaatgaagaagctcgcaccgaaatggatgagattgtgtcagtgcagttagctaaattcgtgaagtatgtagaaaaatgtggtattaatggtttggaattacatcaccctctgtatgatgcagcacgtgaagctatccaaatctcctattgtctcaataaacttaaactggactaatacatgaattttattattctgttgattccttcacttatcgttgtatttgttatgaaatacatgtatgataagcatatcagtactaaagaactgttgattcatattggttgttgtatagttggtgctgctcttgtgcttggtatctcatatgcaatcaactacagtcaattgtacgatgtagagattctaaatgggcaggtaacatccaaatataatcacaaggagttttgtacacaatcaagctcatgtaagcattacacgtggcacgagaaatgcagaactcgttatgatagtaaggggaaatcccatcgtgaatgtgagtcctataaggtatttgattatacatacgaagttgactggtatgtaaaaagtactgtagggaagtttgagattgagcgagtaaaccgtcagggtacaatgacccctccacgctggcaacaggttaacattggggattatgctgccgcagaaagttcttatattaactatctgttcgctgataaaaactcattgttcagtcctgatgaattacatgaaatgtatgatgagaaatatcttagtactttgccagaatacccaagtgtggaagactattataaaacacaccatgtaatcaacactactaattataatgtagatggttatgatgagtatctgcgtgaagaactaaaaactatgggtgcacgtaaacaagtcaacatccaagttttggtttatgattataaaaacgtagattttgttgaggcaacactcgcttcgtggcgtggtggtaagaagaatgatgttattatgttctttggaattgataatgatgggaagattgttaaatttgatagcacttcctttgcacaaggcatgaagaacgaagaactacatgctaagatgcgaatggatgcattaaatgaaaaattaactcttgacttgttgagcaaagaagtgcataatattgacacaaagtttgaaagactctctaataaagagttcgagtacttgtctgtaaaaatggaaccacgtaaagatgttctcatcggtgcttctattttgttactgattgtatctattttcattggcctttatatgagggataacaagttatgaatggtaaaattattgcattgattggtgttgttgttgtattcggtcttatggcaattggttcagttagtatgtatgtagatgcacatgaccgtgctgttgaatttgaaggtaatattaaaaagtactatgatgcatcagaaagtcaactaagtaaatacactcttgtcattcaagaaaaagtacaagtttctgataagtataaggatgcattgaaagaaaccatcgaaacatactttaaaggtcgtgaaggtgttgaccagaagtttgtgatgtcacagattcaacaacaggttcctaatctcgatcctaagatttatcaagaacttatggtaactatcgagtcaggtcgtacacagttcaacaatcttcagaaaatgaaaattgaccagtgtactgattatgctaagttccgtaaaggtttctggaactccaaaattcttgatactaacacattcccaagtgaaaatattgaacatttatgtacagtagtttctgatacacgcactaagaaagcaatggatactggtatccaagaagtaattaaactataatctaaatctgggagtacattgtactcccattttctataggatttattatgtttttaaaaaatacttatgaattaattaaaatgcttcaaaatatgaattccggtggtctattcccgtatcactctgaggataaaactcatcgtttagttttgaaattggatgaaactgggcgtattgcatcactaagaacggctgttccaaatatggatcaagtaattatgccaacaactccgaattcaggaactgctgttaatacaccacaaacatataactcaaactaccaacacccacagatgtatacgattagtactccatctattacaagagttgcttgtattgttaatataaaagtatcaaaagactatcgtgatttaatatcagttgaatggtcagaactaacatcagcatttgcaaacaatgggcttgttgtaacaaatgctggtatagcagtcaatgcaaaagaaggacaacagataatgagtgcgttgtatcttagcaatggttatgttatcattaatgggaagttgtattgtaacaccgaaccagaagcactcactgaagatttgattagtgaaaaagaatatagtcttcttgaaattatgaatttaaaaaaggtatatgaaaactgtgattagatttgatcaagacgatttgccatttatccaagaactagaagaactagttacggtaacgatgcaagaaattagttcatgggatataacagtttttaggaacgtatatgataaaatcacaaactttaaaactgttaatattgaggtaaaacacaataacttttacttaacaaaagaattcattctaaaaagaccaggaacgatccaaccgaaacgtaatttgtcttttgctcaagttctcggtgctaagacaaggaatgatgatgatttgaatgaatcaatatattcgtatattaaagagcgttctcctgatgttaagaagagatattacctttgtgttacatttgatgaacaaggaaaactaaatgcgtattctataaattcatcagcacacgtcccattacattcaacgtcaattggtgataaacaattactaatcgattcatcaacatggcataatagggaaaattttgccactgaaacttttcagtactcggtatctgaaccaaagtctccatttgattttgtgtttgttaataacatccttttacgttttggggtaggagtctaaatatgctaatttatttgagtgttcttgttatttcattactggtatcattagcagtactgaaagttgttgctatcggtcgtagtaaaattgatagtactgaaaccagttctgtgttggacctaattatggatcatccaaagatctgtactgggattgttgtgtttgttcttcttataggaacattcattgtacgtacacatttatacaatatgtatgactgtagtgttcgttcaatttcccagggtgttgaaaccgactatagctggtactatgagaaatgtcgcttcaaaaaccagcatggtatttgggttgattttgataaactgcgtggcacaccatctggtgaagaagagtaaatatgtttgaatttcctgaagagtcatttaactatattgaccctactgatgatggatacagacatattaatatctatagtaaagctaagactaagttaggtagggatctatctaacttttcttcacatagttttaccttagaaccatatggttggttcccatcggtagaaactttttatttttggtttttaacaggccagaaacatgatgatcttagaaaagtgtcgggtgctgctgcaaaagcagcggcgaacaagtatatgcacgaccgtattgaaatgacacccgaatgcatttctcgtatccaggatgcaatatgtgctaagatatttcaaaacccagaacttgctgaacgattgaggaaaagtaagttacccttctaccattactatgtttatggtgggaaggtggttgatgtatctgatgaacatgactggtttgttaaaacattcgaagatataagaattgttttgaaggagaaccacaatgagtaaatgttgggtatgttgtaagcaattcgatgaagattatattatttctttggatgtattaccacaacaatacatgatatcttcagggaaatataaactagatacagttccaacacctaagtatatggggatttgtccattgtgtaatgggaatggtgtatcgaatgaagttgttgcattcacaaatacacaaactactgattgtgtcgccgaatatataagaaagaatctggcttttttcactaggagattatatgaacgagagaatctttaattcatatgatatggaagatttactaataccatctgataacagtactgttaaaatgcaaatgtattggttagtaaaagatggcaaggtattccagcacaaagttacaaaagtatggcaatgtaataaaaacaaatctatagttgatgcactcgcaactgcatttcctggggtagaggttgtatttttaccatttgcctacattaaggaaataaaatgaaaaaaacatggacaatattcgtaatacctactataatgttgttattgtcacttacggtcccacaagggcatgttgtggcactgaatgtagtagaaattattcttcttgtaattcactgcattgtatgttactattatgtaaaatcggatgataaaaaaatatgaagtactatcgaattcgagttggtgtaagctctggttttagtaattgggtatattatgatttacctgatagctatacagtagaagatgtaattgacttatattatgaagaagagtcatgggtatccagtactgaaacgcacagtgttgtgtgtgaagaactgagtgcagaagattacaagcactgtacccaagcaatgaagtattacagtaaagatgtgaagatgttatctgaaaacgtttttcctgtaaaagaatgggcaattttgcttgcaaatcctaacaaacctgttagaataggttatccaaacaatcatatggtttatcgaatgaatgataaacgtgagctaagttatcagtatgattacagtgcttctaagtttggagttctaaaacaatttatgaaggcttatccagtatgagcaaagacagcaaagaatttatagtacgttgtagttgtggcgataatacggaacattgtgttcacatttcacaatttgacttgacacaagaacatggagatgataaacttggtcaatgtctggtgagtatgcgtttgaatcctttcttaaaatggtacaaacgtgtatgggttgcattgaagtatattttcggcgtaaacgactttcaatacaaagaagcaatgattgatgttgacattctgaaagaagttgtgttacaattagaagatacccgctccgatgaggtaaagaaagaagaaagagataaacgtactgatgtgaaggttttatgagtactgaactttatgaaaaaaagtactgtacaaattgttcaaccgagtatacaatacagggtatgttgatttgtacaatatgccaatcaaaacaggctgccacttttatcgttgatgatagatttggtgatttaaaacagtttaaagcaaaccgtaatcttcgtaactttagcaatctatataaggaataacacatgaatatcgaacaaactatccgtaaccactctttcttcgctgctgaaactggtcgtgtaatcgagagcgtcaaaattgaagaactgatgattaacgttaaatataaagaactgaatgatgagattagcagcaccatttgtgcactacgtgctccattccgttctgaagcagctaagaaacctgcctatgtactggcgattgcattgggttgtgtagtgaaattggtcgttgatgaaaatcaactaaatatcaaagttgctccaaccaacgatgaagaaaacatcgacctgaactttgaaggtaatctggctgaagacgtattcttccaacaatcactggtttatgatttccgtaccgtaactcatgaccatatcaactttgtcaatgagatggttgacattgtagctgaatacgaagcttccctataattagattaacaaaagcctccgtaatgggggctttttccaacataaggatatgaataatgattgaccaaattaaaatcctaactgaacaaatcactgattttctagcagataaagatggattcagtactgaagttaaaatgcatggtgttagtctcgatgtaactgcaccaaatggcgaaacaaccaattatgatatgtatgaaatcctactttatacattggctgttcgtaaacttggtagtgatattgaactaaacattaacaaaaccgagaacggtgaccaagtgtttgcaatcaacactggtgcacaaatggacgtagtagctgaaatcaacaagatcaaccaatcttttgtgggtttcaagcatgatacaatccaatctgtgttcctgggtaaacaaaccggaaacagccactgtccttattgtgtaatgacaacctataagaatggtaactggatgcgtcaggatctagtacttgctgaaatgctttatgcaatcacacacggcctagaacttgaacttgatgatagcgtgtactgtttcaagaaaatccgtaaactttctggtgatatacaagctcaattaaaagaattaaatgataaataattataccagtaatggttaacattatcatgttactttcctcccttacataataatgatgcgttattattttattgtattattccccttttttgtacaaaatctttttcgtactgccccgaccttcggggcatttttcttgtctaaattttgaggttctatgaataatactattggtagtacaatatctaaaccactctcttattaccttgaaaaattcagaagtacaaatggtaaaaaatttaatggtatataccaattctcacataaagaatatatcacagttgattgttattcaaataaacttatgtacgtagatgatactgaatactgtatgaaattgtcattggatgtagaattagataagttaacatttattgaatatacatccaaaagaacatcatttcttgggaaatgtaatcgaaaagtatataaaaatatttcaatgtacttaaatgaagattaccacttccaacaaacaacgattgaagatgttctattcagtactgaagaattaaaagaactgtatcactggtatgagcaattcacatcttgacattatcgtgggtttatcctacactgtaagataatgatgcatagaggataaatttatgagtttagatttacacaatgaactaaaatattatttcgatttggatatatcacatttgaaccaacacgtcaattatatgactagtactatgtggtggtctgatgagtttgatgaaccaaaaatccaactttgtggtgatttgtctgttttgaggttgtatgcccttgattcggaaatgaatatcaagatcacatacatcccatacacaaacaaacttattgtaaacaccctaaacccagttacagtattgaaaatttctgatttggacacgatacagtcagatgaatcactattccaacgttctttgattgatgatatgggtggtattttatatgaagatattgtacaattgaagaagatgtatagtgaattgttagaaatcaaaaacacagtaattgtggggagttgatatggtaagtttacttgatattgataataccagtctagtactggtaaaaaaaggtgtaattcccaattctacaaaaattgattatgcagttattgaaacaaatattggaaatatctggtatgactccgaaagggatattgtgattagctgcgtagacagtgaccgtgctgagtgttgtagggttggtcatgacgggcaatttattttcgaattccgttactggaaagatgctaaattccatcatgagcatgtttcaattacaggtgtgtattgcaccgaagaatcgttcttcatggatagtactgttaatgattttggttcagttacatttgagtactataaaaaagtatacaacgtatccagagaagtaatgcgaatcatcagaaatgctgagggtcaagattaacatgactactatatatgacattctaggtgacttgaataaaaatatagataatgattttgttattgttggaactacacaaaatataattcatcttccattgggtacaccagataggttaatttttaatatatacggaaaaacactgacatacctacacaatggtatggttatattggtacttgatattaaagaaaatacaatacgatatgcatatgacttcaacgtttattggtcaagaacccatagtattgatattgaggaagatgaactatttcaattgtcgttaatcgagaaaaatgtaataggagttaccgaaattggcattgttaaaaaagcattagaatatgtaaatagcaaattaaatggtgagctatgatgaaaattgatgaaataaaaaatcattttgaaattgcattgtcttgtaaatttgatggctcttatgttcctgatccagaagagataattagaatttccacatcgttttcggaagagtacaatgttcttattgagaaaacatatgacagcactgaactcgcattttacaataagaattcacgaacaacaacatttatattattatataacacaacatcggatataatagaaattataacatgtaaaacaagcaaagatatacattatgagactatctcaaatgtgagttattgctcaactgaagaaggatactttcaggaatctcttgttaaaaatttaacagttagttattctgatttaagcactataatcaattgtgttgaaaagctaaatgatttcgttctatctatgtttgatatcacagagcaaaacccgaaggtaatcaaatgctaaatccaggacagtttttccatttacttagtattacacctataccggatttaatcagtgaagcgaatcaatatcgtgatataggaattgtatccatggaatacgattcacctgactttaaatgttacgtaacatatgatttggtagcctatgagtcagaaactgagataaaaaaaccatcatacatttttgtgttcatatataatgattatagtggtgatctactcaatgatgttcatattgacacatttgaaatatgtgataatattataacagatgattcatcatgtacatttgatttatcagaaaacagtactgaagaggcactttttcagttgagtacagttagtaacactgtactaactcttgatgacattgagaaatttaaagaacagcttgacgcagtacgtgagatgttctataatcattatccagaaatcgagttcaattaactaaaggtatcaaaatgactaaagtagttcgtaaacgccacatgatgaacatggatgaatttcgcacctttgacaaataccgtactatgtttcgtatgctgtcacagggccgtccaatcgtcattgtaacggagaattacatgtcacctgagatgatgttcggtaattatgaaaaggtcttcatgttgtacgtgaagcgtgaattgctaacgattaagaccttagctgaagctacagatgaagatatggatggtgataatacatttaccatgtgaggaaatatatggcagaaaaaccaaaagaaccattggaccgtgatgatgatttttacgacgaacgatatggggaatcagatgagtgaacatcgtgcagtacttaaaaagattttaaatgaacttggtgatgagtttaatgttactgaagaagaatatcaatgttggaagaaggattttgaactccttgctgagtatgataaagtcttcaatatgattggtgtcattagccagtacttacttggtggtggtcgagccagtgcttcaattcttgcatctgcgatcatctgtacttctcgatggtgcgatgaaaaaagtctggaagatttctaaataagatgctataatgtattatagtaccttaacaatataaaaaggattttatatgaaacctatcagagtactatctctttttaatgggatgagtgttggatacatggcattaaaagatgctggtatccctgttgaaaaatattattcatccgaaattgacaagtacgcaatagctgaatctcaagctaactttcctgatatcacccaattgggtgatgtaactaaatggagagaatgggatattgagtgggataaaatagatttattaatgggtggatttccatgcttcccagcaggaaccatgattatgacagacaaaggtcataaagatataaaagacattcaagttggtgacatggtactgacacatactggtaaatatcatccagttttgagtactggtgttaaaaatgctcccactcgtattattacaggtcaaggtcaccttccaattgaatgtaccgataatcatcccttttattcaagaaaaatgacgtctatgtggaataacaacattagacaatatgaacgtcatttttctgaaccagaatgggtagaggcacatgatttgacatatgaccattttctgtgtaatgttaaatcagttgaatctaacaaatcaacgaaagatgaaaaattctggtattttatgggtcgctttatgggtgatggatggacatataaagtaaaacgtaacaatagaaccaatagttaccaatatagagttttcatctgttgtgcacatgacgaatttaatgaattagtttcatgttttgatggtatgaatatgaaatataattatgttaaagaaagaacttcatataaattttatatatgttcaaaagatctggttgaattttgtgaacaaatgggaaagggtgcttctaacaaacaagtacacccagaactattcacggaatgcataaaaaaccaaaaagcatttcttgatggttactttgacgctgatggacatattgatagtggatgttataaattcactacaacatcaataaactgtgtattgggtattcaacaattgataatgaacgtatatggaaggcattccaatataagctatactcaaccagagaataccaaagttattgaaaatcgagttgttaaccaaaaaccttattatgttttgtcatataaaaaagaccaaccgaaaaaagaggaagctttttatgacaaaaattattcgtgggttaaaggtgtaaaaactagcactcatactggagaatggaaaactgtatataattttgaagttgctgttgataacacgtacactgcaaataatatagttgttcacaattgtcaagcatggtcagttgctggtaaacagggtggtataaatgacccacgtggaatgttgtttcacacgatggttgatatctaccaacacataaaaagtttaaatcctaacttgaaagttctctttgagaatgttaagatgaaagaagatttcaagcaatacattaatagtgttcttgagatgacaccagtactgataaattcccagcattatacgcctcacttcaggcagagatactactggtgcagtactgataagctttttataccacctgcattatcaaatgcccctaaacttcaagatatcattgaagatggttggtacactgacagaaccgcttcgtactgtatagatgctaactattttaagggtggtgattgtaaccagtactttaataaatcaagacgacagttggtgtttcgtgacccaacgattaatcgtcaatatctattggagaatggtaaaagaaatattgactatagacacttaagtgtagtagaatgttgtaaattacagggtattcctgtagatttctttaaagtgtctagtaagacacaagcatacaaaatgcttgggaatggatggactatgccagttatatcacacttactaagagagatattcttttgaacatgaagaactaccccacctatactgaacgtgtaatgaaatttaaacgtcagtcatcaaaggaagaattagaatttaatttgtactatcctaatattggtgatgaggtattgtattgttctcatacaaagaatggaactttacttaaatcaggtaaaggtattattgttgatatgattagcactttcaaaggtaaacttaatgtaatgtacaaagtgcaagattcaagtactgagtgcattcatgaagtgatgcataatatacctgcatatccaaaggattttgttcagctaatatctcgaagggagaaacctatgttttattgtggttatgacaaactaactaaacaaattattcctaatactatgggaaaatttaaaagtgatgttaaaactgaacttaataagaaattaggttcatcatgggaaactgaaaacgaattcatcgaaattcgtttgattgaagttactatactcgattaattacagatccacgctatgcgtggatttttcataggtgcatatatgacagttccttatgttaaaattattaataatatccttgattcgtgggaagatgttattactggtaaaaagaacccaatgccagtacacgcactaaccggaagactacaattcagaacgtcaaacaaaactgaaacttttaataatcatgatgttcacattactataaaatcacctgtacgtattgtaattaaaaaaaatcaagatgatgaattgacattatctattggtagtgataaaatcattttgagtaatttatccgaagaagaattctttcaacgaagtactatcatcgacatgaaagatttcacatatgaatcattggttgaactcaatacacgttttaacgatattcaggaacgattgcgaccaatcctccgtaaagaagaggaagcctggaatgaaagtgtgagaaaaatgcaagaaatgttgaaagaaaatattaacaatctgggtaaagctacaatggaatttggtaaatcttttaaactgaacttttcaaaggatcgtaaatgaaaattgataatttgattcatgtcttagggactttacataaaatattattacatcttaacgagatgaataaactaggttataatgtccataatgtatttgaacctaaatttaaatataactcaatcactggagatggtaattacataatcggatatacatatgaaaccgatccagacttctatcctcttacgttgagaatttgtccaacaaatccgataggaattcacgacatatcatcaatttcacttgatccagatggtgaaacgagtattgaatacaataatgattcaaaaccagaattttacattgatgacattaaaaagttgttcaatgatgaatatgttttccaacaaagtactgtgcatgatgttggtagtactaatgggcaattttggaatacatgttattatcatatgattgaaatttgtaaattactaaaaggtgatgaataatgaaatactgcattgaagtacattacactacaggtgatactgaaaattgttatgacgttcttaatacaattgatattcaatggtcttctaaagaggaagcagttgctgctttacagtgcctaaaagagcattgggtgttttatatgaagcaagataattgctatacaaaagaacacgaaaccatcgtcgaaaatgctaaacaaaaagagtggtttgacccaatatcaccagcatattcattcttggttaaagttggtgatgtgacagttccattacgaacaccgtggaacggctactttgaaactcttcataatgcacgagttgtcgctgttgataaccctgaacaaatcgatttcgattcattggactggaaaaaactatgacagtactgcaccgattgaagaaggttaagcatccagttgtatatgaggactggaaacgacagaagttgctgggtgatttttattacccaaaatctaaagttcttgcgaattcaagatgtacatattgcacacataatgataaagatacagtatttgtcgaaatgggtttccatccatcaaacacaatgttcactactaaccaaacgttttacaaatttcatatacaaaaagattcatttactgttatgattataaaacatttagttgaagatctggcattcagtgaagaacgagtacctttatcattagtggatattccttataataatttagaggaaatgtacttccaacagttgacagttcaagaaaactttggtatgataggacttcaagagttagagatgattaagcaaattcgtgatatctactttaaggagtaactatgccagtacgtaacagtacttcacgctttacgcttcaacagaaagcagatcttttgaatatggcaaacaaagattctaattttcactatctagtactcggtggttcagttgaaaagattgacctacgtgagtgtatgaaataacaaaaaggggactaaaatgagtctacgtgaatcatgggaacaatttaaagaagagattggttatcgtggtgtcaaaatcgaaccaatcaatcgcaccgaaagcatcaaagctagtactaatggcactacctatgatcgttatgatgaaaagtatggtttacattctaaaccaaaggatactgagtaatggtgtatactaatatttttactggtattatgcgtaagtttgttaatggtattggatatgtatccaatgacaacggtagtacatgggtaaaaaataatacgtccgaatctttagtgaaaagtcttccgtattataaagcttgacacatgaaaatggagtatgtataatactcctatattaatcaaatgaggatatacgaaatggctggtgttgataaaatttgtgaattcagtggcgattatgaaggttacgaaatgtacaagtccaagcgtaatcacattcaagttctgtcaaagtaccgtaaagaattccgaggacacaaagcaacattgtatgtttttgagaatggatattctgaagttttcacaagtggtgggtatagtacagctaacatggcacatatcaacccaaacccaactgaagatgattggaatagtggtaacgcttttcgtatttcaattttgaataaaatgaatcgttacgaccgttattgtacattctttgaaaatatttcagaatacaagcaagcattaaagaaatataatcaacgcttactaatgaattatgattacattcttcatgttcctactgttccaggtcaggttaatggtctgtatacgaacagtacacatgatttaactgcggtgaagcgtcgcctcaaacgtatgttgggttgtggtaatttaacaatcaaggttatgcgtaatgaatcaatgtataactttttagatgtactacactacacattgaaggataataatggaaacgattaagtttatgtttgaggtgtttgttgcattcatcttatgtacagtgtctattgctattgctttaatcccagtaatcgtttaccaagttaatggtggcacagatccaaccgttgatatgtttcttcgttgtgttggattctgcatatggttggcgatgtttatgtactatatgcatcgtgaggattgattatgtcattggaaaaagtaattatttctgaattccgaaagtacatcattgattgtgatagagatacccagcgtaagtttaatgagtatcttgacaaatatccatatttgatgtttaatccatttgaaactcaatccgatgttaatcttggattttacattgggtttacacaacgaattctacaacaatatgagatgaaacagccagaggtttgggtagaaactgattcgaatgagacgaatgttttttatctgtactcttattatgcggaaattgttagttcaatgacgctagaagggtatctattcgtcaaaacatctgatattgaaaaaatatacatagaacctgtatctttggatatggttgcccagcttccaacagtccaatatttggatgcaaatgagttgacatattcggtgtttgagtaccttaaccgagacgatttaactcaagaaattgagaatatggtttcaaaatataccacaactcttatatctaatggttcacttaggatagaactgatttacgacgatactagtactaatgcagtacgaggaactcgaattggaaaagttactatacgtgttgtccacaatgatgtagtacttggttatatttcgaaacgtggtcatgacttgtgtttgattaacacatatgttattgaacgaaaagaatggaacgatttaatgcaaaaaatcgtagacggaagtggaattcgtgatataatgtgtaatgaggtagtgacaatgaatcctgacattgactgtgatgtatatttcccagttcctggatttactctacgtaattttggtacattaaagggtacaaaatgaaaattttagaagtcttacttgaaaaggatgcatctggtacactttatgttggtaatgagtttagtcctgatccaattgatatgatgcagcgtattcaaacactgacccaatcaatttcagttaaacaacctttcctaaaagaattggaaattcactgcgataaagatatggaacaacatgataatgttatttctgatatcaaaagtctattgacaaagaatggatatgtggagtataatccatctatcgtaatcgttatccctgactttatgaaaggaacattgtaatgactaaagaagaaatgaaatctcgcctaagcgaacttaagaaagaacgaaaactagtccgtttggactgggacattcttaacaaaaaagatgagcaatcatatgctaactggagttccgataaagttcgcaatgaccgttccaaagaagacgctaaacagtttatggcattgaccaaacgtttatcttcactgaatagtgaaattacaggtcttcaaaaggaaattatttcaattcttcaagtggagatcaactcactgtatgaataaacatgaagtgattcattttgatggtaagatttatgtaccaaacaatgatagtactgaatcttattatctaatcaatgcaattatgtcagaaacacaaacatgttgccgtcagcgtccaggtgagtatgagttcagtcagaccattggttattatcgttatactgaacttgataaatttacaatcgattctaaaaaagttattgacgttctcacgaagaacggatataatgaatccgaatcaaacgaaacagagttaccagtcgtgaaatttgttctaaaaaataattaattttttggacaacgctgaaaagtatgatataatgtatattatcggcagatgtaaaaatgcaaaccattcctcaatgatgagggtttttcataaaaaagaaataaaagggtattaaaaatggctaaagaaactaacgcacaagtactagatatcaccgacttcgaccgcactgtgaactacattcagttccagattcatcaagctgaagaagctctcgcagtattccacggtactggtgcaaggccaatccgttttgctcatctaattgcatggaagaaaagtctacaagaagaactttctgaactgcgtaaagcaaaccgtaagtaattgatttcccgcctcatcaatgttgatggggcttttttaataaaatacaaactgaggatatattaatgagtgttgaatattggtttttgtttatatgtcttggtacactagtactgattacattgggcttaatcttcaatacggtaatcagtggtagtaattacttcaagtcctttcaacttctaatgtcagctatgcagtacagtgacgaggcagaagaaaccactacgttgattttgaataaacttgaagctaacttactggttgctaaaatttcttcgtgtaatacaaatattgtattttatgcatcacccgtggacattaatgggtacgaaatgagttaccagcaagccgttgctgaaatttttattggttctaagtattatgcatatggatatatccgaaagtattatgctgaagcaaaagccgagctaacaaagaaacgaccgtcaattaagacattaaagcgtatcatcgcactagaaaagaaactacgaggtgatgtgacaaaaaatgattcacccactgtagtaaatacaaagaaaaaaaataacgtagtggagttggattaatggacgcagataaatcaaacagtattcttattgatgtacaaacgaagttattagatgttatggaaatcatcaaagatttaccacaaggatatcagtttaaagttcgtgaaatgttatcttgtgcagaaacggttgtgtctatgtacaatgatgtgcagcatggaattatccctgaaattcttcaagatggtctaaatattaacacaaagattccagcacaagagcctgaagaggttgttcagcctgaaccagaacctcctttacgtaaagtactggatctgtctggcatcaaaattaacatctaaaatacattcaataggagaagtaatgagtaatcaaaaaatcgacttagtagtaaaacttcaagagattcaagcactagctaaagaacttgatgaaaaacgtgcagcagcagttgattcactgtgtgcaacaatcctgactgtttttgcagattacgaaaagaaatctgattaaacattactttatacccagtctagtactgggtatttttttaaggaaataaaatgactgaatcagaactacacgaaatgatagatttcatttatgaaatggtgcaagataaaaataacattaatacaattactactggaccaagacaaattggtaaccaaatgacatattcgttgtccttatggttacacctgaacttcaaggttgattgtttgtattttcgtcctgatattgatgcttattattttgtaaatcatgatgcattaatgaaacaaaataagcgtccagttgatgatgcctttgtgacgcacccattttatctggctgattccgaagaagatgagtttcaaaagaacacaacgttaagcccaggtgaaatttgcatcccttacacactaaaagataaaatgatttacatcacagaagttatgaaaacatgctggtaagtgcaatgaaactctgatataatacaaataatgataataggaggatatatgaaatttattaattttgatatttctaaggtcaaagaattatttgtaagaaatgcagtttatgatatcatgttacataacggtactgtacaagaagatgttgtattgttttcgtttgatggtgaaaatctcgaaactatatataaattcagcaacttagatggtgacttttgggaaaccagtgctgttaaaatgtatagaattaaaaaggagcattaatatgccaattcgtggtgtaccagaaccattaatggaagttattaagcatgttcgcattggagatcgagaagttaatcttcgccgtaataacattttttactttaaaacttcagaaatgagttctgtacagattgtcattggtacatctattaagattactcgtgtacttgatggtagtactgacttcgttgaatataatgtacttactaataaagtaacgcatatatataaatggagtggatataaaactattaagtatgaatttgacttagctagaactgaagaagaacgatttatgctgagtactatcaatccagcaaactatccagatcttgatgcatttttgctaattttaaaattgtataatgaagcaaatcagaaagccaaagattatgatagaatcatgtatgaacgccgaaaagaacgtgcacaaaaaagttatgatctgtatgatagcccatacgtgaaatatagtaaggggtcgaaatgaataaagaagttcaaattatcataactccacctatacaaagtctggatgtatccattagtgattttttggaaaaatactatgaatggtttaatgaagattcagtaatttataaccattcattacctgatggtttcgttgagcttattgaattaaatgccaaatcactatacttctgtgttgaatacaatgattatatgacagatgaagcatatcgttttcgttgtacattgtatcctaaccatttttccattggttataaagactatcagtactgcaccacaaactatagtgtagaggaagaatttcagtacaatttactttatcctgatttagattatacgattgcagtactgttgactaaaatcatggatttttataaagataaatataatgcagcctaagacatttgatgagtggtttaatctacactataatataaatgcagcagatatatactattttaagcttgaattgtatatatcattgcgtaacccaatcgagtactataacaacatcatcaattgtgatgattttggtacatattttagtatagcagtcatggacaaaataagatgacacacaaagaatggttagatgaactaaaatttaaacataatccttttggttttggtatacgccaaggtcattatgatgtgttaaaagataaacaacttttcaaatcatttgtacttgatgaggatagtaatcactggtacatctatgagcttgcaattaggaaatagaagatatagtatgagagtagttattaatccagctaaaagttttatgtcatcagtttacggtgctgatgatattatccgcatgttaccacgtattgaaattgatggaattgaagtgagaaaatacggtaatatatattatgtagtagattccaatggtaacaggatcagtgatagcttattcttcagtactgaagaaatgaaacacctaattataattgattaacaaaaagcggcatcgcaaggtgcgaggtgaaacttccgatttccaccgctggtcgattcgatttcgacatgccgctcaaaatttacattgacaacctcagtatttttgctataatcacttcagataaataaacctacgaggaatccaaagtgactaaagcagaacttgtagcattattggaatcatatcccgatgatgcatgtattgaagttgttcatagtgcgagacattttgacattgaagatgtagtacgttataatgacaaaggtgtactcatcatggaactatgtattggtgaaatgaaaactgtacccgaatcagaaaaacgtaaaaatagtaaaccgtaaggaaaataaaatggaatccactctattagaagttattaactcattaaacgctggtgacaatgtacaagtactccttcagggataccgtgtaaaaggttcagtagttttagttaatgacaccactgttgttgtaactgaaactaacccgtttgacagtaatgaacttgctaatatccatattgatattaaaagtattattggtgttggtgtaactaaaccacaagaataacaattgttattggggattactatgcattttgaaaaattcaatacaaatcgtaacacacgcattgacgttctttatgaaacacaagactcaataagcattcgtacacgtagtggtgatgtatatcatgactgtactattggttgggaacagggaagaacacgtagtgcagaattcgataactatcgcttctaccaaggggacaccacaatctggcctgaagacgtagaagatattgcgtttgaagatgaacataaagatgaatttataaaatttaatcccgaaaccgaatcataaatattggaacagacaataaagatgtgatataattattagtactggtgaagtggcagagcggtttaatgcacttgtcttgaaaacaagagagggtgaaagtcctccgtgagttcaaatctcaccttcaccgcatacaatgggctattagttaagtggatataacacatcccttctaaggatgcgtcccaagttcgaatcttggatggcctacttattcattttataattaactgagaaataaatacacatttttaattacaaggtttaataatagtgaaatgtaaaaattgcaatcatgaaacttctaataaaaaattctgtagtcgttcttgttctgtatcatataataataaattgagaaacgtaaatggtaatactcgtttattaattcccaatacagaatcatctttatctggtgaattttgtataaattgtaaaaacaaaataccagaaaatcatagaaatgaaaaaatatattgtaaccgaaaatgccataaagaacaccaatatacacaaaatgtagagaaatggttgaatggtgtgcatcctgggtataaaggtaaaacattaaaattatgttcttttgttagaaaatatatacatgaaacacgtggtactgcatgtgagaaatgtgggtgggatgaaaaacatccagtagatggtgctgttctaactgaaatagaacataaagatggtgatgcatccaataatgaactttctaatttggaagtactgtgtccaaattgtcattcaaaaactgcgacatacaaagcaagaaataaaaattctgctaggaaacgatatttataaatatgacacatgaagaatggatgaatgacattaattttagatattcgggtatcggtagttggcgaaaatatcattgggaaatcatatcagacccagcagaatacgataaacttataaatcatgaagtatatccgtggttctttattgaaataatggtacgattatgacacatgaagaatggatgaatgacattaattttagatatgcaggtataggtaaatggctaaagtatcattgggaattcatttcagagccacatctgtatgataaacttatcaacgatccagcatatgaaatgtacttaattgaactactggtacgaatatgacacatgaagagtggatgaaacgccaaaaaataaagtggaatccttattatctaccattagattctagaatgtgggatgttatacgtgttaagtgtatgtacaaagcatatttaagtactgaactcgacgaagaagtactgatgcatatcgtgcaccgatgatgaaaaaaagcttgactaaatagttcagtactgtataatgtttgtatcttaacttatatggcaacttgatatgtttagtgaatatgaagaagcaataaaacacaacattgaagattcacataacttccaattttataattgggatgaactaatgaaattgtacttacagtgggttgaagatgaagaaacttctgaacaacgaactgttatgttaaacatttcgttacgtaaagaccttagttcaattagtgttgaagttatgacatcttcaacttatgcagaagaagatcataatccctatgtttcaggtacttcccctgtaccaaagactgaagaagaatattttcaaacgagtactgtacaagaaccattattcacttatgagttctataagtacttgattggtaaattcaaaaaaatgcgaattaattcttgactaaatagttcagtactgatataatgaattccacaatcagacgtatatggtgtaagttacgtcgatgcaatccagaacaagatctctggtactctaggaagtggtgaatgttaacctagacgggtttgataattggacgataggcaattattacttgactagcaggtttaaactagaggtgactatccttcaccaaactggaaaaccggatggacttttatttaaaagggtaaggcgtggaaatatgtttacagtagttgcttttagtagcctgtacgccccacgtcaaggtagccgaaaggcaagttaaaacgggtggttataaagctgaattaacagcggcgtttaaaagcgtcaccatccattttttttaattaaagttggtgatatgagtgttaaagacatttcaaagtatgaagagtttcttaaatcatttttgaattcagaattaacgaccgaccataccaataactggtgtcaagataatccaggaattcgcattttcaatggatatggcgaatcatttcttataaatgacgatgaaattgtttttgaatcatttgaagaaataaaaaggacaaataacactttacgtgatataataaatagtataaatgctcaatacattactttcaagctatgtgctgatgaagcagatgaattccagaaacgaacagtatatgaaaatgtgtttccacatgaattaaattctgtgatatttaaagtgttggagaaggttttttccgaaaatagtactggacagtaggattaagtactgatataattaatttagtgagttggatgagaggttgaaatcactaccctgctaaggtagcatatgcgaaagcgtatcgtgggtccgaatcccacactcactgccaaattgttccgaaaagaattgagtactaagatttgtaataacttgtcttagtataaggtttgtgtattcatgtgatgttcgagtataatgattctatatgatattgttattatatgagtactaacgataatgaaacactaggaacaccctaaatcgggccattagctcacggtggttagagcaaacgactcatcgacaatggtgactttcatcagtaatggtgattgaaaaactggatgaattcagggaaatcttaacacgtaatggtgatgacaatcctgagccaagcccaataagcgtattgggaaggtgcagagactagatggtacagtacgatcttactgtgtaatacatcattagcgtccggcatccctatgggatgatgatatagtccgtactatagtgaaaactatagattatacgaatcgttaggtactgtgttcgactcacagatggctcacaaactttgaaataaaataatgaaaataaaattagaatatccatacagtaatgattgggagaatgggtatatacagataaaccctgaaggtagaagaactgtaatactttataattctccaactgatcgaagcagtactcaatatgccagatatctattagcagtgcaagaaggtagatatttaactgagtctgaagaagcagaccacattgataatgatttcacaaatgatgatttgtctaatttgcaaatacttcatgttgatgaacatcgttataaaacacataagaatggagtgcaagcacgatatgattcattcatatgtgcttgttgtaatacggaattttcccgtaggcatactaaagtgaaatcatatacaaaatattgtagtgttgagtgttcacaaaaaatggtcaagcctccttctaatacaaagggtggtgaaacatccgttgattatgaaaaggtaaaagaatatattaaccaaggtttattagatactgaaattgctaaaataatgaatatcagttcaggttcagtactgaattatagaaccaaaaataacataccatctaagaaatttgcaaatgtaaaacgtttagaagataacatagaagatatcaaaaaacgtttgactaatggtgagaaaaaagttagtatatatgaaagttatggtgcttccaaaaatgtgtttgctagatttttgaaaagacatggcttataaataattatgactctgtagcacaataggtggtgcgattaactgttaattaattggttggggattcgagttcctccagagtcgcaaaacgaaattacaataggaaattaaaatgcaacttttaattctttcaatattgtatattatcataatggttccgatatcaatacgtttaattacagttgagcgatttttatcgaaacatggaaatagatctatgatctttgtaccaatcgggttgatattgtttggtatatttgcatttataatgggaattgacgctacattcatttcataggaaacaaatatggcatacttcttgattcaaagcgatgctgatgatgaattgatcagcaaaactgaaattgatattaatgattaccgaacagtactggaagctatccattactatgatgttcgacacaaaggtaatcgtgaacaaattaagaactggattgaatcgggtgagtacaaggttgaatctttatccatttcaggtgatgaatctgaagaagatggttttattactgaaactatgtacttaactattgaaaatcatttctaggaatttaaatgaaaaagaatgacttgatcaaaatgcttcaagagattgacggcaatccagagattgttatttggaatggatatgtcgatgattatatgaacatcgaaaaaggcttgactaaaatcactctagtcaaagaaactgaacagttccttctggaagcattagtgcacgaatggtgcaaacgtaacaatacattcgacattccagaagatcaacgaactaagattgcagaacaagctaaaaaacttcataagaaacgtgaatacgatttccagaacccacatgttactgaagacgaattcaatcactggtacggtaaaaagaaattgacaaagtatgtcctttcacctaaactacgtggtaagacttcacttggtgtattcaacgccgcagacttaaactactagcgggtattataaatgaaagccaaagacctaattaaaattttacagaataatccagaagcagatattcttattcatgcttcgccttatgatgactatgcgattggtaaatgcgaaactgctcattcatatcaagcaggagacacagatgttgaagagtctggtgaagatttttcagattcatttgatgaagatggaaaactattagttccagtaatcattctctcttaaaataaatgtagacggtcgtattcagtactgaatcactaggggaggttgtgattcccgattcagtagcttataaagcagttccgtgtgtgcctatacattacaactagtgcacatttaaaacctagacgtaggatgtaagtattcagttctgaatagctgaaggacttattatggtacaatacagtgagaggtacttcgagtatgctcagttcacctttagctaaaatggtcactgtaggatattgttgcaacagtatccagtactactttttaattgacataacatcatttgggtgtataatgaagcgtaaacaaaatcttagtaagaaaatacaaccgacaatgacgaaagaacagttgtcaacatttatcaacgcagcattagataaaggtaagagtaaaaatgaaagtaagtgaagaaactgctaaacagcttgaagtactttgtagtgctgcacctatgccgacacttggcttaatcaagttgatgcgtagaaataagcaaaattcaaaataataaataatacagtaagatggcctcccctcaatgcgaaagagtatagagacttgttggttatcttacttctcaattagccgacttagctcagtaggtagagcaatccccttgtaagggaaaggtcaccagttcaattccggtagtcggcacaaaataaaggtacactatgacatacaatattgttaaaatggtgaactacgtattaaaaaacaacgcatggtcactacatgatgtacaattaaaagatattggtgaagttagagttttttgctatgatgacgatttgaacgaaaccactgatttgagtaaatgttttaatatttttcttggttatgaaatcaatgatcatttttattcatttgatgtgtattttgaacgagttaaaatcagaattgctaatgtaaatagtgattattatgacactagaattgaattgaattttgacgatattgatactgaagaaaaattctttcagtactgtctcgtaaacgatctaaatggattgacatttgaggacattagtgctatccttaaagttaaaagattgattccagaagaggttccaaatccgtttaatgactagtttaataactcgtataatgcaagatgttcgtaagaatctttatactggtaattttgatgatcctaacataaaaatattatgttatgatgtattcacactggtacacccatctgaatctaataatatatacatagcatatacttctcctgatattttgtatcaatttgatttatatagtacaatggctaagataacattagatgtttctaaggagccattttttatagcagtacatgattttgagtacacagaagagtcattattccagtactcaatgataaatgacattggtgacttgacattagaagagataaatgttatagttggtatgtgtcgtttgattaatccataactcggagcttaatatgtttttgaatttgttatgcttatttggtcgtacattggttcttgctattattcatacacctggtattcgtaattatgtttcttatgaatttaagaaaaattaccacaacaacactgaagttcgtgtaactaagatgtgcaaagacttcaatggagatgttcaaccattcaagaagtacgaggatgaataatggaatttattgttgcacctgatgaactaaaaacattcacgttccgagacaataacaattatccttatataaatcattcattgaattcattgcatgttatttcaatcattgcatctgaatacagagacaatccatgtattgattttaaaaccactgatggtgaatatacatgggtagttccgaacgataaatacagttatcagggtgcagtggagatccgcgactcgattctaaagcaaatcgaagatggacatactcatcctaaactaaacattcagtactgaaatcgtttaaccggatagcttctagtcggtactgaattcctccttttgtctagaagctatcactcttttaaagtataatgatatcaccctcatggttcattatattcgttaccctctaacagtatctttttattcctctttgatactgtattttagaagttgacatttttacttctactgtgttatacttaaggctgattcattatgaatcggccttttttcttgtggggattaatgtaatgaataaagaagttattagtttaatggatgagtatatcaaccaaacatatcgttttatattttttaatgcatttgagtccagaacaacgtattattctggtgaatatgagataacaactggatcatttcttaacggatacatggagtttttagaacaaagcgataatgtgttcatttccatatctgaaaaacatggtaatgattatgttattgatatgtggcctaataaagtagagattgataccacgcacaaaatcgtaattgatgtggatgaatcgttgtatgatttaactgaagatatgtacatgcaactaaatttcgtatacgatctaactaacataagttattctaatctaagtaaattattttatatttgtggtgtactacataaaaacgtacaacaaaataaaggataaacatttaaatgttgaaaattccaccacatacactatctatgctaattgaaaatggtgtaatattttctgaagggtgtagttcattctatacatgtgacgtagacccgtacacatttgaataccgttcatttaaaaatacattgagtgttccatgtgaagcttctgacaacattgtacttgtcgtttcgttctatggtgtgccaaaattcttcattgatatatattatgattcttttaagatacaaacatacgatcattttcttggtgaagtgctcttgcaaatgcagattgaacatgttactatagatgaagatatgttctttcagctatctacattatacgatatgtgtggattgtcagttgacaacataaatggtatctttgaaattgccagaaacctacgaggaagatgatatgaataaaattttaagtgataatgaggttgatattgtcattgatgtacttgaaaacagtaatacatttgagaagaaacacagtactgtatacttcggtaaagattgctggttgagctatatatctgataaatcgtttgtaattcgtgttgaaacgggtgatattggatttacggtacatgcggcacataccaaaactgataaacaaatcactgttattgctgacacgccaagtatctgggatagaattgatgattgtgaacactacacgtcaacattctatgatacagaagaagaacatttcatggatagtactgtcaaagaattaactattccatttaaacttatgaaagcagcaagagattttattttaaaatacaaaaaaataggagttgatgatgcaatttaatggtaaggtttctaaagtagggttagtgtttgaaaattgtgagttcattgagattgatgtaaataatgttttatggatgcatattgctgacgagaaaactctttgtagtcgtttttccaacggtctaactattagtactggtattggtgagtttggtatcgcattacgtagtgacacaccatctgatttcatggataactcaacactgctagaacgtttaaaaatgcgtgatgttacacacgtttatctttatgatactaatggtgttgaacagcgttatgtagttgaatgggctaattataaccttagtgatcaatattcagtaaatcagcgtgttcaactcacccctggtggtaatattgtgtttgttagttcaactggtaaattaatcagtgtagatgatgaaagtatcgatctattcacttcatataactaatacgagcttctatgaaaacaatcaaaacacatttcaaagacacgtggacagaagtaagtactggtaatattcaagatattttatacagtactgaaactattcatgtctatgacgaagagacagcagcggagcagttaaaggtattgcatcgtagtgtaggtgaacacaccgagtacggtgttaaacatactcgtgaattggtaagtactgatcatattgacacctgtactgtaaatctatcaaaagaagaaatgtttgattttattctaaacaatactttagcaattcaaccagatggatcactatacagtaaaacagatggtacacaagttcataaaaagtacaatgtgaccatcaatgatacggtgatgtctggatatgatttagttgatgcaatcaacgagtacgtttctatgcagaggttgaaatgattaatctgatatattttacaattatttgttgcattactattgtactttgtcgtctaattcatgtatactcatatcataaagcgttgagaatcgaacaagagatttacaacgaaaaccgtgagccagagccagagaagctaccccgctcaccatatgcattgaagcgtgagatgcattcggtgagtaagaagtggggttatgcaataaccaagcatggttatatcgacactgctgcaaatggctattttcaaattcactatgatctttcagttgcgattggaattttatgggaaatggaggatataggtggttatgagcgtactaaatttccaaacttacaaaatgatccaggaacttcatgaagaacatgatataattatagatgtagaaaagaaggaagttgttatgaagaagcaagcatatcgcatcttagacaacttctatcttaaggcatttataatgaagttctgtgcagacgagcacttaacaggacgtgttatacgcaaggagtcgtagatggacatgagttcaacaattcagtacttgagcgagaattttgtcaaaatcatttcacgtggtggtatgattgtgcatcctgaaactgaagatatgtatatcattcgttttgatgcaaattcttatgatcaagtacttgttgtttcgaaattagtaggttataatgaagtcgttcagttctgctgtaccgagttcggtatctatattaaagatgcagaaaccaatgagatttcacattatttttatcacacactgatgggtactaaagattttggattgaaattcaatgatttagtacaatgtcactatatgtttaaaacatacccagcatctgggaaataaggagtaacaatatgagcaacattttcttcaaaaccatttccttcaaaaaagaatcttactcttacgcttatgtagatggtaaagtactcaatatcgttctagatggtgttgatgtacctgttcaatttaccagtcatcaagaagcgattgagggtgctcaagagcttaacagcttactaaatggtattgagaagaaacctgaagttaaatctgaccttgatttattggttgacaaaattaaaagtggtgaactattatcaattctagaccagtgtaatgaaaaattacgtggtgttgttggtgatgtacgtactacagttcaagatcgtctacttgagcaagcattcaaagaagttactaagaaaactgaagaacgtgttgaaggtatgattaaaaacattgatgacgttctcactcatatgttcaccaagccatacgaatcaagtactgattctgaagagaaacaagaagaaccagtagaacgcaccaagccatctattgttactaaagttaaacatagttttggttcactggatgatgtatttggatttacaaatgtacgtgttagtactgatgaaaagccagaagttgatgaaatgctaattggtgacatgacaactggacaactcaaaaaagagattgatacctttgttaatgaactaattagttctgaacgtgctgaaaatatgtttagcagtatttcagcaaacttcggcaaagaagcaactgagcaagcagtacaggcattcaaagatttgatttacactgttgcaattgctaatccagaacaaacactggaagaagttctacgtaaaaacttcaaataagatatagataaaagaaaagccactacaatgtagtggcttttttgttatgcgttataaccaacaatgactacagtacagccaaatggttcaacaacttttaatacaacattatcatctgcatcgagttcaacttcacttagaacaacatgattgtcttcagcatagacttgaacttgtgggaggattttaccatgttcactggcaggaatggacagtgtgtacatagaatcaccatcatctacccaatcactatcggtatacgtgttgtgatatggagtggtgtgtaaggttttaccaataatactcaatgagattggagcatcatcagtttggtacacagtaatattaccgctaggatctacaatgaggtctactccaatttggaataaatcaccactgtatgcttgtagagcaagatactcaccacgctcatgcacagtagctggtacagttgccatgtacataccatcggacgtgtactcaaactgagatagaagtactgaagtaacgaagtcaatatcttcgccgctttctagtatatcaaattcaatagttttggtattatatatgtcagaagtgagtatgatggttcctttaccggattgagtaattggatcaactggaatactaaaaacatcatcaattatttcagttgtgaagtactcggttccgtcaggaagtataacggtaatacgcattccagttgtatcaatggctgaacctgcgttgaataggcgaccacctaccaaatcatcacctacgaacgttgggaacataaggatgttgtctatatctctatctataatcccgcattcaggggtgtacatattatatagtgtttgtgaactacctagaattgttttgtgcaaaagaaacttataaacaaacccagtaggatctgattcaagtttagcatctgagtatcctcttacgaaaaaagagttctttcttatttcacttaggatttggtcattagttttgttgtagtttaattgatatccttgtatatcctttgttgtaatttgtactttacgataaattacattacttacaggatcaagtaaatcatataatctatggcttctctgacgatagattcgaacatttttgagtttagtcccatcgttgagaattgcaccatcgactacatatccacattcaccgaacttatattgtccagcagcagcaatcgatcttgacattcatccctccattggtataattattataacgtatttattctagataggtaaaaaagcaccttgactgagttgtgtttttgacgtatacttaaatggaatatcaataaggggattttatgagtgttgtagaaaaacgtttagtactgaaaaatggtaaaatgactgttaagctatacaaagatgaagctgattcttttatggctaaagcaattgagaaatccgaacgatataaaattcttgcacctgcaagtaaatcgccgtatgggtttaatcacgctcacagtcattttgttggtatggtatgtgaacatgcgtcatttgtactttttaatgaaattgaaaactattcaggtattgcacttaacatcgatccagcgttccaaagtgatggacgtgatggggaatgtgatatcaaagtaaacaacttaagaattgaagtgaaaggtatcaagtacaaatcgtggaccgactttggaccatgtgttagtactcgacagttaaaaaatatacagaaaaaagctgacgttattctgtgggtattgtataatgagaaaaagcaagaattcacatttgagggattcaactatgtaaaagacattagttcgattccagttgtgctaacaggtgtaccagggcgaccaatgattgaaaattacccagtactgagtattattaaaccacttcaggagttaccattatgattactgccactgtatatcaagatattaatgaagaatttaattctaaaattgcatttttatcctttcccgtattgccagctattggtgctacgttgaatcttacaatctgtggctttgaaaaatactatgaagtactagatattgagtactgtgcagatgtttattcatgcaacagtgagattgctattcctgtgcaaatcgacattattgtaaaagaggtttgattatgtttaaacactttctagtaatcactgctgttgtattcagtacttcctgctttgttgcaccagcacatgcggaagaccagcgtcaaacaatcaagacgattggaacagggaaagcccaaccaaaacgtactgctgaagaatacatgcctgagatgcgtgaacgttaccgccagcaataccgtgaaaaacatgctaatgataagcctatcaaaaagcataaatggaatgtagtcaaatcgccatcgttttgtgctgggtatgctaaggttaaggatgaacagcacaacaagcagtactataaagatatgtacacattctatgattcgaggatctcggatgatgacatgatggctgctaaggttgataaagaagaatttcttcagggttatgacatgaccgatgatgacaagatcatggaatgtgttgaagtatatgaaaaatacagaggttttgaatgacacttttgctgattctaggtttattagtgtcatatttcatttttcttaacatcttctctgatgcatcacattttgcatatgcttcagctattgcaaaacacgaagggataaagaagtatattcctgcattattattctcatatatgtaccatccaaggtactttacactaggtgtgtttgacaagtatgtatacgacgaaaaagcactcaatgaaattgaatactttgtaagaactcatgctaataacaatccaaaatcattaattaactttactgataatcgtaaacgtgttagtattagaagtgacttgtatcctaccagtcactttcatctaggcgattgtagtgtgtacagagttagatacgaaaacttactgttaccaccatcacatataagatacatagaaaaatacttgttgaaatgtgttgactccaaatagtaaatggcagtataatagcttcgtaccgcaataactgacaagaggatttaacgatggacgtattagctactcaattcaaacgcaacgcagaagaacgcgaaggttacctaagtatcagtgaagtggttgaagatggattcaagcagtacattttcaacagtgcaattaaactgaagccaacaaaagattcaccggaccgcagcttcaccaagtggattgcattctgggaagagtcaccaattgcaatcgaaatgactgtcatcactacagtactggaaactggttacatcaaagcaattgtagttgataagaaagttcgtactcttgatgaaatcgggacatacccagcacgtaactctaaagtatttgggtgtttagttgatggtaagtttatgattgaaaaagatgctaagaactatgtaattgataaactacaaaccagtgctactaaacaggaagtcgtagaccagaacgcagtggatgctattaaaaaggcatggggactgaaataatgttcacacttagcaatgcaaagtatctatttcttattgctattattggttgggtgatggctccagtgcttaatgcactggggtttccagacggacataaacttgtaaatgtttcaggtgtaatccagggcatttcatggtattcattgattatattttctatatgtattcatacatttgcacgtgattgccgccgtctgctttttgttaaagtattcggtactaagagatatagttttgtgtttacatttattttaataccatgtattatctatgcttttgtatttgattactatacttatctgtgggtgtgtggttctatgtttgttatgttggaatatacaagaactgttcaaaactcatatgctgatgagtattatgatcttgaagttaaacgaaaattaacggagctaaacaaatgaggggatacaataagatttgtatttttgtatttttagtttgtgcaatcaatacatttggatattactgtactgaaaacaacatttcatatggtttaaatcctttgtggtttacaattgccaatgtagcacaaattggtattgctgttatgtctgcacttatgttctgtccaattgtcactatggatactgatcatgaaaggctgggatttatccgtgatactggagacgaaccttacagtagattgattgtgttgtatccatttattgttattttaggtactaacatccaacttgggtctttatctaatagtgttacttcagcagccattacagtctttgcattcacattttttatggtacacaaatcagtactcaaatggcacatcaccaaaatggcagagaaaggtacggaaaacagttgacgtgagttattgagtcatatataattattacatcaaaactgggaaatggtaaggaacttatgaacatcgataaggtcaaagaacttagtgaagcactaattaagaacgaagaagcttgtgaagaacttcgcagtactatcaaagttgagtacgcacgtcttcaagctatgcttcaggaatccgaatgggttgtaactaaatccatcgaagctatctataaggatatcgaatcccttttcaataagaaaagtaagtttgaatctgattcatataacaagcgtaaagaattgaattcagtactcggtatctaaaagtatttgacttaaccaataacatggagtacaatgtgctcctaatttaattatcagctaaaaaggaaaacattatgaaatctattctcgttgcagttaacctggttgcttccccatcttttaacttcaaacaagttgttgctgataatctgaagaagatctgtccaaacgctgaattgactttctgtgtgtttgattcagaaaaagtaactaaagttgatctaaactatcttttgaacgaagtacaaagtatttctggtgaaagtaatttggaagtactgaactccgttgctgatgactatgatgatacacttgtaatttcggagaaattctaaatccaatgcttagatttatacattacttaatcattaaattttttggctattatacgattgaaggcaaagcaacacttgcacataaaatcttcgtagcattatgtgtgggtgattatgacgtattgaataagtactgtaaatctaaagcatgtattgacataatggcacttcgtatctatgagtgtcagttcaatagatataatttttaccagtataggtatgataaagatgattttgatagcattgaaatgctaaagaatttatgtaataataaaaagaatatgcatatatctgaatttgttgcattgtatttttatttaaaaagaacattatacatgaatacactacaacgcttcgatactagaattaatgtattcaacgataaagaattttatatctatgataaatttttagataaggtttaatatgacgactgtttatattgataaacgcttgggagtattacttactgattcaaaagtgacatcaactgagcaacgttcatttttgaacctattcccgttgaaaccaaaacttagttatggtgtagtttcacaaaaaacattgtatgtacacgaccgactgtttagtgcttgcggtgaagttagtgaaattaacaaagtactgagatacttgatttcacaagagactgtgataccagaccgcaaggcgagttgtcactgtgttcttgtgtcaaaagattacatcattcacttaatcgttaataagggtaagtttaccaagaagattgattatctccataatgattatacattctttatgggtagtggtggtgaatacatgcttgatgcagcagtacatgaagagtataatactcttcttgataaggtatttgataccttcatggatgttcataaccatgatatctataccgatgataatattaatgtataccgtttttgatttcattcattaggagaagtaagtgaaaaagcttgatttaaatgcaatttctgaagaagtactggatgagtgttttgctagtgaagaagtcaacgtattagaggctcttgattccgtcgaagaagaagaacatgacggtcacaagtttgagaaaccaaaaacaggtatcagcttaactcgccctaaagggataacgaatgaaatgctcatccaagcagtgcgtgatggtattgacgttgaaaagaacattgaccttttggttcgctacaactatccaatggtagtaagtgttgcacgtaagtgtacatgttttatcccatttgaagataagatttcttatggtgttgaaggtttaatgcgagcaattcatacgttcgacactaccaaaaatgttaagttcattacatatgcaacaacaactgtgtacatgacaatctataacgaaagtgcggatgctaattatctagtgcatttcccacgttatatgagtgttaataacatttatgtgaaacgttgtgtagaagagtatcaaaggaaacatggtaaatacccaactcctgagatggtgtcaaaagagactggtattgagattcaacacgttaagaattgtctaacttacaataacacttatgtagcattgaatatgaaaatgagtaatgattcagaaagtacattatctgacattgttacttcagttgaacctgattacaacttaggcgtagaaactgtcagtaccccactggtacaaagtattgcagaaatgatgagtaacctatctgccaaagaccgtgaattgattgaacgtgtacatggtattggttcttattctgaaccagaaacgctacgagaaatcattgagagtggttttgaagatgagaagggtaaggtcatgactgctcgttcttcattgcaccgtaagtactctgaagcaatggaacgactgcgtaagatggttaagacaaaaggttatgagttcgaagatttttaagattaaagggttgacttctgtcagccctttttttatgggacaaatttagaaagtgtgatacaatgttagtatgataaccataattggaatttaatatgaaattggtatatggttttggaatcaatgatgttaaaggtgattccacaaaaaaaagtgtttatagtaaagtttattatacatggaaagatatgataaaacgatgttattctgaaaagtatcaagaaactcataaaacatatataggagttattatatgtgatgaatggaaaacatactctaaatttaaagaatggtatatatcaaattacaaagatggttatgaattagataaagatttaatcgacggtaataaaaaaatgtattctcctgaaacgtgttcatttgtaccaaatgaaataaacatgtgtatcattgacaaaaataataaaaatcaaccacttggtgtttgttatcatatacctaaaaaaaatatgagttttgaatacaaaaatccatattatgcacaaatttacaaaaataagaaaaaatatcatttgggtgcatttcccacacctgaagaagctcatgcaaaatggcaattagccaaaaaagaacatctttctgatattattaaaaaatataataatagtgtgtcagatggtgtaataaaaggtttacaacgtagaattgatgttttaaacgatgatttacttaatggacgtattactttgtcattaagtaaaatttagaataaaaaaacctgtagctcatttagaataaaataatcctgaaccactcgaaaatgtagagggcagatgagttgtgctacagaacgaacagcaaaagaaacccgttagatgtgggcatagtttgaacccctcacggcaatgcggctgtataaagaactccgataagcataccgagagttcctcgatagacatataaggatcgcaccttaccagatttgctaagggagtcttattataagacagcttgaacttcgattgtatggagttttaagtgagtgctgggatgaaaagaccagtgaccgttgcatatgaccgtgatagggagaaacagagcaacccgcttccgtttgcgattgatttattacaatatagatccgtcctaaattcacgatacaataaaatccgatgccatcagcgtcaagaagagacttaattgtctcttttttcttaccatcattcccctgcgggggatgaaaaagtcctcaacaaatcccgtcaatattaaaagtccttgacagttatgattcattcgcgtagaatacgtagtattagaagcgaacagacatctcgaagagatggatgattaaccattgaggaatatctaatgaattcagtaatctttttaattattgctctttcaccacaatctggtgtacaaatcaccccaatgaattcaatgaatcaatgcattgcagtacgtgacagtacgtatgaacaagtaaacagcgtttcacttagaacagttggtgaagaattaatgtggtgttacaatagtaatactaaagaaaaactaattgttggacaaacagaatgagttatggtgttcgttttgagattcgttctattgttatcaatgacgagaaacaagcatcgttggatggcgatatacgagaactatatctatcatgtatccgtgcgaaatctgaagcagatgccttagtacaaatgtatagttttaacttttcaaatggattctatttggtacgagttattgaagtgaaaaaagtacttattggttgtagcatagaaggattaatgtaaaatgagcactatagatgaattgattcgccggaattcaaaatactacgaaccatcagaaattgtaggtaacattactaaatctgagttcggcccaaatacgcgaaaactcattcatgtcatgtcacgtgatggctttaaatgtgattgttgtggtgaatccaatgtggtattcactttgcataagcacagtactaaagaccgttataaattcagaccttatgtacttaaagggaaacgccttgtacgcatgacgttagaccataatgtactggactgtcttcagggcggtaatgataaagagaacatgactgcattgtgtgaggattgtaacgcattacgccagcataagtttgctgagtacagtgagtttaagcactggtatcataacgctgatcgttctatcgagcctgacgttaactactgcttcattgagtatattaagaatgctaaagatacgtacattcttaaatcaatgtcacctattacagtgttccctgagaatctattcagtactatgcagcgtgattttaaacgtggtatcattaatgcattcaaacacttagacaaaggtatcaagcactttgaggtgcaatgcttaaatgaaggaatgaatcgtttagtacaaatttacatgaaatcatttataggtgttgacaccgcacgtctaccagtatataattactttattgaaagcgaagaaggtgaatcacttcgtgattatggtgaaagatttaataataaagttttcactgcaatcgcaaacgagattcattactataaaccatatgtaatttaattagaggatatatcatgcaaatcatcattcttatcgtaggtatcattcttggtgtatatggtcactcctggatgaacgagaagttccctaatacccgtcctgagtattgcagtactaatgtattccagaatagtgataaaccaaaggatagtattggtaagaaactggacaaagtacttgatgtgatcaatgaataagggccataaaatgtttaaaccgaattccaatgttggtaagaaagttcaattacttgaagatgtatccaccatgagtggaattttcccaagaggtcacatcatgactattattgccgaaacatcccgtgggtgggatttggaagatgctgatggtaatcgcatccttgaggctggattctatggtcatcgtgaatataaaatcgttgattaaggatacaacatgactttagagattgtctttggtattgttagcttagtcgtctgtgtgggttggtttgggtatctgatgatttttagtgattaatacttgacataagatatcagtactgtataatgtttatatcaaagggaatgcagtgaaaggaaagacagcataatcgtactcgtcactgcgtttccacccttttaaattaaatgaggaattaactatgtttcaaactaacactcaaggttttgtaatcatcgatttgaatcaaaacagcggttattggatgaaacctactgacgcatgtatgcagtttgattttttaggttacaggattagtgtaagtactggtgcttctgtactgcgtggtggttgtatcgttgaacatctggtaacagatcctgaaggccgtgagtatgaaatcgcaggtacagtacaagatgccattatgtttgtatgtcagcgtgaaatgaatagttcgaattcaaatgcactgactcatgctctttctcaagaagtggaggttcgttaatgaaatatatcgtagcagtactcgttgcatttttactcactgcatgtgacgcttcaaccaaagaacgcacaagtgattttattctccctattgaattacaagaagcaggatgtaagatttatcgtatgcaaaaggacggtgctggcgtaaccttgtatacagtttattgccctaatgcaaataccagtactggatacagcagcggtaaaacacgaatcaatacttcagtgattaatggagtacaacaatgagccttgatacaacccgtgcaacagatttattaactcgtctgaatgatgaagaatatcttgctgaaataaagtacaagcttgaaaaactggcacgaagtgctgaagtaattgaagttaaaggtcatttaccaatggatcaattccatacgttcaaagaagatatggtttattatccaaatgaaatttggattgagtgggaacattggactaacaaaactctacgcattgaaggttattgtgtaaattatgaatctgaacctggtgatgttacaaaaactctctccatttggtgtacaaaaacacatgaactagttgaggtgaatttgaattatatgaaaggtttccgttggggtgatgttaaatgattaaaaaactattgacagttgtctttgtattctgtacaatgtttcttacagcatgtaatgatgacggcaggtttagtgcaaaagacttagttcgtcaggattgtacattcacaggtaagtcttttattaacgagtatgaggtatgtacacaatcaggccgtatctgtggtggttgggaaaaacgtcaggagagattgtatgtatatgaatgccctggacatctcaacaaagtactaagcacacacaaacttgtttatgataaggttgaataatgactaccaatgaaatgatgcaggttatttgggataaaggttgtgatattaaaatcactagtactgatgttcgtgtattcaaaactgaatatatggagtacttggaccttgatacttataataatattgtatcaattgaacacaaaggtgcagtactggaaacaattgacgtagtactgaagaaagctaccgacctattaggagcggcagtgtgaataacaaagagtgggaagagtaccgtaagaagtgcagggagtttgataaagaactaactggtacagtgaatgcagtaatgtgggtatctgtaattattgcagttcttgcgttgggtgctattattattaataaattattctaaggcgaatatcatgtttactttatatctcgcagtagttctcaccaatggtgaaatcactcgtactattggtgaaggtgtatataaaacaccagaccagtgctactctgctactcattggcctcgcgatagtggggatgaaactcgcactgaattcaaatgcatgaaagtaaaaggtgattttgttaaccttaaggttatcgaggcgaaataatggatattaatgagttgttgatcttattgtttaatcgtggaaatgatgtaagacgttctgaatatcaagacaaaattcacattgaaactgataaatgttatggtaaaacaactcatgagattttgaagccaaatggcaaacgatatgccgatgtatgttttcatgaacgaagtgatggtagtgaaataatcagtactgaaattgtgttttatgatagatcctcggcaggttcatatgaaatagaacttatatattgtcctggttcagaaacacacaataaacgtcagatattatatgatgtttattcacctccaggtgtagctgaatattttgttattgattacagtactgaagaaagtgtgtttcaacagagcttgattcataacttccagttgaatgatccaaatgtaccaaagttgttcaaactactcgatactatcgatgttgattttgaatttgttatgtgcattagaacttccttgatcgatgaatttatggaactacttaatatatgaatcgttactacattgtgtatgagcatgtatctgagtttaataatgtacgtggttataagaaagaagtacagaagattgatgtatttgttcgtgcattaagtccaacacatgcactatttaaattgtttgatgaagtaactaagtacaacacatcaaaaacacaacgctatattgaaaccgtacatagtgtaatactcactgaatatgatggagaatgacaaatgaatttatatctagttgaagggtcagtacaagtatccaagcaagacggagaagggtatcgtttttcacatgtagaatattttaagtacgtagtaaaggccgatggtcagtacgacgcagaaattcaggttgaagaatcattccgtaagaagaatgatattggtaaatttgaataccaagacttacaattcactgtatggcctagcattaatgactaagatgtggtggtgtcatggtgtctacaatgtctatgaatatggtagattcaaaatgagcagtacctatgccagacgtgttaatgcgaaaacccaagttgatgctgcttgtgagattgtagagtggtatgcagagttcaatgcaattgaatatatgtacgaaatagttacaataaatgttgtacaaattgacacgtctttgatacaataactacatcaaaaaacaactgcaactgaggatactatgttagttaatcttaatttcatcacttctcctgattgggcattgcgtggtgtttctttgaactttaaagaaactaatttcgactacattggtgattgtactttagttaatgctaaatttgatcctcatcatcctttttgggctgaaattcatgatcctaatgtaaacaatagtacatctgttatgactattgtacagacgaaacatacaggtatcctcgcaggttacagaccaggacatgcgattgaacacttcactgattatgatgtagtagatgagtatccaatgatccatgaagatggaagtttatacacgtcagaagacttccgtgctatctttaatagtactgatgacattctaccaagtggtcgtccacgccctgagtcttatcttatgcacccgtcttatggtgtagcagatacgcttgaacagattttgacgaagtacaagaaccttattgaaggtccagaagaaattgtcatgtgctatcatccagtactgaagtctgaccaaccagagagtggtgggtggcgttggcataagaatggtgagtacatcggtactaagcgtagtaaagaacgctttgagtacttgtatgatgagccagatgttgaggttgttttatgttataattttcttaaggtgaaaaagaaatgtatctagtattgagttttgtagttgcattattgtttatgttctaccctacactagtagcagtatccactaaacgaaagctggtcgatatcactactgtattcttgctaaacgtggcactcgttctagtgtctattatcattcccgtgagctttacggttattgatatggcatctgtattactattcgtcttcagttatcttatgcttattgaagaaaaacctgttgacaaacgtttgtagtactgtataatggttcttgtagtctgaaacttaatacttaaacttgaggatataaaacatgttatcagttctaatttcaattcctgctctaattcttttatgggttatggttttcgaattcttcattaaagacgaaaccgagcgtgatgagtttcgtgagttgtttatgttagactcacccattgcacgaatcttcttcgttgtagtacttatttcagtactgtcatggttagtaagttttgtattaccaattggaccatttgtttcaattatgaatttagttgagaatattgttggtattgtgtggtttactaatgcattgtttgctggtaaagtttccatttggtttgatatcttgaaagataaactaactagtaagaagtaacgttcaaaaagtctctaatagtattaaatacttctgtacaaataatatgtgcagaagtattgctcgttagaggttataatgaaaaaatatataagaatgataatggtgtttgtagtattcatgctttattcaaggttcagtacagccgtacctgcttaccttgattgtatgggaaacgatggaacatggcatgtactttctaatgacccacaaaatggaaacaatattgtcgtagataatgaggtttaccgtttcagggccgagagtttgattgggaataaaaaggtatcgacatataaatcagatgctggtataatagcaaatgtaatattgatagtggaccccaagacaaaggatggggatatttacatagtcatttatcaagattcgtcgaacaaacgagatcaggatattgataaattgaatattcattgtgaactttaaggtataacaaatgataaacgatgtaatgctttatgtattaattactccgattcttattaactcagtaataactatttcaatggcattatttggtatagtagcaattgtttatgctgttagtactgggaagtatactttaattagtaaggttgattcatggttcgcatctgatagtgtaccatattcagctagtatgattacgattggtattctaatcgttgcatttacactgcactgtattggttttattaatacaagtattttttcagtttttattggtctatcattatatccatcactttggtacttgatttatgtaatcgatgtttggtataataactacaagcagtacaaagtgaagaaatccattttatccaaaggaaacaaatcatgaaagtaattttgttttttgtttgtatgattatatgtggtatgtcttatgcagaaccattacctggactagtctgtactgaaggtgatattaaacacatttaccaaggtgatagcgatgaccatactatcgtttatatcgatggtgaagtgtataattttgttaaagaacatactgtaggtgacattagttttacgcagtactcgaatgatactgatcccaatatgcttgcatttcttcaagttgatgtatatgaagaaagtaatggcgaacagtatgttaaacttgtgatgatgcgtaaggcagcaaaacaggcaacattcggtggcttgtgtaaatatgaggactaaacatatgttgatttatcaatgtgataacattcgtgttatgcagcatccctacttacaaatctttgatgtgcagcagtatatggaaggtgatgacccaagaacctatggtcgtaagtatgattacttcactgtaaagcagtgtcataacatcaaagaagtacgacaattagttcaagaacttcaaaataaaggttgacttcggtcaaccttttttgttataattacttcatcaactactggggattttaattatgtacgtatatcgttttgaacttaaatttagttatattgaacctaacttagatccagcaaaaacagtagggccgttcgcacggtactcttcacgatacagtactaaaaatggatttgaccgtttgtgcatgaccacgccttcgcaattccaaccagaaccaactgaagatggaatctattcaatcaaacgtggctgggcatttggctgtcagagtttgaatgatttgtttaagttctttactatggacttgggtaaattacatcgtgcagggtttgctgtatcagtatatgaagttcctgataatcatgttgatattggtcgtagtcaggttcagtactattgtggtagtgccaatctggttcgtatcatgtcagtgagagaagcaaaggatgagtaccgccaaagtaaaagagtaatacatgaggctgtatatggataagactcaaatagaattaaaaaaatttgtatcgttttatctacccaatgacccacgttgtacacaatatgatgttgcatattacaatgcattgactacacagttaatgaagaaattcaaacagtgtttgactgtattcaatgatgatggtagacgtagatatagaatcattccaatgaatgatgaaaacgataactactttgactttgatgttgtttttcgagcatacaatgatgatagaataaaattaaattatgagcaaataatgcttgacctgagtgttatctgtgttataattactcacatagccgaacttgatggtttaaaagaatggtattgacttagaggataatcatgaagcttaaattacaatacaatggttttactttgcgtaacgacactgagtttgtcaatcgttcaccaggtgctgagttgtttttgcgagagatgaacattcctttcgtcatcactactgttggtgaccgtgaagttctacaatacgcagagcatccagttattgatgttccgtggtattctgagcatatgtatatggatatggcccgtcgctacgactgtaagttcgtagttgaaggtgataaaattacagtccaattcaaataaggtaacttaaaatgatcagtacttcagtaaatgcaagcatcgtggcagacgtagaaatggttaaagaatgtacagacgaaacccttcgcaacatgtacatcaacgatttgatttcagagtacggaagtcgtggtgttcatgagtttgttcgcctgagcggtgaattgatgaagaatcgcattcgtatgaagcttaatgtgcatccaaaagcatgggcgtttacaatgaagtacgctgaagatattgttgagttgcgtcgcaatgatgtgtataatcgtttacacaatggtacattaaaggactaaattataggaatccgtaaaaggattcctgttcttgtttataaggtaataaaatgaaatatctagtactagtactgctattcttgagtactgttgtatgtgctaaaccaaagtatcatgtagtgtgtgtcaacgagatggaaggatatactgaaacatataagaaccatattgttgaagttgtagcccaaggttcctatgggaccatcatcttccttaagaacaatgtgattgttcagtacaaaaaagatttaaattgtaaagtgactaaaaattgatctagatcaatttttcagatattctctgtaaataatagcagtacagaacattattttacggaggtgtaatatggaaactataatcatcatatgtgcgttgtttgcatttttggtaatgtggtcaccaacattcattgcatataaaatgaaaaataaaaatgcagaagcaattatgtacgtaaacttatgttgcatactaacttatggaatacttttattctttactgaatttacattaatcccagtagtagtttgttcatcaatgtttatttggttgtttctgctttatgaatctattaaaaaataaaagatatagggttggtattataccaacccttttactttatgtggggtttaattaatgaatagaaaaggtaagtttaaagtatcaaagaagaacaaaagttctagatttagacctataactttaacttttaaggctaaagaagaattaactgcacttagtaaaaagataatttcatctaagattaaagtatcatacgtaaattcggtaaaatttcaaaaggttaaaccacaagacaaactcatttcacttatatttaaacgtaatgattttgagatattagtagatgattatagagcctataatgctggattcattcatgtcacttgtaataccgaactaactgagttggaacatttgacatgtatcataagtgtagataacggtgtggtgaagtacaattttttacattttaccaatatcaataatggaatgggtgtacaatccagacctatagtacctacatgtgatgtgactaatttagcaaagctcaatgaagaagaatattttcaagaaagcattgtaactgagcatttagatgctataatagcaacttacaaagaatgcatatgggttttgaatgcgataaagcccaaattgtgtttaatgaatgtcgttcgtgatttataactggagatgtagtatgggttggattaacgttaatagcattgaactaaatgagcaagacatatttgcctttgttaatttggtagagaacattgctattaaaggtcgtctgtttagttcatcggacggtaaaaaatggatctctcttatgaacgacaatggcgagatttatgacgttatgcaactacctcattgggcttataagaatacatctgatgatgtgatggctgttgttatgcgtgagtttgaaaagctacctggtggtgatattaagaacatcgtattcaatgcagaacgcatgaactacacaccagagcagcttcaacagttggcaattgctgcctatcaagctgccattaagaaacctgaatgtgtgatattaaagtacacaccggacttgaagttaacactagaagatatggattcctttatcttcagttcagaacttaaacagtactgatttgactttgggacaaccatttcatacgtgatataatcttaagtgtctgtagaaataaacagcacatgaagtataaatattaaagtaatttatctctttaaggagtatgaaatggttgataaatggaataagaaagaaccatcgcatgatgtggatatttctggaattgataatgcacataagcataagtatcttgattcactaggtctactaaagcctgaccaaagtcttgcacatgaacgcccatcccgcgaaaaagctttcagtctagaatctctaggtcatgaattgaatcgtgtgtttgaagatcatccaatttatgtacgtgatatcattgagaaacttaaagtaactcctaaagagcacttcaaggcaacagttcacgcagcattccgtgcactacatgaagcactaaagaaaactcataaccttcgtaagattcatgatgatatcgtgaactccattgaaggcgaaaaacataagtttaaaaaataattaaaaggggttgacttcggtcaaccctttgtcgtataatattttcattgctagaacataaaccaactgaggtaatcatatcaaacatggcagaaacattatcactatatgcattagtactgttatttgtcggatttcttcccgcacttattacagccattcgtgttgttaaacatgaagctgcgttgattgtatgtagtattggggcgaacattctattgtatttctttttaacaaatactattgtactgtttgctttctttatcatgattctgtgggtacttgtaattctcggattcttcatttggtcattggttgatcctaagaaaaaatcaggagaagtgaaatgaaattaattatagcaatgatgatgtgtttggctttccctgtactggctactcctaatctggactgcacactgttgcgttcagatggtgacacacttgaaatcaacacatatgaactctttggtccaagtgtagtggagaatgaaaccacctacaatttcaaagctattgactttgaaggtaatgttccagtactaactacaatgactattgacaaaactactttactcggacgaatcaccacacaacgcggagaaagtaaaacattcacagcgaaagctgactgtaactactaatggtatagtacagtgaaaatagggttgacttcggtcaacctttatatataatggaagaaataaacatggaacgttggaaattaattcctgattatgactattcaatttctgatcatggaagagtaagaaacgaacaaactggatatataagttgtggatttaaaaataaagatgggtatgtgaaggtcgatttgtatcgcaattctgtgaaaaatagaaaggcgattcatcgactagtagctgaagcatttttaccaccacccacacagaaacagttacagtgggcgagtagaacaaagaaaaaagttgttcacataaaccataaagattgtaatagaagtaacaatcactatttgaatttggaatggaacacagcatctgaaaacgtactatattcttattcacatggtaacgcaacaactaatcgtggtgctgctgtgttgacatgggaaattgtaagagaaataagaaaagattataatgataattataatgatgtgatagaatatgcacgagaaaattgtaataaataccgtgtatgtaaaaaaacaatacgtcgtattttatacaatcaaacatggatagaggattaaaaatatgccacaagataatggggatatgaaacatcgtaatggtggactagtacaacactcggataaatgggccgaccgaaatcgtcgcaaaaatagttatcaagcaggacaaggttcacgtggttccagaaaccaaggaacgggacttgctggtaaggcaaacgcagaagcaggtgaagttaagagtactgagaacatgactgagctagaaaagctacgttatgttaactcattaggtcttcttaacgcaacacagaaacaacgccttgctgatatggaacgtgctgagaaagaacaagcagttcctaaagaagaaccaaaaccagaagctcctaaagcagctaaccctgttaaagagttatctgataacttaggtaagatctttgctaacaaaccacagttgattcagggtattgtcaaagcactacaggcagttcctaaagaagaactcaaaggtaaagcacaagctgtacttgatgcaatgaaaggtaataagagcactgaacccactgaagtattcagtactatcatgcgtgcacttgaaggtacacacaaagaagattcgaaagaacctgaacagggtgaagcaagtcttgatggtattcttaaaacttttgatagtattggtaaagcagaccgtgctaatgcccagcaagtcaagaatgtactggctaaacttgaaaaccgtttagaaggtaaagacgataaggttaagctcgtatcagatgcagttaacagtgcacttaaatcttatgtacaaggtgagaaaagtcgcttcgtaacagctatggatgctgcattcaacactatgaaaaagtatgaagcagaacatggtgagaaagtgacacctgatggatatctactacgtaaagatgaagaaggtgcagttgaacagaaagatgttgagcctgagcaagaagatctacctgataccgttgatatggatggatacattcatcgtcctgagatggaagagtttgggtcagataaagaaggttttgaacatgcaatggcagtatataaagcattagttaaaaatccagattatgctaaccgttttgttgacgagtttgatagtgcatggaataactacaaagacaaaggttatgacttccctgctgaagctgcattctttgagcttggtaaactattagctaaacatggaattgacgttggttcatctgattctgacggtatggaagaagaacatcttgaaatgactgataaatcaattcatgaacttcaaaagttaatgatgtggatatccgagattggtaaaaaagcaggtgctgatagtaaaagtatcaacaaaaatattagttggattaaagataatgttggtaacaatgctcgtaaacttcaaaaggcttgggctgatttagtaaatggtgcagatgatgttgacattgaatcaccagaagcatatgagtatgcactaactacacttgataactcattcaaagagtacgaccgtggtgttggaaaggataaaaaataaacatttaaaaggttgacttcggtcaacctttttttgtaaatattgtagagggttttataagaggtaatgcaatgtttaaatatgttttattttcacttgtattgtttactgatatagtttatagtactgaataccagtgctccattgctagacaggagtttagaaacggtgagttctataccaagtcttacactagaggattggtacatgacaataataaaactatagggttcaatgcatttgataacaaagatcgatatacgtctggaatactatctgaacgtaagtcggatgtaataggtatcgagcatgaattccggtgacacagttgcaatgtgtaatgatattgagagtactaagaaagtatatactaatggtgaaaatacctttagattccacaatcttactaccaatgaaagtttatacttctacaactgtaaagagttcattaaaccagtggataactcatgtgatatgccggagccaaaaactgctgtatgtgaggaaccacaatctaaagctgacaaagattcaaaagataagaaagacaaagctgacaaatcggataaaaaagaaaacaaaaagaaagctaaagatgacaaagataaaaagtcccctaagccaaatccatttgaaaagccaaagaatgaaaagccaaaaccagacatgacatgtaaaatataaaagaaaagggttgacttcggtcggccctttttgctataatcatttcatcaactactggaggcaaacatgttaaaagttattttccgttctatcagttcattacttcttatgattagttcagtttgttatgcaaatgaagtatctgatttatttgagaagaattacccaactgcacaagagatgggtgttttaaaatgcaagctaacttcgtacaacatcatcggcagtaaacttgtactggaacatgaacgtagtacaccaatccctctagtggaagaaggtagtacctttagcttcaagtacggggtgaacgtaacatctggtgtaatgaacaatgttgatgcagagactggttcaacatggagtactagcggtaaagatgtgtttcatcgtatgcgtgaaaatggtaaactgaagtacgtatatgaaagttcattcatgaaacgtgtattcattataaaaaattgtgtaccagcataaggaacaacatcatgcgttcatttgaaaacttaatcggtaaacttcgcttacaagaaattgaagctgatgtgaaggaattatctgaaactaagagttacattcatttagatgaactgtttattaagtacggtgagattggttttaagtcattatttgtttttgtcatgaaggtgcatagtttaaagtactttgatttgagttcaaaagacggacaaccaaatctgtattacatgtattatcttaagaacatagacgaagctaatacattattcttagaatatatgtaggggtaaaaaaatggaacaacattttgaattaattaataaggtatcattttatgtagtatcgttaatttgggtgtattacatgttttgttacattattgcattcttccgatacacaaccttgtatcaacgcatatactacgatgcacccatttcatttatgatgttctgtgcatgtacgtgggggataattgaagcactggtatgtggatatagtactgacgaattaaatctcgtatcggggttgacattactaacaattgccattatgatagctactgttccgatatttttgctctttaaactgcttcatcgtattgatgagtggcgtaataaaatccctgaaaagtaaggattgataatgattttttacactaatgcgtttgcttatgccgttgcatcaatctacattgtgtacttcttatttggtggagtactgtacttctttaagtacaaaacattgtttaaccgaatgtatccaaatcatccaattgtagtagtacttttgattatttctgttgctcacatcattgtagaaacaataatgggtatcagtgcagtacattacagtctaggtactattgtaattattactatgatcatcgcatacattttagtaatgggttttggtttatttggaaatgtagcaatgaaattgtataatcgaatcaaaacattacgtgataatctaccggagcgttgagatgtataatttaaatttaatatcatatatcataacaacactgtttactttctatgcctttgttggtatactcctactgtactttaaattttataaactttttgatcgtttttatccgtcattagaacatgaattcttattagtgatgtacatatgtctaacgtttatgtctacatttttcactgcatctgatggtatcattggtattaaagctgaagaaccaaatgggagtacgcttgttgcctttattctggtaggatttttcagtacttatattacctttatttcaggatgtaccgtgattgattatcttgtgaaattacgagataaaatgccaaatcgataggggattgctatgtttgcattgcgtaataaaaatacaaaagagattctgtgtatctcaacagtatgggattgtgcaggaagtgaaatgattctcgaagaaggttcaggtgattgtgtttatgttgttcaggatcgtcgcgaactggaatcattacttgacgaatttaatgctggtggaaatcctaaaagtactcaattccctgagatcaggaatccagcaccaccaggctcttatgaaattctacaactgtgggagatggataagtaatgcgtcaaatgcacaaagtaaccgagctaaacaagcgtctgattaaacaatcaatgtttgaggataaagagtcttactataacttcaaacgtgctgtagaggctgctatccacattcagaagcttaaagatagtggatgtattattctcgaaaatggttctgacgttgttactgatgacttcagtgtagcgtttgatgtttcacctgatggttcgctatcaatgccacgcttagggactggtgacatgactgtgtgggtaggtttcactatggggaatgtaccaggcacggtgtatgtagatgatgctgagttgaaagaaagcttcaaaaacttatcgcatcttgaaccacaatacgcacatgaaatgaatttattttgagttgacaaactcattcagtactgtataatgtttatcaaggggatgaacttctcatccccacaatcgaggaattgtcatgattacttttaagaaaactgctaaccacatccatgagatctacattaacggtcacaaagctggccttcttcgtttactcgcccatcatccgaatattaccgtatctgataatgaacctaaagattggcgtgtatttttctttggaaagtacagtgattttcaagtatcaaacctgaagaacattcgttcagtatgcacgttaactgaaattaaagaaaagttaaccaaagcattgaaaactaatccagatagtgttcttccacctgataactttgtagagaaaggatatgccttagctcatttcaaagatggtactattcaatctatcagtactgaatatgaatatcaatggactgagtacttcacttatggatcgcgtaatacattgatggcttcacgtagtaaattggaagaagtactgactcgtatgaatgaacgcgagcgtcaattttatgacaaaccagaaatgtggagttcaacttacattgaccgtattgactatgttattatgactccaatctttgacactgcttactatgattctttgaaataaggataacttatatgaaactacgtactcgtaatcgactgatggatgcactgtaccatgaacagctttcactgaatgaagtgaagcgtcagttaatgtacacatatctgttacccgctcgcattgcaaaacaacgtgctaaagaattgctgaaagaagcaatcgagtactgggaatctgattcgtactaattgtcacgttaattaagtgaggcgataaatgaaaattttattagttctaatactactaattgttttaccatttacgttagtatcccctgaacttgcatggaatgtgtttgtgtacttactatgtactattgtagtaaatggtatcttgttcttagttctattcataaatggaataggacacggtggaggatggaccagtactgctgatattgtacataaaaccactatgtttttatttgtagtgtggaatttattctggacttggtttgtgttcttctaattaaaaaatatgaatatagatttgaaaggtgcaacacgaactgtcatactcattggggaatatgctatcaagattccaacctttaaaagttggaagctattcttaacagggatgatagctaacctcaatgaacgttcttggatgcagtactccaagcgtagtgatagtggtatctgccctacgttgtggtcatcatgtactggactagtaactgtacagaaacgttgtaagccagtacaacataggggtttattttgggtggagttgtgtgctttagcattgaattcagagttaagtacggacttcttctattcagatgcgaagccagaaaactttgggtatctgggtggtaagttagttaaacttgattatggagattaatatgtacgcattgtataataaagaaaagaaacaatttgctaaggttgaaaaatcgattgatacatttggtatgtttaagtactgtattgtgacggacccatatgctaacttgtattacgttcttgatgacgtatcagtactgcaacgattgcttgaacgttccaaaagccaatgccaaggtatccatgatggttcatcttatgaaacacctgagtgttcctacggatatctggacgggtatgagattgtgaagttgggtgcattgttgaataaatggattaaacaatgaagaagtgtatcacgtgtaatattgaaaaagacattagtgaatttggtataagaagtgatactgggaaacatagaaatacatgcaaaggttgtagtaatattaccagtaagaaaaccagaaataaaaaaactgatgtcaaaaaacttgaagaaatcaaaagaaaaaataaagatttcatgaccagagttaaatcattttatggttgtagtgtttgtatgatgaagtatccagaatgtttagactttcaccatcttgaccctaataccaaagaacatacaccaagttctatgttcagtaaaagtcgagatgctatcaaagatgaaattcgaaagtgtatgattctttgttctaatcaccatcgcatggtacattctggtagactatgtttgattgaaagaaaaaagggttgacttcggtcagccctttttgctataattatttcatcaagacgagaggatgaatacatgacccgcattaacttagtacacgttaaagaacttgctgaccaacatctgatggctgaatatcgtgaacttccacgtgtatttggtgctgtacaaaaacatgttcaagcacgtaagcgtgtacgtgatttcaaaattaacagtacttacctgttagggtcaggtcatgtaacattcttctataacaagttattgtacttgcagaagcgtcatattgatattattaacgaatgtttgcgtcgtggtatgaagattcaaaacattgaagtaaatgacattagcagcttccctgtggagttttgccaggactatgtaccatctgattcagagattaaaatgtctcgtggtcgcttgattgagaagctccagatgaagcctaagtggtataagtttactgatgttgaaagaccacagtacgttaaagatatgttgaatgaaattgaagaacaaatcaaagatgaaatgaataactattaaaggaaaaggttgacttcgatcagccatttttgttattatagctctatcgaaacgaatgaggattaagtaatgattaaattaaaatttgaaggtatgcgtgatgggaatggtaacctaatcattacacgaggtactaaagcagtacgatgcttagaagaacatggtattgaatttactattccatacgatgatatcaggattaaagaggatacggtactggaattgccactcactacatttgaagaattgaatgcattttgtcaaacatataatgcactaatcactttcgatgaacgtaattgtgttattgaatttttacaacactgatgattaagtaataaagaaagggttgacactagtcagccctttttgctataatcatttcatcaactcaagaggataaagacatgacccgtacagagaaagcaatccaaatcgaaaagaacttcaacagcatgatggcagctttggaatgtttacttgatacccaagctaattcaatcaattccctgattgtatctggtaaaggtggtgttggtaaaacttacaacgttgaccagcgtttacagaaagctgatgataactttgaaatcgactacaaaaagatcagcggtaaagtaaccactatgggtttgtacgaagcactgtatcgttcacgtcatgccgcttcagtactgttacttgatgacgttgatatcttcagcgatgaaaccaacattgacttgctcaaagcagtactggatacttcagagaagcgggttgtatcttatgcaacttctgggaagtacctggaacagcagggtattcctaagcagtttgaattccgtgggaaggtaatcttcctgacgaacaaaaacctgtcacagattgctaaaggtaaaagcagtttagcaccacacattgatgcactgattactcgtagtatctttgttgatcttcaactgttcactaacgaagaagttatgattcacattgagaacatcatgaacaagactaatattcttgagaacaatggggttaataaaaatggttgtaaagctatcttaaactggatgctgaacaacgaagaaaagctgcgttcaccttctctgcgtatgccagtactgatcgctggtctgtataataaatttccatatgattgggagtcaatggcagaaaacatgttccttgagaaaatttaattacatgtattgacagtactaccatagtgtagtactataacttaatattcaccaccggagatacgatatgcttgagttttttaattcagatacaatgcttagtattactttgatttatctgtttggttttttcttttatcatgcactagttgtagtactatggtattttaaaaagaaaatcgttttcaaactgtatccagatggaaacatttcactacttcttttcactattatccaaggtatgtttgcaattgttggtcttatcgcacacttaatttattgtgctaatccacagttgaactcaatatcacaatatattgtattccttggattactaatttgggtgctgatatacttgtttaatctcatcattaatggtagagcaaaaattcgtggggacttctaatgtttgaatcttatcttgagtttttaaagagtagttctgttgcacattcaattgcagggattattctaacaggttatgtaacattttgggtagttatgtttctaggatggtattttaaacgacaattctataagcgtaatggtgaagattttatgaattctttcattaatcaatcgttgtttgcatcaacattgatagcatgtattgtagcaacactcacaggaaaggtactgccagcattctccgcagtttacggtacattgggtatgatatgtgtagttttttacttgaccattaaactttgttgtgttatgattgaacttcgaaacaagataccggaggaatgatggaatattttttacacttactgccgtggtatatgaaagttagtactatgtactatattcttagtgctggattctactttctgatgtggttcttcaaacgaccaatttcagtattcatctctgataacatgctgcaattaaaattacacgcatggtttggtatggcgatgttcatgcctattattcactactcaatgattagtagagggttccgttccaatggtgaaccaattatattagtattctattggggttcaactattgcatatgtaagtttgttgttgttcccagtcgttcataaattacgtgctaaagttcctgatgcgaggtactaatggatatcgtaacattgatttacacgctttattcattctattttattatgtgttgggttggatttttatttgtattgaagatgtttgattatgttgaaaaacagataaaagaattttgtattccttttattgggtcaaccataattagtattacttgtttgtattatttggtatgtgatccacctgttaatacagcaatgattgacttcatgttattaccaacaactgttatttttttatgtagtctgatgccaccgattagtcgtgggctaaccgaatttagacgcaatcaaaagcgtaaacgagggatgtgatggaaattgcacttattatattaactgctatgttgtttattagtacaattattattattggcaagttcttctgtgtatccttcttggaaaccgtatacgatgaaattgctgatgactatgaattacatgcaggattttttactacgtattggattagtttattactacttatcactgtatggattttacatatattcaaatggggtatggtagatggtgatccagtactggcaattatctgtgtagtgtttggtctactcggtggtgctgtccataatctgatggtaatttggcaaaccgtacaatatgtattagttccacattttcagagaatgcaccaaagcatcaaataccgcaaacgaggataacatgaataatattgaattatcttttattggtcaattaatggtaatggcgtatacatttatcttctcactgaatgcattcgtgtgcattgggaaatacttccataagcgaccattatcaaatatatatgatgtaatacaaaggagatatagtccagcatatactgtgttgagtacagcattcaattttggttttattctaatgctggttgcattattaacatcgagttcgaattttataacatttggtattatggtatgttcactaacactgtgtgtggttattctctgtgcatccgttgagttgctggcatggatgtataataagatttccgaaatccatgagcgtttaggtaagaggaactaaaatgagttatcgtgaaatattttcgatcattactattattgtgtttagtttaggttctttgatttggttattgtattacttctgtgtaaccccagtatataatatgctaaattcagttcgctctgatgttggtattgtttcttttattatcttcatggcatattatatgctaagtatcacaggtttggtttcggttgttatctttgaccttggaccgaatcaagatggcatggagatgtgggaattgataacgatgtttgcatgtatgatacctgcatttggtgtatgtgctggtgtattttatgggttgtgtaaaggtgtgaactacttaaaaaatatccacaatactattcgtaaaaaacgtatgttataataaaaaatccagtactattatagtactggattaattttaattatctaagccaccaattacactgaactgtgtggtaacgtctgaactattatctatgtaagttcttctaccaacataatatagttgtccttttgcttgaataaaagttacggtccctgtattaaatgttataatatcattgtatgtaatgtcaggtggtagtgctgctgataacgcagtaactttacttaatgcagcactagcactaccatttataccagcagcaatactaccagtaccataaaacttattgtctttaccaatgaatacgttggctgatgcttgtgtagctgatgttattgccttaccaatttggaatgcggttcctatggtgtatggtgtgctcctggctgcgtctatgctttgtgtatatccatcccctagctgaccattaactttgtcaccacatgagtatagagtaccattgaggctgtatatgatcccacggttagagaagctaacagcttctgcattagtccatactggtgcggtgtatgagtttacacgactagcggtagttcctagacctagtataccatatgtgttatcaccacatccaaaccattgtccagaactgttttgatagaacactgaacgttctgttgattccatgcgtacagcatccactgcaccacttttaacgaacgaagtggatgaggatgtagtaccagtacccaatgcaccataagtgtttgtaccaattgcatagattgatcctagtggtgtcataacacgtatgcagttatatcccaattgaactttacattcaaataggttgtcccatatttcaggaacagcagaaaataaatttgtaatttcgaaccatgatgttgtcgaaccagttaatcctatactgcttttgtttcctaaaatgaaaacagttttgtccttcttaattatccatgatgataggaaacctatctcacagctatgaacatttgattctatttgggtaatataatacttaactgtgttatcacccatgaatgccgcagcaccggatgcaaaaaggtttccagtttgtttatttaaaatgtagttattgtttccatatacactaacatcataagaactttcttgtttttcgttcccataatcaaataacaatacgaaaggtaacatgataaaatctccttgtgttatactttatttaatcattttataatgtacaattttacagcgatttaattgaggtactcatgaataaaagaagtaagttctatcaagaatatttcactaaacagaaacgattcacaaaaaaatactttacatcatcccaccgaccagttgcaagttacaaacgtcctaatactaactatcttactggtggcaagtttagtgcaaagttcgaattacttatgcgtgaaaagaatatcaagatgcattacgcagagacaagaaaaaactcatcaattatttgttctgattttcttgagtacggttgtttaacttataaattcaatgatgtatatttgtatattgatgttgattgtaacagtactcgtatgtttgagatttcaattcacccaaatcacaaccgttatataaacattaacttgattgataaagtactgattggtatatctagtactgatggtggtggatttgaatttattgcttcaaaatatgatgaagatttcgtactttacccagtacttgatgatgtgaaagatattctcaattcattaaaaggaagtcaatacaaattatctgaagaacagctatttcagtactcacttattcatgatgatatcgagatatacttagagtgtgcgagaatcttggagtatctttataatgaactaacaacaactggtaaagtgactgcattcacattatataatatgtaaaagaaatgccagtactcaagtactggcattatattaaactggtggagaattatatggctcaaatttataagttgtcgtagtagtaccagcaaggacgttgttagctagtatgtacatagtcctatagtcgattaccaccacagtaatactaccaccaaaagcatagtctgttatgttattagtgttgaatggtaaattagatgaactggtccatgtattagctgttgtgctatttgtaccattggaatagccgacactagatgtaccagttgtccataagtttccatcattgaacaacgatacatggttagctgtatatgatttattaggtatagaactatcaattggtgtattccaaactttagtaacccatgttgtaacttctgtattattagtaacgccattacctaatgaaccatttattggtatacctcttatataagaagtgttagtactagggtcaacatatccaaatgtatatgtatcaaatgcaaattgaaaacaatcagaactcggtaatgtgtacagtaatgtatatgaagacgtgattattgatgaatttgtaccgttcacgccttggtataaaataccactagccaatatttgattattattttttgttatagtgttcattaacggatatgttttcacatcacgcacatcattagtcacatatgtttgtgttgataatgcaactttactaccattacctataacccctgtatttgcaccactcccataaagtgaaccatcattcaaaagaaccattgtaacattaccatttcccgtaattgttttaatattactaacatccaccgcagcaaatacagttgatgttaaatcagtgaaacgtgtataatgtgatgttgtgccattgattggtgtcgcaaaaccagaatacaaaattctattttctttggtaataaggaatgttccagtaacaccgtttgaatggatatcaataatattatcgcccattaatacccaatcacttacgaatgttgtaccagcacccaacccatatgtcgtattgtatccacgtccatacactctaccatcactgagtaatgttaccacacattgaccaccaccaccaagaataactttttggatagtactctttacttttatattcccgtacttaaccattcttgcaaaaggtaacataattccctcctttattctttatactatatttaatcaaaaagaaaagccagtacagagtactggcattatttcaataaacatcccaaggtaatggtgtctttctaaacccatacctagttgcgttctgtccatctccggcaatgtatctacctgtaccacactgatataattcagttctgctacatatcgccccaccaatagtaccactaccccactgagttatattggaatataatattggcaaatcactacatttagtatataatgcaacagtagcagaaccactcgcaccaccaaaaacacttcggttcgaacccgtgtaatatataccatctgtactcataaatgcggaagtataagttgtactacaatctactggacttcttatattattaagatcgccagggtatgacctagcaccatatatgtatacattaccagcagtagtaataccattacctaactgaccattattttgtccaccacatgtaactaacgttccagtaccagaaacaatggcagtagcattataagaagtacctataacaatagaagctcccgccaataggtatggggcaggaactgcggctgcattcccctgacctaattcaccattgacatttcttccccatccatataccgaagtggtatcaaattggcatagtactgaaccataactaaacgtatatcgtatatccttcactgccgatggtaaaccactaactttagttgcaacactaataacgccagaaacgcctgtaactccagcgttcgcgttcatattacgtccatatgcatataaagagccatcagcagttttaatatgcatcattccatctgtgcaatataatgtatctatactatccactgcaacctctcccatactggacgttatatcgttccacccaagcgttgtagtactagaaattaagttatttccggcgtaaagtattctattatcatttgtaacaactacagtatacttggcagtaacgccaattaatctaacattctcatgtatcttagaccacattcctattgaatttgtgttagcatccagaaatcccaactgtccagcggtattatatccaataccccacacatcacctgcactgtccaataacacttcatgggtaaaaccaatcttaaaatcttttatcgttggtgttacagaaacaggtgcaatattcccgtacttaaccattcttgcaaaaggtaacataattcctcctttattcattatactatatttaatacaaaaagaaataccagtactcaagtactggcactatattacaacaaatgtatttctgtgagtgttttatatgttacaccgttgtacacacctctccaatataccttaccttcagtactaaccactaataatccatatgttgtaaacccaagagtaccaccaacatacatagcatctttgattttaataccaatgtcttcatatgtaaatgaagcgactgctcccgcttgccatgaaggactagcatagtaatcactataaacaccatgtgctttatatattgatgttgttccgggttcaaaaattacagttccacagtagttcattcgtattttggtactggatgaagttggtacatttatacctacatcatctttaatccctgtagttaggacgttaaaaccacccgctacacggttgacgtcattcgcaccgattagaaacataccaacgttgtagcccatataatacacactacttgtaccattccagaaagaaaaaccaccaccattaccacagcgatatagttgtgaatttgagcagtacatcatgccttcgtttgttgcatcaaaatcatctgctgttccgattacagccactgtagtccacgttttaatcacttggtttgcactagctggtttttgtataccatatcgtatattatatcccttacctacgattgtaccatcttctaatttggcataaagtacaggtctattaccaacaaaatacacattattatgtgacatgtctattgtgcatcttaatgaaactgctttttgtgtcaattgattatctttagtaaaagaagtaaagtagtaatctgatgtggagttgtttattaaaccttgaatcgacaaacccatataccagatttcaccattagtgttcaatgctgccaaaatacaactatattttgaagtgtatgctggggttaatgatgttgctggacctttatcatatccaccacagaataccatttccacaacttcacctacgttagaaaaataagaagatacatttataaaaccatttgttataaaaggtgctgtagttatacttgaatgtgtgaatattccatttgcatatacaccacaaaaatgatatgaaccatcattatctttaactattattccatttgatagtagccaaaaattgtttacattttttcgtactaattcaattcctgtaccattggttggtgttataccaaaagaaacatattttgtttgaccaataccatataaatttccatttgatgctaatattgcaccaaaccaattaccaccaccattcctatcatgacccataccaatggagtcgaatttcttcacagtagaggcttcaactttatttccgtacttaaccattctcgcaaaaggtaacatatttcctcctttattcattatactatatttaatcaaaaagaaaaaccagcactcaagtactgtcctatattttagactgaattgttaggataatatctcccacgtccccatattattcgcacagcaccaccaccagcagccgtataagaagaattatcccatatactaccaccaccaccatacctcttaccagcaccaccactaccagcaccaccattatatgaaccatcgtagctaccaccaataccattagcaccttgaacaatgataccagtaccaccacccgtaccaatgaacgaggtattatagaatgcaccaccaccaccgcccccgccagcaccattttcaccagtgattgcatttccgttaccacctttaccagtgtagccaccagcaccaccaccaatgactgcaccaccacttaaaccagtgttgccaccattaccaccaccatcattaatagcgttagcagcttttccacctagtccaccaaccgtatatgcaccagtgaagctaacacatagtttttcagttccacgatataaaccgttttcaactgctgtagtagtgttgggatgtgtaagagaaacacttaagtactcaccaccagaaaccggaatattattacgccacgataagccgccaccagcaccacgttgaccacccggcgaaataacacatactgatatttcggtaacgccagcaggaactgtaaattgtgttgttcctattgtcgtgattaattgttcaccatgtggcttatattcaattttattcccgtacttaaccattcttgcaaaaggtaacataattccctcctttattcattatactatatttaaccaaaaagaaaagccagtatcaagtactggcatagttttatagtatcatttcggcattgtaattgcagaccatgagttcgtggtaataggacctggcacatacaaaaaagaaccacaagcataaatttgattattaccatcaaatactatactaaaatgtgcacatgtgtataatgctgatttttctgggttgtaattatctaaagttagtgtattggtgaatctactcaaaccaccaccagaaccatttctaccaaattcaccagtactatatatctttccttcggttgttgcgtacatattaacatatgaagttgcacttgattttgatattttatgtatcttactatcaatagtagtggagttaaatgcaaatggatatacttgtgtgttcgttgtaataccgttaccaagttgaccaacatactgccaacctgcgttgtatatattgctggctgaataagcaagagtaccagtactgttaccaccaaacagtttaacactggttaacctgcttactagtgtagtatactgtcctggacttacgccaagtgaaccatacccactactcccacaaccgagaagttcaccatttgatgttaatattaatgtgttattctgtgtacccaatatatcaaggactgtattagctccggtattaataactgaaacaaaagtactctgagccgtggaatacccaccaccgagtgaatagttaccattcatgcctctaccataaacaatcgaaccactagcatcaacgtagtgcattgtatctgcaccaattgatatcattttaatattagatatatgtggaaatgatgaagtaacattcaaaccacccaacggaatcgtagtatttcctgtgcctaattgtgtggtaactccatgcattatgaattcttcacccttgagataaagaatattgtacgccccagcccatgcatcagtaacaccttctgaaattaatatccaagtatttcttgatgctgtattttcaacgtttaactgaaaatacttattaaccccacaaccataaagtgtgccatcgccatagaggacgtacacattatcaagaccgcacagtactttctttatctttggagcttcaggtgcaaaattcccgtaatcaaacattctaacaaaaggtaacataattccctcctttattcattatactatatttaatacaaaaaagaaaagccagtactctgtactggcattattattacaattgtctaggtgtatccagaacatatctatttgtactaggaccaccatcatcatcagttccggttgaatatatatttttcatatcagcagttagaaaagtacgatacgaaccgcgatatatttttattttatcatacatagaatatgttatttctgacggcaatggacatgcagtaaatttaccaatggtaacattagaaccaacacccaatgaaccagcataactactcgcatttaaacctgttgcatatacacctgcactcgtaactagtatactagattgtgttgggtatccagtatgaattgaatacaaagtaccgggtggtaaggttgtactattgtttcctaaaactccattaacagtgctagatgtagttttatttccccatgctccattattagctaatccaccaactagaaactgacgtgctcctgtatttggattctccactaaagcactgactgaacccattgagcatacagtgtattgtaacgtttttgtgtatgttgtacttggctgatataaagtatattcattatataaagtccatgtagtgctggaacttataccggatgtaccaccaccaatatataaattattttgtgtatcaaaaaacattgcacagctatttgttgacattatttgtgcaatatcaacatctggtgttacagctttaacaaatgaaccatcggttatttgttgagtttttccagtaccatcagcgaattgtttagccccactccaaccagtactccatattgtattattatccattaagaaagtaaaccacgtagtacctgcatacatctgtttaattgaagtacttgaactttgtgtgaacaaattttcaatatattggttataacttaaaaatgatgtagtactagtttctaaaccaatataaccaccatagccacaataaaaataatttaagtcattatcttgtactaacaaagttccttctgacatccaacaatcgttaacgttttctctaattaatgtccatgttcttaatgcagtgttgtttcctctcccaaacttcccaaaactattattgcctcgtgcgtatagttgaccatcagtataaagtacaacaacatcactattgattggtattattttcttaatttgaagaggctcaggtgtaatatttccataatcaaacattctaacaaaaggtaacatatttccctcctttattcattatactatatttaaccaaaaagaaaagccagtatcaagtactggcattttaattttaactcggtgtagctatttttttaaataatcctgtatttccggcattttcttcttcatacggaattgtacccataccagcagttctactaccattccctgaaccatataattcaccgttttttaacatgaatgttatcatataaccatgtgtcattttatcatatggaactggttcagttatattagtacatgcacgccatgttttaataacattagagttcgttataccatcacctgatgcgtttcctcgaccagcgaaataccatatcccacctgctctaacgtgtgcttcataattagaaccacctatgtaatcaatcgcagtactaggaatactagaagctggtatctttgtgattgttctaagactgttaatagtgccattagtgccatcacccaactgtccatatatttgtgaaccgaatacatatagcccatcactctttaatacgtaattagcactatatgcattttctgaatttatctgtaatactccagtagtaacagtagtcaatgtggtagttgtaccagtcgaaccaatactagattttctcccactgtactttaatgttccatcattgtataatacaaatgaagcattatcagcggttacaccaatatctttaacatcaacttctggtgtcaatgtcagtactgatactctttggtcaacagccccagtaaaaacttcaccgttgttattgtaacccatcttatataatttattatctattgttaataataatgtaacgccgtttcctataactattttactatatgtgtcattcacatcatcaaaaataccaagtatttctgtccatacacgataagtagtattaacaccacgatagatttgagcaccagtactccaccatgtgccatcatttttacgtactaatattccgtgtgtatcaccagtagtccaaaattcctttacatcagttaaacataaaacccactcttcaactatagtaggagaactactaccagtacctaattgatagtaaaaattatgccctctgccatataatcttccgtcttctaataaaataaacacattattctgtaaaacttttaccatctttataccaactggcacaggtgcttcattcccataatcaaacattctaacaaaaggtaacatatttccctcctttattcattatactatatttaataaaaaagaaaagccagtactgagtactggcattatgttattttggtaaatcaattttagtaaagactgcaacatctgcacttgcacctggaatggcgtaatacgatccagtactatatacattcccgtctttggttaataatgatgttaatccgtcactaaaagcaacatcactaatctcactaacttctacaggtaattgtaaattagcagcactatagatagcacccgtttgagaattgtttaatcccaaaccacggtttcccccaatacctttctgatatgcactagttaatataatttgcgtagccgtgcttgttgcctctgaatttgtcataaacaaaggtgaattattaggtaaagtacttacaggaactgctttgaatgtagttgaatctgaactaagaaagtttggagttgtaccacatgtgaaatatgatgtaccattatagtaattgagtgtatgtccaccaatagctgcatctattacaccagaactacgcagtccccaaatgttaacttgtgttctgttccctgtgccatgtaatccacggtcattccaccctgaactatataacttattatctatgctaatatacatactatcattaggagtagacataactttacgcacattagaagcacatagtgttaatgtatatattgatgaagaaccaatccccaatacaccacggtatccattagccccagcaccatagagtactgaacttgaactgagtacaaatattacaccaccattgcatacaatattagcaatttgattcacaggaattgtatttgtaaaccactccgttctatcaactaatgtagtactgtaagaagttgttgatgatgatgtgaaccaaaatgtttcacctgatgtccatatcgtattgtcatttttaataataacaagtgttcgtcctccagtgaaaaaattagaaacatctgcacttactaatgtccaagaagataccgtgtttttattaccatttccagattcaccaccttgatttctgcctatgtaatacaattcatgtgaatcagtcaatacgaattgaccttgagtagaagcaccaactttaattatatttttaggaccagaggtaatattcccataatcaaatagtcttacaaaaggtaacataattcctcctattgttgccataactgcgttggcattaatgttagtgtagtaatactataagtgccatcaggcaatctaccaccattaccattccagccaccacatgcatataatttataatctttagtaattattatggttctgctccaatctttagatgccataagaacattgtcataaccaccacttattgcattaagattaaaaatattccaagtcctaactaatgtattagttccattaccttcttgaccgtatccgttagcaccactacttctaacatatggtgcgtattgtgccataggtgcaccaattgatgcatttcccatattacttggacaaataaaagtactgtacttacttacatcaaccgtgaccgatgtgttcctaattggtgttagctgatttaccgatgtagtcgtaccattaccagcttgtccagtactgttagccccgctataatatacaggcccaccagtagtaaaccaccaagtaacatcgtatgtagtactaatatgttcgacatcactcatcacatatttgttcaaatatatcatagaactagcaccacaaccactttcaccgaagttatccttaccacaaaaccatgcctttttatttgatttaatatagtgcgttccttgtaatgaacatgccatatgtgtaacttcacccatattagtcataactgttagtgtaggtatggaatttttattaccagacccacattcaccattggtatttcttccacacgcaattactctaccatcggttaataatattctagtactttcacctatatatacactcttgatcatattagccataacaccaattttattaaatgaagcagtaacatcagtccatgttctcaaataaccattaacaggtgttgcatctccataatcagaaaatgcaccaaccgacccagtgtagtatattttattatccaaatcaatcatcaatgaaccttgaacaccaagccaaaaggctgcacatcgcgttgcaagtactgtaaatgacgcaactgctgctgaattcccaagaccaaattgataaaagttattactaccataaccatataaagtattagttgggctatgtagatacatacagtttgtataatgttgttgtaaaacatttgagaactctattggttctggccctggtgcaatattcccgaacttaaccatcctcgcaaaaggtaacatatttctctccttaatttaataatgcccctggcaaatccactttgacgaattgtgcatatgctgaggaattttgatcaaagtttggtcccaatccataaaatgaactaatgccgtcttcatttgtgtatatgatataaacgttctttacatcatttccgcatataaatcgaatgtctttcgtttgtgttcccgctggaacattacgaaggctcacaacgccattacttgcattgttatcagccgacgcaattggtaattgattttgaccatattgaccacgcatattttttgcatctaaaccaaacacagtatttgataccgcaccaactttcaaaaatgaaccaaaactatcattgaatgactttggaaatgtaccactataaacacttctcccatctgataataccgctgaactgaatttcccgctattcgatatggcaaattcattgtaaggaagtgtacccattttggtatatacagaatatgaattagtggaagtattatcaataatcttattaccattatatccaataccatactggtatccggcagcatcttgaactatacacaaaggaccacttggggtttgtgtcaacgaaacaacacgttcaacttgtatatcaccaaatgtatttcttcgccatgttctatctattatagtactagtattacccaaagataatgccacactatataatagattgttttcagtaaaaatagcaacataatatgcgtggttaataacttgcttcatcccagcatcaataacttcctgtggaatatgatcagtaacatccaaccaatcaaggttagatgatgcaatatctggagtactgaatttgtcaaatctagttgagccacaaactagcactctatcatcatttgtcaaagccactgtgctagtctgcatattagaactgaatgatttaacattactaatgttagacttgtaccaacccttatcagcagcacccaccgtgaatccggttcccattgatccacttgtacttgtaccataagcccacatctcaccactatatgttagtgccatacagtggcttccatttgcttggaaatcctttataccagtgggagcagccactgtattcccataatcaaacattctaacaaaaggtaacataactaaaatctccatataataattataatgtatttacaaaaaaataccagtacgtatttcagtactggtatcatttatggtaaaatgatagattgtaacgaactcatacccaatgctggaacactagcctgtggtgctggagaattcaattttggtgatgaaaccgcatatgacattcctgaacccaaatctggatcaaaaactacagcacaataattcccactatatataagtccattcttgtatccttgtgcactttgaatgcatttcgtgaacgtaccaatatctgtagtggcatttattaagttcctcccacgataaccgcaggtgtatatgaaattatcatcgcctatgtacatactagaatccccagaagaactacatagatacgacatataagcacgcatgtttttaggagcaactgacaatgtacctgcatagaatttattattcgaacccgtaatatcaccatcacccaatgcaccaaaacttccccagccagtaccgtaatatctcttatcactcaaaaatataatacagttgttattacacatcttgatttgatatatcaatgaaggatcaaccccagggaatgattcagtgaatgttagaacagccggattagtactagcattacattgatgatatgtattagtaccagaggacaagattgttccattagctaacagaattctcgcagtaccagtaccagcagtacaagcaactgcacttcctgcacttccggtgtatatttgtctgaaagtaaagaaaccaacagtcttactcccttgtccactccaaccagtattatcaacaccacacgcccatactgaaccatcttccatgatcatcattacaccaaagcgttctattgaaatataaacaatctttttagtatcaactgcactaaatctagaagttatatcagtccataccttaacacttaccgccgtcgcactggaagtaggatcttcgccagctatcattatcctcccattgttagtaatatacattgtctgactataaccagtactacaaaacttaacactatcagtctctggaacttttaaccactcgttgtaacgtgttacgttagtgttattacctaactgataatagttattcatcccagatacatataacacaccatcctttattaaactataagtcgcactatagtcactacaggtataacgtgtacccacaggtggctcaacgacgttcccataatcaaacattctaacaaaaggtaacataataaaatctccgtaattaaattatatagtatttacaaaaaataccagtacatatttcagtactgacctattattaaaagggcaacggtaactcagtaaaagtacttacgtccggtaaaccatacattgaattagcatcaccacagtaataccacttcgtagaatcatgtaccaccgataaacgctgtgcacataacacttcctgtatgtcattaaacaccatcgggactaactcatatgtatccgcaaatgaagatacaccaccactcgcatgaaaagtattcgtcccagcataaaataactgattagtgttcttacccacagcaaatgtcatcaatatcccatcattctcccataaagaagaaaacttatcaacactaccagaaagctctgtgtaagtactcgtaatggcactcgccgccgccgcattacccatctgtttattcaagttctgtccagcaacaaaatatctcgttcgtgcactagaatcagtatataataacccagtgttcgtagtcatcattccaatgtccttaatcgtcataccagtctgtgaataacttagatacgaagttaaattaacattattacccaacgaaccgttattactcatgccacaacgatgtaatataccacttgtatcaacgtagattgtactaactaaactcgtatacaactcttgtacattattcgcaataaacacagcagtcgccttattactaaccccaccaaatgataggttattatcaaatcccttagcgtataattgattattagtagtcaataatccaatcgtgaaataagacattttcatatcccttactgtactcaaatctactgccccaaattggtgcgtaacatctgtccatgtgaaaatctgtgcactaccaacaccacccgtttcaaaccatttgtcaccacagtacaatatgcgattacccttagtaaagataactgttaccaatccactaccacacgcaaactttacatcacttctaaccttcgtccaatcaagtacataggtagtggtgttattacctatacctaacttgccataacggttataaccacgcacccacaaatcaccactcttagtaattaatagtaatgcgtctcctgtacttattattcgaaatcccgaactaggatcaggtacttcatttccataatcaaacattctaacaaaaggtaacatatttccctcctttattcattatactatatttaatcaaaaaaaagccagtatcaagtactggcatagtattcaaaaaggtaaatcaatttgcttagtaataatagtatctaacggtattgaacacaatgagaaattctttccacccgtatttttaggattatatccattcatccatacattattaccttcgtaaaatgtattccagtataaagacccactatatcgtgtattcgaattgaacgatgtaaaattgaattcttgaaatgctgtaaaatatggaagggaacttgaactacttcccgtaaatggggtatgcccagttgctattagtttgtttgtatctgcattaattaaaaatacaacactagtcgaatagtccaacgaatcaggatgatatatattactcatatcacctatagtaaaaccaacatcaaaaactttactatgtgcaccactaccagcaaaagtagttccagttgtatatattttattatcagatgttatcattacaaaaccaacaccagaaacaaaactgcattttatatcataagatatatcatcaaacaatggaacaaatgtggttgcatcacccgatgttgctttaacttgaaagctgttatttgaacctgaaccatatacagttccatcgtttttaatcttaaacaatgtatcgccaaggtaaccgccaaatatattatccacattagtgtcaagtacagtccacgctgaaacataatttttatgtcctaaacctaatgcaccacttcgattccatcctcttccataaagtgttccgtctttcattaatataaatgctgtataaccacataacatttttttaatttgggtaacatctataccagtgaaatacacagcccagtcagtatatccttgttggaaagtattagaattcgtgaatgcatatgttgaacctgaaccatatattttatcacctttaataacaacggaaaacgatcctaaccataagtcatcaacttcagttgtgatttgttgaactgctcctaacgatacaaactgtgtacttaaacccgcattatagtttgaatcttcaccgtgtgcatatacagtgccatcttgcattagaaacatttcaagagtgtaactaacaacacttttctttatattattaacttcgattatattcccataatcaaacattctaacaaaaggtaacataattccctcctttattcattatactatatttaatcaaaaaaagccagtatcaagtactggcatagtattacttgggtaatggaattgtagtccatctattagcggcaacgccccctggtacatatgtaggactaccacttacagcaatagtttcattaccatcatacacaacactgaagtttgtgcacgtatacaacgaggcggtatctggattgtaatttataagtgttaatgtattggtgaacaaattcaatgcagtagttgacccgtttttaccaaacgaacctgtcgtatatatctttccttctgttgttgagtacatgttaacatatgacccagatgatgatttggcaattttatctatcttgctatctatcgttttagagttaaattgaaatggatatacctgtacgttagtatttaaaccatttcctagttccccatttaactgccaccctgagtggtacattcccgaatttgtatatgcaagcgtactatacccattaccacccattattttagtgccagataattttaaaccaaacgatgtccacttaccagtttcatttcctatagaaccataagcacttgtcccacatgctcgaagctcaccattcgatgttagaagcactgtattattaacgttaccaaatatatcaataacctgattagttccattattcagtactgaagtgaaagtactctgtgagtaagtactacctgaaccaacagaaccatttgtgttcatacctcttccatatacagttgtaccatcagcactaacatagtgcattgaatcaaaaccaatggatatcattttaatattagagatatctgggaatgaagaagttacatctactccacctaatggaattgtagtatttcctaaacctaatgagacattagttccgtgcattataaatttatcttctttcaaatataaaatattgtatccaccagcccatgcattactaacaccattggtaattagtacccaattattttgtgtagcagttccatcaatatttaactgttgatagttattgattccacaaccatatagcgaaccatcaccatatagtacatataaattatcatatcctgctataactttctttatttttttcggtactggtgcaatattcccgtacttaaccattcttacaaaaggtaacatatttcctcccttattcattatactatatttaatcaaaaagaaaagccagtactcaagtactggcatattaatttactgcacaaatgacatatcgacttcggtataattaagaacgttctgtccagcactaccacccattactacaaatgaagcacccgaataatataacttcgaagtgtcagtaagtataagtgaaccagatggagtgcgtaccaaattacacgaagtattaaacgaagtatatgaaggagtataggagtactgattattattattaccaacacctaattgcccaaacctattcgatccagaagcatatagaacttcatctgctttgacaatatgtgatgccatgttaccaacacaagtatatagagtataaactcctgaactcaataatgtcggagttttatattgtatatcagtagaagaggaagtaccacctgtaccaaattcgttcccatattgtgcaccgaactgtttaccacaagcgtacaaattattatccgatttgataaaaaatgtactctgataacttacattaaaatctataacatccgaagaaatcatcgtcaccccagtaatagaagtttgattacccgtgcccaactggtagtaatagttaaaaccacaaccccataaagtattatcttctcgcaaataaatgttaccaacagcctttttaaccgaagttatagtatctactcttttgaatgtgttaaaagaattaatgtttgtgccatcaccaaatgatgatatgttcgatatattgttagctccacaacaatacaatgtaccatcgtttgttatcaaattcaccttcatatcataaaaactactcatgtattttatatcagaaacattaataccacttgatgttatccacgtagtacagtccgtccacaaagtttttctatcagttacaccagtgaatgccttcgtgttaccagtgataaaaacttttccagtacctttctcttcatacaattgacaatcactactgttatcttccaatagaactcgtttaacattctccgcacaaagaataaatttactcgatgctacacctgtaccagtaccccaattcgtcgcagaagatgcatacaacttgtcagtataagtatcatgaagatacatagaatcactggaatatattttataacgaccagagttggtaggagcttcatttccataatcaaacattcttacaaaaggtaacatatttcttctcctttattcattatactatatttaatcaaaaaaagccagtatcaagtactggcctgtatttaaattattgttggtaatggtaatggtataaattcatcactcgatggcaaaccatataacgtagttgcatttccagaataataccatccagttgaatcttgcactatagatagataggctaacgataccacctttgttatatcattaaacccgtgtggtacttctttgaatgcagatgaatattcaacaccaggatcaccagaagatagtgtattgttagtaccagcataatataaagtattgccatctgtgcttatagcaaaagaaagaacagagttattcatatcactccacaaatccctaaacctgcctctatatccggttaattctgtatacgaatatacgtttgtggtatttgaagaagaattacctagttgataatttctattttgacctgctataacatatctagtgataccattagtatcataatataatataccagagtttaatgttgacataccaactgcacattcggatgcaaattcgggagcagagtacaaattataagaagttaaatttgtgctagtacccaatgctccagtattgcttataccacagcggtagtaaccaccagtagtactagaatatatagtaccagatagagaagtgtagaattttgaaacatttgacgcaactaatacagcattaactttgcttgttgaaccaccaaatgattggttattatcatatcctttagcatataatctattatcatttgtaagtaatcctacacaataataagttattttcatttcacgaactgtgcttagattcactgcactaaattgtgtcgtaacatctttccatgtaacaactttagaacttgtcgaaccaccagattcaaaccattgatcaccacagtagaaaattttattatctacagttaatacaacgcatactaaattactagcacaagcaaacttaacatcatctctaactttagtccactcggttacattcactatagtatcagttcctatacctaatttgccatacgcattattaccacgcacccataattcattatcctcggttattagtaataatgaattagcacctgcaacaatatcacgtatttttagtttatctggtgctacgttcccgtacttaaccattcttgcaaaaggtaacatatttcttctcctttattcattatatcatatttaccccataaacaaaacccttgacatcctttcagtactggtgtactatagaaggaatataaggagaactacacatgacaactacacttaagtactacattaatactctaaatgaactagagtacgcgaagtttctcgcggatagttcgtggctaattgagctaaaaacacataacagtactggatgtaaccttatgctgaacaatgcacttgatacccttgtagtgagtattttacacagcaacatctgcggggatagaaacaaacaattatcaaaaatcttagtactggtagctctatgaacactatgaacattgaacagtactacattgatgcactaccaacaaaaggtctacgagaaagcctactgtatacacaaaataattatataaacttcaaacattgtcctaaactcttgaactcgtacaggaataataaaataagaaatgctgtacaaggggtagaaagtatgcaatactattacatcctaacccttttaatcgagcactaatatgtataaagaattctactgtagtaagctgcacaaacaatacagagattcatttcccaatgaacctatggatatgaatagtcccttttataaatttgacttcagactggagacacgttatgcaatagaacatcaatttcctgaaactaagtacttatctatcatgaacctcatctttaagttctaaaagagtactatatgaaaactatgaacatacaccagtactacttagatattctccctaaagatgtacagttcatccttaaacaagaatacagtactttaaaaaaccttaaaagaaccgcaccgaatgtattaatcctgcaccaagaaaatgtagttgataatattctacaaggtgtaccagggaccgagtactttaacataataaggctaattacattaccatgaatcatatattatactattacctaaatctatccgacagggacaagtacacccacaactccaatgtatggtactacaaacgcggtagtataagagacaaacaacattacttctgtattgctataaaagaagcactaacaactaagtacacgtacagagcagtactgaaacttattgatatgtaaagagaatactatgagcattgaattgtacttaagtaaattacagcctggcgatagattcatctacaataagttcgaattcaactacaagaacgggagcaacaaatatcgtcgaggatacctatatacaagcacaagagatgcacaaaacgaaaagtacacatacagagaagtacttaaactaatctctatttgaggtaatatatgaatacatttgagtactatacaaataaactatctatgcctgaaagatctcatctctatgtgtggagaaatgcgtataaaagcggcaataaacttatacaaagaaattcccttacagtgtgtacaaatttaattaaagcagagtaccgcacatatattgaagtactcaaactgatagcaatataaggtactctatgcaagaattctatatctataacctccagcacaaagatagatacaactataataaattccaaaggtactacacagtcgggaacaaaagagataaaaaacgatacctactatcatgtacagtagaagcacaaaacgaaaagtacacgtacagagaagtacttaaactaatcttaatctaaggtagacatgaacacttttgagtactacttcaataaactaaaccacatagaacaaagtatgcacaactcatttattcagcaccatcaatcatctgcacatggaattaaaaatatatcgattgagattataactaacagtgtactgtcatctgaacgttatatgtatattgatgtactgaaactcatctctatgtaaggaataatatgcaagaatactatatagaacacataagcattaaagaccgtttcatgtacgacaccttcgtgaagtactacaacctggcaactaatagtactaaaaatattaatctcaatagctgcattcatcacgcactaacaaaagataatcccttcagggaaatcctcgctctcgcatctgtataaaggaaaccaaatgaaacttaatgcacacagcaaacaataccttaagcactttaaatacctcaacagtattcaagcagaatcttttacaccagcatatacttacgacccgtgtgtactaaagaaacgaaataaaaagaaaaaatccagaaatgttctcgtatctaagaacctcaaatcgtacttgaagaaaaatagtatagctcttgcttttattaatatctataatactgatttaaaagtattgagtactaataccagcacaaaagcactaaagaatgcatctatctatatacacattgataatatcccgtgcactataattctatctttcgggaatgtatacttcttcgggaattgtaatagcacccatgatatcagtactgcacaattccaaatcggcgatatgtactgcatgggttcagaaaaaatacacgataacatgcacttcttccaaaagcaacgtactaacgaaactgatacagtactgcaattcctcaaagaactagaacacaaagaattatccgaggaagaagaattcatgtacagtactgtaatcgataatccagtactctataaaaacgcgtacaaactaacaaaacatgtacaggaacttatcagtactgataagaaatttcatcacctcattacgtatatttcaaactatgtataaagaatactacaaagataaaatcccaatgcatctacaacaccatataccaaacgttgcattgggtatcaaagatcacgcaaagataatgttccaacaatgtactaagtacacgctcatatacgatgataatccatatatgctgtactacgtagtaatgctaacattcaggtactaatatgaataaccacaaagaatactacatagagaaactcccaatgtatctccaacaccttgtaccaacaaaacaattgagtatagaaaaaaactacgaaaaagactacttcattcagtgcactaaatcctcgatatatattgaagactataatcgagtactgtatcacatccttttactaatatataagtactaatatgaataaccacaaagaatactacacacaatttctccccagtatatttgaacactacgccagatataaagtccccctggataacgatttggcagtactgggattcaataactgcatagaacgaagtatagaaaatagtagtagctatgaccaattatataacactatcctacttatattcaggtactaatatgcataaagaatactacacaaaatatttcccaatgtacctcagtactaatctattagataaacctcgcggtatcattgctcaaaacgataaagtgtactttactatgtgcactaaagcatgtatactagattttgatagctatcatcgagtactgtatcatacactgtacctagctttcgtgtactaaagaggccaatatgaacgaacagaaagaattttacaccaaacacctcagtgcatcagaacaaatgtttctaaggagtgtatcacctttacgtttagatacaccacacgccagagattgctttaataattgtatacgtattactctagaagctttagaaaacaataacaaaaatattcagtactcaggcgtaatgggactaatattcttctatgaataacaaagaattctacttaagccatctcgatctaaactcccaaatgcttataccacaaatcccaatagatatcaaggataagaaaaataccgaacaattcatttaccatacacagtgtgcgatcaccccaaattacattccatatgaattgtactacattctcgccctaatatataggtactgatatgcatagagaatactatatacaatttgttaaagggaactacaaacacgccgtccccgtaaaccccttaaattctaatgacccaagtactatattcttgttccatgacgcaatacgtacagtactcaatactaaagatgaacaggtgcagtacgtattcttactattacttatatacagacactaaagcactaaggtactaacatgtataaagacttctacataaatcatcttagtcccgcaaaacaatcattctataaagcaaaaccaatgaaaatgaaattacaaaataatttcacaaaaatatgctataataactgtatacgtagtactatacaagcactgcaagaaaatgacaacaataaactatactccaacataatggcactcctatatatctatgaataatgaacataaagaattctactccaaacacctcagccccacagaacaaatgtttctaaggactacagcacctttaagtactgatacttatttcggcagagtatgctacaataattgtattcaaatgactttaaacgcaaacgacaataataacagtactaacatgtactcaggcatcataggtatactattcctatatgaataaagaacataaagatttctatataagtaaactcaaagaaaacgaacaagcatggattaaatctataatagtactggcatttaatagatatgcatttaacagatgtaccgagattgctatagacccttataatattaaagagaatgtacagcataatcagtactactatatgttaatgcttatatataggaagtaattcaacatatagacaggggaagctccgcagggaaagcattggacaggctttaacaattagaagaattgttaaaattcagcctggacaaatgcgatcactaggattctccccaggctaaaccccgtaatcatatgtagtcataagtaatcatatataaacatttaagtaatcatttataatcattgtagtgatcagtagtgattagtattgagtactagagtgtaatagagtacaggaatgtgctcgtttaatcaagaacgtagcagtactgccccaaaagtattcagtactgaaggtgtcgatatcccactaagtaggcagaggaagtctgccgtactttgtagtttccaagctccttttccattttgcaaattacatcaaatgtaatcaaaagtaatcacgcaatgattagtactgaattattactttcaccttaatttcaattacccttttacccttcggggtaaaattccattaccccttgaggctccagcaaaaagttggttagtactccatcctaaatctcgatcctaaaaattttggagtacctatgaaataaaagttggatcttcgaaagttgaaatttacgttgggacgtttatgatttgtgggatataatgatgtttgtagtcgggtagttctcaagaatatcagtactgaaagatgaaagaaagttattgactttatgattcagtactgtataatgttctacatagggtgatgagttgctgcgaacaattcatttactctacctcctgtcgtgagacagtacagaaaagatagttcctcctgtggtggtacatctttttgaacctcatatgaaaattgtaataacagtactgatattaacagaatggtgtcagtactgattttaatctaaagcattatgccaccagcacaagctggtggattccctctaacagtattcagtactacatgatagtcttcatcctcctagggtaacatagtgagggctatgatggcagtactgaatactccctttcaaaagatagatctatggtatagtactgacggattttgggtgtagttaccctggatggattttgggcgtagatatgtctagccctccagaagtctggaagtaaagatggctatccctctagacggctaaacggctagatttctagatggctggatggctagacgtttggacggctaaacggctagatggttacacgtctggaagtatagacggctagatggttacacgtctggaagtatagacggctaaatcaaaattttattttgttttttccaatagttcccctaccgcgtggggatagtttccccgcagtatctggtgttagatttttctgaatctggaatcattatcttataaactgggggcaatgtcaacgacctggccggatatttttaaaaattttttcagtactgaattttgggcgtaactatgcacccggtgggcttgagtctttctcgacttgatggaactattatacagtactgaaaaaataaagcaataccctggaagcaaaaaaattttaattaatttgcttgctatcctgggccggactatggtactatatttcagtactgagaaagtgcgtttgcctgctagggtgcgggagtgtatcctttttttatatcttgaattttgggcgtaattccgcaccccctggggtttccgctttctcgcgtctatgtagtaattatacgctttttcaggatcaagtcaatacctaaaaacaaaataattgaaaataatttaacgcgtttaaacggcctgtaaagccctggaatgattagaatggtgcaaatgtatagcttaggtgtattaatgcgttagaatgcgtctgaggccgttttacgtggtatagggatgatttagtggattgattcagtactgaataatagtactcctatatagaaagatagttcccctggattgattcagtactgaatgatagtactgaataatagtactcctatatagaaagatagttcccctggattgattcagtactgaataatagttcccctatatagaaagaaaactattttaaaagtatttgtagaaagtacttgcacagtactgataaaagcgtataatgttttacatcgagagcgggaactcttcccgctacactttaagagagattaaaacgatggcgactcaaatcactaaaatcatgcttaacctggtaaacttccgcgatgagatgaaaggtcttccggctgaaactgtaaaaattcacatttcagcttatcgggaactgattaaaatgctgccgcttaagcgtgaacaatacgccgccaccgtgatgcttgatgcgatgatccataagatgatcgccagtgatttgacgatggtttatcagtatatgggcgaaaatttcgcggtgtatagtaaaccagtaccaggaatggaaagtacagaagtattacacggtttaaatttaaaacaagatgcatgggataatatgcctcgtttcctggtatgggctgacagtggcaaaatttacgaataagttaaaaattagtttgacagtactgaataaaagcgtataatgttttacatcaggtggggaatagttccccactatataagaggattaaaaaaatggctaaatcaaaaatgttcttaggtcacaaaaacaaaactcattggaatgtaagcctgtggattaataacgatgagggattgtatcggttggccctggactttattgccgccaatactaatcgcaatgatgcggcccgtcatatgatgttatttttagaacagactggacaagataaaacaccggacggattcaaatatagcactactgcgatccgtgcggcgatggttggaatgtaatagaatttatttaaagtactatcatttttttgcgatagtactttataatagtgttttattcatacaccaacgataagggcaacagcatgaatattaagtattttgcgacgcgtgaacaagctcggatcgccgcacgccaggacggggcaacgtttaaagatatgggagcgggatcggcaagtggtgaacgttgggccattgtaaagcgtagtaccgccagatcattacagtactggttagcggccttaaaaagtcccgatagtgtatcgcgtcgagtggtagaaataaagctcgaccgaaagggcaaccgcgtgaccgtgtaccgtactaaaaagattgcagtacttgaataaaaagtacttgcaaactatgtttaaaggcgtataatgttttacatcgagagcgggaactcttcccgcctataacctggagtaataaaaatgactaatatgtatatgactgaagttaaaaccgtaaaagatacgaccacgaataaaactacttacttacagcggatctgcggtgtctggcaacgtatcagcaaatcaaagtactttaaattaacatcatatgcaaatcgttcggattcattcagtactgaaataaaaggtaataaagtacaccaatacggcacttattattattacagcctggacgcgattgcagtataatcatattttttgaaaaatttacaattgccggtattgacttattttgtcagtactggcataatgtattcattggcagataagccgtagcccatagggtgcgggagtgtatctatagaaaaattagtttagtatagttacaattgtgcacccggtgggttagctgttgtcatggggtatattatgccagtacttgataatgctgtcaatacttttttttactattcagtactgaaaactatttttaaaaagtacttgcaaactatgtttaaaggcgtataatgttttacatcaggacggggaacgaaataagtaacttcccgttaagtagcccaggagtaaggcacacgaaaaactcccctgataaaatagtttgacaatagaaagtaaaagcgtataatacttacatcaactactgaatgagggttacaaaatgaaaactatcactactactacttttgttatggctttttctgaactgaacgcagaacaaaaaacggcggctatcgaactggttcgggatcgtgagcgttcatgtgaggataatttctttgctgagggcgttatcgagtactataaagaaacagtactaccagaatacggcatagatgaagctgaattgcaatacagtggtttttggtcgcaaggtgacggtgcatcaatcagtactaaccacgttgacgctgagaagtttttacgcaaggttaaggcgttaacgaaatatcgttcaatccgtcacctgttgaacgaggatgatttagaaatgagtgtagaacgtttccaatcacgctacgcacacgaaaacacggtaggcggttgcgttgatactcgctacactgatttaactgtaaaacaggcggcattggctgaggatctggaaaacttactgactgatgcgatccgtgaattatctcgtaagttgtaccgtgatttagagaatgcatatgaggatcaattcactactgaaaacattactgcactgattgagggtaacgaatatcaattcaatgttattgatggcgtagtggaatcaatcgaatattaatttaaaaagtacttgtaaactaagtttaaaagcgtataatactttacatcaaggcggggagtggttccccgctcaaacgaggatttaaaaatggctagtactaaaacttacaacggacacaaaaacaaaactcattggaatgtaagcctttggattaataacggtgagtcattgtatcggcttgcacaaagttgcatcgcacaaagtagaactaaagatattgctgcggaatgtatgtaccgcagattaaaagccaacggtactactacaacgccggacggattcaaatatagcactactgcgatccgtgctgcaatggttggaatgtaatattcagtactgagataatggcgggtaatagttcccgcctttactgagggttatctaatgagtaaagtaattagctatacagcactaagcaagacattatctattagccagtgcaccgatggttattggctatttgatgaaacgcgtggcatgaatttagccatgcgtgcaaaaactgctgaggcggcgtatcttgaagccctgggatactatcaggaacgtttaaaggaagtagaaaacgattataataacctcaaagcgaaagtagatcatttcgtcggtcaattcatcgaggaagacgacgacaaacaatattgaaaaagtacttgcaattagttttcagtactgtataatggtttacatcaaagggagaaatggttctccctggtaatgaggaaaataaaatggctaatatcactactctttcccaactggaagcactggtgaaatctatgcgggaacatgccgacaaaatcggcgtacaagatccagccgtttgcttctgcgacgaattgaatgatgataccctggtaatagcagaagtccaggacgtttctaactattcctcgcgttacactggcggcgtagcaaataaaggcgacttctgtttataccttacaaaataacaacgcggggagataataactccccgttactttttcagtactgaatcatttaatagacggatctcggcacccggtgggttagtgactgtttcgtcttgatgtagtaattatgccagtactggatgcacttgtcaacaactatttttcaaatactttttattaaaatacttgcaattagtttttaaacgcgtataatgttttgcatagggggagcggttcccctggtaactaaggaataagaaaatgctcgaacaaatcaaatcaacgtctgcgaatgtaacgggaactgtatcacactttactagtgatgaatcgcgtggtgttccttattgtgctgtacatgtaaacactgatggcaactactggtttacggtttacggatcatattcatttggtcggccaggacatcaaaaaatcctggataaagtacaaccaacgggtaaaaatatcactgctgaaatgttttataaaaaatcataaagtacttgcaattagtttttagtactgtataatattttacatcgcaagggggagcggttccccctggtaactaaggataatcaaatgaaaattgcaatcatcgccctggctttatctgtttcctctttcgctgttaatgcgtcattagatgtaagtagtgaaaaattcattcagtgcactgttgcaatgaccgatgaaagtactggcgagattgtattaaaggatttagagatccagccggaagcatttaaaaacggcccgttccatgaagaagacgaaaacggtagcacatcaatttattatgatgccagtactaaacgcggttcggtcactgtaatggatactgagaaagtaacgcataccggcgttattaattgccgttgatagatactacggggtaacatcatgactaagcgaattgttttacttaacgaaagcagtactacacaactacttaacgccgtgcattatgatattgaaacgagcgttaatcttatcagtactgtagtactgttggacactggaaaggtcgccaatgataaagcgattgcaggtatattaacggatcttgagcctggtagcagtgcaccaataacgttaaatggtaaagtgtttaatattttactcactgattaagtacatagcccacctggtgggcttttttgtttttgtagtatcgtagtactgagatactttgaaaatagttttaagaaagtagttgacattaccgcagaaaagcgtagaatgtattacatcaagacggggagcactaagtagaccgccagtaagtcgaacgaaaaactggacacgtcgagaaaactttgatacactaataaacgtaaactaaactttaattgaggatttataaaatggctaatgtaaaatcattcatcaaagcactggtagcaaatgaaactaacgtcgcggctgagatcctggctgaactggataacgataccatgaaagcggtacgcaataattttaacatcgcgtacagtactgcgaaagaactacaagcaaataaagcggcagaaattgaagcggcaaaggcggcccgtgaagcagaactacaagcaacaatcgaaagtattcaggcggcagtagttggattggatctggaagtagttacagagatggcacaaaaagccctggcggttaagtatggggaagacaagccagaagtaaaagccaaaaaatggattcaggaacgcgtagcggtagtactggacaacggggaagaatatctgattccggtgcgtggtaactgtattgcggtagtactggacgcgattaacgccagcggtatgactcgcgaggaatggatcgaagctcatcgcaaagttgaagcataacaattaaagtactgaatcactagcccaccttaaacggtgggcttttttgtgtttatcgtttactaggccaccagcaccgcctagaaacgattctaacgcattaaactgattatatgatgcattgccctagctggtggcgttaaggctgtaagaacgcgattgagagcgtttaaagtattttgcaaaaagtaataaaaagtacttgcacgattaagagattagcgtataatgttttacatcgagagggggaaacggttctccttccaaatcaaaggaatcaaaacaatggctatcgtaatcactcgcgtaactggtactactggcaattcttctactgttgctgtaattactaataagaaaggtgcggaattgcattttgcaatggataaatgggccgttaatgagttcggtcagtgggcaagtgtagcatacattgaaacctatcaagtgccgtccattagtttaacggatcttggtgtggaacgtatcctttgcctggatgaattaaattaaaaaagtacttgcaaagtaaatcgaataaagtataatacattacatcaaggggagaaatgattctccccactacaaggaaactaaaatggctatcattacaaaaatcaaagtaaacaaatcactggttaaacttgaacaagtgaaagcaggttataaagtatcatggtgcggtatgtatcggatgttcgcagatattgatgaagcgatcaattattacagcgacacggttaaaaactttgttcgtgctgatgtggttatggcagtggcaacgcacaaagcagcgaaagaaagtaagtaaaaaatattatcacagtactattgacatagtttgatagtactggcataatagattttatcaagacaacatattaatttaaggaatacaaaaatggctaaaactatcattaacaccgtttccctggctgcaatccgtgcaatcaatgttgacggtaaacgttggactgatacatatggcaatacttatcattcttgtcatgtaagcgttgaactggataacggtttgaatgatctggcggttgaatcatttacttatggctatgaagatgcatatcaacaaacggcgtttgatctcttaaaggaatcagtagacgggcttaatactgatgaaacctcccttactcgtttgtgcaatgatcttaatatcccgttgcattgtactgtacagaatgttaaacgcaaaaaagatctttaattagctttactttatgcagatactttgatatagtgtctgcataggggaaagaaattaaattaacttaagaggatcgtataatgaaaaatatcagtactttaatcgatacaggtacacatgttcgtcggtctatggttgcaaccagtactacaaaacaagtaccaagcggttatcttgtatttgtgcacgatggtaaactcctggaacgttcaatcaattttgatgttaaaaacaattgccgttttattatctttaagcgtgagcaatataaaattgatttaccagcaaagcccataagcggggcgtacaacgtgcaatcattgtttgaacgtcaaccatacacctattaatcaaaacatcgttagcggtcgatttaggccgcgtatattggggattataccatgaacgttaatcaagtactgaatgagatcgaaagcatcattaaaccgatgaaacgtattgatgctatgttaacaccggaagaatgccgcctttatgagcgttacagcgtcgaacaagtccaggcaatggctatggctaccccagccgttaaaccgcccgagaatgcgttacaggacgttacaggggattatatcatcaatccagtactagggaagatggctgcaatcaccgaagatcaacaagattacttaagccagtacttaaaaggttaattaatatgcttactgtacaggataagatcgacatcttaaaggttaatatgtatgtgattgatgcagtacctggtaaagtatttgcatacgatgacaccactgtaagcgatttctactatagccaggacgtgggggatctttctgtacagtcggttattgaaaacgtagttaatgcagcgtatgagcatttactttcaaatagtacttgcaattagtttttagtactgtataatagtttacatgaaggggagaaatgattctccccaataacaggagaatcaaaatgaaacaatacaacgctcggtttgatgttactatcggtaacggcaaaggtcaattctctttcagtactcgcaaaagttatacagaaaacttcctgcgtaattgtgcactgaccgccgctcaaattaaacattactttcaggctaaaaaatgaaatcattagcttattacagttcggccctggtcggtgcattgatcgcggcgttgttagtaacctatctattctttatggtgatttaaatgaatccaacaggtttaaactataaaagttgttcggcgtttacgaaagcaaataaagcgaatgcacgtaaagcgaaagtacaggaatacatcaagcaaggcatgacgcgaaaggcggcgattctaaaagctaaaaataaatgaaaaaagtggttgactccggtcagccattttgctattatatattcattgagagatagctcactagcccacagggtgcataagtgtatctattatataagtattgagtactgaaaactatttttaaaaagtacttgcaaactatctcaataagcgtataatgttttacatcgaaaggggaaacggtttcccctggtaataaggaaacaaatcatgactatctcaattgctcaatctaactttgaaaatgaaatcatcgcccaggcactgcgtttaaatgatggtcgggccgaaattaattacggtttcaattcaggcggttgtgaaaaatatcagtactttgtaacggctgatggcggtgtcctgggtatcaatacagttattgtaaacatccaaaaaattaatttcaatgatccagacgatagccagttttacatcacgggcgtagacgtgaactatgaggatgactcactaactgatgactacactggtgaaacgttagaaagtgcatactgtgaagataattatcagtccggtccagaagatttcgaataatataaaaaaatagtttgacaaagtatcttagtactgtataatagtttacatcaaggggagaaaaacttctccccactaacagagagagaaaacaaaatggctaatatctgcactgtactgaaaaatgcaaaacaagttttattttctgatcacgatcatttaaacaaggatattactggcgttcggaatatcaaagatgtaaaaagtgttatgggtatctattgcaatatgaaacgtgttggaacgttcggcgacaaagtactataccaggcaggggattacaattttacactgactatcattaactaaaaatattatcacagtactattgacatagtttgatagtactgacataataacttttatcaactacactactaaggaattaaaaaaaatggctatcatcgcaaaaactgaaatcgttggtctggattctatcatcatgaagtttgaagatcaaaaaggtatgtatgtagtttcctatggtgattatgttcgggagtatgaaacggtagacgaagcctttgaaagttacattcgccaggtgaataaagcatacgacgactaccgctaaaatatattaccagtactattaaatctattgacatagtttgatagtactgtataataagttttataaacagtagttaacttaacctaagagagaaataaaatgatcgcactggattcattcatcgacgcagtactggcaaaaactggcgaggatctgaccgcagaacaatacgcgatgatcgagcaatgtcagcaagacggtaaatcggtgaaagatactgttaagattatccgcaagatgtaatattatagcccactggttaacgctggtgggctttaatcctttaccctatacaatgccagtactaaaccgtttatctcgttagaatgcgttacaggacgtttgacaatgattagcatgaaatcagtacaaaaacaaatagaaacattatcaaaaattaaacgtaaacagtactcgttagaatatacaatcaatggtaactatcatctaatgaaagagaataattttattgtcattgatgaatcattgcaaatcattagtatagtactgaaagcattaatcaatgacagaaaaaaattaaaataatctattgacaatatcatccagtactggcataatacttacatcaagacgagaacgtcactaacccatagggtgcacaaatgtaactatataatatcgatcaataaatgatcaaaaaagtactggatcaatttaacataaggagtataattctgatcaattcttgatcaaaggcaatatacgcagttgaatttatatagacattacaaaggcgttactatccgtggtaatgctggtggatatgtagtatcatatggtttgaataagattagcactagtagtattattgtagcattacagtactatagtatttttataatgaggttaaaataatggcagtaggatacgcgaaagacggggccgaacaagacgagattcacgcagtgattcaggccgccgttaaccacgctagaaagcaaattaacgcacaaagagaatcactggtacattgcctcgactgcggcgataaaatccctgagaatcgacgccaggccgttatagggtgcatgtattgtattacttgccaggaggatcatgatagtacttttaaaagagatccgcgtaattgttggcatagatctatgagataaaaagtaattgacagtactgaaagatagtgtataatgttttatatcttaactgaggatagtaatcatgggaatacaacatatcacgcgtactactgacaatggagagatcactggttatcaggtacagggcaaagtagcacaattgcagtactggtttgttaaacgtggaatcattgcaccggatcttgattatgaccgtgtagcgtttattgattctctttatgttgatcctgatagtcggggcaatggtgaaggtaacgaattaatgaatgcctttattgatgccgccggtgattcagattgtatcatcttagaatgcgatacgggagaagataacgcctttaatcttaaagaatggtacgaaggtttcggattcgaggtattgatagatagtgaatatcctatcatgattttatcatagtacttgcatttagttttcagtactgttatagtacttgcatcaaggggaaacatcttcccctactaagaggaaataaaaatggaaaacttatcaagtattttagtacaagatgatttcggtaatcctggaagtgctatcatagttcggggcaataaagcaacgtttgttattgctggtggacccgtacagcgtagcattaaaaataatgctgttaccattaataatgtacgctatcgcttagataaagaattaattcataaactgtaaaaaagtactggactatagattatagtactgtataatagtttacatgaaggggagaaatgattctccccactaagaggaaagaaaaatgcaaggtcaactaatcgttcgtaaatggtctgatactaacgaaaaaccgtgggatgtacaatacatcgcccccaatggtgaatccagatatcaaccaggtgtaacgcatagtactgagcaattcgccgccgctcatcgcgaacgtctgttacaatcaatgcagcaaggttccggtgaaaaattcattcaggatcattggctgaaagattaaaaagtacttgcacagtactgaataaagtgtataatgttttatatcggaaggggaatagttcccctcctaacaaagaggaaagtataatggtccaggtagaattaacagtactgaaagatctgttttatcggattggtactactggcgaatatccgaagggtgataatgcaaaactttacgtatcactcgacttatggcaacgcgaacaattagcaacggcctggcgtaaaggcaaagcagatagcgaggtgaaagcatgaaagaattcttatttgaagtagtgtttcaaggtgtcggcaataaccaactgattcgatcaagtggttataatgttaaccaggcatggaataacattaaacaaatgtacccgaacgctatcacatatcgcaacccgaaagaagtttaaaaataattgaaaaagggattgactccggtcagtctctttgctattatatacgcatagaaacaaatcagcccacagggtgcataaatgtatctattgtttgctggtggctagtactgaaaactatttttaaaaagtacttgcattgtacttagaatcaagtataatgttttacatcaggacggggaacgaaacaagtaaccccgttaagtagtccaggagtaagacatacgaaaaactcccctgataaaatagtttgacaatagaaagtaaaagagtataatacttacatcaaccactgataaggaaacataaaatgcggattctactgacagctaacgaaatcgaacaagtactgactactaatctggaacaatggaaacgtgaagcagatatccaggcggttcaatgccttaacgataagttaacgccagatgacttaagcacgattgttgatccacaattttatgaagatattcacgacacgtctttaagcgtcgaggatgtacttgataagcactacattgatactgatttacatgtttacggtcgtaaaaactttatcactattcacgacatttacaatacttaagtattgacaaagtatcttagtactgtataatagtttacatcaagtgaggggaaacggttcccctcctaacaaagagagagaaataaaaatggctatgatccttacccagaaagtagaactgaaaattgtaacctcctttgacattaaagttaatcggtggactgatagccacggcaatacttaccactcctgctacgtgtccgcattgattggtgatcgctgggaagacctgggacacgaaacctttacatacggttacggcacacaaggcgagtacacgggtttagaaatcattaaggaaactgtaataggtttcaacccgaaagagaaagtactacacagaattgagaaagagatcaacaaagaaattacagttcgctactacgatgtaaaacgtaagaaagacatgtaatagaatccatttaaagtactatttgacactgtccagatagtactttataatagactctaacttgtagtactaacttaactaataggatgtataccatgattactaaattctacgctacccgtgaagctgcccgtattgttgcacgcgaggaaggagcagcatttaaagacatgggcaaagatgcaccgaaaggtcaacgctgggccgtacagttcaaagaaatctctgatattgtgaattcacctgagccagtactgacagcagcagaactaaaacgagcggatatgctggcggcaatacttgacgcacaagatgagtttatcgctgattctgatatcaaagtacaggtaaaccacaccgcatggactcacgcggatcacctggcaaaccttaagcgtccaagtgaactgattcgcaaccagaaagagatccgacatgatcgtaagggtaatcgtgtaacagtatacagtaaacgccgccaggttatctaataacttgcagtactaacaacacggcgggatcagttcccgccttttttataggacaaacaaaatgattaaggttcaactatataaagataacatcttacgggtacactataaagcacctcagagaatcatcacaaaggccgtacagcttgctaatgagtttattaatggtaacctattacctaggaagttaagcaacgggaaactgcgtgtaatcgacgtggacactaacaacaggatagtaatcgagaacacaggaaaaataaacctatatgaacaccaacaatacaataagaaaataaaactaatatcacgttgacagtactcaaatgttttagtactatatgtacataccaacacggggagaaacaaaatgattactttagaaaaacgtatcaaagaaactacatcgctggtggaactgttaacagtactcaaatgtaaaacaacaccattcagcacgccggactttattaatatggttgatgttaatggtcgctatcgtatcacggtcgaacaaggagcggcgaaagtattcagtactaaaaacacgaaggaagtgatccacgccgttaatgttaaaaaagtagttgccattgatagtaaagtactgtaatatagtttcattaactcaaggagaacatgcaatgcgtaattatactaaattcatccgtacactgtcagaaatggaaaccaacaaagagatcctttcagcttgtcggttagctggcctggagatcatcggtgctggttcttcccgtatagttttcgctttaaatgatcggttagtgattaaggtcgcgaattgtgatgcgggtgtagaacaaaatgaaactgaatgtaaggtatggaattttttagactatgaccgccctcagttgaaacagtactttgcaaaggtgcatatgcatttatgcgatcctgataatatgtttatcgttatggatcggttgagtactgtaagcaaaggtagtgtgaagcacgtaaacgcgtgcaatgaattaaagtacaatattaaacgtaatgcaatcgaacgccagcaccgctttgctgatgcactacaacacgtcgatgatacattatcaacactaacgataagagatatcagaaaacagaatatcggccaggatcgtaatggccttattaaagtacttgattacggtttaacctgcaatatctggaatcagtactataaaaagcgtgatgtggcagcaagtaaggtaacacgataaaaaagattaaaaaagggattgactccggtcagtccctttgctattatatatccattgagagataactcactgacccatagggtgcggaagtgtatctattatataagtattcagtactgaaaactatttttaaaaaaagtacttgcaaactatctcaatgagcgtataatgttttatatcgagagggggaaatggttctccctggtactaaaaggaatcaagacgatgactactatgacctgcgttcacgcttcaaatcttctgaatgatgcagcatttaaagcaggtgaagaatatcaatgctatcgggatgaaaataaaaaagtttttgtagttgataataatggtttccatattgaattacaatccggcttcactggtttaaatggtaaatcagtatggggcgtacaggatgataaaggtacatatcattttaaacgtaattaagtattgacaaagtataccagtactgtataatagtttacatcaaggggagaatagttctccccgatactaaaaggaataaaatcatgactactactaatcaagctcggtccatcgccaacgataaaaatcatgctgcaatggtcgctggatacattgaagctatgcaattcagtacagtacacaatgagaacggcgaatacctggaagattatgaattatcagagcaagcccatcaagcggcagtgattgcatgttcacgcttcattgagaaacacggtaatgatctggaagtagttcgcggtatgtttattgattacggttatgagcaagctggtcatgatctgttccttacccgcgaaggtcacggaactggtttttgggatcgtggtttaggtcactatggcgatgtattcacgaagtactgcgaagaaatcaacgtttgcgatccctatgtaggcgatgataacttaatctatatgggcgaataaaagtatttgacaaagtatatcagtactgtataatagtttacatcaaggggagaatagttctccccaatactaaaaggaaacaaaattatgtccagctactccgctactatgatcattaaagatatgaacggtaatcctacttctgctggtggatcatttggaaatattaacttagatggtcggttatccatgcaatcagccatcgatgcagcgaaagaagtttttaagaatgaatgtactgttagtaagctggattatatgggtttcgctatcgagaaaactactcgttttgtgaactacaaaaatccactgatgattgatactaacctgaaagctaacgaagtggggtacttattatgacagtagaagaagttaaaaaagtactggcagtactacgatatcataatcattgcaaggtgaagaatgatcctgattatcttgattcatccctggaggaagacaccttgaacgctataagccatatgcagacattagcaaagcgtatgactgaaaagaacatgccggactatatcggaaataaaatttaaaaagtacttgcattgtagttagattaacgtataatgttttatatcgagaggggaaatggtttcccctcaagtccaggagatacaccatgaacactatcgaaatcactatcaatgcacacgtatacgacaacgtagtactgcaaactactatgaccgctaatgataccagtactttgcggaatactgttataagtgccgtatgtgctgcaatcggtgatgtatccctggaaaatgcagaacatatcaacaacggtttcactatgtgcaaacgtgataacgcaaaacatatcatcgtggaaggttacggcggtgtagaatttaatgtttatatgaaaaaagtacttgcataataactaaaacaaagtataatgttttgcatagggggagatatcctcccccactaaaaggagtaataaaatggctaactttgatgaactgatgcgggaagtagctaacctggcagaagagaagaaatcattcgattctatctataacaccgctcaccgtgattcagtactggcatgtaaccaggatcaaaaagatgaactaatcgccacctatcatgaagcatggaagtactaccgacaatgggagaaagaagaaagtgagtctaccattgacggcctcaataatctaatctaaaagttttatttcagtactattgacatagcttgatagtactgatgtaataactttactaactacaaggggaaacataatggatcagttcacacaatatcgggtaaatccacaagctatggcctacgtagaagaccataaaacgttaccagtactgcaaccagttaaaggtcatatctatcgtgcaatgtcgtggtgtacggcccaggatggctataaaaccgtagccgtgggtatgtattgcggtgatggtgaatggtttgatccaaataatccggtatccattgacagtactgaaaaaatgaaatgggccgactacatcgaagatatttcataaaaactatttgacagtactaaccagtaatgtataatgttttacatagagcgggggaatagttctcccgccaatagtttggagtaaaacattatgtctatcgctatcgccctggttgaagtactgaaagaagctaaagccaaccagatcgacgtaaagatccaggaactacaaaacggcttaatcggctgtacttgtaccgctggtggagcggcagactatcgagaacacgaatcactcgaatcactggttcggtatcttaccatgatcgttgatgattggagaatcatctaacaaaaaagaataaaaaagggattgactccggtcagtccctttgctattatatacgcatagacaaaaactagcccacagggtgcataggtgtatccaaaatatatagttaataacggtgagtaactatctgctggtggaactaaaactattaataaaaaagtattgacaaagtatatcagtactgcgataatgtatctcgtagggcggcaatggtgcgacggcccacaaagttaggagattcaaaatgactacttcaatcgctatcgttaaagctttcatccgtactaatgtttcctctgtattcatcgacggcctcgaccgtaacgtagaatactcaatcattatcgcacagactgcaaacggtgctcggtatatccttaatgatagtgaagcaaatcactactacttcactaaagctactggctttaaacgcatcgatgccgatgtagtatacgctaagatgaataagctggtagacaagattaacgctaaaggcactattgatttacagtactggaccgaaaccgatccagaatatggatctcaccgctacctgaatccaggtatcaaaatgtaaaaagtacttgcatagtacttagattcatgtataatgttttacatagagagggggaaatggttctccctcctaaattaaggagttttatcatgaccttctcaatcacttccttcgctgacgttttagtactggctcggacttttggtaaagatgcactggagtacgctgtaagtaatggcactatccaaccggaacagtgccgcatcattggaaactgcaaacgttgggcggtatccaatactattgcactggaaaacttccacgatggcaacaatgaatattctgttgatgtagtattcgaagccgttgatctcggcggtatctataatactaacctgaatgatgtgcgtatcgttcgcacggggttcggtgcttatgatgtatctattcgtgaagctatgaacaccggatacattagcaaggacttacttgctcacatcatgcagctgtgtacggaacgtaacgaagcacgcactatcttaggactgtaagcgttacacatcaccaaacatcataggccgccttgtagcggccttaactatatcaggagtacatacacatgatctatacagtatacagcgttctaggcactattgcaggcgtacatgataacgtcgatagtgctatgcgtacagtactgcaacaggttccactaaagcctaagaccattaagcagatgacagcacgactaaccagggcggcggctggcgtggaagtatggcaggactatggggatacacagataatcgtaagcgtaacgaaggattaatgcgtaaagggatttagtactgaatcataaaaaagtcaaggcatttttaaacttcagcgttaaaaaaatgaataggtaatcccttgacttttatgaataaaaagggattggactatggcaaaatgcgtagggggctaaaccccgatgtaccttagcgacttcgataaaggggtattactaataaaatgttattgactttttatatgaatcgtatattattggattgatattatttttaatggagtccctatgcgtattttaactcaagaagagcacaaccacacactcatatccaacatggcatatatcctacatatgtcacatatccgtggtattgcatataagcgtgaattggttatgaaacttttcataggaactaactatcaagaaaagtatatcgaactatacttacaagaattacgaagggacataaaaggtagactatgaaaatgcgtattttaaataaagaagattatcaagaactgttacgtaaaaacttcacaaagatttataatacagacacacacgtagaacgaatggagttataccttttgattactttgggaactaactatcaggaaagatgggtgattcaatatatggatacattgaaaaataaaatattgtccacgaggaaacaatgaaaactataacacgaacccaatcatacggtcttatcttgaaatacatggacttaatggtggatatcagcaaagaccttcctaaaactgaaattgaagttgcaagagcagagatccgcatgttggctgtactgaacacaaaccatcaggaagattatgttgaagaatggattcgatttttaatagaagatattaagagtaaactatgattattatagaaaactcatatcaatatactgaacgcatcgctgccagacaacttgaaatcctcataatatacgaagagacttctgatttaaacttagccgaggtattggaaaatgatatactattgggtgcattcttaggaacagactatcagaagtattatgaggaaaagtatttgtcatctcttgagaaatatatcaggagtaaactatgaaacatttaagtaactccgagaagaataaaattatcgttgctgcaataaacaaatattttacacttcctcacaaagaaggtttatgtgtaagggaacacaggataaacaccaccgatatacgcatgatggctgtactggggacgaactttcaggaagagtatgttgttgcacacatagttgccctcactgatttaataagggacaaatgtaaatgaaacacttaacactagatgaatatgaaactatagtccgtaaactaacccctatagcaaggatagtctacgatgaccccagccttacatggaaccaaatgcgagctaaagttaatgagctatggttgctgtactttatggggactcaatatcaagataagttttgcacagaatatgtcgaacaattaatgaatgttatacaggataaactagatgattattggctcacaaaacatgaagactttatcaggagttgagtatcaaactgccctattctctttaatccatacccttagaaaaaacaatgccttacatggtgcacctgaagtggatgttatcaacggtgaactttatcttatgaaattcattggaacaaactatcaagatctgttcattcaacgatacttatcagcattaagaaaccaaatccaatataaaaccatcaaggccaaaaaagaatggaataaacaatgcgacacagactaaatcacaaaatgtattgtaaactattgcagacactggcacagaaacactatgacacggtatctggaatattaccctatgatatcatgcgtttacttcactgtgaggccgagatgcttgtactgatgactggcacagataaaaacgcaattgccaaatcaatagatatgttcatcgaagcacaatgtcaacacattagaaccgagatatggagacgagataatgataacactaaatgagtttcagtatcattggaggttacaagctataagaaggaaagttgttagtatttctttagacaagtctattgcacaagaggacaaatgtgttataatagctgaaatgaagttgggtgctttcgtaggaacaaactatcaaaacatctacactgacgtattgttaagtgccttacgcaacaaggtgttatccagagaatgagaaaattaataggttacgaatatgttaagcttctgaaaacatgtggaagcagaagggtaaagattcaatctgataagagcatccctgttgacctaaagcacgcaatggtagctgaactatatgtaggtgcactattagacacaaactatcaacaaatgtatgtagacatatgggttgatgctctactgaaagaggtcaaaagtaaatgcaaatgattaatttaagtaaagatgaatattatgatcgtcttgcaaaaacttatccagcacgtaacgccgtttataatagtaacattattgaacgctcaaagaagttgattgtagtaaatgagattaagatgctttcagtcttaggaacaaactatcaagagaatgctgtactgttgttaattgatgccctcaagagagagatacaaagtaaatgattcatttatcttaccatgattatcaaaaacgtttaaatgcatcaagggatataaggattcaaatcttcaataacaaaaaattgaaacctgacaccagaagaatcaaaataatagaagcagagttatgcaacttcctggggacgaactatcaggctggtaaggtaaaaaactatcttagagaactcatgaattgtatcgaaaataaatgcgatgctttggagaataaaaaatgatcaatttaagtaaagatgaatataccgagcgtatgacaaaaattacgcacaaacgtaatacaatctatcacaaccaaaaaattcaacacagtaagaaaatgattatgatgcgtgagttaaagatgggtgtattattgggaacaaactatcaagagccagaagtaaatcgattaattgctgagttactattggaaatcaaaggtaagctgtactttcatcaaatgacccaaccactgaggtgtactgatcgatgaatattgtgaataacatcagattagcagaaagatttgatagaattggggaagcactgagaagatacgataaagaatatggtcatttgagcttttctatagaaaagagatatgaacttgaagcagagtttaaaatgcagagtctcttaggcacacagtatcaaaaccaatatattcaaacttggattgattgtttaatccagacaatttatgtaagaaacattaaatgcaaaatgaaatgatatacttaacacatgatgagttaacagaaagactagataaatccaaaagacttacaaataaacttcctcacaacacaaatcaaaccccagcagaaaggagtgcagaagttttattccaaagattaccataccttcttggaactgataggcaggagtatcacattgaacgtataattaaatatctcaaaattactattatggaaaaagtaaatgcaaaatgaaatgaaatacttaacagacgatgagttaaaagaacgattaattaaatccagaaaacttacaaatcaacttccaaacgacacaaacaagtcacatgccgaaaggagtgcagaagttttatacattcaactcatgtactttcttggaactgacagacaagagtattacattgaaagtatgcttcgatatcttaaattctctataaaggaaaaggtacatgctcaagaatttaaaaatcctaacagttaatgaatatcataagattgtaatgaaaactaatagtgcaatttctaaaaaatcaaacccaaagtacatactggaacacacatttgaagaacgtcaattagctttagttgaagcaaaacttcttgcactcctaggaacaaactatcaagaagaatacatcagaaaactaatataccttttaactaaacacatcaggaaaagaaaaaaagcatgaagcacttgacagatgatgaacatgggcttatagtaatggatgtaatcaacatcgaatacaaaaagttgatacgctaccgttgttatgaagataagcaactcgttctagcagaacagaagttgtttcgactcttaaacacaaacttccaagcacaatacgaagaatcgtatatgtaccttttggcgaggaaagcaaaaagagattatgaaaattttaagccacagtgaacaaacagaattgttaatctttaatacaagtaaattcttaaacataacattgtctgatatagatgatgatgaaaaagagttacttagagcagatttaagagttcttttatatattggaactaacttccaagagcaattcgttgaggactacatcgatgaacttaaagaatctgtactgagaacatgcaaatgaaaatcctttcaccaagtgaataccacgaactattaagtagtttaattctaaaattgtatatagcaaagaccacagatttacctgatgaagaaaaaaatattctaatgagtgaaatctatgttcaagctacactagggactaactatcaagaatacaatattaatgattacatcaaagaactaaaaaggtttatccaatgcaaatgcgaatcattaccgacgaagaatacctaacaatcataaatggactgttttcgaagttatttgccattaacacatcaaaacttccagatactgataaaattattaagttaaatgaaatctatgttcaagctactctaggaacaaactatcaagaagccaatgtgtataattacattaatacactcaaaagggtaataaaattaaaatgcacagacagcacacacacaaacccctaggaacagcagaatacattgaactaatggctgcaaacttctcatcctttaagaaactgatgcaagaatcaaataatgatttagaatatcatatcaaaagttcagatttaagattactactcactctaggaacagacagtcagtacaaattcgtacaggagcatattagagcacttttaaatggcatcaggaataaactatgaaggaaatatcaaatcaagagtacttggcactattcaggaagaataatcacaaatatatggcacttgattcaagaactgatataagtgaacttgaaaaaaatcttttgcgttcagagatgatagtactgacactcttaggaacacagcatcaagagagaactgttaattcacacatagagattcttatgcagatagtacaggaaaagatatgagaatcataacacaacaagatatgcttaagttcgcaagggttcatgcatctgaatttgatgctatattatcaaaaagttacaacaacccttatacacaagatgtacattacagtgaattatggatgtatatacttttaggtacagatcatcaagagcaagcagtacagaagtatttgagagctttacgtgatgctacagctaatggagtacatgtttaatatgaaagtactgacagaactagaaatgtatcatagatttaataagtactatcatttacttaagcctatacagaggattaaatgtccatttgaagagaaacaggttttaatctatgagcacaagttctttgtatcattaggtacagatcaacaagagtactatttgacgttgtacttgattacattaatagatttcattcactataaaaccatacaatctaaacaagctaataaagagaaatatgaaaactaatataaaactcaaggtatataaatcatttgcaagtacatacatcaaatgccatacatgtaactctgtaatggactacaaaggtaagccatttgcacttacttatgataatacaagaggtttttatatatcttttgctgattacaaatataacatatacgaacacattgacattgaagatttaagtactttatctgaagaggaatactttcaagaatcattagtatgggatttagagtttcctatgcaattggtacataagatgatagtactaataaaagaaactaagcctgtacctaatggttatgaagaacacatcgttgagtactgaacaatgaaaagattatcatacaaagattacaccaatcttatcaggactttacaaccagaaattaaaagaattaattctcttcctatatctaatgcagaatgttcagtactgaaagatgagttaatcttaaaacacacagtaggaacaaactatcaagctgatagtgttcgatatttaatttgggggttgatgtgtcaaataaagtccaaatgcaaatgacagtacttgacagtacttttcattactgatatagtacctgtacgttaatcatggggtaaaaattatgttgaattttttaaatttccttatcgtcttgtgtattgttcttgggattgcaccatttgtattgtatttcatggattccgatgaaatgcaaaagatgaattgcacaaggacaggacagagtagagaagagacgattcttatgcctgttaccgttattgttaataatatgcccattacaaacatggtccctaccctaacgacaacgtaccagtactcttgtacagacggagagaggtggagataatgcgtaagttcaattgtattgtacgtaagaattcatgttacattgtagtagctgattttaatacaggttctgggttgaagatatttgagtacagaaaatgcaataattcttttcattggaaattgggtgcaagaaaagcaaaaacaatttcagtaccgaatttatgttctatctctgaagaagaagagtttcagtacttgttagtacatgatgattctttaggatttacattcgaagacatgaaggaagtacaaaagcagatgattgcacattgtcatcttccaaagaattttgttcaacgcgtatgtgaattctacatataagagaagaatatgaaatttaatatagcaaaaacatcgagtatatgtatttttggttatgcagaagagttaggtgatattgacattcttttgtacaaaaaggatggtgttatgcatttcaagtaccaggatgaagaggcagacaattatcttttacctaatttagtacaaatgcaggaagaggaattattccagtacagcttaatatacgacattgatatttgcacgatgccagtattaattagtattcaaaaagtgatgctcgatatgtatgataatttaatacaaggtacagcagggataacaaaacatgtcaatatataaggagatacctaatgcatatgaaaggaagtacagtgaattataaaaagatagatgcttatttgtatgcttcaaagtacagggatttttgtttactagggggagaggaagatttcgctctgagattatctgaatttaatatgttattttatcttggtacaaaagaacaagaagatacagtacagaaatttctcaatcatcttgtacaaaaaattaatatgcaaattttgatgcttaagttacagaatgagataaatgaatatgaatccagaaatagttcaggatcttaaaaaagtaatatttcttttgaatacaagaagctctttatataaggagttcaaggatattaaaacatcgtgtaatatagatttttttgaaaccatacatgcggagtttcgtgatttaattgcagatgcattgtttgaattttccgagaaatatcaaggtacagtcttcggtgttaagaatgttcttcgtccaagtacaggtacaaggaagattgagttatcacattttacaattggtaacaacgatttattcagttataaccccagtacatctttatattttattcctgtttcagacaaaaatcatcctattcattacggatggattatatcttatacaataaatggaacggagtatcctatcaaatggcacgattagggtgtgaagattataatatgaatatattattaagaaatattaatgcagattattgtgtatgggttaagaatagagatatgatgtattatgatttttatttagattgtgccattaagcgttttttaagagaagaacattaatgaaaaccgttaatacaaaagcaataaatgatttaaatgattctttattagtaagtatcaagatgtattacaatatacatttaaagggtggaattaatgattttagttattatcttagtgcaatattacaagagcatttaaattatcaagatcgtttattacatccaaatccaccaaattaaggaataccaatatgaatgatattgagttattggtacataagatttgcacaatatcgttagtgctgaacaatgcgaatacagtacagagttcccttttaagaattaataaagttgggagtagtttatatttggaagtaaaagctggttttgcaggacagtacaatgataaaatcaaagaagaagttaagggaagtgaagatgctttcattgaggtttgtgcaacagttggttcaaatccaatttcagtatcacctttaatgcgaaatgaaaatccagaatttgattccagttttgaaggttatttgtggtataatgctggtgatgattattttatatatcagaattccaaaacggattacagtgaagaaggtataaaattcattgatccttacaatgaagcagaagtatttcagacatccctaatatacaatacaaatcaaatgaagtatcttatgttgtttagtacaatgcatttgaatccattgaaagataatttaaaatttttcattcgtttagaattagttgatattgcttatgatcttttaatgcaaggatatagttatgaagtataaaatgctttatacattaacattaatatatgtagaattagaatccgattcagatgcatggaatttggttaaagaggtcgataccaatcaatggtacatggcaaatttagacgaacatgttgttacggattattttatagattttaatacattatctgaagaagaatttttccaaatgcaattggtggttgacatgtacggacatgacatggtaacattaagaaatatacagaaagtcgttaatgaattccaaagcaagaaagattatgaggtaggagcgtattatgacatcgacgtacagtgaaagcaatgagtacaatagttatgatatataaaatgctttatacagtatcggggatatatgtagaattagaacccgattatccagatggatggaattttattaaacgctccgaaagaaatacatggcacatggcacatttggataatgatgtaatattagacaattttgtggattttaatacattatcagaagaagaattcttccaaatgcaatgtgtgcttgacacaaaaggatttgatgtgctattattaaagaatatacagaaagctgttaacgaattaatatccaatgacgagttaggcaaagtgaggctgttccatgatctcgaagtaccgtaccagcaatgaaattcagtacacctacgctaatgcaatcctaatgggacaaaagttagcttgtattgttgcagccttatataatgtacagaaagcagcacataataaaaaaccatatacaagaatcattaacgcattcatggggtttaaaccatctgattttgattcacatattgcgtgcacaagttattttgacaaacgattcaatacacctgataccccaaatgcccgtagtgttattagtcttttacagtttaatccaacaccagttatatcaaagaatgatttggattacacatggtacaattcaaatcaagattcattttattctaatgatcaatcgattgaatataataaaattaaatcagaggaagcattttttcaggaatcattatttacacatgaacgttctttgttttatagtgttatgttgagtacagtatacaatagtccattacacaagaataacaaaattaatttcagtttttgtgcttatgcgattgatggtgtttatgagtgcttaatggaggtattgagttatgaagctgaataatacatttgatattaagaagttaagagaatggttttattgtattgaaattgaagccattccttatcatagatatgaacaaagtgatttacaaagtattgatatcagagagttgtacaaatattatcagagtgcattagagatgcataagttatttttgagtacaaatttcattctaaattacaattttgaggaatttaataattataatttagtacagaaatatgtagtagcaaagtacatgtacaatcaaggtatcaaaggttgcctcgaattcaataccgttcttattttagttgaggtaattttatgcaccgcatgtctgatttagagttcagtttagatgcagcctttaaatttgttcacaaaatagaaacaatatatcgtacattatgtgcaataacatcagtatcaaaagagtttgatgttggtgaaaggtatcaggtgtggaataattcattttataataaagaacattctgcgacggcattttatattttagcatttaatcccagcaattgttacccagagaagcatgaacgttatccaaatgacgacatgtggtttgagtacaaagaagatgctgatatgtcgtcaattgtagttcctgatgattttaatgcacaagatttaacagaagagtggtggttccagcagaatttagtacatgatgatattgatttatttttctatattgcatttaaaatgctttatcacaacaatatagttaaagaaagtttatcgttttcatgtctatatattgatgatttgtacaatgaaattcaaataaacatgcctgtatcgtaagatatgggctttttttgtttgacacgagtacaaatgcatggtacaatgtgtaatcattttaaggagatacctatggaaaattttaaaataccagatgatttcaattcagtacaagagattgtacacaaggttcattatatttttaatgaattaattattgccaattcatccggtatgcaaattgagttaaaggatattgatagagaagaagtttttttgggttccccaaattcaatcattcctttttatggacaagatcgtttatggttcatgagagataccccaattgacaaatacaaaatacaaacaaaatcagagttttattctatttttgaatatcttaatattacattacaaacacaagatataaatgcaataagtgccatgagtgctgaggaatattttcagtacagtacattgcatagtccagaagaaatctttgttgttatgctcgttgcaaaattaagtacagttgattttgggacgtacccaaataaatcattcaagattattattcctgcattcgttgatctttatgatttcattgtgcacaagaaagaaagtacagtgtacgatatgtatcgttcaaatatcagtttatttgcatcaagtgcagtaaatgcagtaagtacaggggaattaaaatgaacatagatcaattaatgaagtacagtacaaatacaattccagtgctaagtaaagcattagatgcattcaatgctaagtaccctgaaggttcagagttaaatattccttttaccacagaagggattgttgatacgtacattgtaaacattagtagtacaggtattgttttgaagcaaaagtacggcgaaagtgaatattatggattgaagctggataatatttcaggagagaagacaccttatgagttttgggttccgaataacattgagtcatggaaaacaaatattaataagagagacgaaagaaatttacctcttgtacaaaataatgaaaaaagaattgacgtattttattattacagtacagcccagtttccagaagagatttatttaaattcagaggtgcatgaagatattgaatcatttttctttcagttatcattggtacaggatatcatgcctgtgcaaatgttcagggatgaaataaatttagcgaaagagatttcggatatgttacctgaatttagtgttctttatggtagtagagctttacctgaaaatgtatacaaagaatttatttccattgttaaagggttgaatatataatatgaccgcctatgatatcaaacaaaggcatacgaagtttgatttgttcacataccaattccacaatgatatagtgaattttaacatcaatgcgtcattgcatcctacatcacaagatgattttgacaacagagtgcttaacatagtggtgcttttgtgttataatcatttcagtgaatattttaacgcaagataaggttgaggcacacatgtatcagaccaagtacaatacatcatatgcatttgcacacaacattttgaagatcatcaaagcgttagagggtggattgcttagtcataagtacaccagagtaggtgacaattggttcagaaatgatgatgagttcagaagtcataagattgtttacagcaataattacagtgccaattaccttcaggatgttgctatgtgcttcgaatacgtgttacagttcaggggttccatcagtcctgagctaagagaacacatgtacattcgatacctaccaatgcttaaggatgagaaaacaatattcaattcagtacaccacagagtacgagagagttcattcagggatattttagaagatgaagctgcatattttcagcaatcattgattgatgctcctgatgtgttacagatgcagaatttgtataatgtattagatgcgaacatcgatacggatatctttttttccttaagtgattatgaaacatttttggaagatttaaaggaaataggattatgagtagattttcagtttatatagtaaacgtcagtaaagatggtagtttaattgaatttgatgtagttgataacaatgataattcatatggtattaaggtacaggacaatgatgtacagttcagtacaaggacaaattttgagaatccagtccaagttccgaatttcatagaagatgaagctgcacgattccaacacgaaacggcctttgataatttagcgatgcatattattatgcaacaaagtttagttcaattgcttcaggtattcagacaaaatatatatgtacatgaagtgtcgattaatggcattcaaagttccgttttgttcggtacaaatatgtaaggaaagtacaatgagttcaatgagtttattagatacattagaaaaagacagaagtgccattaagagattaaaaaatcttgtccagagcattgcgaacaacgctgagaagttcagtgacaatcttagtataggtgatataaccagtacaggaataaaatccctgaagtcagatttattaattcataaggaagataaattattatttgtattgagttcagatttaatggttgcaagtaatggtttcaattcgatatcaacacaagatggtagcacaggattatatctatcacagggtgtccagacgaacgaacaaaagtgttttagttcaggagtacagaatccctttaagttcagtacagaacaagctacagagcttttagatgcttcagaagagatgttatttcagtacaaaacggttcatacagattttgaattacgatgcatgtacgctgttaagcattatgttcaggagttcgagaatttaagttatcttttagttcgaacgaatacaaagaacattggtgcattagaagattacatttcaaaagttgttaaagggtgctaaagggtgctgatgaaagagttatttgaagaacagttcgatacatatgtggataatattctttattctagccgtgctagtcaacgaggaaatcgtgcttacaacatagattacctattaacggtgctgagttcacagtacacctacgctgtgcagaggacatattatggataattttagagatgttgcaaaggagaagttcaagtgcagtttagattacggtttaaatgattttcttacatgcacaatgtacaggaatgatgattatgagcaatccgtaatttcgttgcacagatatttcgaaataatttacgcaagatatcttagaaggttagatgggataggggattggaattatgattacgtttgaagagccggaagtggtatattgctttagtgtgaaaggttttgaatacatgatggattcgattttgaaggatacatatttggttcagattgcagttgcagataatgcagataatgcagtacaacgagtattgaatatatacgagaagacgagtcgaaggctttgtgcgatgaaaaggattgaagaaatcaagctttctaggctgaataaatagagacgtttcgttttcacaggccgctggggggggtcaaaaaaattttcgctgaaaaacatgttaaggtatatgtttaatataaagttttaaaatgtgttcacagagtacagggcatgtttcctgaaacaaaaaataccccgccgccgaacgttctccttatattggagtttaccttattaattacagtagagattacaattgaaatgaagatgcaaataaaatataatgaattttgtgcaaggacattcatgtacactggtgaattagttacgcactgtaacttatacaagggtgatttaaactacacaagggacaagcttataaaatacgaaaatgctgacttttatttgatggattttagtactgggtttcatgggatatctatcgagatgccggactataccgtacttgatgatgctgcactgttccaggaggcattagtatttgataagaagtacatctgtacattagaagagatagttgaaatccagcgtagaattaaatgtattcacagggaatgtatagatatctggtgcaggactggtgaggctgatttctggaaattttttaatctaggggaagagtgatggaatttgattttgtaatatcagtacattcttttaagaacagttttcagtacttttgtaatttaaacaaaggtgatttaatgcacagtgcaaatgagtatattcattacactcagggtaaatttacattcattgattacagttcattacagtatccgcatcaggatatgccggattatacagtacttgatgaggctgaactatttcaggagtcattagtatttgataagaagtacttgtgtacatttgaagaagttattacaatacaaaatgaaatgaaagagtatgctgctttgaataaaggcaatgtcagtattaatcaagaatacaaattcaatataggaataccagaatgacatttgatttttctatgtttatacctaagattagtacttcactggaagtgtattgcaatcttaacaaaggtgattatgaacacaaaatggatcagtacattaaatttaatggttcttactttagcttaattgattatcagtccaaaggaacagttgtagaaaatatgaaagactatacaactgtacagggtgaggctgaaatatttcaagagtcattaatatttgacaagaagtacttgtgtacactagaggaagttattgaactccaacagaagatgaaagaatatagaagaatctgtatagagactgattccaaagataggtatcgttttaagctatgaataaaaaagaacaaatggaatcatttatgaatacaatacttcgacgattcgtaattgatattagacataacaacagcattaggaatgatatgtacttcggtacatatgatattagtgaattgtatatagctaatattcacatggtgtaccgagattatatcaagacacaacacaggagttgatatgagtttttcaagagcattagaaattgctaagttaattgatttatgtaaaaaggaaatagtactcaatcgtctcaatgggtatctattcaaggaaggtaaagcttattgtcgaatgatatacgatgacagtagtgagtacattcacatgtacgaagaaaacttagattcttctgaaacaacactcactgggtgtgatgttaatctggaagataattacatgtacttctgtcgtgatgctgatattttaatacaatatactgacattataactggtgcagcagaaaaacatcttccgtattcagtactggataatgactggggaagtgaagagttctacttccagcagaatactatctacgaggaagatgtgcttgacaagcttgtcattctgtggtactttaataataccgattgtcctagcttctatctggacttggatctactacgagttattaaagaggacatatgagtttagcaagtgcacttaaactggcacacaaaatacatcacttccacgataacataaaaagtggtttctatggtgataaagtaagtgcatttggtaatgagtattatctatcaggtataaagacatactctgatgatattttacttatgggccatgataaagacaatactatacagtactcttcatgtagtcaatatactagtttttttgtatatattccaggtactaagattacttttgacacatcaaatacacctgttgacttcggggtacttgatgggccgtggggtactgaagagtattacttccaacagagtcttatttttaaccaagaaatgcttgattccttggttgttttgtggtactttaaggaattgaattttgaacccttcttccttgactggggagcatgtgaatatttaagtacaggggattagtaatgtcattagaaagtgcattgaaagaagcaaagaagattcatgagtacatgattatggtacaagaaggttcagtgcctggtgtgtataaaacatctgatactgaattttttgtaaaagaccgcgacgattatgatgagtcagagatacatgcagaaacgttagtactacacactgatacgggtgatactgatgactgtgaatatcatatgtacaatcagtacattagttattgtagagatgaagatattataatttacagccctgattacgatgattatgacacaatcattccgtattcagtactggaaggtccgtgggatagtgaagaatattacttccagaacagtactgtgtacaatgaagaagaattagatgttattgtaattatgtggtactttaaaagtattggttctgatagtttctatttcgattatgaatacgtagagaatactgtaggggaagataatgtcattacaaagtgctctggataaagcaaaagaactacaggtatatattgaagatatcaaggctgggaagttcgaagaagtatacaaagccgagaatggtaactactacaaggatagtgattttgctacaactacagttattcttgggttgaattccaaaagtccagcagatttttcacaatacaatccagatgcagaatttatcagttatgcttatggtggtgaaatccgtttttataatcgcagcagacctttaaaattgaactttgaagtactgaaaggtccgtgggatagtgaagagtattactttcagaatagtactttgtatgatgatgaagtactagatactttagttattatgtggtactttaaaagtattgatagtacaagattttatttggattatgaatatgtacgtgaagcatataagggcaaacgatgaaattcttatacagtattcagtacattcctggtacaattagtttaatgtatttcttcgatcatgataattcattattactagtagtaaagaaatgccaagctagttcgtttgttgaatacattcacgatagtgaatatatacctgattttttagagtgtgattattacgatgaacatgaatcatttattttccaggaatcgctcgtatgggataaatcgatcaacttctttacgttaattgaattacaaaagcaggtgaagtatcattgggattacttaatcacacaggattacattgagatagaaatataatgactaaaaatgaatttgattcactttgtacggtttataattcattgacagaaatacctgactttattaattacatttcagctttaaatcaaattatgcatagttttgaacaatcgtttgaagagaaatatgctggtagaaaagttctatgtcatatgtcaatgtacttaggagatggtgagccttacactacaagtactacatgtgaaataaaaaaatataatatattattggggacaatagaattcacagtatccaatggtggtagttatatgtactttagagatggagtggttgaattattatgagttacgaaagtgcaatcaagaaagcaaatgatattctaaaagttgttaagttcttcaaagaaaatcctgaatacttagctgatgaaaaccatgatcacgatgagagattgagacgaaaaattgtcgctttatttgacactaatgcggataattatagtctaattgagttagcatacgattatatttcattgaaccgaaacggcaatatagttaatatagaaagacatgcaactgaagaatcttttaaaatttcagatattttatccctgagtactgaagaagggtacttccagggtagtactgtttatgaacaaggaacacttgacaatctatgcacattcagtgtaatatatcagaaatctgtactagatgtattttatcttgacactgctgaactggatacactaaccaattttgttactactgaagaggctacattcgagtgatgaaagttaacatcaatgactttgatgcattacaagatttatcaaaaaagattcgcaccattgtaccagtactcaaaaaatgcagtactggtttaatcgatcctagtactgttgttaatgagaagtacaattttcattatgtattacagggtactggggcattccagaagtgtgaggtgcgtgagacacacgtaattgatacatcattgttttctattggtgcacctgtgtcgaataatactattttcctagacaacgctggaaatattcaacaattaacatttaagaaaaggaaaaagaacaataacattggtgtgtatgcgatgaagtggggtcaacctgatttgaagcgtgaaattcacatccctaagtacaattacaattcatttgaagaacagtgctttcaactatcgtgtactgaccctgactatgctatcattcagttctatatggaattgcagatagcatttaacagtaatttgtatattaacttattggcgattaacattgatgaagtattcaaaaacatttaatcgaattgaatacatgagaactgtgagtgcagtgtgggacatatggttcactactaaaccaaaaatggattaccatgactttaagctcttgttcttcgttggctctaacaaacaggatgttgcaatgaagggttatattcagcacttacaggaactaatccagaagagaatatatgaaaataatagaccaaaatgacattgctaaactattcaatatatattctactagaatgatgcaaacagactcatatagtactgaattcaaagaattaaaagtactgatgtatttgggtacacagtatcaggaacaagttgttaaagaatatattgattctttacagaaggatatccgttatgtacaatgaacttgacttggcatttgagcgtgtaaaaatcttcattgaaacttatcttatgcatgaaactattctgaaaacaaaaggaatatggttcactgataaatttcttactatggctatgagaggtggaatgaaacatctgtactgcacagataatgcaagaatttcagtactgaattatgatgaaagtgtagcatccagtaccagtttaatttctacatacaatgataatgttattagtattatgtacaataaaaattttccatctatttctgtatataaaaactgtgatagttcattggagtacaatattgaccagtacttatatcctaatacagaagaaaacatctttcaaaatagtcttatgtgggatgatacatctactaaatgtatatatctttataataaattaaaagagtacattgataatagttcaattagtattaatatattcagtgttactatttttaatgatactcgtgaagatatgagttattacagagatattgaaagaatacacgcaatgtataaggaactactatgttattaaatgcaaataaatgttgtagtactgaggttcatttgtattttaagaaattagtaaatgatattatctatatgcaagaaatcatttatcacaaaagaatggacttagaaaaagcattagttcagttctcttttaatctcgatggtgatatatcgttagatggtgatggattgcgtatagcatttgacggtaatacaaatagggttttcttttggatagataataaattatgttcatcaatatgtcgtactttttataaccctatttcggacttatatcaattggtgtattctaaaggtgcagtagaagcatcatatggtgatagttttgcactaggaaagagtatcccatatatgcaatcatttgatattgaattagtcagtactgatgaagatgcaatgttcacaaatgatataacaatgcagcgtgaatctgcaatgagatatattacaaattctatgcatacattcttcgaaagaacagaatttgataatgtgtatatagcaaatgtaaatatttttactgacctttgtcagttcttgagaagtatttttgatttcgatgaaagctattgtaagctttatactgaggtttaaatgccatgaataccattagtattacgtactacacaacgaaaaaatacgtttgttgtttactttcgttaaagacaaatgatgatatgtttgaaatggcggcatatacattagaattgtcagaagatggatttcaatgtcatgaagcgtactcaaatacacgtgaagatacgattgatactacgctaatgcaagaagatgagttatttcaagaaagtttagtatgggataaaaaatactattgtgattttgattttctttgtttaatccagaaagaaatgaaaaggatctttgataattatccagtgaagagattgagtactaagccggagcagtacaggagaattttattccgttatgaagactgaattagtaaaaataatgtacgcaactcacaccaagttattattggttaaaatggattacaacagaaaacataatgaattctgtacattaatccgaaatgaggaatcatttacattgcgtacaaatgataaaccaatgctaactataaatgatttttcatgtttagatagaggtgaattgttccaggagtcattagtactggatagtagtatacatttggaaattgaagtactacaacagtaccagatacttttgaatcgtattatggaaaattatccatgtgaagttggtggaccaatgcaattcaaatcaaagatgtatgaactatgaggttagtatgttagataaaataaagaacgagggaactctatacaatcatcttaagttggatacgttcgaggcagcaatcagtactgcacaagatataattgatattatcaagtacattaaagaaaataagttaaaatgtgtgaaaacgccagaacgtacaatgtacaaagatgacgaccttcacgtatcagttgttaagccaaaagataaggatggtaatagagatttctataataacagtggacagtatgaatgtgaatatgatcccgaatttagaggtggattcatgtgcttcagtaaaacagatacttcacatgttgagatacgtacattaaaaggtctaggtagatttgagttcatggatatagaactaaatgaagaaaactacttccaacaatcattgcttaatactgaatacaatttaggtgtactattcatgtacaagtacttatatgagcacttaaatgaagacttctatctggacttagaagattaccatttagtatggaaagaaatggtagaacttcgtgagaactcttgacaccctccaaatctttgtgatataatctctttatattaattgtaaagggttatatcacaatgtccaaagacctatcatttagcaatgcacaaaaaatcgcacaaaatatcattaaacttcataagttctttgtaacagttaaagagaatataattctcccacgatgcacactttatgcgtacaaaaattatttgtcaatgaatagcaatttgttgcatactacagatgagaatttctgtggtattcgtgtacttgacgatgcaccatcaggggattttatctcatattgtttccatacatctgatagttttaagtactatcatcctagatctaatgatgatgatgaatatgacacatttaatgttagatcagaagaagaattcttccagtacagtactttgtacaatgagagaatcgtgtttagttctacaatttttacagtacttaaagatgaactcaatgacgcaaatgatagattctatatggatttaaacttcgtagatgaaatcattgagtacatggagaaaaatggtctatgattacaacagaatatgattacgatggtgttttagaatttgcatctaacattcatcgcattgcaaaggagttcagaagtccccaacctttcggtggaatgccttttggtggaatgttccaatatgaccaattcagttcaaccaaaggaagacttgcctttaaaaataataggaacaatcaagtatatgctgagtactttgacgataatgactttcattaccgaggtgaatatatttcatattgccaagggtccgatttgaagattaacattgatacatttagagattcagtgcttatcccgtacaatgagttagttttcatgacagaagaaatgtacttccaagggtcttgtattcatgatgcaaaagtacttaatgctatattactttatggtgcactacttcatcattacaaatacaaacaacgattctttttagatttagatgtataccataaatgcgtggattatttgaatgcataatacaacaacaattcagtacatgacggatggcaatgattgctttgcttcaagatatgaattcatcaagaaccatttcgtaacctatatccctgaatataatcagttttatctacagatttaccaaaatgattcatatatgaatgagatatattctgaaataaaaatccctgattatagtactttatcagaagaggaattgtttcaggagagcactgtcaatactgaagatgtgtacgaactaatggaataccagaaggtacttaaatatttccatgaagttcaggatatacaaatggatagtgttaaaacgtggaattataacacatacgaggtaaattatgttcgaaatgattaaaattatgcaacgatgcattgttgaccaacatggaatatatggatatagtttaagtgcagctattgacaaaaacaaaacaacatatgtaagctataatgcagtacttggatttgatatattcattaatgccaacaataaaatcaaacaagccaagtatgtatctataatgacccctgatgagctatttcagtacagcttagttctgccgggctgtattgatttacaacgtctaggagctatacagaaggttttacgcgaacataaagtacaaccaaataaaatcacacacattccagaaacattctacaaacccatagaagtagaaacacctgattatatgttaagtgatgatgagttctacgagaaacacttccctggtaaagtttggagagactaatgaaccgaaagtacagagaaactatgaagttcgctaaggagtgtttagagacaattgatttcattttcgataacaaagactcattgggccgtcacttcgtaattacaaagaaagaagataagagatacaagtgtcaaagattaggatacagtattgagttctccttaatgattgatggaatgataaactccttatatgaatgtggtaagactgattacctttcatttagatcatggagtgaaggtataatgttcttgagaacaaatggagttttatatccgtttcttgatgatttatcagaagaaagttactttcaaaatagtttaatttacacagaaaaagaactcaaaatgctattcattatgtcgtactttaaaaagtactgcaattttaaatcattatcttttgagatctctgcaatgaaagtattcattaaaaatgtaaaagaattaaagaggatttacgatgtacatagaaaatgataatgtaaaatgcattgccgacatttgtataaaattaatgaaaattttcattaacccaaaatacaagactcgttctttacgtcataatattgatgatcattgtgtaattactattctaggtagtatgcttgatgtaaagtacgatggtaaattgttattcggtgtccgagaagagaagatagatgatgtcttcttttctcctaaagataagtttgaaccttctgattataaagtaattattcgtgatgatagtatctacattagtagtgactattcaaattacgtcgaacagatatacactgaaaatgttgactacaaccatgaactactagatttaccaacatcagacatagatatggagagaatcaaattttcattatcaaataatgagtacttaggtatacaaacatattatgcacttcagggaaagattccacctgcgatgtatataagaattgagattggtaatgtattcagtgataaggttatatcaattgctgataaaatattgaaggtacaattatgaattttactaattataatttacatagacttgaattacgtcattgtattgcatttgcaaaaacattaatgtgcatcattgatacactatcatctaaaacttttcgaaaggataatgatttatgccttaaaaaaggtaaagtacaaaaatacgattatacagatgaaacgtattttgatatctttttgggaacaaatgatagtacaagagtaggatgtttgggaacagttggaacacattgctttacatggaatccaaattcaagtaaagtacgaattgacaccaacgatacatgttatgctgaccttagtactgaatctgatttagaatcagttatgttccaacacatgttaattcataaaccaaatgaaattctttatcttgttgcttttagttatctcttacaaaattataaatcaggttttactattgaaactgatatcgatgagattatggatgagattcctagtttaaaagatattcttgaagaaaaagatttctatcaggactttagagatttatacaacatgcacagggagttatctgataatgaataaagatgatattataatcagactaatcaacagtctgttaaaattcgtgaatagtgatagcgatacacacaatgtaggtgacattgaattcaaatcatttggaatgtacggtgaggacaaaacatattatgcatcaatgaaattccaagattattatgaaatgttaattgcgttaacatacgaagatgtggacccaagatggagttccagtatttctctgtcgagaatggtaattcgtagtaaacatcgttacatttcgtcaaagaatactcctaatttgtttgatattgttttgaatgaaaataccaaccccccatctaaacatgatatcgtacctttaactgatgaagtacagtttcaattgagtaccatttgtgatgaagatgtgatatcatgtatgaaaatttacgagaagattggtagtgatatccctaagaactattacataacaatagatggaacgtacctccgagatgaggctctagcgtacattgacagcgttttaccaaaataaagacccttatgggtctttttgtgtttagtgctttacatgagcattcaggtgtgttataatcgcttttaacagagtacaggagaagatatgagtactgaattaattaaagaaatcacattgttggtaaagcattgttttaagttcattgatgataatggttcacgccagtacattaaagttaatgagaaatataacattaacgtcctaaagaatgaaaccgttaacagttatgcaaacattgagttagatgttgaggacaaatacacagcattacacatcatttcgtccggcgatagaaaacaagattatcgttctacattttatgatgatagtgttactgttggttataaatttcataaaaataaaattgcctataagaatacatacacacatgaagtaattgaagagtacaattataaaattaaaaaagacgagtacgatgtacttttattcaatgcgaacctcatacacaacgctggtactgttcgcaatcttatgttagcatatgaaattccaaagattatagaaagaaatgaattgataatctttaaaactatattaaaattcagtacagaagaattagaagatctagtactggctcttaagacgttcatcaatgaacatgtacctgttaaattagaggacttataatgcaagctgaacacattcaaattatgaaattgatgcatgacattgcagaactatctgaaaagcacaacattactgaacatgagcctattggaaattatcgtcttaaagttgaatgcaaatcaccatatcgttttggttcagtggtactggagtcttataaagtttacgattatggaacgacgaaaaaaggttcatgggatactatggcaatgtatatccgcacatcaaggcatagtgagcaaatcggttccattactccaaacatgatatttgtcaatgctttacacgaagatattaatcgaattgttgaatctaattttgatgacgtagagaatgatacatatttggaaattccaccagaactagatcaaagtgaagacattcgtgaagcatacattttccagagatcagtacttgacaacttcaaggagtttgagtacatgatgctttgctatgaaactgcaaaagctattgacactggtgaattggtaatgtatagtaagttcattaactgtgaatccgatacattaaaagagctacacacggcaatcaaaaacatcaaggatacatatgaacgcttatagttatatgacagcccgacaccaacagctatatgatatcagtacaaaaatgttatccattgttggtagtaccaaagaaattaacaaacattacctcatccgtgatgtgtcattttcatctaaatgtatgtccaaagaatattacgatcacggtattcttactatttctggtgccaggaatatttgtattgtaaaaaataatagtaaaagtaatgaagagtgcgagattagatccgatatccaaattaatgatgatggaagtttttcacaccatccatttcatattagtactgttggtgttttatataatattaaagtactattcggtgataaagagtatgagttaaaagctatcaatccagaatcaagtacagttgatgaagaactgtttcagatgagcaccgttatacctgaagaggttcttagtactgttgttttttgtagtttaatttatcacacatacaaaaagaaccagttcgatttccacattttaggaacgtacatcttcaatactttgaatgttgatcgtttaaaagcattaaatgcagcaatggataaggtgctataatgacaagagatgaatgcttatacatatgggatataattgaaactacactttcaaatccagaaaattataaaacaaaaaaaagacaacgacacaaaatatggatagatttgttcgttgagggcatacacgcagtgactatttgggattcaccgagggcatcagcatctactctattggacaatgaatgcattgattgcattaattccacttttcatctccacggtactacatatttcggcacatttctggatattgaaagtaaagatgatattgattcacttgctttacaatacggagtactgtatgatactaaattaatttacgatttagtactagcttcttatgtgtataaattaatgatgaaatacaatcacatttctgtatcagtttatactaaggatattctttcattaagagacaaaacatgagtttattaaatgcacgagaattagttgagatgtatgagatcttatcttcatataaaaattctccaaaagatgtaattctcaacaaaaagttccatgtcaatgttgatcatatgaaaaatatagatatgatttctttagtggaaagtcgtaaatcgggtcttgagcgtgatatcatgcgtttgatttatattaccaaaccatcatatactaatgatagttcaattagtaatgagacgtttttgtacactccgtatactgattgtactttagtttcaatgtctaatcagaaacaagatactatccaattcatctataccagtactgataacattgaagaaaatgaattccaaaacaatacattaagttcatgtagtagtgttataacctttatgtcatatctgtggtcaatcaaatcaaatgcaattgtatatagttcgaactatacacaagatagtattattgaatcatataaagttctaaaggaatactatgaaaataactaatgattttattagaagtttagtgcaccctatgcttggtgttatgaaaaaactacagactctacctgaagacaacgattacaagtttaatcgttttgattcagatgtgttttgtattgaaatggaaattaataacaatcgttattttcatttaaaattcaaagaatcgtcgagttcgcactacaactttgacatgaattttaaaataagcaatgatattgaagaaaacgcaatatcaaatcatattagcagaaatggtttcatgcatagaactaatatgttcaataataaaaacagtattatatctaaatggaatcatcaattcactaaaatttgtgatattgattgcagtacatttggtgaagaacaatactttcagttaagtacaatacatgaacttccaccatatgattgtgtaaaaacatggtttgatattcataatataattgatgatgcaatcgtacttccatttgggttgtcaatatcatatactgcacctaaaacgtttgtttttactgaagatatgattatgaaaaagatatgggcgatgatgccacttatgcttgattaacggagaactacatgaatcaaagtcatgatctttttaaattatgtgcatccaaagatgtggaaggatgggcagataaaatcaatacaattattaaccgttatcttggcttctgtgcacgttcacgaagtgaaggttcaatgcgtttccaaataaccatgacaatcgatggtgttacgatttatgtcaacgaagagcgtcttggtattattctctatgatagttgtgtctttgctattcaccagaaaatgggggcaggttggggttgtaataatatgaacaccaatgcttttttctgggtaggtcatagcaacacagtacttgatccttcaactaaatcaaacctattagatgataatatcgtatatgatttagcttactctgaagaacaacattttcagttaagtacattgcataatttagtcaaatatgaagattgtatcaaagcattagaattatgtcattcacttgaacttaaagaaaaattgatacaaatttacccgttgttcttccatgaagaagtacaatgcacactagatcatattttatatgggtacgacaaatgagaaaaagttatcaagaagctattgacctaattcgagaagttattaaggtggctccaggtgcgatagcattcaagaagtccctatataaaaagtatggtcccatgtcaagtatgtgctgtcaaaagaaacgtagtaaattcactacgcagtactggcacgattctattatcctgagtaatgaacatcagcgtattttaacaatcgaatcagtagataatcaatatggtgaaggaatgatttgtgttcctgactatgatgaaattaatttcaattgtggagattatgagactacattattattacacgagggttaccctgaaggtgatgtaatggcagagtataatccaaaaaccaaaatactgaaacgtggtatgcagttcaaaatcaataataaagatgatgtggaagcagcactaatgcaacttagtactagtgaacctatcactattgatgtagaagagttatggggcatcatttcagtacttaatgagtgttataaaattaaatcattacataagtactctatcattttacatactaattgtgcaatgataaaatgcaagaacatacaagagatggtggttccagatgaactttaaagaaatagaaagtaaaattaagtacttcaaagatattaaaaaactttgggaaaagtataaaattgattatcagttccaagatgatgatacggttcaaagcattgatattaatattcgtgtgaagttcaagaatggcaattatggcgaagttaaattgaatgctattggtgtattccttaaggattatgatggttcttcactcgctcaattcgatttcacacctgatcctgatgatatgttattgagtgaagtcaatgcatatgaccttgagatgtttaatggtaagcctttactattccgcttatcagatgaatgggatgagatttactacacatacaaagatggtgtgattcgagacgtaaatgatgaagtagtaaatgatatggaagaacgtctatttcaagaatcatgtgtgtcatcggagtcatatacatatcaatatctacttgacatgttcgatttgtatagtaaaatagattcagaaatgagtacattgggtattcattatcttacaatcaacgactttgcacaacttgaaacagcggttaaagccctggtcaaagtactgaaataggaatgaataatgaaactgtatcatgcgagtactgtaaaaattgataacttttatatcccgtatggtggccttcatatgggtggcaaacacagtgctttagaagctgcattacgcaagctgagaagcccactaaacgtcgttgacgctgatacagtatacttgcataccttagaagttgatgttgataacgtgcttctatggacggatatgggcagtgatgatgaatggcgtggagtgattgagtactgcgaaattaacggctatgatgcagttcagtacaaaaacgattacgaaccagatacagttcctagctatatgatttgggatacgtctaaggttcgcattattgaagttgatgcgatgcatatggatgaagctgaaatgatcatcgaagatttccttgatgaatatgaaggtgtataatgattgaacctatgacagtactagcacgtactaaattattattgaagattgctgaaatattcactgatattttgtacagtgggattgatatacatgaattaggcattaagcaaccgtgggaaataaccaagttctctgcgttctatcgtttatcatataaaggggaaacggttctggactggaaccccaaagccaaattcatggaatatgcggcgttatatggaagcaatggtggtataacaaataattttaaagaaattaaagtcctaattgggtcaaaggaatttaatgacttactatatccagaatttaagttaacagaagaattaatatttcagtactctttgattttaaaagatcttaatgatttagagtactacaaagagttagtagatatttttattaatactcgactttcaaaagatattgtgatattctggtatgttgagtatgttacagaggaattgcttgaagaatttcttctaaatatgacagcaatcaaggagaagttatgttagacattttaggaaacatcgatacagcaaattattttattcaagatgcactgttcaatacagtacagaaagggttggatgtacgatgtaatatccatcagggagatgggtattttgttgatatcactgaaacacgcaagaacgtgttcaatttaaacattacaacattagcacgtcagaacattgcacgtattttggtcaataaagatgaagtagttcttcgttataattccaaattcttgttcacatacaagaagaagactgacatgttcagtactaatttgtttgatgaacccttcaatctagaagacttagaagcaactttctttacacagaacactatgctgaaattcaatgatgttgaattttcatcagaagtaatgtctgaaacactacaaatggttaaggtagtattctatgcttttcaacagtttaacgaagaacgaaacaaggttaacaacaacagatgaactaatcgcggcgatggaagccaatgagaaatacttcaatgggattcatttttttaaaacggatcttagtgaaagtaaagtactatatgtgtatggctcccagtactgcattgttacaaagcaccccgatgcgatttcactaaaatttaatacttattcaaaaaacttcttgacgattaataaaagcaatgttactatgtctggtatagggaatcaggttgttgagattcctttggactcaattgattctattgagttctttcaatacgagtgcttaggtgttgattttggtactatcagtactgaatctatgcgaacattaattaatttacttaaaggtaaagttcatgcttttaactaattcgatgaagaattcatttaaaattatcaaagaaatactaaatgggtctaaaacttctagtgggtcttcctttactagtgaccagatccaacacagtacaaaggattgtactgtaatctttgggttacatagtctgcgtttgacaatagtaaataatattgataacacagcaattgtaatcaccccatgtcatattcagaatgaaacaggtgcatacttacaccacatggggactgtattctacatcttcaatactgaagaaaacattgttaaagaatacaacacactcaatgttgatgctggtgagttatttcaattgagtactgtcaatgattgtaacttcagtgacaatgcagaagtacttccattgtttctaagtcttctcaataacatggaaaaactaaccattgcatacaacaaaagtgacgtaccgcaagcagtattgaatatcaccgaagcaatggagaaatacaaagctgtgacctaatatatgtgcacatggagtgcctttgaagatcaaggatagatcatggagattaaaactgttgagaatggaaagatagatgacattttagagttggacgcattcaaagttcatgatataatgttcaatgtaaaggacataattcagtgcttgtttttatgcagtaaaactatgcacgacttctttcacaatccaaatactaatgttttcatactaacaatcagtactagcaattcgtttatcatgggtatgcctatgagtacaaatgacgagtacattgattgtgttattcgtaacaatgataatattaccccacaatctgaagtacgtaagaaaaagaatacatacactatcaaagatgataaaatcatgatgtgttgtagtaaagggtttattttggataattttaaagtgaaagatagttatcatgattttaaagtatcagagacacaaactaatgtagttctttacgcaagtgatattgaaagcctaacagttaaagatcccgatttggagtaagtatggctaaaaatgtaaacaactttgtacaaaagcatatgaatgagtttaataaacctcaaacacatgtggaccgtaaaaaagcaatgaagaatggtaaagtaaaacacaagtcgagatttattctcgactaattttttgtccgaaatttaggagattgtattactatggatacaacagattttcgtaatttcaaaaagtattcagtacttgaacatatcaacgaatttctgaattccccaacaccatttggtatcagtacatcagatacatacgatggatatagaatcttcccatgcaatgactttcaaatttctgtcaaaaaaggtacttcaaaagaagttgcaattcgtgatgtagtactgtatagcgattcttctatttcttactgtcatgatgtgaacaattttgaatctctttcaacacctaattgttcatactacaaatccaatgacacaatgatgtacaaagaagaacacatcaatggggtttcgttttataaaaccgccagcgataaatcatatacatctagagatgattttggaaaatatacatgcactatgtatacggatgatgaaaaatcaaatcgtattcattcattcgaatccaaattcaaagagagtgaagtttcaaatatcactatacaagaaaaggttaccgaagatggtgttgtgtatcgtagtactgcattcttccgaatgaaagatattgcaactggtcgcccaatgatgtataatattgacatatccatgaaaggtgaatcatcggaagtggtgattagtactggttatcgtgccaactacacatttacaatggaatcacctgatgcatcgttggtacatctattcaacaatttatatactttccaattcgaacttggtttcgaaacaaatgaatatcaaatatttgagatattaaaaagtaatattattaataaattggaatattacttcaagttaggcgggaagatgaacatagttaatactatgttacgcaacataaaagaaacaatgtaattatggaggatttggtatggatactaaattcaaacttattttcaaaacagagccgtatatcccttatactggtgaacgttttattccacctggtgcagtggagattgagcatcctgatggaacaaaattgtatctcagcccatgttcgtatgaacgtgcagttgaaatgctaattatgaatagcaaacattgtagtaataagtacattagctacttatacaatgaacatattctagaatttaatgatgagaatatgtttatgctggctattaacgatttcaataactggcacgaaagtttgactatttgataaaatattgtggacatgacaattccttgtgatataatgttcacatctttttttgataaatggagatatagatgaatacaagttctgttctaaaagctattggtggtgtacttctgattgaagtccagggtgaacctaagctatgttgcttagaagacatgaagaatactaaacacttcagtactggacagattttcaatgctattcgtgttcttaacgacaaatcagtacaagactatggaaaggtatggccttcgtatgtaaatcctgaaggtgaaattagtgctgattggaaaacaattgaagcagtacgtgtgagttgtgagagcgattgggatttcatcgttgaagtatgtgaccaactgcaagaagatgtgccagagattgtcgaacgtaaagaagttctagcacaacttgataaattccgtcacggtactctaccgtatgacgtaatctaatctgatttattaaaaactatctaactaacattatataaaaaggaatttaaaatggaagacaacggaatgaaatttgatatgtttgaaggtgtaccagagttcgatttcaataaacttgaaaatggtgaagaatcagaactagtaagtgttacactaaaatcagataccaaaaagaaatattccccatcagttcgttcacgttctcaagaagaatatgaaaccttcattgagagtattcattacgaaacgttccatcatgcagatggtactccgtatgaagttcgtgttacagtactgaagcctgaagtaaacccaatggaaaatctacgtcctgtttactgctgggcaagccactaataaaccaaaggacgggatataatatctcgtcttttttttgtctggagatatatgaaactttatctaatcgttgactacagtaagggttattatgactacgcagaagaatctgaagggtattgctcgtctactaatgtcaatgtacgcaccagtagtacacatgagcatgaatctattacaaacctaaaagatttcatcgcatatcgagcatttgaaaagaatttctgtcgtagtagatattcgaagtttgagtacccgttaattgatgttcctgatgaattgatgttccagttaatgctcactggagtacaagattatgattacatcgcgtacaatcacattactaagaaaatttatatcggtacgtcaacacatgacatttctatgtatgcatcatgccccgatgatcgaatgaatgatagaaaatcatactatagtggatcagttggttcaaatgattcattaaataaaaatttattctttgagtacgagtttgataatcatcgagggaaaaaagaccgtgaagattattgtactgaaattgatagtaccaatggatataaagaagtgaaagaagcgataatgccatatatccttgacaagtactacaaagattttcagtactgtagtaattttctaatccgaaattctatcataatcggtcgtcaaagtactgcattctctaagtactacaatgattcccctgttgctgaaaatgaaaactgtaatattctctcaatcccacgtgaagatatggtattcatgttcacatctttcattgaagatgatgagttgtatgtaaaaaacaagcacaggttcatgccaagtaagattgaggttatgagaagcttaagtgcagatatcttaactaatttcactgaaaaaacagaatacaactgagacagaccataagtttatgatataatgtaaggtgacagcagaatatgttgtcgcctttttttaatcctgaaaggttccatatggaaatgattaaagcctaatccactcgaaaagtagagggtagatgagtagtcctttcagtacgaacagcaaatgaaacccgttagatgatggcatagtttgaaccactcacggcaatgcggctgtataaagaactccaataggataaagagagttcctcgatagacatataaggatcgcaccttaccaattatctaagggagatttcatgagaaatttgcggtcgtaatttatacaatatagttgcgtccaaaattcgtgaaatcaaatacaagtactggtcatatggtatttgtattatgtgtcgggatgtttaagaccgacgaccgttgctacaagaaccgcgatagagagaaacagagcaacccgctttcgcctcgtaagagaattcgtgatatgaactatgtgaaggccaccagcgtcaaactttttttgttctttcccctgcggggggatgaaatatgctcaagcaaaccccgtcaatattaattaaaacaaatcatagtactttcgtcgaattagacatgcgaagcgtgtataattcgactggtgttgatgtagtcaacgccatgtacagtcattgatgtttccataggaataatgatgtcatttattaaaaaatctaaaagcgtcacttataaaacactagaatttgttgattcagacaatcagaaccaagaccaagagaattttactggttttgttgtgattaaagaaaagggatgtatttcgtacatcaaagagttgcagtgcggttttgaattaacttccattaagttattcccaaccaagaagcctacaattcttaaagctcgttatgggaactactacgaagagcgttatagtaataatctaatcagtgcgaaagcacatatcgtcaatggagaactagaaggagaatatagaaaattcaacccacaaggaaaatgtgtttgggttaaatacttcaattcaggaaatgatgtaacatcagaaattattgagtttttaggtttaacatgtacccttgaaactcttcatgattacaaatttaatactgaagaagagtttaatctgatgatgcgttatggatcacattttaaatttttatatgaatacaacattgacagtagtacttttgatgacatagtacttaactgtctataatcaaaggaaattatgtatgtcgaagaaggatgcttcgaagaaaaaacaatcagtacatttcagtactggaaaagataactggactacgccaaaggatttctttgaagatttggatgaactctgggaatttacactagatgcagcatgtgttaaagaaacagcattatgtgataagttctttacaccagaagacaattcacttgaacaggactggggaaacaatatcgtgtggctcaacccaccgtacagtgatttgaaaacgtggttatctaaggcagttgatgcatataacaaaggtgctacagtactgatacttgtaccttcacgtaccgatacggttgcgtttcagaactatgcatcaaaagaatgcgactgtatttgttttataaaaggtcgcttaaagttcggtatccctgaagaaccagataaaaagacggacagtgcaccattccctagttgtttaatagtactggataagtatttgactccagctaaaatcaaccatcttaaatctattggtcaggtgatgaagaacatatgaaagaaacatgtatattatcatttatgagatgtaacccaatacatcacggacatggcttagtgtttgaaaaagcacaagcactacaagaacagtacaatgctgatttgaaaatattccttagtaccagtactgatgccgtaaaaaatccattacctcctgaactgaaaatgtactatacagaaggattttacccatctgtacaagaaaatatctatacagaagataatctgtttgatataatgagagaactggatggaaagtacttagatgttcttttcatatgtggttcagatagacttgaaccattccaaaaaacgcttgacacgtacaatgggaagttatataatttttgttcaatcaaggcagttcaagctggaattgaccgaaatgtaagtcgctacagttccacacagatgcgtgagtttgccaagaatgaagattttaatagcttcagtacttttctacctggtaatgatgatgcactgaaaactaaactatttgaagatgtaataaaatacatgaggaaataactatgtcagtactgtttaaaactaacccagtactttgggatcagaatgaggattcgggtttccgttcaatccatgttgaattcaaaggtaatactgtggttgcaatcattgcaaacaaaactttaggaaactggactgcacaagttgttcctatgggatattcttatacatctgatgaagttgcgtttctggctgaattatatagtcaaacaccattcatcatcaatgttctagagcgtagtgataagttaacacatgacgaagtaattgacttcctggcgaacttacatgtctgatgattcgaatctaacaggttattttttgcaagaatcaatgatcaaattcagtgaacattttccagtactagatggtgtggagatgtggcatgtaaaaccactcttccacactttcagtaaagagcacaaaatgttattgactgaggcttgtaaatcgaataaaatatgccaaatcaagattggtgtaaatgtctacaaaggattagtcatcttttttgacaacaacagcaactacttttatattgttaaggagcattacgttgacaaatacaaatactaatacatactttcgttatcttcgcctagggctttccctacgcccaatccgtggtaagtgtgattcacttgctgatttggattatgcatatgcattacattgtgatggtgttcaatatgtattaggtgactatggtacattcgaagaatccaatgatagttcagaaattattgctggtgttgctaaccttaacaacattgccaatagttctcgtcatgaaatcgttcttgaaattcagaacttcattgagaaaaatactggcaagcgtacatggtcatatgtcggtactgatgcacaaatgtcaatcgcatttggtaacgaagatggttatgtagcacaacttattatggatttcgaatcagaagagaagttccaagaatacaaggaaaaaatcatccaatccttgttgcaaatgggtattcctgaagatcgtatgcctgaaattcaaagcattgatgagtttgttcttatcccatctgatgaaaaatatgatgcgatgtttacagcactgaacattcctggtgtagttgttgagtacttcgatgatggtgatgctgacatttacctagatctcgacgacgatgatgacgatgaataatacatatagcccgacataagttcgggctatttttgtaaggggaaatatgaattacaaacatatacaattcacttatatggaattatttaaattacaccacaataatagaagtactaacacaattaataaaaatgcgtttactcataaattatggtcaaattacctagaagaattacgtgagtattttgttaatactgaaattttcattaatgggaaaaaacgaaagatacttagagtcgatagttttggtggtagattagtactttcatataaatttgataacgacttattctctgttgatttggtgaatatatgaattataaagatttccaattaacattcaagtgcattcgtgacctagcaaataaaaataatagtatatttttagaagatacatgtattgaaatgattaatattgtgcgtgagaggattaaagaaaaattcagtactgtaattatcaacaacgatcatgcagaattttcagatataatcattaagctcgatggtaaagtagaggtacagttctacactgattttggatttactagtgttcctctggagatattcccttgaaattaaatttgaatgatgttcaattcacgtataaaaatatatatggtttgtatgacatgtatctttttagtgggaagattggtgaacatgtcgaatcaaaagaatcaccatttattaataattactatgattacttagattctattgctgaatatataaaaaagaattacagtacttacactgatggagatgacatatttgaatattcaggtgtaaaatcttatcaaggtattatccatgtagaatttcttgaaaaaactgatagggaatttatggagattaaagttatgaagttaacggacctataagagttgacatatgaatattaatgatatacaattcacatacaaatttttgtatatccaatacgctacggttgatgctggagaatatctctacggtgaaatctatcgttcggtatggataaagtttttggatgaattacatgaatattaccatgatttgtgtatagttcgtgataataaaatatacaaattccgcgatattgttagtcatagcggtaagctacgattggaattaagtactgatagtgatgttgattggattggtgtaaatttactggagttggagaatgaaaaacaacttaaaggaaatacaagtattttataaattcttatacttgtcatatgtaaaagatgagtactatgaaagtgtgtattattttttatggcaacattaccgtactatagtaagagatttatatataggaaaatctatcatatccaatggtgaaaaatgtatatgtactgatgttgttagttttaatggttctttaacactgtgtttcagatgcgataataatagtgggtcattttcattaaacgtatatcagttagaaggaataaattttgaataaatgtccatattgcaacagtactcaattagctcgtttccgtttagattcagactgggccactggtgctggtgatttctttcccatcaatgatgattcggaatacactgaagaacagcacaaagattttgataaatcatggcgacctgacattgagtgcatttattgccttaattgtgagcgtatccatgactaaagctgaactgatcgtgcaaacactgcgtttattgagggatttagatatcccacgcatggatgtgaaacttgaagatatctacattgaatctcttagtgaagattacaccgataccgtaatgatgctgaatgacgtgctttatcgtatagatagcatcagtcgtcttggaagtactttaaaagtgaatatggaaagagttgataaactttacgtacatatatatagtctacccatttcaatattaaactatagatgaatattatattgatacaaaaaacatatgcactactgatgaaagaatatactaatacttatgatgaaaagtgtagtgcaaatcataatatgtataaattcattactcaagagtacagggattatatatattctgcgttagttggtagatatttcagtactaaaaaacgcaaagaactaaaagtagtagatgttacttttttctacaacgggcatataacaattcatggtgtggaacataattcacaaatatatcgtaactactcatctaacgaggtaacatttttatgagagcattattttttatctggagtcttttctcactggcagtaatgacagtaagtgggatttgtttactttcgacaatgaatgtctggtacattaatttactattcccattcgtaggtgtaggtttaggatttactttacatagtgtttgtaaggattttaatggcaaataaatcgaatgaagtacttgttgctgaaacaatgaagatggtttatgccataacaactagtaatgatttgtcttacccaattaagtccaagttgagggatataactcttcaggttatacagcagtactacaaatctaaacacaaaagtattattaatgtttatcgtgactatgaaaggattcgcattttgttctggaacaagaatcgagttcgttcggaatgtgatatagaaaactttaaatttgagaatctttattgttacaaagcgggacaatagctcaagtagtgatataatattggatgaggaacaataactaacacaaggagtgtgtaaagtgccaagtaaaaagccagattctgaagtattcagactggttgatgaaaaattgagttcaattggttcatcggtaaaaatggatagaacaacatacactaccatatcaaacaaatcaacttttttctgtgatgaatgtgggtttagtaaaaatatcatagttaggaatgtgtacaaaaatggaatattttgtcactgtcccacatttaaaactttatataataaattgggtgaaattaattcaaaattaaaattagatatttccacattcacaactacaaaaaacaaatctagattttattgtgacgattgtgcgtttgaaagatatatgacggtgcatgatgttaagtctagaggttcatattgtaaatgtcattcatcatattgtatgccgttagatatcgacgaagcatataacactattatgaataaaataaaagattactcaaatacaatatatatggataaatctacataccgtggtacacaatataaggttagattttattgtacagcgtgtaattttgacaaatatattaattatagtagagttaaatgtggatctcttaactgcaattgtatcagaccaatcccagaagaaagtaaattatatattttaggaatatatgaacaaaatattttaataaaatataaagttggtatttctacaaagattgataaatcaagactaattagtattaggtcaaaaactaattttatagtagaggaattatattattttactggaaataccgaagttattaaaagtgtagaatataatattttaaatctgcttaaaaatagtagcaaattgccaaaggattattttggtgatgggtatactgaaacatttgatttacaagacataaatcttgttattgattatgtatgtaaatgtttactcgaaaataaattaaaaatatgaaaatcataaacacaaggaatgtgaaatgatagataatataaaatttgcaacagaaatggcgaatcaactcaatattgcaaatttccaaatgttaacatctgttatggctgaacatgctgcggcatattcatctagcggtatacaatgtggacaagctaacatgttagctgatgtttatagtgaagttcaaagcaaacaatatagcgtaaagacatacaaggataagttcgtaagtctttatatgaatgaagatatttcagaagaccgcaaattcaccgattttattattgaacgccgtattagtggggtttcagacccaaatgcagcaagtgatattgtattcgatgaggtattaaaagatatatatgaaaatgaattgaaaagtaagaatcattacaatgtacgtgaaactaacacagtgctagttggatatgatgaagacgaccaatatttctattttcgtataacagaagttccatttgaatataaaaaaccatccactacatcggtaaaagaattcactgagaagagtaaaaacttcaagaaacacgaaggaaaccgttgtagtattaatggttatgatgaaaatggtgttcatgtttatgaatgggtttcacctaattgccaaacatacacccgttgcttaaagaaacgttataatttagaagataatgaatccttctactttaaagttaagaaacaaggttatactcgccctgaagattcagaattgatgggtatggtatatttctaaggaactgttatggatttttacctacacacatggtcatacgaacaatacattacattatatcttgagattattaatactgatcccagatatggttgtgatttcgctgatattataaaagagtatgcagatgagtttgagtttgtatatcttggttctgaactatttgtaaagagtgtaggtaaaaccggacgcatcactgaaattgattatgatactgttagtgtgaatttctatgttgattttccttgtgatggcgttgatggtgtatattgtctatatgaaataatttaaattttaaggccagcgtttgctggtcttttttcgcaggtacaaagatgctcagtatagatttaattaacagtactataaagttgttggctgatgtagtgagagaatccaatggaactgtatacacatcaatgtataaaatctttgaacgatacatcaaggattgtaatatatccttatatggactggctgttgatatagacgaccactatgaaattaatacttcatataaaactggaatgttaattggtattaatataaagatgggatattttccttattactgtataggattaccagatagtactatggtagattacccagccagtactattaaactaagaggatagaattttgattaaagatttagatagaattttatatgtactgaggggtgtaattacattacaaccacaggttaatcgtatggagagagcctttgaattaaaatcaagagaaactccatattcacatatatcaaatgtagttggttcattcagtactgaagactccccttatgatatttgtgtatgtgaagtacagccaagttcggatcttggatttgctatttttgataaaaatcaaaatctcattagtatgtttcagtgcaataatcctttttggaaaacatgcctgtatccagagtgggctatgcttgatccaacaaagagttccattaaagtatatgactctaaaaatgatgaagtagttattgatttggatgagtcaatgtcagaggaagaacactttcagctaatgatgatgcgtgctatgcctgagtacaatgcaatgattaacactgtgcaatacattaaggataccatgccatcattgcgtacacatgagttttgtcatcctgtattgtatgaacatttagatttagacttcagtactattaaatttaaagataatgttattaattacattcaagagaatgttaaatttgattttattagtacaatgacaaataatccaaatactaatttcaggcatcagtacaacgaatctttgtctggtggtcgagatacaacttggtggaaataatatgaatatccaagatagattgcaattgttgcacacacttgaagaagctgcttttgaacttgttaataaaattgcacaagaactcccaaaaggaaccaatgttcgtgttcgtgaaatggattatttaacgggagaaattgaagaaaaagtactaacagtgatcaaagtaacatactatgaatctgagttatcaattcaatgtaagtcggataatggttacgtttcctactatggcacagatgtagatatagaggtaataaaatgacccttttacaaatatatgaagatttaatgcacttatgtaatacccgtgaggctttcttttatgtggatcataaaagtcctgctggtggaacgtatcgtgtattcagttatcgtctagcatcatataccgatttcatgcaacccaatgcattagaaacccgtggttctatgtgggaaattgatgaagacggtgatatgattcgttgtgctgctcgtactatgaaaaagttcttcaatgcttacgaaaatcctgctacaatgttccctgctgataccacatttgatgacattgcagtagtgatgcctaagttggatggttcagtaatcagtacttaccgtgataatgatggtgtaattcgttgtaaatcacatactgcattgtattcagatcatgcgattaacagtactcgtcttctcaatgagaaccaaaatttgtatcaatacattgacgaagcagaccgtttgggctatactgttaatctggagtatacaagtccagagtaccgtattgtattaccatatcaggttgaacaattatttgtactaaacgttcgccatcgcaagagtggtgaactattatgtggttctgcattacgtaataagttcccatgccttgcacagtacagtactaacatgacaggtaaagatattcattacactttccctgttcgtgctacattcaaagaaagtgttgaagaagttaagaaaatgcttgacattgaaggttttgtgtgcatcctaaataatggtacaatgtgcaaaatcaaaaccgactggtattgttctttgcatttaacgaaagacagtattataatcgattcccatttatatgaagctgtattaaatggttcgagtgatgatttaaaacagttgttcagtactgacgaatatgctacccaaaagatattaagaatggaacaactagtcttttcgtgctataatgctttacaagatgaagttgaaagcttcgtcaaggaacacaagcacttggagcgaaaggaattcgctaagaaggttcaggagtgtttaactaatgaactgaatcgtcagggcttggcctttgcaatgtacaacaataagaaagtcgattacaaagagactcttattaagtacatgaaaattgtactaaaggatttttagtactatctagcctagcacaaggagaagtgatggctaaacctaagtataaaaatgacggttccctttctccgacagaattcaggaaaatacaagaagaaatcgatgaattacagcacaaacgctctaagaaaggtgtgaagattagtaagaaacgaatcgagaatactttcgggtgtagtagtactgaatacaagaagaaaaggaaaccaaaaggtgttaagccgagaatacatgatgtaactcgctatacatcgaaacgtgaaaataaattcgacccactataaggagttcatatgaaagttgaagttcagcatagtattgttcaggatatgtcagtagttaaatttctttccttgaaagattatttcagatgtatcaagttcctagaagaatctcaattacagtggaccccattaaagtacagttcatccaatgatactgctagtattcgttgtgatgaatctgtacttgtgaaaatctccaagcgatttgggattgggaaatacaatgataaacaatgcacaaagtaatttactacatataaagtcagtagaagctgaaattaaaaattcagtacaaactatcgtcgatgaaatgattgacaaatacaagagcaatcttattggaaagaaagtcagatgttgggattacacattcaaacactggtacgattccgagattgtgaaagttgatgtgaatactgatagtaatggtctaattaatggtattgcgtatattggtgttttaatacgcaagggaacaccacaggaatggctcacctatcataatatagagcatgttaaatttttagaggattaaaggatgccaactatcacactaacagttggattgcctgggtgtggtaaaactacatgggcagtgcaagagattaaaaaagccaaatccaagactatcaacatcaaccgtgatgatattcgtgaatctatggctgggtcatatgatgcttacaaattcaacgatgctaatgagcagtacgtaactaatttacagttcagtgcagcaaattcagcagcagagaatggttggaacatcatcatatccgatacgaaccttaaaccacatttccgtaatctatggaaagtatgggctatagataatggttatgattacaaagaaaagaacttctatgatgattttgtacctaatgaagaccgtggtgcagatgttcatgaattctttaagattaaagagttcgtaaaactttgtaagaaatataacctccagcgtgagcgttcagtacaagataatatcattgacactatggcagatacgtactattatagtaaggcattacacattggttactatggtaattcacgagaagctgctattatcgttgatatcgacggtactcttgctcataaatggggacgtaatccgttcgatgaaagtactgtactccaggatacaccagacgaagaagttattttatctgttatcgctgaagcagtttttcataaacgcaagattatcgtcatgagtgggcgtactgaagggtgtcgtgctgataccgaacagtggttgcaaaattggggtattccttacgatgaattgcttatgcgaggcatcgatgataaacgtccagatgatgttgtcaaattcgatttgtatatgaataacgttgttcaacgttacgatgttaaaaaagtatttgatgatcgcgaacaagtaacatttatgtggcgtaagctacttggattaaaagtgtttcaagtagattttggacgtttttgattacaattatcaaagtggtagcgggtcatatttggacccgcaccacttttttcacaatggggacatattcttgttggaaatattcttcctttgtttgcagcactaaccaatgattttgaataatctgaaagttttcttgctttctgtttagcaacccatgatggattattacttcttttttcattcattgacatgatggcttctggtgacattttaacacctaatttagatttactaattttttgtttagtttcctcggacatttttttgccagtatttctttttcttattttttcaacctgttctttagataattttttacctgtcattccactagactttggtctattagcactataatcttttttaatccacccataaagtttattattttgacgtctccctgatggtgctttggtcatcatatatagtgcatattttaaccctttatgttctgggtgaattttaactaacaattgatgggcaagataatgttcttctggtgttaaattgataatattacttttatcattagttccacccaaacactttggtaaaatgtgatggcgttcaaagtaaccatcatttagtatgttagttctacgtttcaaaattaagttatcataaattcttttataatccactttacatacctccatttatatagtaatatatttataatgttaaattatacacatttttcaatatggtttggtatgttcgcttttaaggtacattatagtgaatgtaacaacacagatgttattacgaaaagactacttagaaaatatagaacgatactatagattgtctttgtatgaaaataagcataaaaattatagttttgataaactactaaattccagtactgaagattatctattttcatcaatgttttatgtgagaggattattgtaatggataaatttcacacagatatatcacagataggtaatcttatcaatagtctaattactgatttagaagaaaagtacttaggacgaaatgtatacgtagaaatgtatgaaacatatggtgttattgatatgatttattacagtgcattgaagtgtgaaatacgtttaactgtgtgtttatttgaaaaagatacattcaaaagaagcaatgcaattcgccaagttggtgtcgatgacatagaattatacgaggaacagtgatgtttgagcaggacttagagctattcaaaagttatattgagtacttagttacattagaaaaagagctaactacaaagtataaaggtattcgtgttctgtgctggtatgatgattcggattataaacgtgaactcaccattgaccgaatctatttcatcgacggtgtattgaatgtgcatggtgtaaatgattttggacattcatatacttattcgaataatgctaaattcaagaggattgacaatgaatgagtactatcaaaatttattcaattacataaaactaatagatgatcttgctttcgattttcttagtactgataaagttggtgagcctttatattatgattgtattggaacactttgttcagatgaaaagaatatattagaaaataaattaccgtcatcacagtgcttaatcaatggttccaaagcagtaataactgatgttaatttttataaatctcttaatactatttgttatgtattcaataacactgtgtttaaacatgtaacaagtaatattaaatctgtgaggttcttatgaatttaaaacttttgttagaacaggtacatttattgtatagctgtgctgatggttctgataaggagtatagtgaagtttatttagaactgtttagaacggaaatacacataattagaagatacttaattgataataaaactgaagtaaaagttagataccataatggtactttaaagaatggtataattattggattgatagttacacgaatgtctagtaatattaaagttgcagttagattaatgactgatgatggtgtctacaccatagaagtaagtgagattatatattaaatggatatacaaacattcaataaactaaagtactatgacaatggttttgctattatcattgatgaactagatggaataccatatgagtatcataatgatgtaatgaagatagtaaaaagtatttgtaatgatttaaaaactattttcgaaaatgaatatgtatacgacttaggtacaaacaataagaaactatacataaagaaattctgtggaacgcattgtggtctgatggttgatttaggtttaagtgctgacaacaatttcgaagcatgtagtttatatctggatgaaatgcgaatctcatgagacatatcttgattcggtgatataatttcattacaacactgacatggagtagttgttgtgaataacgattttttaaaaatgtgggaaaaaacaatagtaattctatctagttcattaatgcacattttcagtactgataattcttatagttcacatacatttgatttaatatgtaataactatgtaaatggtatatcagatgaaatatgtccattcattgcgtattcaccggatggtattgaagaatttaatatatgcaacatcagttgtggttttggtgattggcttgtgttaaatgatcgaggtggaatctgtttatacatcgaagatattggtaaatttatatacaaaggagtagaatatgttaacaatcgcagcagttattagtgtacttggtgtagttattgcaggtatgcttgcatacaattacaatcgtagtgtcatcggctatgtactacttagtctagtactaaccccagtactcaccatcgttactttattagtgctaggtgaacgtaatatcaacttcatgattaatgataaacgtgatcaggttacaccagaacctgaaaaagttattcctgaagaacaagaagaacttaaagttgaagttcaaatgcaagtgaatgttcacaatgcacttgatgaatctgaccctaaagttaatgttactgttgttcctgtagttaatgaagtaaagcctaaagcagcaccgaaaagtactaaagcaccaaagaacaccaaaactacaaagaaagctcgctatactaaagggaagaataaaaacaaagtccaagatgaaccacaacaaggtacacacgaccatacttcccatattattacagcagccaccaccgcagcaatcgtaacgccatatatctcttctgctgatactggttcatatgatagctctagctgtgattcaagctctagttcaagttccggtagctgtgattaacaatgaaacatcttgcacaatataatcagtacttgattgcgttgcatcaaatgaaagatgatttctataaaagtgcagaaacagaactcgtcggtaagatagtaaaatttactgacgagtacatcaaccgatatgtggaaatggacgatgatccagagtatgaatcatactaccagtacataaatcccaatacaagaatgagaattattgaaattatgaaatacggtatacgtttaattgcacaagtagaatacatcaatcacgatgcaattgatgccgctgcctttggtgatgaaataagtttagaatatcttattcaagttgatgaggaataataatgactaccttttttgcaatatatggatttgttgcgttttgctcatttatatgtaactttctagtactgaattggaaatgtttcaaagatgaaatcataactgcaatgagtctacaatcagcagcactaatggctataatttggcctgtaactatggtgtatctttttatctcaataacaaaagatattatatcccacttcaagagaaagaaataatgttcaacaccgactattcacatttgcgatatcttatgtctttggctgatgattttattaatgatttgaagccgaagtatattggacaagaagtacgtttgataaactcaaggggttatgggaagtacattatcgctgatatgtatattagcggtggtgaatggcgtttcaaagttaatacgtatcttaaagatggttcttttgcaggaacacaagttacaaaacacactaacattatatggttgaaataataatgaaacattttttggaagttacagtaccagatgataaagatttatatattgttgctgatattcatggtaatgggaaactgtgggatgcgactaagaaagaattcggtatcactaacgaagattacgtgttttctgttggtgatgttatcgaccgtggtgaccagaactccaagcttctgtttgaattccttttcggtgaaaaccgttttatgattgctgggaaccacgagatcatgtgtgttgattctgaaactcaccgtgactggcatcactgttgggtacaaaatggtggagataagttcattgatgaagttggtgagacaggtatacagttcttccgtcagtacttaaaggatctccctatgattatccaaatcaatcaccgaggattgaagattggactagttcatgctggtgttccattggaatatacaacatgggaacgtttcgttagtgggacaaaatcacttagtgctgatataattgagaagtgcttgtgggaccgtgatacattcgactactgcaagaaaaataacacatattacgtccctagacttgatggtatccattgcgtctttagtggtcataccgcagtatccgacccactcatttatggtaatcgtatctggattgatagtcagttcttaactggtgatttgactcttgcaactccccaaagtggagggatgagatacttacgtaaagaaaaagatgagtactcatttaaccgcttcaaaaggtaataatatgcatgatgtagttaaaaaatacttaaatgatagatatgttgtgaaaaaatctattaattctatcctaaaagaccatgctaatcaaatacgtgagattctatcaaagaaacttattggttgctcagttgaattcaactacaaaaagaatgtggtgaagagtggagttattagtcgtgtagttgctgtttatgaccaaaatgtatatgttgaaattgaatatgatggtaaattattacaattaagtactgaagatttaacatttttataaggaaatagaaatgaaagtaattcaagttatggtagaaaaataccaaaccgaagatggtcgtatttttgattcagcaaaacaagctgaacaccatgaacgtattatttctggagcacgtaaagtgtgccctacatgtaatggtacacaacaagttccatctgaagactatcgcagttattatgcttgtccacattgtgatggtaaagggtttgtagaaaaacaagaggtatggaaatgaaattatattgtttaacaaacatgtactgtcaaggtgtccaagctggcattcaagcatttcatgctggcataaaactaattaaagaatatgaaggtactgctttcagtactattattgatgaatggtattatgaccacgaaactattgtagtactaaatgctggtggtcatgataagttaaattcttattatgatgacctttgtaagcagggaaaagtgccattcgctaaattcaatgaagccggattaaatgatgcaactacatgtgtagcagtactatgtacatctgaaatggttgaagacatgtctgttatgcgtatgaatcttcctgctggggatcaactaatgcaagtaaaatatggtgcattgtttaacacattaaaagcaattagcacaatgaggactgtttgatggattttccagagaagttatttgtggctcgttcatatgtaaatcataaccaagttactggttacattatagatgctacaaaatcacacacaaaagcgtttgagactaaaaaagaaaaggccgagtactgggctaagaaaaacaatctaccttcactatggattgataatgtgcctacaattgggttttctgtggtacataagctcactgttcatgctcgcgaaacatacttcaccatacgtcaccctgaaggtttcgagttcttggtgagtgctaaaaacatgaacgaccttatcatcaacaatgatattgttaagggtgtatttgttgaacctatgttctttaataatagtttagaactaattaacggtaaaactaaaacctttgctaaattggttgagaaggagaataagaaagaacatcaaaaagaagttgtttctaaattgaaagtgggtgatggttttagacacaataaagaagattactattattgtggtaaagtaaatgctatttgtatcaagaaaactaaagagttttcattttctgataagagttccaagtatcacttagtatataatactactcgtgggacatatgctcttaatgctcgtcttgaaacttatgcgttagaaccaagtgctgcactgaatgtaaaaactacagttcaagatgaaattgttaaagctaatgcattttggcaaaatatccaacgaaatcgcagtaacccattatgtgaaagcaatattccagtactgataagtgataagtcgtttaaagcacaagatttgaaagtaaagtacgaagaaatcaatcctgatgatataatgagtaatggacgattcaacgagaacgtaccgttcatgtctaaagttggtgaccaagcatatcgtgtgttctttggtgtaaagaacaatacaaccagaagttattatggtcgtaatggtgattatattcttcaatacactgatgtatttgaacgatacagtgcgtatccagttgaaattgatgctaatggtattatgacaatggatgtggatttaaacgaacatgtatcatttggtacatggaacggataccgttctcctttcttcccaccgactgaaaataaaacatcatcaactcgttactaccaagaaaaaccttgtcacgtccagctatcactaagtaataaactttacattggaaagtattatctataagggaatatacaatgagcattttagcaattcttgaaagcattaaaaatactagcagtactctgggtaaaaaagctatccttgaacaaaacaaagataaccaaacattacaaaaagtactgaaatatacactagacccttcaattgtaagtgggtataaaaagctaccacaagctgatgatgtataccaacatatgacattagacgaagcaattgataagctagaatgcatttacaaacgtggtgtgaccggaaatgctgggcgtgacttcattgcttcagtactgggttctgtatcagaagatgaccaggaagttcttcgtaaaatgctcacgaagaaccttgattgtggtatccaagaaaagaactgtaatgatgtgttcgggaaaggtttcatcaaagacgaaccatacatgcgttgttctttgattgacaaaaagactgcaaagaacattaacttcaaagattatggttatgctatatcagaagtgaaaatggatggtcagtacttgaaccacgttgtcattaatggttcattaacttctacatcccgtaatggtaaactatacgacttcctgggttgtcgtgatggtgaaatggcaggtcttgctcacatcatccaaagtaaagatgaacgttttagtagtggtgtagtctttatgggtgaaggtgtagtactggacaatgatggagaaaccattctcccacgcactactggtaacggtatcatccagaaagctggtaaagatagcatcagtcaaaatgaagcaatgcgtactgtattcgttctatgggatgttgttccatatgatgcattttgtgaaggtgtatggaatgtaaaacgtaaagaacgtcgcgagatcttagtagatgcaattaacgaactcggtagtgattttgttcgtatggttggattccgtaaagttctaaacatcaaagaagcatttgagtacaatagtgaactaatgaacgaaggtgaagagggttcagtactgaaatgtgaagatggtatctggaaatctcacacttctcctaagcaattgaaaatgaaacttaaaatgcaattggacttgcgtattgttggtttcaatgaaggtgaaggtaagcgtaaaggtagccttggttctttacaactggaaagtgcttgtggtacattgaaagttaatgtaggtactggaattaaagagaaagacgcagaatggacgtttaaaaccatttgggaaagaatgcaagaactcattggtcaagtagtaaccgtggagtgcaatgatatagtgtatgatcagcgtactggcgaaccatcactatttttacctgtgtttattgaatttagatttgacaaaaacacttgtgacgactatactcgtatcttagaaattcgtgatagtgctgttgaggtgttctctaacagtattcttgaatcacttggttaatcatttataggaggtaattatgaaatttgttgtaacgtcagacgtacacggtggcactgattacctccatagtggtgattacagtgatacagtactgattattgcaggggactttgatgaagctaaacgcagtcgttatcgtactggtatcgaatccctgtgcaagcagttcaagaatgtagtactggttcctggtaatcatgagtactacggttcaaacattcacaagactaataaagtactgtccgatatgcaagatgagattccaaatttcatctttttgcaaaacaaatttgtatggctggatggtgtactgctggttggtagtacactgtggactgactttgataaaggcaacgcactggttaagtttgatgctcaacaaaagatgaatgattataaacacattcgtcatggaacaacatcagaaccgtggaaacgtaagctcaaacctgatgatgttgagttcatgcatcatcaggataaacaatttatcaaaaattcagttaacacagttcgtctaatgaatgataatgatttcaaggttgtggtggtaacacatcatgcaccaagttatcagagtgtatctccgagatacaagaatgactcactgaatggatgttattgttctgatatggaccagtacatcgaagatttgggtgctgatgtttggattcatggtcacgttcatagtagctttgactacaacattgggaaaactcgtatcatttgtaatccaagaggttatgaatcttacagtggtgcatgtgagaacttcgattacgatcctaaatttgtatttgaaatctaaggaattactatgatatatacattgcatataaaagacaaaatgcttgttatattgggtagctgtcatttgtatcgacgatggggtatgcgtatactatgctcttgtgataaaacccaatactacaatcctgatggtactggcactgtgatgaaagataggtataacttcatcagtgatgtaagacaagcagacgatttagattcatttgtgggaaaggtgtatgttcttgtacatgatgatagtcgtggtgatgtgttaccattcagtactttatcaattaatacgttctttaaatacgaacaattttcaaattcactaagaatgattgagattgattttgatgacatcccagttggtatacgatatgaatttcttcatgctcttgcgtcaataggggtgtactgatggaaattatcgttgtgtatgtaattgagtacaatgatgaaataaagtacgtaattaatggttataaacattactccagtgcagccagaaagctggaaatatattcaaaagaagcaagaaactactgcttagatgatgattcagttgaggtttggattgatggaaatgcagagtactttgatgctaacttctatgaagatcttgacaaatcaaatgaattatacatttatgtagaaaatgaacgttattacacaaattctttgtttgacattaccgactctacagtgtatgataatgctatggttaagttgttaaagattgaccacgatatgtttaataaacatacaatttgcggtttactagattttttaagaaacttgtagacaaatacgaacactgtgatataatactcacatcaatgaaacaatagaggaaaataacaatggttgaacaattcaattttggtggttatgctggtggtgaagttcgcaacatttctattccttatgtagttgaaaaagttaatggtgcagaacgtacttatgatctagcatctcgtatgatgcaagaccgtatcattttccttgatactgacttcaatgaccaaatggcacatatcatcaagcaacaattggtgtggttggattctcagggttctgaaccaattaccttctacattagttctcctggtggttcagtacacgcaggtttgggtatcgctgacattgctaagtcttgtgtatctcctatccgtacagtagtaatgggttatgcagcatcaatgggttgctatacacaatcagtagttggtactaaaggtttccgtctaggcgggaagcgttcttacttcatgtgtcaccaagtaagttccggtactcagggtctgattactgatcagaagatttccttgaaacactctgatgcattgaacacactactgactaaagaaatcgctgaagcagttggtgtatcacacgaacaactactgaaagatgcggatcgtgacctgtggttgaacgctgaagaagcactcgaatacggtgaacatggtttcattgatggtatcatccttggtccacgcaacgaagaaggtaagtatctcgtcaaacgtcgcggtggcgtggaagagtgggtctaatttgaatttcatttgatgcctcatttggggcatcttatttctggagattatatgaaagttcttgtacgtaataatactgaagtcaatgatgtgttagatgattcagagtttattaataccgcattagatcagattagtgcagtggctattgaattgaaaggtgttgacgaggcagaacgagtactcaatggtttttctattgttctcgaattaatgcaagaagttcttattaaaaagaacattaatccaattaacgcagttcaacacgcagaagctatgcgtcaagaacatggcacattctcagataaaaaattcatcagtactgacgaggggaaagaataatatgtctaatgcacaacacaaagaaggttatgacgcgtttaaaaacaaagtagtttttaacaatgaacagctaaaatcacagcacccactattccgtcaaggttatcgtgctgcacaagctgaagctatgaagaaaaacaacgtaacattcccaacaactctgtatgatattcctaaacgtccatcagtgtttaaagcagaatggactgacattatggcagcaactaaacgtcctcgttttgctaaacttcacactgatgaaggtcgctggatgttgatgggtattatgaaagcagttgagaaacaggcacataccatcgttcgttacaaagacgttgtagctatggttgataaagcatttgatcttattctccagtacggttatgatgaaggtgtactaactgtatctgattgtgagcagtaccagtacactccattccagaaataattacttagggaatctttcatgattcccattttatttgctgcatatcaaaaattattgatcaaaaattgatccatacgaggtaaatacccaaggtgtaattgttaatagggtaggtggatatgcagcaatataaattatctgaagatcaaagtgatttatttttcaatcacatgtttttaattgatgaatttgccgaaagtttaaattcagaggcaatagctatagttaaaaagtggtattcttgctataacaagaacaaaaaatggtattggttgaatagaaatttaaaatcatttctaggaacaataaccagtactgatagtatcgaactaatacagagttctatcggctatagttacttaactaagcataataatacgtcaatagagtcaggcttcaaaatcaagcgtctaatgaaattgacgggattgagtgcagaaactatacaaaacgatacagaaaagttatggaagattatagaattcatgaataaaaatatgtcatttatatatagaaactatgatatattcactgatgtacgacaattttataagagtccatattcacttaatgagaaatatttccgacacatggaattatgctcaactatggttgatgaatggaatttagaggattaacatgaaatacactgctcttagcccaactgatactaaagtatggttgagtaatttgaacaaaactattattttaaccaaaaaagtgatatcaacagctaattcattagatgtaaatatcaagaagaatgttcttaaatatcatcaccgaagtaaacttagtacttggtactccaatattttttacggtttatatgggatggatgatattttaattggtacatcatctgggtcatgcttcagtgctggtggtatgtgctttatggatttgggtttttcattatccgatgaagaaatcgagtttaaacaattccgtgacaagttgctgggttcgggtgatatcaagttctgtacagaattaatgggagcatgggacgtttatgcagcgaaaccatttgacattgaagaatcagatgtgctaaactaccttcgtattcttaaaatacattcgaactgtaaagccaaattacaatccctaggagtatatgatgaaacacttgacgtggttgaagatagccgaagtcgtggctgaagagagtaaatgcatttcacagaaagttggttgtgttattgttaaaaataaccgagtattgagtactggttataatggcactccatctaaacaacctaactgctgtgacgctaatgagcacttagttaatggtggacgtttccaagactgggttagcgatgacgcaaagcatgaacaccatgaatggagtaatatccatgaaatccatgctgaaatgaatgcattgttatacagttcacctactgaacgtcagggagcaacactttacacaacgctacaaccatgctatacttgttctaagttgatcgcgggaagtggtattgctcgtgtgatttatcacaaaagttatcctcgcactccaccagaagctattaaagtactggaagatgctgggattattgtaaattgcatttatgataaggggttgtgactttgactttcttgattttaggtattccatttgcagttgtgattcttgtacttttagtgatgatggtattaaatgataatactactccaccagtgatgcgtaatcatgaagtacgattttcagcacaaaacaacaagtactacattgtaaattgtttaacttgtaaagagtataaagtaactggtaaatttggttttaccatttattataaagaccgtgttgaagctgacatgatttgtcagaaacttaatacggtagaataaaaaaaggagccataaggctcctttttcttttgttagtacacaacttgtttgaatttcaggaaaactcttagatccaacaacgagttcagtactaccacgttatttgttgacaaaagcatagacattatacattagtgtgatataatcattcaaaagaaggtaaaaaggtgggatgattctgcgagggaatttgtgaagtgatataaaaaacaatttgacttaagttaaatcttcctataatattatttatgtaatcaacaatcaactaagagggttaaccagtggtacgattatgtaaagaaatcatccaatctattattattattttcttaattaactttcctgcaagtgcaggtgttgaaagaaaaagtatcattagatatgatgaatctaaacagacagttcagttcatttcaactttaactgaaaatactgtaaaccgtttgatggtttttagagaaacacacccatttgacactatcatcattacgtcacctggtggtgattttgatgcgggagtggcactaggaaattttgtcaatgataacaatttaacaattattgttgaaagatattgtaattcttcttgcaccatcgcattctttagtgctaatatagaaaacagatacatgatgaaggactcaattctaggtctacataatgtgtcaatcgaaagtaaagcacgaaatagtgatgacacatacatatcagttcgccaaatgactatttttgctgaacagctatcaactaaggttggtgttctatttgcgttatatgcagcgaatggtattcctccagaattgttagtggaggttagtaaggttcgtggtaatgaagttgtgagacttacaccagatcagttgctcgaattaggtagtatcattcattagtattagatacaaaataataatattataataagaggatgataaagtggtacatcctttgttataagttcacatacactaggagtgtataaactatgacaaatttacaaaacttaacatctcttgttttttatgcaacaactattgtatattgcattacattagttttaacatgtttcatctataaagactacaaacgtggttttatggattccagtacacacaaatttgcatccaagttagaagcaggaatttactatgttctatcaatttcttatggagcatgtttacttacactagggttgctgatatactggttgtatacggcactttttgcacaaggagttatttcatgacttgtattatcgctcacacaaatggcacatcttccttcatcgctggtgataaattaggaagcaatggtttcactaaagcagttcaaactgagcctaaagtgtttgagaaagagttcattaaactcaatgaggatggtctttctcgcactaaagaagtaatggcgttggggtatactacctcgttccgcatgggacagttgttgaattacaacctaactctaccagaacaagttgcaggggaaaccttttcacagtatctagtactgaaagttattcctatcattcgtcaaatgtttaaagatgaatggggtgctcgtgatgcatcacaagacgttggtggtggtcagttcattattcttcataatcatacgatttatgaagttcaagaagacttttcagtactgcaacctaaaacacaaattactgctgtcggaagcgggacgtatcatgcaattgcatccatgtcagcgtatattgaagttcaagaagaaaccaatgttcagttacatgaacgtgttgagtctattttcaaaattgtcagtgataatgtaacaagtgttagtccagagtttgatattttgaaatactgagaataataattataaagagagagtatatctgatggcatacatagtttacgagtggattttttgtttcatagttgtgtttatttgtgtggtgttatacaatcacactaaatgtttataccgagaattcaggaatggtaataaacatggacacaataagtggatgtatgtctatcatattgctatcattgtatttttagtattaaacttgatactttttatataaggagtaaatgtgatatcagataaatttatacaaaatgctgggaacaaaagattacttataatttcaggggcaggtctttcagttgagagcgggatggaaccattccgctttgatgatgggttgtgggacaatttcaaaattaatgatgtttgtaacattgcacgacttagttctaagtatcatatagttcattcattttataatacattgcgtgtaaaattaggtgaatgtcatcctaatgaaatgcattactttattaaatcattagaagatttgtatggtgattgttttttacacatgacttctaatatggatgatctatatactagaattggtggaaccgaactcaaactacacggcaatttatgtgaagtaattgagaacttttctattaataccgatgattacaatgtcattaattgtggttatgaaccatatattcctaaagctgatgtttttgcaaaaccaaatgtagtgatgattggtgagtatcatcgctacgttgatggggtacgtctccccatttatgaagaacgaaatgaagtgttgaaaagtctcacagaaaatgatacagttgtagttattggaagtagtgatacagtactggtatggtccagactagctggtgcacttccatgctatagtgtaaatgttaacatccgaaaaaatgataatgattttctttttaatgagaaggtcatcaaacctgcgacgaaagcaattccacaggttgaaactataattaaggaacgaatgcgatgagtaaagcggaagtaccactggggcaaatggggaaacgtgcaaattttgtattctgtttcttttatgcaaaagaatgtaccccatacgctggttatgaactaaatgatatcccagtggttaacttagtactcctacaagtgcgttgggatggattgttgggtgcatgtggtggaacagttgaaagtactgataagtcattagaagatgcgttacagcgtgaagttcaagaagaaatgaactatatcattgacaaaagtcgcctgtctccattaaaatcacttgaattgtttccagagggtcactcacatagctatagttatgaaatgacttatgctgaacttaaaaaacttcgtgataattatgcatatggtaaacatgctagtgcagaggtggctggtgtaaatttatgtcacatctctcgttattcgaagggacaaaatcgagaagcaggatataataatcttatgacacagaacttcatcggaactgcgaaacaagaattagagtatcttgtacaaaaagaaaacttactaatcagctatctataggagtttaaaatgtcgtactcaatgttcttggatgatattcgtgattatcaaaaatattatcctgaccaagagatggtattgtgtcgtgactacgaaagtgcatgtgaatacgtaatcaaaaatggaattccagcatttgtctcatttgaccatgacttgggtgacttgagtacagatgagaaaactggatacacttttgccaaatttctaattgattatatgtttgagcataatattaaagaaccatttaaatactatgtacatagtagtaatccagtagggaaagttaatattgaaacttacttaactaatggatttaatgcactatgatttcaacacatatcaagagtcttatttgcacaacgctattaggtataggtgttctgtggttcagttcatatcatgagaacactaaacgttataatattcattacaatacagcgactgtaataggattaggtcaatgtaatgaaaataaatgctcatttacttataaaaccagtactggtgaagttcgtaatgggttatcaagagatccagtatcaattggtcaactagtttaccaggaatgctgggaggaaaaggctcgtggttcaagatgttatgttgaatacgaaccgagcatgtaatattccgagtcactaaccattagtcgagtacaaggatgtactcataactcaaggatgttttatgaaaaaatatacaattgacacaaacgtactactatctgatccaaatgctaccattgcttttgatggagcagctatcattctaccgttcacagtacttgaagaattggattcaatcaaaagtcgtaatgtggatgtttcccgtgatgctcgtgttgcaatccgtaacattgaagctatcctttctgatgcatcacatgaagaaattagtagtactggtgttaatattgctgatgttcatactaatatgagtcctgaaagcaaattgtttgttattacaagcgaagaagctgaagcactaattcgtacttctggtaacgaagagaaagttcaaactcttctaaaaagcacagtaccagatgataaaatcattttagtagcattagcaactgattcagtactggttacccgtgatatcaatatgcgtatcaaggctctaagctacggggtggaagttcaagattatcgtcatgatatcgtgattggtgatagtgatttgattcacattggtcaacatcacctcgacctagatttttgggatgtgtatagcaatctaactgatgtgtctatccaatctgaacaagttggtcgccgtttgtatcactacattccaaaaagcttgattgaacatatcatgccacaaaatatttgcatcggtgattacctatatgataatgctgatgttctatttgtgtacgaaggccaatctgcaattgaagaagatgatggtactttaacaccagaagatattcttgtattccaagatattggacaatctgttgctctacgtgctaaagtgtgggatattaaggctaagaatattcaacaagcactagcaatcaacagtatcctagatcctgatgttcatctaacagtactgttgggttccgcaggtactggtaaaacattgatcaccattgctactgcactggagctagtactagagaagaaaaaatacgaccgtatcatcttctccaaaacacaagattcccaattcgaagacattggtttcttgccaggttctgaggcagagaaggtgattccattctgtggagctgccattgatgcgttggaattcctacacaaagatgatgctaacccacaaggcagtatcgaagaaatcatgaaacgtaatgttatccagttcaaagcactaaacttcgttcgtgggcgttctttcacaaacactatcctagtagtggatgaattccagaacattactcctgctcaggctaaaacaatcctgactcgtgcaggaatgaactgtaaagtggttattatgggcaacttgagccagattgactctaaattcattagtccagttaacagcggtctaacttatgttaccgagaagttcaagaactgggacggttgtcgtgtaatcgaactagaaggtgtcgttcgttcaccactggcagaatttgctgaagaaaatatgtaataatgtatgggacattcgtcccatacatgatataataatattgttaacaacaaaggagaaatactcatggcaaagcaacctaagaaaaaagaaccagtagcaaaagttaagaaagttaaagttattcaatctaaagaacaacgcaattacgaactacgtaaagcagaaaccctatcaggtcttcaagctctacgtaaacaaggtgttctaactcgtgctcaataccgtgagatagtagcacaaaaaggttattggggcatttgaagcaataaactggagttcatgataaatactttcataagaataataatactttttattaatttaaaaagaactccatgtgttatttcagtttttcatccctgtcttttggcagggattttttctttctagatattgacaaaaggattaaaccatgagaacatgtagtaaaggaagaatgacattccattaggttagattaacttaaaggagaaatatatgttttttgaccaaacatttacgttgtaccatgattcaggtaaagttgagattattgataatctgttagagtacattgcggaacatgatggtatagaccgtttccacagtaagatgaaattcgatttagctgaattcaacaaaaagaatcgtgaaacattttggaagaaccgtttctattacgaaatgtatccagaaaagttcgtaggttattatctttgtactgaaaaaggattacttgtatctcctgatttacttatgggtgaatacatgatactctataatgagtactgggaacataaacgaaatgctcgtgacactgaatggcgacacaagcgttcgtatatgtacaaacataaccactccagatacaaacgtcctaagacccaacaagagcgtcgtatggcttgtgcagtactgaaggatgagggggaaccggatatacgtggaagacgtaagatgcgttcactaccatcgtattgggatgatatcagaacaagacacagtactggctggaaatacagtactaaacgtaaacatcaatacaaagggagctaatgctccctatttttataggtgaactttatgaacaaaattgaagttgctgtcgtgattggttatattgaaattgaacagtgtggcattgatacctttattgacactcttaataagtaccgttcagagtacaagggatacattagtctttcaccagacggtggttacggtggttttgatggatacaatgtaatttggaccagagaagaaactgacgaagaataccatacacgaattgaagctgaatctaaaagatgtgaagaaattgaacgtcaagaaatgaaacacttgatgaacaagaaatcattaagtaccgaagagaaatctaaactgctaaaatacatcaaggattacatgtaatgaaaaatgtatcacaagaaaagattctgtaccttgcaaagtacaatggtggatttaatattaccagttgtgaatatcatggtaacatggctaaaatgattgacgagctactagaaggtaaaatgctccgttgggagacaaatgacctttggtacgaatatgcggggcaacaatgggaaagtgactacgataatgcaattcatacgtatacaattactaaaagtggtgaagtacgtttggctatcatgcgttgtgcttacacttacaaaaatttcaaagatgaagaaaagcatattcgccgaaatgaagagttagtacacctaatgggtgaagctggcattacaatgcatgacttgttcaatgaaccaagatggactgggaatctgaccaaagcagaatacaaagcattgcgatacatccaagtaccagaaatcaacaaaccaaaagaatgtattgatgatggtgaactaagcctcaaaaatgtatttgaaagtaccagtactgaaagtattgctgatctttcagttcagatggctatcaaagctatggaactattcaaaacacaatatgatggtgttaattcagatgaagtagagcgtgagttcattacgcgagtacaaattgcattacataaatcaaaaaagtaggtttatatgaaaattttacttgtactaatcgccgttcttatggtattatcttgttcagttagtaatggttcatacatttacgtatcagtatttgaagaagcaccaacgaactgtgtattaactcacactggtattgtgttagttaatggtaaaccatcaatcgagtacttatgtataggtgattaaattatgaaatttatttctattgcaattgtttcagcactcatgatgttagtaacaggctgtgcacctaaagttcaggaggctgatgcagagttcattcgccagaatttgactaaacttaactatgatggtacattgtatgcttgccatgagatgcgtcatgggcaagatcttaatctcatgtgcaggaagattgtaaaccgtacacgccttaacactattccaaaggatgcacaatgaaagaagttattggatacaaatgcaaaaactgcggaaaggttcagggtaagcacggtgtagaagggatggcttgtccgaagccaggtcgtggtaatttcagagggttcagtactgaaaagttttatgaacctgattattcaaaaccagtaatgggttataagttatgagtgattatgtggcatttcttcatcgctttggtgaagctcaagaaaaacacattatccgtatgccagcttatggtgagaaagactggtacaaaagtactatcagcgtaggtgcatttcgtgaccgtgaagctggattctttcttggtaagcaacacgaacgtgaagttctttgttcactgtggcgtttagatgaatgggatagtgaaaattccagaacggctgaatcaatgctggcagttgttggtattgcgaactatcagcacccatatgatctgttacctacgtttacccatgcttctttgtttgacttttacaaagaagtagggtatgattataaaaagaaacgttacatttaatatcagtactgttatagtggcagtactttttaattggagattacaatgactacatctgaaaagtactggtggattatgggccatcctaaattcatcaacggagatatggttcccccaaggattgatatcgaaccacatgatgtgtgtccaatcacaaaccgcattgaatcatttaaagctctgaatacaaagacacaattttgggtagaattcatctcacctatgtacgttgagcaagatgatgaatgggtacaagcacatgattatgagttagattgcggtggtgatacttatgatgaggcaattgatgaactatacaaaaatgtgctaaacacttatggtgattacacccaacaagaagcagacgatacgtttaataaggtccatgcagtcatttctacatcatcaatttacaattcaacaaaaacaatttgggaagaacgtcacgggtggagttcatctgtaattgatgctgaagccaaagaaacaattctggaagacataaaaaatataaaaagtacaatagaagccttgcgtgaatttataaagtcttgtacaatagaacaacagaaagaggcaaagatgcatttggagagtgaagaatgtaaactttgggttctagagaaatcatttgaaaagggtatagacttagacttatgaatcgtaaacttgagaaactaattgaagaattgcgacggatgccaccaatcgagcaagtggggagctacaaacgttatcgtgtgattgctcgtgcactagataagaaaggtcgtgtattggcaacacgtactaattcttacacacatactcacccagtacagaaaggttttgcaatccgtgctggtcgtccacaaggtgaatttcttcatgctgaaatctcatgtttgctggcttctcgtgctgaagttcatactttgttgattgcacgggtaagcaaagatggtattccagtccctgccaagccttgtgagatttgtacgttggcaattatggaatatggggtaaaagaaattattcacacatgaaaaaaattgaacgctacaaaaaccatacagttgtggttgaagatgatatattttattattatattttcagacccacacactgtttacaaataacgacatttgagaatgataatttcaaagatgtctacttccatataacagatttagcaaaatattccagtactgaacacttttccaatttagtatttgtgtactgtataccaaatcataacatgtaaggagtcaacaatggctaaaactatttctttaaaaaatgaaaatgtaggacaacgcaaagatgggttctttggattctcatggacaacattgttctttggtccgttccctgcattgtttcgtggtgactttattacgttcatcggtgcatttgtaatttacttcatcctcaacgcagtgtctttttggattgtgggtacaattgcaatggtcgtgtgggcatttctttataataagtactacactgtgaatctcatcaagaatgggtataaattcaatggtacacatgaagaaaatgctgaagctgcaatggcgttacaaattgatctaaacaaaaacaatactgtagataaaaacaaagaaccagaataaatggatcaatatgagataacttacgattattatggtaaggcttatatgaagtgtggtcgttttgttgaactatcgaatagagaagcaatcgacccacaaaccattatatccatccataactcgtactataaaaaaattaccatagctaattttgcaacatacgtagataatcctatatttccgtatattattcatttctattgtagagctataaaataaggtaaaaaaatgttagccgatgattttatgtctctaagtatcaagatatttgatatattaggtgatgaaatagatttccatctaaatgataaaactataccacacgaagatggtgggtattttttcgcatttggtaataagttcaatgttgatttttcaattccggttcctgatggaagtgattatgcagcatcaacatattttgaatttcagttcagtggtcataccgagcgtttactaacatatagacgtaaaggaaatttcagtagaccagagattatattaatggattttgaaaaacgcatagaacttgcaaagaataatccattcatcagtacttatgcgttaaaccaattcattgatctatttgatggatatccagacagaatacgagggtcgttacgtataaatgcagaatgaaatctttaaaaaagaatacgatggtgaaagtctctatgatattcagcgagacgtacacgaagcattcgattcaagatttaacccattagctggacaaatcccacaagatgagtttggtttccaagaaggcacatttactgtgactattacatggagtccagatgagactagctaaacttgtaatcggatgcagtgcacttgctgcatatatgttttttgccgctccaaccaatgtagtactgctaatttgggcagttgttatattagcagtactttaaagcaccctagtggtgctttttctatttgtacaaatataaagcaatgtgacctttccttgatataggtcaagagaaataccctgttctgacgctataatggtgtctggtgatgttaaataacttcacccccagatgatttcaaaatacaaggaaagtaaaatgcaattagataaattatatactgacgtaacaaatttcctaaatcagagcaatactgatcttacaaatgaaaatgcaaacaaagatgcgaaatgcgtcaatacacacagggatttacttgctggtatcctatctaagcattattcacaagatgttattccaaaggagctaatgcagtggcataatgatggttatggtcacattcatgatttagactactatatcagcccattaactaactgttgcttggttaactacccagatatgctaaaaaatggatttaccattggttgtgccaaaattgaagaaccaaagagttttagtgtagcatgtacagtactgacacaaattgtacaagctgttgctagttcccagtacggtggtcaatcgttggctcatatcgacgttggtcttgaaccttacgtccaaaagtcgtgggataagctattagcgaagcaaaaggcatataatctcagtactgaatacattgaggaagaattacgaaaagaagtctatgatgggtgtcaaaccttactttaccagacaaattcaatgattagtacaaacggacaagcagcattcatttctcttagtttaggactgaatacttcaaaatttggcaaattaatcactgaacagtacttaaaagtacatgagaatggtttaggtaaagataagattactcctgtgtttcctaaagtaattttctttttacaggacggaatcaacatgaatagcgatgattgcaactatgacatgaaacagttggcaatgcgttgtgcatcgaagagaatctacccagacttcgtttctgtgccgttaaatcacaaagttacaggaagtagcaatacagcagtaactccgatgggctgtcgttcatttttaggaaaatgggaagatgccaatggaaatgagcaatataacggtcgtttcaacttaggcgtggtttctatcaatttacctctaattgctcttgaaagtgcctcagatggagagttttttgataaaatagacgagcatatgaatattgcatatcgtgctcagatgaaccgcgtagagcgtttaaaaggcacaaaagcgaagcagaatccgattttgttcatggaaggtgcactttcacgtttaggggcagaagaaacgattgacaagctattttataacggatatgctagtattagtattggttacgttggtttagttgaatgttcagaaattctgactggttctattagtaaggagttctgtatgtcagtactgaattatatgaaagtaaaatgcgatgagttcaaaaaagagagtaatatagcttttagtctatatggaaccccagcagagtcactttgctacaaatttgcacgttgcatcgaatctaaatacccttcagtactaaaacgtgattacatcacaaattccttccatcaaccagtatgggtagaaagttcaccatttgataagtggagttacgaagagggctttgcttacctcagtaatggtggaaacatcagttatgttgaatcacccaacctaagtaacaatattcaagcacttgagactttagtcaattacgcttacacaaagattccatattttggcatcaaccagccaattgaccagtgttacgagtgtgggtataaaggtgagttcagtgtagataaacacggttttcattgtccagtatgtgataatcgcaatgagaagacaatctcagtgattcgtcgtgtttctggatatttggggaacccatcgtctcgtggattcaacaaaggtaaacaacaagaatgttgtgagcgtatacaacacaaataaggtacattatggatttaaagccaattgtggttaacaaatataaagaaccgtatgatgtgtatatcggacgtgggtctaaatggggtaacccatttacccatatcagtgataaacagaccaaagccgagttcattgttgctacaagggaagaatcaatcgaaaagttcagagaatacattattgagcgtatttctaatggagaagttaagattgatgatttacttgagttagtagggaaacgtcttggctgtttctgtaaaccgaaatcatgtcatggtgatgttctcgttgagatagtgagtgcaatattagatgaattaccagacgataattaaagacgaccttattaatggggagggagtaagatgctccctttttgttagtgggtgcagtcatgggtgtgacggatgcttcaacgaagaagcatgggattatagaagtgggcaagagttcagtactgatacaattgatataattttagctgaactcagtaagccttacgttagtggattatccctccttggtggagatcctctaatgaaaaagaacatagaaacaattttagaactatgtaagattgtaaaagagacttatcccaataaaactatttggtgctggagtggatatactttagaagagattatgaataatcatgctaaagacattcttcagtacttagatgtattagttgatggcaaatttgtttcagaagaaaagaatttaacattaccatttcgtggttcaaataaccaacgaattttatataaaaataaagacttttaaacacaagcacccaatgggtgcttttttagtacattcataaatacagtaactaatatctaaattacaagggatatgtatgtcaaagaaaatatatggtggcttagacgtcactcataatgtgacagttaaagggaaaagaacagtacagggcatcaatgatcaagtatttgatgctaatggtaatatggaaattgatacatattccaaagaagaattacttgatgtaattagtttacttccaatctcgcattatggtacacataactatctacctgctggggtatctggtgattttaatggtgcatcagaaaaccaatcccaacgtagaaacaaaatacttttagaaaacgatggcacattagtactattacgtagtggtacaaacggtagtgttgaaggtttgttttattcttacctagcgaacgcattatcagttaccgatatgagtcgtactgttaatacaagccgtcagtataagcctggctatttcggggcaaaccgtgttgctattggtttatacaatacagattcaaaaatactgttaggtcgttacaatgacacatctaccagtactaatgggttgtttatatcagtaacgaatggtacattggatgatacacaacatagtggtctttttgtgaaccaagctgatatttcacctgaaggtgggattgagtatgctatgatggcagaagacggttcagtttatgtattcaacgtccttaatgaaaataacaaactacagattgttgttgttcgtgttgtattgaatattgcaaacggtacttatactgctacccgaatcacgggatggacaacaaaaactttttacaatactacatatactgctcagaataatataatcattactgaccatgttaactcaactaatattgctgataagccatatgtctatgtaccacaaaatttggctggtatggggatttatatgacatctatcgatttatatgttgctcaggagccaggaacgcagaacttccgattcagaatgaatggtgacgcatggttcactatggcaaactacaatacccgaccacagcatagttatagtttcaattttaacatgagtactaaacaatgtaacttggataatgggaacgatattggtacatttgctgcaccatttgttgttacaaacacaggttcggcattggttgctactggtaacgttctcaataaagatcctttatatgaccatagcggtgcaagaaacatttacagttcatattattatctgaatacaggtgaagtattctgtactgcatgtccaaacttagcagaaccattgcttatgcagaaagctaaatttactgcacaatcaatatatagcatgattaatgtacgtgcgaacatttcaactgatttcagaaatggcatagttcgtccttcatttggttcagcagttggttcttttattaacggcgtagaattgcttccagggaacagtactaaacagtattctcgttcaactggttctggtgcattccgtccatcttatgcagttcataaaagtactcctaactttacgtttaattctttatcacgtggaaccatacaaggttatgagccaacaacagaacgtgctaatgtgacagacaacacgaacaacagagtttttattagttccattagtggtaataatgttactacaaatggtgggatattcatggtaggtcagagatattcccaatcactatcatatgataaaaatatgaacggaactggtatcattagtgttactcaatctgtacttgataatttcagaaatgctgaatacgctaaagtactaagcgactggaacttatctcctgctgcttataaagatatgacattgtttgttccacaacaaactgatatacctgcatttgtaatgctaagtacagttacaaatacatatcagaactatattagaattgctgaagtgaatgtgaatacacgttcaggtaatatcactacaattaccttcaagcgtttagtactagaaaacgtttacggaagtaactttacaccaaatggtaatggtagatttgcatcgtcttctgttggtttaacaatatatgatggtggttctttctacttcataggtggttctgacccattatctcacccaactattggtaatacaaacacacaccaattcagagcatatgtaacaaaagcaactgcacaatttgatagttttattatcagtggttcacatgcaagttatgagcaaggtattcaaccagcagctttacctggtattggttttggttatttccaagcaatagactatgtaaacaaaatgactttccaaaaatgtggtactacaattgctgattataataactggactgctcaaggtgcaccgattaacgtagtttcgcaagacgttgctcaaggttttattgtgtacttcacagaagaaactccagtaattctttctggtaaagcatttacattaccgattactagtataaacttaactacagttaaggctaatccagcaaacacattatttcatgtatatgttaaaatggagcaaggtaatgcaaaatatgtaataacaactgatgttatttcagaaactggtactacagcatataatattttctggataggtactatacaaacaaactcactacaaatatcgaatgttaacatacaaaaacgttcaagattggatattttcggtgcgtctcttgaagctgctggttcttcattcccagttagttatggtctaccgtcacaaacaggaacaatcaattggtaaaggattaatcttatggcatatattataagatccgatatcgaagctatgtcacaaatcgtagtccagaataaaaacactggactatctattagtgatattaataacgtatcgtatcgaacagacgcaaatggggaatgtacattccccctcttaacaaaagatgaagttgattctattgttaacggtttaccattatcacaatatggtgaactttatgatgtatctagtttgtcaattacatcaagcggttattcattacgttttaaccgagagatacctgcattcatcggtggctatctttataattaccctgttgttgcagtgacagcaccaacagtggcatcaggaacacaaacacaatatgtatacgctactttagtactgggtgagccaatttatagaatctcaacttcacaattacaagaatcacagattataatgttcatcgggactattaccgttggaacttctggtattactgctatcaacattgaacgcgtatcacgattcaatacatacagaccaagtacaacacaaaaaggttcatctttcccagtcagtactggaaatccaacacaaactggtacattagactggtaattaaaggaaaaagaatatgtcatccaaacgcatcattggtagtgctgctatacttggaaatttggcaattggtggaaagtacgctatgcgtacttttaatggtacagcaccagacgcaaatacaggtattgttgactatccaatttataaaagatcagaagttcaaaaaatacttgatatgttaccaatctcccgtataggggaaatggattatctaccattaaacgttaatggttcatacgaaggggcaacttcgctttcagatgtataccgtatacaaccaacaatagtcgaagatgatggtactggtgtggtattacgtgctggtacaaatggtagtactgaaggatattactatgcattcattcgtgatttgcgtaatattgatgcactaacacaaagtagtgttatcagtactacatcaaaatatcatcctagaattcttccaagtactgaaaatgttaaacagttcatcggaacgaatggttatgaattactagttagcctatctagtgcaagtaattatattttcactttaacaaatggtacatttaacgaagttgcacatattcacgcgtctatcgctgctacatcatttggtacagataaaccattgtatgcacatatcgttaatgataaagtatttgtgtggtgcaaagacactactatagcaaactatggtttggctttaacattatatactgttaatacaagtagtattaaagatggtactatgagtacttttgctaaggttactggtttttctggtcaaaccttaagaagtgttaactacacaagtgctaataatttgaaaatttatgaaagattatattcagttggagctagtagtgattcattatttgaatgcacttcaccagttacacgagttgaatattcagattatacagattataatatacaagcaaacgtaagtccagatttatcaaagattagaattacattattcccaactaaccgtttagtatcaaatctatatattagtacggtttatgctagtggttttagttttgtttatactatcaactctaaatcattatcaattgatggaagtagtagagcaccaatagcagcggaatataatgatgttgatggattcacattaacaaacccatttgccattgagatgaataacttcatgggtacacagttaggtgttccacaattaggtaacgttggttctatatgccaaacctatgacggtttagtattcagcacacgtgctcgttggagttcatctgaaacctatggtattcaggtttataacgtaacaggaacaccatatgatagttgggttgccagtactcgcaaagcgaacaattctaaatttgtaaacgttgccccaacatatggtagtgcagtaggtgaaaaccttttaggaacacgttttattgataaacgtaaagtactgttagcatgttctggaacatatgaagatgagtcatatggttataacaatacagtattttctgaaattggtactgatccaaccttccagttcaattcagtacaaaacaatacaacacttaatggttttgcaccacaagctaaccgaactcgtcttggtgatgatttcaaatatacaggtatgatttcactaattgatactgacggttcagttaaatgttatgggacatcattcattgaaggtgtatacaaaacaagtggtggtttgtttaacccagatactttagaatttgatggttcttatacaattggtgataatgttctggaaacattaaaacaagaaatacttgctaatattggaatagtaggagcattttctggtaaaattgctttatattatgtaccagatagttctttctcaaaatctattgcggtggtttctgcatataacttaggcaataaccaaggttatgcaatatattctgaagttgatgtcacggtttcaggaaccaatattagttctgcgtctatggtgagttctagatacgaaacacttaattctgtaacaggtattgtaaccgaagcctatatgaaacgctgtactggtctaactgttgctaagtattctggttttggttatattggtgttgatgcagttcatcagttttcacaacctggtaatgctcaattcaaattgatgattgctaaaacagtgaatggtttgattacatctaaccgatttatgtcatccagctacatagataatggcaatgcattccagcacggagttataccaggtgttggatttggatactatcatcttcaagattcagattatcaaactaaagcagtgtttagtctctttgggacaacatcagcacagatggatgggttgattgatggttcaggaactgtacaagataaaatcgtagtagtttcacaagatgttgctgaaggtttcactgtgtacttcacccgtgaagttccggtattccttggtggtatgtttgatagattggcgattcaaaccatagaattaagtactattaaaactaacccagctaacagtactttttatctttacattcaaatgaaccgtaacacaagaaaagcggcatacgttatcagtacaactcttcttgatgagacattgacaaacgtatatgttggtgatattgttactggaagtacgagtattacgtcaattgatactgaaaaggtaacacgattcttgacatacagacctagtacaactaagcgtggttctgctatccctgcaagttctggtgttccttcaagctctggtacacgttggcattaaaaattttaagctcgtctattgacgggcttttcttttatgtgtataatgctagaaaatacaggagga

(19) United States
(12) Patent Application Publication
(10) Pub. No.: US 2024/0366695 A1
(43) Pub. Date: Nov. 7, 2024

(54) COMPOSITIONS AND METHODS FOR INHIBITING THE PROLIFERATION OF PATHOGENIC ESCHERICHIA COLI

(71) Applicant: iNtRON Biotechnology, Inc., Seongnam-Si (KR)

(72) Inventors: Seong Jun YOON, Seoul (KR); Jee Soo SON, Seoul (KR); In Hwang KIM, Gyeonggi-do (KR); Hyoung Rok PAIK, Incheon (KR); Hyun Joo IM, Gyeonggi-do (KR); Beom Seok KIM, Gyeonggi-do (KR); Eun Kyoung OH, Gyeonggi-do (KR); Soo Youn JUN, Seoul (KR); Sang Hyeon KANG, Seoul (KR)

(21) Appl. No.: 18/772,220

(22) Filed: Jul. 14, 2024

Related U.S. Application Data
(63) Continuation of application No. 17/060,175, filed on Oct. 1, 2020.

(51) Int. Cl. A61K 35/76; A61K 9/08; A61K 9/107; A61K 9/14; A61K 47/36; A61K 47/42; C12N 7/00

(52) U.S. Cl. CPC A61K 35/76; A61K 9/08; A61K 9/107; A61K 9/14; A61K 47/36; A61K 47/42; C12N 7/00; C12N 2795/10132

(57) ABSTRACT

A composition for treating an infection or disease caused by a pathogenic Escherichia coli includes a Myoviridae bacteriophage Esc-COP-23 having an ability to lyse the pathogenic Escherichia coli and a pharmaceutically acceptable carrier. A method for treating an infection or disease caused by a pathogenic Escherichia coli includes administering to a subject a Myoviridae bacteriophage and lysing the pathogenic Escherichia coli by the Myoviridae bacteriophage.

This application is a Continuation application of U.S. Ser. No. 17/060,175, filed on Oct. 1, 2020, which is incorporated by reference for all purposes as if fully set forth herein.

### INCORPORATION BY REFERENCE OF SEQUENCE LISTING PROVIDED AS A TEXT FILE

A Sequence Listing XML file named “20001_0057C1.xml” created on Jul. 14, 2024, and having a size of 362,166 bytes, is filed concurrently with the specification. The sequence listing contained in the XML file is part of the specification and is herein incorporated by reference in its entirety.

### BACKGROUND OF THE INVENTION

### Field of the Invention

The present invention relates to compositions and methods for inhibiting the proliferation of pathogenic Escherichia coli, more specifically, a composition containing a Myoviridae bacteriophage and a method of using the same.

### Discussion of the Related Art

Escherichia coli is a Gram-negative, facultative anaerobic, rod-shaped, coliform bacterium of the genus Escherichia. It is serologically subdivided according to whether it contains a somatic (O), flagellar (H) or capsular (K) antigen, and these antigens are known to be associated with the pathogenicity of Escherichia coli. Pathogenic Escherichia coli refers to Escherichia coli that has acquired a small number of the virulence factors capable of being expressed in Escherichia coli, and, depending on the onset characteristics and the kind of toxin, there are five types of pathogenic Escherichia coli, namely enterohemorrhagic Escherichia coli, enterotoxigenic Escherichia coli, enteroinvasive Escherichia coli, enteropathogenic Escherichia coli, and enteroaggregative Escherichia coli.

Pathogenic Escherichia coli causes various diseases, such as food poisoning, acute pancreatitis, urinary tract infection, septicemia and cancer. Among pathogenic Escherichia coli-associated cancer, colorectal cancer is one of the most common cancers, accounting for approximately 10% of all cancer cases and approximately 8% of all cancer deaths. Also, colorectal cancer is very common globally and develops through accumulation of colonic epithelial cell mutations that promote transition of normal mucosa to adenocarcinoma. As one of major causes leading to colorectal cancer occurrence, colonic polyp refers to a condition in which the colonic mucosa grows abnormally and becomes a wart-shaped bump that protrudes into the intestine. It is often divided into neoplastic polyps that are likely to develop into cancer and non-neoplastic polyps that are unlikely to develop into cancers. Among various types of polyp, adenomatous polyps are more likely to develop cancer over time. Although diarrhea caused by pathogenic Escherichia coli is a notable disease, colonization of some pathogenic Escherichia coli is related to promotion of colorectal cancer development by promotion of the formation of adenomatous polyps.

Generally, vaccines and antibiotics are used for the prevention and treatment of infectious diseases of pathogenic Escherichia coli. Here, the effectiveness of antibiotics has been continuously decreasing due to the increase of antibiotic-resistant pathogenic Escherichia coli, and the development of effective methods other than currently prescribed antibiotics is required.

Recently, the use of bacteriophages as a countermeasure against bacterial infectious diseases has attracted considerable attention. Bacteriophages are very small microorganisms infecting bacteria, and are usually simply called “phages.” Once a bacteriophage infects a bacterial cell, the bacteriophage is proliferated inside the bacterial cell. After proliferation, the progeny of the bacteriophage destroys the bacterial cell wall and escapes from the host bacteria, suggesting that the bacteriophage has the ability to kill bacteria. The manner in which the bacteriophage infects bacteria is characterized by the very high specificity thereof, and thus the number of types of bacteriophages infecting a specific bacterium is limited. That is, a certain bacteriophage can infect only a specific bacterium, suggesting that a certain bacteriophage can kill only a specific bacterium and cannot harm other bacteria. Due to this bacteria specificity of bacteriophages, the bacteriophage confers antibacterial effects only upon target bacteria, but does not affect commensal bacteria in animals including human being. Conventional antibiotics, which have been widely used for bacterial treatment, incidentally influence many kinds of bacteria. This causes problems such as the disturbance of normal microflora. On the other hand, the use of bacteriophages does not disturb normal microflora, because the target bacterium is selectively killed. Hence, the bacteriophage may be utilized safely, which thus greatly lessens the probability of adverse actions in use compared to any other antibiotics.

Owing to the unique ability of bacteriophages to kill bacteria, bacteriophages have attracted attention as a potentially effective countermeasure against bacterial infections since their discovery, and there has been a lot of research related thereto.

Bacteriophages tend to be highly specific for bacteria. It has been shown that the attack of bacteriophage is specific, meaning that one species of bacteriophage targets only a single species of bacteria (or even a specific strain of one species). In addition, the antibacterial strength of bacteriophages may depend on the type of target bacterial strain. Therefore, it is necessary to collect many kinds of bacteriophages that are useful in order to get effective control of specific bacteria. Hence, in order to develop the effective bacteriophage utilization method in response to pathogenic Escherichia coli, many kinds of bacteriophages that exhibit antibacterial action against pathogenic Escherichia coli must be acquired. Furthermore, the resulting bacteriophages need to be screened as to whether or not they are superior to others from the aspect of antibacterial strength and spectrum.

### SUMMARY OF THE INVENTION

Accordingly, the present invention has been made keeping in mind the problems encountered in the related art and is intended to solve such problems.

In one embodiment, a composition for preventing or treating an infection or disease caused by a pathogenic Escherichia coli includes: a Myoviridae bacteriophage having an ability to lyse the pathogenic Escherichia coli, and a pharmaceutically acceptable carrier.

In another embodiment, the Myoviridae bacteriophage has a genome including a sequence as set forth in SEQ ID NO: 1; or a genome that has (1) a sequence having at least 96% query cover with at least 97% identity to SEQ ID NO: 1, (2) a circular genome topology, and (3) 587 open reading frames.

In another embodiment, the Myoviridae bacteriophage has a concentration of 1×10¹ pfu/ml to 1×10³⁰ pfu/ml or 1×10¹ pfu/g to 1×10³⁰ pfu/g.

In another embodiment, the Myoviridae bacteriophage has a concentration of 1×10⁴ pfu/ml to 1×10¹⁵ pfu/ml or 1×10⁴ pfu/g to 1×10¹⁵ pfu/g.

In another embodiment, the pharmaceutically acceptable carrier is lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methylcellulose, methylhydroxybenzoate, propylhydroxybenzoate, talc, magnesium stearate, or mineral oil.

In another embodiment, the composition further includes one or more selected from the group consisting of a lubricant, a wetting agent, a sweetener, a flavor, an emulsifier, a suspending agent, and a preservative.

In another embodiment, the pathogenic Escherichia coli is enterohemorrhagic Escherichia coli, enterotoxigenic Escherichia coli, enteroinvasive Escherichia coli, enteropathogenic Escherichia coli, enteroaggregative Escherichia coli, or carcinogenic Escherichia coli.

In another embodiment, the infection or disease is food poisoning, gastroenteritis, diarrhea, urinary tract infections, neonatal meningitis, hemolytic-uremic syndrome, peritonitis, mastitis, septicemia, Gram-negative pneumonia, shigellosis, dysentery, or cancer.

In another embodiment, the composition is a solution, suspension, emulsion in oil, water-soluble medium, extract, powder, granule, tablet, or capsule.

In another embodiment, the composition further includes a second bacteriophage having an ability to lyse a pathogenic Escherichia coli or a non-Escherichia coli bacterial species.

In another embodiment, the Myoviridae bacteriophage has major structural proteins in the sizes of approximately 50 kDa, 69 kDa, 128 kDa, and 150 kDa.

In another embodiment, the Myoviridae bacteriophage has a latent period of 5-25 minutes and a burst size of 910-995 PFU/infected cell.

In another embodiment, the latent period is 10-15 minutes and the burst size of 940-965 PFU/infected cell.

In one embodiment, a method for preventing or treating an infection or disease caused by a pathogenic Escherichia coli includes administering to a subject a Myoviridae bacteriophage; and lysing the pathogenic Escherichia coli by the Myoviridae bacteriophage.

In another embodiment, the Myoviridae bacteriophage includes a sequence as set forth in SEQ ID NO: 1.

In another embodiment, the Myoviridae bacteriophage has a concentration of 1×10¹ pfu/ml to 1×10³⁰ pfu/ml or 1×10¹ pfu/g to 1×10³⁰ pfu/g.

In another embodiment, the Myoviridae bacteriophage has a concentration of 1×10⁴ pfu/ml to 1×10¹⁵ pfu/ml or 1×10⁴ pfu/g to 1×10¹⁵ pfu/g.

It is to be understood that both the foregoing general description and the following detailed description are exemplary and explanatory and are intended to provide further explanation of the invention as claimed.

### Advantageous Effects of Invention

The compositions and methods for inhibiting the proliferation of pathogenic Escherichia coli, of the present application have high specificity against pathogenic Escherichia coli, compared with conventional compositions and methods based on antibiotics. The compositions can be used for preventing or treating pathogenic Escherichia coli infections without affecting other useful commensal bacteria and have fewer side effects. In general, when antibiotics are used, commensal bacteria are also damaged, thus entailing various side effects owing to the use thereof. Meanwhile, each antibacterial property of the bacteriophages such as antibacterial strength and spectrum (host range) are different in the case of bacteriophages exhibiting antibacterial activity against the same bacterial species and bacteriophages are usually effective only on some bacterial strains within the same bacterial species. Thus, the compositions and methods of the present application provide different effects in its industrial applications.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is an electron micrograph showing the morphology of the bacteriophage Esc-COP-23.

FIG. 2 is a result of the analysis for major structural proteins of bacteriophage Esc-COP-23.

FIG. 3 is a photograph showing the results of an experiment on the ability of the bacteriophage Esc-COP-23 to kill Escherichia coli. The clear zone is a plaque formed by lysis of the target bacteria.

FIG. 4 is the one-step growth curve of bacteriophage Esc-COP-23.

### DETAILED DESCRIPTION OF THE ILLUSTRATED EMBODIMENTS

Reference will now be made in detail to embodiments of the present invention, example of which is illustrated in the accompanying drawings.

In accordance with one aspect of the present invention, the present invention provides a Myoviridae bacteriophage, named as Esc-COP-23, which has the ability to specifically kill Escherichia coli and has a genome including a sequence as set forth in SEQ ID NO: 1. In some embodiment, the Myoviridae bacteriophage contains a genome that all the following characteristics: 1) including a sequence having at least 96% query cover with at least 97% identity to SEQ ID NO: 1, 2) having a circular genome topology, and 3) having 587 open reading frames; a genome that has all the following characteristics: 1) including a sequence having at least 97% query cover with at least 97% identity SEQ ID NO: 1, 2) having the circular genome topology, and 3) having 587 open reading frames; a genome that has all the following characteristics: 1) including a sequence having at least 98% query cover with at least 97% identity SEQ ID NO: 1, 2) having the circular genome topology, and 3) having 587 open reading frames; or a genome that has one or more of the following characteristics: 1) including a sequence having at least 99% query cover with at least 97% identity to SEQ ID NO: 1, 2) having the circular genome topology, and 3) having 587 open reading frames.

The present invention also provides a method for preventing and treating infections or diseases caused by pathogenic Escherichia coli using a composition including the same as an active ingredient.

The bacteriophage Esc-COP-23 was isolated by the present inventors and then deposited at Korea Collection for Type Cultures, Korea Research Institute of Bioscience and Biotechnology on Nov. 15, 2019 (Accession number: KCTC 14030BP).

The molecular weight of major structural proteins of the bacteriophage Esc-COP-23 is approximately 50 kDa, 69 kDa, 128 kDa, and 150 kDa.

The latent period and burst size of the bacteriophage Esc-COP-23 are 5-25 minutes and 910-995 PFU/infected cell, respectively, preferably 10-15 minutes and 940-965 PFU/infected cell, respectively, but are not limited thereto.

Also, the present invention provides a composition applicable for the prevention or treatment of infections or diseases caused by pathogenic Escherichia coli, which include the bacteriophage Esc-COP-23 as an active ingredient.

Because the bacteriophage Esc-COP-23 included in the composition of the present invention kills pathogenic Escherichia coli effectively, it is considered effective in the prevention of pathogenic Escherichia coli infections or treatment of diseases caused by pathogenic Escherichia coli. Therefore, the composition of the present invention is capable of being utilized for the prevention and treatment of diseases caused by pathogenic Escherichia coli.

The diseases caused by pathogenic Escherichia coli in the present invention include food poisoning, gastroenteritis, diarrhea, urinary tract infections, neonatal meningitis, hemolytic-uremic syndrome, peritonitis, mastitis, septicemia, Gram-negative pneumonia, shigellosis, dysentery and cancer, but are not limited thereto.

The pharmaceutically acceptable carrier included in the composition of the present invention is one that is generally used for the preparation of a pharmaceutical formulation, and examples thereof include lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methylcellulose, methylhydroxybenzoate, propylhydroxybenzoate, talc, magnesium stearate, and mineral oil, but are not limited thereto. The composition of the present invention may additionally include lubricants, wetting agents, sweeteners, flavors, emulsifiers, suspending agents, and preservatives, in addition to the above ingredients.

In the composition of the present invention, the bacteriophage Esc-COP-23 is included as an active ingredient. The bacteriophage Esc-COP-23 is included at a concentration of 1×10¹ pfu/ml to 1×10³⁰ pfu/ml or 1×10¹ pfu/g to 1×10³⁰ pfu/g, and preferably at a concentration of 1×10⁴ pfu/ml to 1×10¹⁵ pfu/ml or 1×10⁴ pfu/g to 1×10¹⁵ pfu/g.

The composition of the present invention can be formulated according to a method that can be easily performed by those of ordinary skill in the art to which the present invention pertains using a pharmaceutically acceptable carrier and/or excipient in the form of a unit dose or in a multi-dose container. Then, the formulation may be in the form of a solution, suspension, or emulsion in oil or a water-soluble medium, extract, powder, granule, tablet, or capsule. A dispersing agent or stabilizer may be additionally included.

In order to improve the effectiveness of above purpose, bacteriophages that have antibacterial activity against non-Escherichia coli bacterial species may be further included in the composition of the present invention. In addition, other kinds of bacteriophages that have antibacterial activity against Escherichia coli may be further included in the composition of the present invention. These bacteriophages may be additionally included so as to maximize antibacterial effects, because each antibacterial property of the bacteriophages such as antibacterial strength and spectrum (host range) are different in the case of bacteriophages exhibiting antibacterial activity against the same bacterial species.

In this description, the terms “prevention” and “prevent” indicate (i) to block pathogenic Escherichia coli infections; and (ii) to inhibit the progression of diseases caused by pathogenic Escherichia coli infections.

In this description, the terms “treatment” and “treat” indicate all actions that (i) suppress diseases caused by pathogenic Escherichia coli; and (ii) alleviate the pathological condition of the diseases caused by pathogenic Escherichia coli.

In this description, the term “pathogenic Escherichia coli” indicates enterohemorrhagic Escherichia coli, enterotoxigenic Escherichia coli, enteroinvasive Escherichia coli, enteropathogenic Escherichia coli, enteroaggregative Escherichia coli and carcinogenic Escherichia coli, but are not limited thereto.

In this description, the terms “diseases caused by pathogenic Escherichia coli” and “pathogenic Escherichia coli infections” indicate food poisoning, gastroenteritis, diarrhea, urinary tract infections, neonatal meningitis, hemolytic-uremic syndrome, peritonitis, mastitis, septicemia, Gram-negative pneumonia, shigellosis, dysentery and cancer, but are not limited thereto.

In this description, the term “Latent period” indicates the time taken by a bacteriophage particle to reproduce inside an infected host cell.

In this description, the term “Burst size” indicates the number of bacteriophages produced per infected bacterium.

In this description, the terms “isolate”, “isolating”, and “isolated” indicate actions which isolate bacteriophages from nature by applying diverse experimental techniques and which secure characteristics that can distinguish the target bacteriophage from others, and further include the action of proliferating the target bacteriophage using bioengineering techniques so that the target bacteriophage is industrially applicable.

In this description, the terms “query cover” and “identity” are related to BLAST (Basic Local Alignment Search Tool) which is an online search tool provided by NCBI (National Center for Biotechnology Information).

In this description, the query cover is a number that describes how much of the query sequence (i.e., the sequence of genome of bacteriophage Esc-COP-23) is covered by the target sequence (i.e., the sequence of genome of the previously reported bacteriophage). If the target sequence in the database spans the whole query sequence, then the query cover is 100%. This tells us how long the sequences are, relative to each other.

In this description, the term “identity” or “sequence identity” was measured for “query cover”, and is a number that describes how similar the query sequence (i.e., the sequence of genome of bacteriophage Esc-COP-23) is to the target sequence (i.e., the sequence of genome of the previously reported bacteriophage). More specifically, the terms “identity” or “sequence identity” refers to the percentage of identical nucleotides in the spanned sequence part of the target sequence (i.e., the sequence of genome of the previously reported bacteriophage) or the query sequence (i.e., the sequence of genome of bacteriophage Esc-COP-23) when the query sequence (i.e., the sequence of genome of bacteriophage Esc-COP-23) and the target sequence (i.e., the sequence of genome of the previously reported bacteriophage) are analyzed by BLAST alignment analysis. The higher the percent identity is, the more significant the match is. From above definitions for “query cover” and “sequence identity”, it will be obvious for the skilled one in the art that the differences of “query cover” and/or “sequence identity” between genomes of two similar bacteriophages make the differences of ORF (open reading frame)'s numbers arranged in the two genomes, then results in the discriminative characteristics (including the range of target strain and strength of antibacterial activity) of two similar bacteriophages.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1: Isolation of Bacteriophage Capable of Killing Escherichia coli

Samples were collected from environmental or clinical samples to isolate the bacteriophage capable of killing Escherichia coli. Here, the Escherichia coli strains used for the bacteriophage isolation had been previously isolated and identified as Escherichia coli by the present inventors.

The procedure for isolating the bacteriophage is described in detail hereinafter. The collected sample was added to a TSB (Tryptic Soy Broth) culture medium (casein digest, 17 g/L; soybean digest, 3 g/L; dextrose, 2.5 g/L; NaCl, 5 g/L; dipotassium phosphate, 2.5 g/L) inoculated with Escherichia coli at a ratio of 1/1000, followed by shaking culture at 37° C. for 3 to 4 hours. Upon completion of the culture, centrifugation was performed at 8,000 rpm for 20 minutes and a supernatant was recovered. The recovered supernatant was inoculated with Escherichia coli at a ratio of 1/1000, followed by shaking culture at 37° C. for 3 to 4 hours. When the sample contained the bacteriophage, the above procedure was repeated a total of 5 times in order to sufficiently increase the number (titer) of the bacteriophage. After repeating the procedure 5 times, the culture solution was subjected to centrifugation at 8,000 rpm for 20 minutes. After the centrifugation, the recovered supernatant was filtered using a 0.45 μm filter. The obtained filtrate was used in a typical spot assay for examining whether or not a bacteriophage capable of killing Escherichia coli was included therein.

The spot assay was performed as follows: TSB culture medium was inoculated with Escherichia coli at a ratio of 1/1000, followed by shaking culture at 37° C. overnight. 2 ml (OD₆₀₀ of 1.5) of the culture solution of Escherichia coli prepared above was spread on TSA (casein digest, 15 g/L; soybean digest, 5 g/L; NaCl, 5 g/L; agar, 15 g/L) plate. The plate was left on a clean bench for about 30 minutes to dry the spread solution. After drying, 10 μl of the prepared filtrate was spotted onto the plate culture medium on which Escherichia coli was spread and then left to dry for about 30 minutes. After drying, the plate culture medium that was subjected to spotting was incubated at 37° C. for one day, and then examined for the formation of clear zones at the positions where the filtrate was dropped. In the case of the filtrate generated a clear zone, it is judged that the bacteriophage capable of killing Escherichia coli is included therein. Through the above examination, the filtrate containing the bacteriophage having the ability to kill Escherichia coli could be obtained.

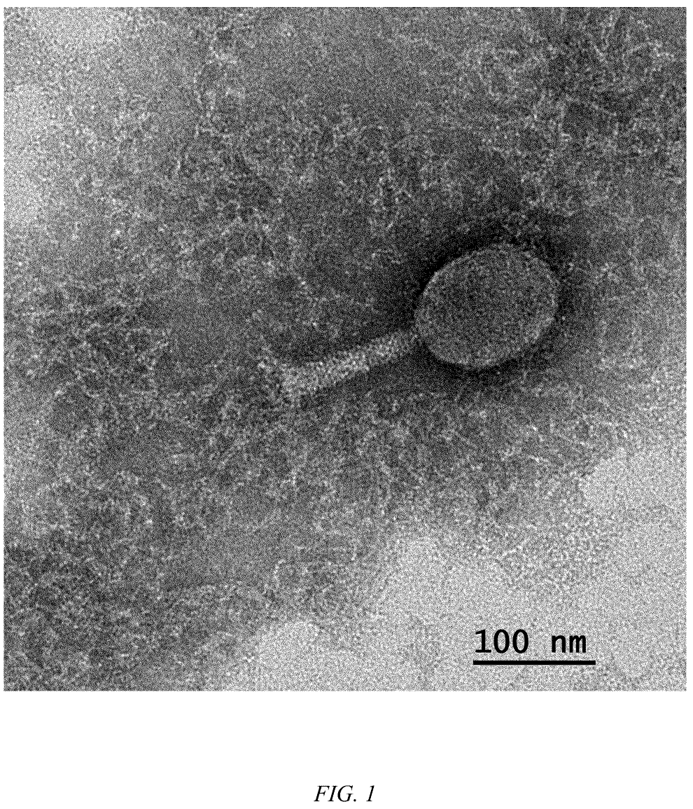

The pure bacteriophage was isolated from the filtrate confirmed above to have the bacteriophage capable of killing Escherichia coli. A conventional plaque assay was used to isolate the pure bacteriophage. In detail, a plaque formed in the course of the plaque assay was recovered using a sterilized tip, which was then added to the culture solution of Escherichia coli, followed by culturing at 37° C. for 4 to 5 hours. After the culturing, centrifugation was performed at 8,000 rpm for 20 minutes to obtain a supernatant. The Escherichia coli culture solution was added to the obtained supernatant at a volume ratio of 1/50, followed by culturing at 37° C. for 4 to 5 hours. In order to increase the number of bacteriophages, the above procedure was repeated at least 5 times. Then, centrifugation was performed at 8,000 rpm for 20 minutes in order to obtain the final supernatant. A plaque assay was further performed using the resulting supernatant. In general, the isolation of a pure bacteriophage is not completed through a single iteration of a procedure, so the above procedure was repeated using the resulting plaque formed above. After at least 5 repetitions of the procedure, a solution containing the pure bacteriophage was obtained. The procedure for isolating the pure bacteriophage was generally repeated until the generated plaques became similar to each other in size and morphology. In addition, final isolation of the pure bacteriophage was confirmed using electron microscopy. The above procedure was repeated until the isolation of the pure bacteriophage was confirmed using electron microscopy. The electron microscopy was performed according to a conventional method. Briefly, the solution containing the pure bacteriophage was loaded on a copper grid, followed by negative staining with 2% uranyl acetate and drying. The morphology thereof was then observed using a transmission electron microscope. The electron micrograph of the pure bacteriophage that was isolated is shown in FIG. 1. Based on the morphological characteristics, the novel bacteriophage isolated above was confirmed to belong to the Myoviridae bacteriophage.

The solution containing the pure bacteriophage confirmed above was subjected to the following purification process. The Escherichia coli culture solution was added to the solution containing the pure bacteriophage at a volume ratio of 1/50 based on the total volume of the bacteriophage solution, followed by further culturing for 4 to 5 hours. After the culturing, centrifugation was performed at 8,000 rpm for 20 minutes to obtain a supernatant. This procedure was repeated a total of 5 times in order to obtain a solution containing sufficient numbers of the bacteriophage. The supernatant obtained from the final centrifugation was filtered using a 0.45 μm filter, followed by a conventional polyethylene glycol (PEG) precipitation process. Specifically, PEG and NaCl were added to 100 ml of the filtrate until reaching 10% PEG 8000/0.5 M NaCl, and then left at 4° C. for 2 to 3 hours. Thereafter, centrifugation was performed at 8,000 rpm for 30 minutes to obtain the bacteriophage precipitate. The resulting bacteriophage precipitate was suspended in 5 ml of a buffer (10 mM Tris-HCl, 10 mM MgSO₄, 0.1% gelatin, pH 8.0). The resulting material was referred to as a bacteriophage suspension or bacteriophage solution.

As a result, the pure bacteriophage purified above was collected, was named the bacteriophage Esc-COP-23, and then deposited at Korea Collection for Type Culture, Korea Research Institute of Bioscience and Biotechnology on Nov. 15, 2019 (Accession number: KCTC 14030BP).

### Example 2: Separation and Sequence Analysis of Genome of Bacteriophage Esc-COP-30

The genome of the bacteriophage Esc-COP-23 was separated as follows. The genome was separated from the bacteriophage suspension obtained using the same method as in Example 1. First, in order to remove DNA and RNA of Escherichia coli included in the suspension, 200 U of each of DNase I and RNase A was added to 10 ml of the bacteriophage suspension and then left at 37° C. for 30 minutes. After being left for 30 minutes, in order to stop the DNase I and RNase A activity, 500 μl of 0.5 M ethylenediaminetetraacetic acid (EDTA) was added thereto and then left for 10 minutes. In addition, the resulting mixture was further left at 65° C. for 10 minutes, and 100 μl of proteinase K (20 mg/ml) was then added thereto so as to break the outer wall of the bacteriophage, followed by reaction at 37° C. for 20 minutes. After that, 500 μl of 10% sodium dodecyl sulfate (SDS) was added thereto, followed by reaction at 65° C. for 1 hour. After reaction for 1 hour, 10 ml of the solution of phenol:chloroform:isoamyl alcohol, mixed at a component ratio of 25:24:1, was added to the reaction solution, followed by mixing thoroughly. In addition, the resulting mixture was subjected to centrifugation at 13,000 rpm for 15 minutes to separate layers. Among the separated layers, the upper layer was selected, and isopropyl alcohol was added thereto at a volume ratio of 1.5, followed by centrifugation at 13,000 rpm for 10 minutes in order to precipitate the genome. After collecting the precipitate, 70% ethanol was added to the precipitate, followed by centrifugation at 13,000 rpm for 10 minutes to wash the precipitate. The washed precipitate was recovered, vacuum-dried and then dissolved in 100 μl of water. This procedure was repeated to obtain a sufficient amount of the genome of the bacteriophage Esc-COP-23.

Information on the sequence of the genome of the bacteriophage Esc-COP-23 obtained above was secured by performing next-generation sequencing analysis using Illumina Mi-Seq equipment from the National Instrumentation Center for Environmental Management, Seoul National University. The finally analyzed genome of the bacteriophage Esc-COP-23 had a size of 359,853 bp, and the sequence of whole genome was expressed by SEQ ID NO: 1.

The homology (similarity) of the bacteriophage Esc-COP-23 genomic sequence obtained above with previously reported bacteriophage genomic sequences was investigated using BLAST investigation, the genomic sequence of the bacteriophage Esc-COP-23 was found to have a relatively high homology with the sequence of the Escherichia bacteriophage CMSTMSU (Genbank Accession No. MH494197.1) (query cover: 96%, sequence identity: 98.2%). In addition, the number of open reading frames (ORFs) on the bacteriophage Esc-COP-23 genome is 587, whereas Escherichia bacteriophage CMSTMSU has 767 open reading frames.

Based upon this result, it is concluded that the bacteriophage Esc-COP-23 must be a novel bacteriophage different from conventionally reported bacteriophages. Further, since the antibacterial strength and spectrum of bacteriophages typically depend on the type of bacteriophage, it is considered that the bacteriophage Esc-COP-23 can provide antibacterial activity different from that of any other bacteriophages reported previously.

### Example 3: Analysis of the Major Structural Proteins of Bacteriophage Esc-COP-23

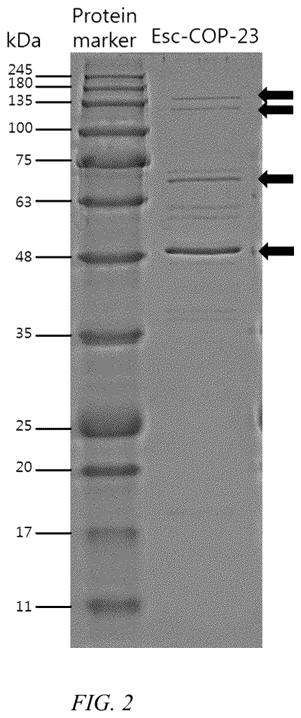

One-dimensional electrophoresis was performed to analyze the major structural proteins of the bacteriophage Esc-COP-23. To obtain the proteins constituting the outer wall of the bacteriophage Esc-COP-23, 200 μl of the bacteriophage suspension prepared in Example 1 was mixed with 800 μl of acetone, which was vortexed vigorously. The mixture stood at −20° C. for 10 minutes. Centrifugation was performed at 13,000 rpm at 4° C. for 20 minutes to eliminate supernatant, followed by air drying. The precipitate was resuspended in 50 μl of electrophoresis sample buffer (5×), which was then boiled for 5 minutes. The prepared sample was analyzed by one-dimensional electrophoresis. As a result, as shown in FIG. 2, the major structural proteins in the sizes of approximately 50 kDa, 69 kDa, 128 kDa, and 150 kDa were confirmed.

### Example 4: Investigation of Ability of Bacteriophage Esc-COP-23 to Kill Pathogenic Escherichia coli

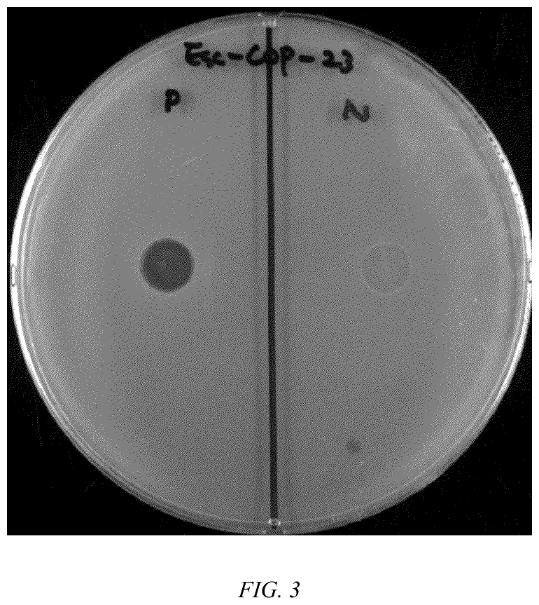

The ability of bacteriophage Esc-COP-23 to kill pathogenic Escherichia coli was investigated. In order to investigate the killing ability, the formation of clear zones was observed using the spot assay in the same manner as described in Example 1. A total of 6 strains that had been identified as pks positive Escherichia coli strains that are positive carriers of the pks genomic island were used as pathogenic Escherichia coli for the investigation of killing ability. The bacteriophage Esc-COP-23 had the ability to lyse and kill a total of 5 strains among 6 strains of pathogenic Escherichia coli as the experimental target. The experimental result thereof is presented in Table 1 and the representative result is shown in FIG. 3.

TABLE 1
Test of antibacterial activity of bacteriophage Esc-COP-23

| Tested Escherichia coli strain | Test result |
|---|---|
| Escherichia coli CCARM 1G931 | + |
| Escherichia coli CCARM 1G932 | + |
| Escherichia coli CCARM 1G934 | + |
| Escherichia coli CCARM 1G936 | + |
| Escherichia coli CCARM 1G937 | + |
| Escherichia coli CCARM 1G938 | − |

* +: clear lytic activity, −: no lytic activity;
CCARM: Culture Collection of Antimicrobial Resistant Microbes (Seoul, Korea)

Meanwhile, the ability of the bacteriophage Esc-COP-23 to kill Bordetella bronchiseptica, Enterococcus faecalis, Enterococcus faecium, Staphylococcus aureus, Streptococcus pneumoniae and Pseudomonas aeruginosa was also investigated in a separate experiment. As a result, the bacteriophage Esc-COP-23 did not have the ability to kill these bacteria.

Therefore, it is confirmed that the bacteriophage Esc-COP-23 has strong ability to kill pathogenic Escherichia coli and a broad antibacterial spectrum against pathogenic Escherichia coli, suggesting that the bacteriophage Esc-COP-23 can be used as an active ingredient of the composition for preventing and treating pathogenic Escherichia coli infections.

### Example 5: Growth Characteristic of Bacteriophage Esc-COP-23

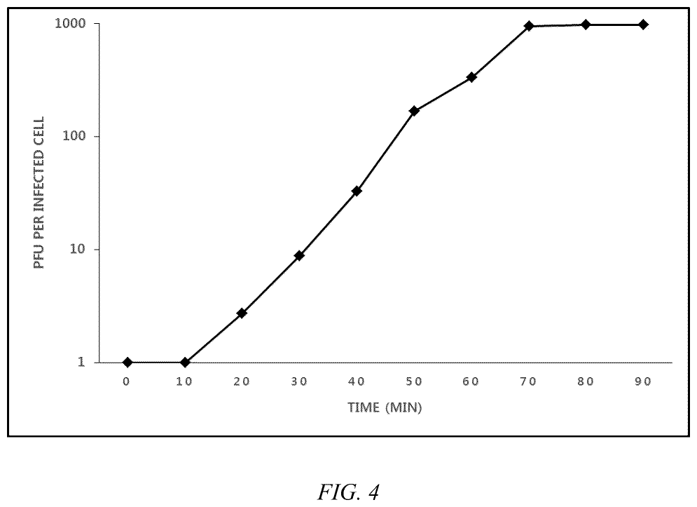

The growth characteristics of bacteriophage Esc-COP-23 was analyzed by one-step growth curve analysis. One-step growth curve analysis of bacteriophage Esc-COP-23 was performed as follows: 50 ml of TSB (Tryptic soy broth, Difco) culture medium was inoculated with Escherichia coli at a ratio of 1/1000 and followed by shaking culture until exponential phase (OD₆₀₀=0.3˜0.4). Upon completion of the culture, centrifugation was performed at 8,000 rpm for 5 min and a bacterial cell pellet was recovered. The recovered pellet was suspended in 50 ml of TSB. The resulting material may be referred to as a bacterial suspension. The bacteriophage Esc-COP-23 was mixed with the bacterial suspension at a multiplicity of infection (MOI) of 0.1 and incubated at room temperature for 10 min, and then centrifuged at 12,000 rpm for 30 seconds. After supernatants were removed, the pellets containing bacteriophage-infected bacterial cells were suspended in 50 ml of TSB and incubated at 37° C. with shaking. Aliquots were taken at 5 min intervals for 60 min, and the titers in the aliquots were immediately determined by the conventional plaque assay (FIG. 4).

The latent period of bacteriophage Esc-COP-23 was estimated to be approximately 10±5 min with average burst size of about 950±30 pfu/infected cell.

### Example 6: Experimental Example Regarding Prevention of Pathogenic Escherichia coli Infection Using Bacteriophage Esc-COP-23

100 μl of a bacteriophage Esc-COP-23 suspension (1×10⁸ pfu/ml) was added to a tube containing 9 ml of a TSB culture medium. To another tube containing 9 ml of a TSB culture medium, only the same amount of TSB culture medium was further added. A pathogenic Escherichia coli (pks positive strain CCARM 1G934) culture solution was then added to each tube so that absorbance reached about 0.5 at 600 nm. After pathogenic Escherichia coli was added, the tubes were transferred to an incubator at 37° C., followed by shaking culture, during which the growth of pathogenic Escherichia coli was observed. As presented in Table 2, it was observed that the growth of pathogenic Escherichia coli was inhibited in the tube to which the bacteriophage Esc-COP-23 suspension was added, while the growth of pathogenic Escherichia coli was not inhibited in the tube to which the bacteriophage suspension was not added.

TABLE 2
Test for bacterial growth inhibition of bacteriophage Esc-COP-23

| | OD₆₀₀ | | |
|---|---|---|---|
| | 0 minutes after | 30 minutes after | 60 minutes after |
| | initiation of | initiation of | initiation of |
| Classification | cultivation | cultivation | cultivation |
| Bacteriophage | 0.5 | 0.8 | 1.4 |
| suspension was | | | |
| not added | | | |
| Bacteriophage | 0.5 | 0.4 | 0.2 |
| suspension was | | | |
| added | | | |

The above results indicate that the bacteriophage Esc-COP-23 of the present invention not only inhibits the growth of pathogenic Escherichia coli but also has the ability to kill pathogenic Escherichia coli. Therefore, it is concluded that the bacteriophage Esc-COP-23 can be used as an active ingredient of the composition for preventing a pathogenic Escherichia coli infection.

### Example 7: Preventive Effect of Bacteriophage Esc-COP-23 on the Infections of Escherichia coli in Animal Model

Preventive effect of the bacteriophage Esc-COP-23 on weaning pigs affected by Escherichia coli was investigated. 4 weaning pigs at 25 days of age were grouped together; total 2 groups of pigs were raised in each pig pen (1.1 m×1.0 m). Heating system was furnished and the surrounding environment was controlled. The temperature and the humidity of the pig pen were controlled consistently and the floor was cleaned every day. From the 1^{st} day of the experiment, pigs of the experimental group (adding the bacteriophage) were fed with feeds adding the bacteriophage Esc-COP-23 at 1×10⁸ pfu/g according to the conventional feed supply procedure, while pigs of the control group (without adding the bacteriophage) were fed with the same feed without adding the bacteriophage Esc-COP-23 according to the conventional procedure. From the 7th day of the experiment, the feeds of both groups were contaminated with 1×10⁸ cfu/g of pathogenic Escherichia coli for 2 days and thereafter provided twice a day respectively for the experimental and the control groups so as to bring about the infections of pathogenic Escherichia coli. The administered pathogenic Escherichia coli suspension was prepared as follows: Pathogenic Escherichia coli (strain CCARM 1G936) was cultured at 37° C. for 18 hours using a TSB culture medium, after which the bacteria were isolated and adjusted to 109 CFU/ml using physiological saline (pH 7.2). From the next day after providing contaminated feeds for 2 days (the 9th day of the experiment), pigs of the experimental group (adding the bacteriophage) were fed again with the feeds adding the bacteriophage Esc-COP-23 at 1×10⁸ pfu/g without contaminating pathogenic Escherichia coli according to the conventional feed supply procedure as before, while pigs of the control group (without adding the bacteriophage) were fed with the same feed without adding the bacteriophage according to the conventional procedure. From the 9th day of the experiment, diarrhea was examined in all test animals on a daily basis. The extent of diarrhea was determined by measuring according to a diarrhea index. The diarrhea index was measured using a commonly used Fecal Consistency (FC) score (normal: 0, soft stool: 1, loose diarrhea: 2, severe diarrhea: 3). The results are shown in Table 3.

TABLE 3
Fecal Consistency score

| | Fecal Consistency score | | | | | |
|---|---|---|---|---|---|---|
| | D 9 | D 10 | D 11 | D 12 | D 13 | D 14 |
| Control group (bacteriophage | 2.25 | 2.25 | 1.5 | 1.5 | 1.25 | 1.0 |
| suspension was not | | | | | | |
| administered) | | | | | | |
| Experimental group | 1.0 | 0.75 | 0.75 | 0.5 | 0 | 0 |
| (bacteriophage suspension | | | | | | |
| was administered) | | | | | | |

From the above results, it is confirmed that the bacteriophage Esc-COP-23 of the present invention could be very effective to suppress the infections of pathogenic Escherichia coli.

### Example 8: Example of Treatment of Infectious Diseases of Pathogenic Escherichia coli Using Bacteriophage Esc-COP-23

The therapeutic effect of the bacteriophage Esc-COP-23 on diseases caused by pathogenic Escherichia coli was evaluated as follows: 40 of 8-week-old mice were divided into a total of 2 groups of 20 mice per group, after which subgroups of 5 mice each were separately reared in individual experimental mouse cages, and the experiment was performed for 7 days. On the second day of the experiment, 0.1 ml of a pathogenic Escherichia coli suspension was administered to all mice through intraperitoneal injection. The administered pathogenic Escherichia coli suspension was prepared as follows: Pathogenic Escherichia coli (strain CCARM 1G936) was cultured at 37° C. for 18 hours using a TSB culture medium, after which the bacteria were isolated and adjusted to 109 CFU/ml using physiological saline (pH 7.2). At 2 hr after administration of pathogenic Escherichia coli, 109 pfu of bacteriophage Esc-COP-23 was administered through intraperitoneal injection to mice in the experimental group (administered with the bacteriophage suspension). 0.1 ml of saline was administered through intraperitoneal injection to mice in the control group (not administered with the bacteriophage suspension). Both the control and experimental groups were equally fed with feed and drinking water. Whether or not the mice survived was observed daily starting from the administration of pathogenic Escherichia coli until the end of the test. The results are shown in Table 4 below.

TABLE 4
Survival rate

| | Survival rate (%) | | | | | |
|---|---|---|---|---|---|---|
| | D 2 | D 3 | D 4 | D 5 | D 6 | D 7 |
| Control group (not | 100 | 80 | 50 | 45 | 25 | 15 |
| administered with | | | | | | |
| bacteriophage suspension) | | | | | | |
| Experimental group | 100 | 85 | 85 | 80 | 80 | 75 |
| (administered with | | | | | | |
| bacteriophage suspension | | | | | | |
| through intraperitoneal | | | | | | |
| injection) | | | | | | |

As is apparent from the above results, it can be concluded that the bacteriophage Esc-COP-23 of the present invention is very effective in the treatment of diseases caused by pathogenic Escherichia coli.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

### Accession Number

Name of Depositary Authority: Korean Collection for Type Cultures (KCTC)

Accession number: KCTC 14030BP

Accession date: 20191115

What is claimed is:

1. A method for treating an infection or disease caused by a pathogenic Escherichia coli, comprising:
isolating a Myoviridae bacteriophage Esc-COP-23 from environmental or clinical samples;
purifying the Myoviridae bacteriophage Esc-COP-23, the purifying comprising culturing the Myoviridae bacteriophage Esc-COP-23 with Escherichia coli, centrifuging to obtain a supernatant, filtering the supernatant, conducting a polyethylene glycol precipitation process to a bacteriophage precipitate, and suspending the bacteriophage precipitate in a buffer;
mixing the Myoviridae bacteriophage Esc-COP-23 with a pharmaceutically acceptable carrier to obtain a composition for treating the infection or disease caused by the pathogenic Escherichia coli; and
administering to a subject with the infection or disease caused by the pathogenic Escherichia coli the composition;
wherein the Myoviridae bacteriophage Esc-COP-23 has an ability to lyse the pathogenic Escherichia coli and a genome represented by a sequence as set forth in SEQ ID NO: 1;
wherein the Myoviridae bacteriophage Esc-COP-23 has a latent period of 10-15 minutes and a burst size of 940-965 plaque-forming units (PFU)/infected cell and is deposited in the Korean Collection for Type Cultures (KCTC) under accession number KCTC 14030BP;
wherein the Myoviridae bacteriophage Esc-COP-23 has major structural proteins in the sizes of approximately 50 kDa, 69 kDa, 128 kDa, and 150 kDa; and
wherein the Myoviridae bacteriophage Esc-COP-23 has a concentration of 1×10⁴ pfu/ml to 1×10¹⁵ pfu/ml or 1×10⁴ pfu/g to 1×10¹⁵ pfu/g.

2. The method of claim 1, wherein the pharmaceutically acceptable carrier is lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, or mineral oil.

3. The method of claim 1, wherein the pathogenic Escherichia coli is enterohemorrhagic Escherichia coli, enterotoxigenic Escherichia coli, enteroinvasive Escherichia coli, enteropathogenic Escherichia coli, enteroaggregative Escherichia coli, or carcinogenic Escherichia coli.

4. The method of claim 1, wherein the infection or disease is food poisoning, gastroenteritis, diarrhea, urinary tract infections, neonatal meningitis, hemolytic-uremic syndrome, peritonitis, mastitis, septicemia, Gram-negative pneumonia, shigellosis, dysentery, or cancer.

5. A method for treating an infection or disease caused by a pathogenic Escherichia coli, comprising:
administering to a subject a Myoviridae bacteriophage Esc-COP-23; and
lysing the pathogenic Escherichia coli by the Myoviridae bacteriophage Esc-COP-23,
wherein the Myoviridae bacteriophage Esc-COP-23 has a genome represented by a sequence as set forth in SEQ ID NO: 1;
wherein the Myoviridae bacteriophage Esc-COP-23 has a latent period of 10-15 minutes and a burst size of 940-965 plaque-forming units (PFU)/infected cell and is deposited in the Korean Collection for Type Cultures (KCTC) under accession number KCTC 14030BP;
wherein the Myoviridae bacteriophage Esc-COP-23 has major structural proteins in the sizes of approximately 50 kDa, 69 kDa, 128 kDa, and 150 kDa; and
wherein the Myoviridae bacteriophage Esc-COP-23 has a concentration of 1×10⁴ pfu/ml to 1×10¹⁵ pfu/ml or 1×10⁴ pfu/g to 1×10¹⁵ pfu/g.
